(19) United States
(12) Patent Application Publication
(10) Pub. No.: US 2021/0040089 A1
(43) Pub. Date: Feb. 11, 2021

(54) KRAS MUTANT PROTEIN INHIBITORS

(71) Applicant: Jacobio Pharmaceuticals Co., Ltd., Beijing (CN)

(72) Inventors: Panliang GAO, Beijing (CN); Cunbo MA, Beijing (CN); Peng WANG, Beijing (CN); Dan LIU, Beijing (CN)

(73) Assignee: Jacobio Pharmaceuticals Co., Ltd.

(21) Appl. No.: 16/986,336

(22) Filed: Aug. 6, 2020

(30) Foreign Application Priority Data
Aug. 7, 2019 (CN) PCT/CN2019/099561
Sep. 30, 2019 (CN) PCT/CN2019/109379
Jan. 20, 2020 (CN) PCT/CN2020/073238

(51) Int. Cl. C07D 471/04; C07D 519/00

(52) U.S. Cl. CPC C07D 471/04; C07D 519/00

(57) ABSTRACT

The invention relates to a KRAS mutant protein inhibitor, as shown by formula (I), a composition containing the inhibitor and the use thereof.

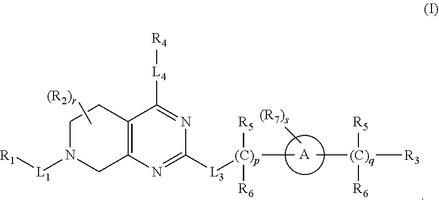

### TECHNICAL FIELD

The invention relates to a KRAS mutant protein inhibitor, as shown by formula (I), a composition containing the inhibitor and the use thereof.

### BACKGROUND ART

RAS represents a population of 189 amino acid monomeric globular proteins (21 kDa molecular weight) that are associated with the plasma membrane and bind to GDP or GTP, and RAS acts as a molecular switch. When the RAS contains bound GDP, it is in a stationary or closed position and is “inactive.” When cells are exposed to certain growth-promoting stimuli, RAS is induced to exchange their bound GDP for GTP. In the case of binding to GTP, RAS is “opened” and is capable of interacting with other proteins (its “downstream targets”) and activating the proteins. The RAS protein itself has an inherently low ability to hydrolyze GTP back to GDP, thereby turning itself into a closed state. Closing RAS requires an exogenous protein called GTPase activating protein (GAP) that interacts with RAS and greatly accelerates the conversion of GTP to GDP. Any mutation in RAS that affects its ability to interact with GAP or convert GTP back to GDP will result in prolonged protein activation, and thus conduction to the cell to inform its signaling of continued growth and division. Since these signals cause cell growth and division, over-activated RAS signaling can ultimately lead to cancer.

Structurally, the RAS protein contains a G domain responsible for the enzymatic activity of RAS-guanine nucleotide binding and hydrolysis (GTPase reaction). It also contains a C-terminal extension called the CAAX cassette, which can be post-translationally modified and responsible for targeting the protein to the membrane. The G domain is approximately 21-25 kDa in size and contains a phosphate binding ring (P-ring). The P-loop represents a pocket of a binding nucleotide in a protein, and this is a rigid portion of a domain with conserved amino acid residues necessary for nucleotide binding and hydrolysis (glycine 12, threonine 26 and lysine 16). The G domain also contains a so-called switch I region (residues 30-40) and a switch II region (residues 60-76), both of which are dynamic parts of the protein, since the dynamic portion is converted between stationary and loaded states. The ability is often expressed as a “spring loaded” mechanism. The primary interaction is the hydrogen bond formed by threonine-35 and glycine-60 with the gamma-phosphate of GTP, which maintains the active conformation of the switch 1 region and the switch 2 region, respectively. After hydrolysis of GTP and release of phosphate, the two relax into an inactive GDP conformation.

The most notable members of the RAS subfamily are HRAS, KRAS and NRAS, which are primarily involved in many types of cancer. Mutation of any of the three major isoforms of the RAS gene (HRAS, NRAS or KRAS) is one of the most common events in human tumor formation. Approximately 30% of all tumors in human tumors were found to carry some mutations in the RAS gene. It is worth noting that KRAS mutations were detected in 25%-30% of tumors. In contrast, the rate of carcinogenic mutations in NRAS and HRAS family members was much lower (8% and 3%, respectively). The most common KRAS mutations were found at residues G12 and G13 in the P-loop as well as at residue Q61.

G12C is a frequently occurring KRAS gene mutation (glycine-12 is mutated to cysteine). This mutation has been found in about 13% of cancers, about 43% in lung cancer, and almost 100% in MYH-associated polyposis (familial colon cancer syndrome). However, targeting this gene with small molecules is a challenge.

Thus, despite advances in this field, there remains a need in the art for improved compounds and methods for treating cancer, such as by inhibiting KRAS, HRAS or NRAS. The present invention fulfills this need and provides other related advantages.

### SUMMARY OF INVENTION

In one aspect, there is provided a compound of formula (I), a pharmaceutical acceptable salt thereof or stereoisomer thereof:

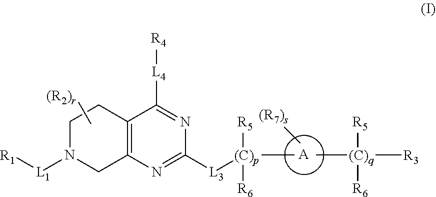

Wherein,

Each of L₁ at each occurrence is independently selected from absent, (CR₅R₆)ₘ, C(═O), O, NR₈, S, S(═O) or S(═O)₂;

Each of R₁ at each occurrence is independently selected from hydrogen, —C₁₋₆alkyl, —C₂₋₆alkenyl, —C₂₋₆alkynyl, —C₆₋₁₀aryl, —C₁₋₆alkylene-C₆₋₁₀aryl, 5-10 membered heteroaryl, —C₁₋₆alkylene-(5-10 membered heteroaryl), 3-10 membered heterocyclic, —C₁₋₆alkylene-(3-10 membered heterocyclic), —C₃₋₁₀carbocyclic or —C₁₋₆alkylene-C₃₋₁₀carbocyclic, each of heterocyclic and heteroaryl at each occurrence independently contains 1, 2, 3 or 4 heteroatoms selected from N, O, S, S═O or S(═O)₂; each of which at each occurrence is independently optionally substituted by 1, 2, 3, 4, 5 or 6 R₁₁ or 1, 2, 3, 4, 5 or 6 R₁₂;

Each of R₁₂ at each occurrence is independently selected from —C₆₋₁₀aryl, —C₁₋₆alkylene-C₆₋₁₀aryl, 5-10 membered heteroaryl, —C₁₋₆alkylene-(5-10 membered heteroaryl), 3-10 membered heterocyclic, —C₁₋₆alkylene-(3-10 membered heterocyclic), —C₃₋₁₀carbocyclic or —C₁₋₆alkylene-C₃₋₁₀carbocyclic; each of which is independently optionally substituted by 1, 2, 3, 4, 5 or 6 R₁₂;

Each of R₁₂ at each occurrence is independently selected from halogen, oxo, —C₁₋₆alkyl, —C₁₋₆alkylene-(halo)₁₋₃, heteroC₁₋₆alkyl, —CN, —OR₈, —C₁₋₆alkylene-(OR₈)₁₋₃, —O—C₁₋₆alkylene-(halo)₁₋₃, —SR₈, —S—C₁₋₆alkylene-(halo)₁₋₃, —NR₈R₉, —C₁₋₆alkylene-NR₈R₉, —C(═O)R₈, —C(═O)OR₈, —OC(═O)R₈, —C(═O)NR₈R₉, —NR₈C(═O)R₈, —S(O)₂NR₈R₉ or —C₃₋₆carbocyclic; each of which is independently optionally substituted by 1, 2, 3, 4, 5 or 6 substituents selected from halogen, —C₁₋₆alkyl, —C₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of R₁₂ at each occurrence is independently selected from halogen, oxo, —C₁₋₆alkyl, —C₁₋₆alkylene-(halo)₁₋₃, heteroC₁₋₆alkyl, —CN, —OR₈, —C₁₋₆alkylene-(OR₈)₁₋₃, —NR₈R₉, —C₁₋₆alkylene-NR₈R₉, —C(═O)R₈, —C(═O)OR₈, —OC(═O)R₈, —C(═O)NR₈R₉, —NR₈C(═O)R₈, —S(O)₂NR₈R₉, or —C₃₋₆carbocyclic; each of which is independently optionally substituted by 1, 2, 3, 4, 5 or 6 substituents selected from halogen, —C₁₋₆alkyl, —C₁₋₆ alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of L₃ at each occurrence is independently selected from absent, (CR₅R₆)ₘ, C(═O), O, NR₈, S, S(═O) or S(═O)₂;

Each of ring A is a C₃₋₁₀ carbocyclic ring, the

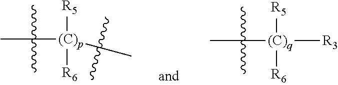

may attach to the same carbon atom or different atom of the ring A;

Each of R₃ is —OR₈, —NR₈R₉, —SR₈, —S(═O)R₈, —S(═O)₂R₈, 5-10 membered heteroaryl or 3-10 membered heterocyclic, each of heterocyclic and heteroaryl at each occurrence independently contains 1, 2, 3 or 4 heteroatoms selected from N, O, S, S═O or S(═O)₂, each of which at each occurrence is independently optionally substituted by 1, 2, 3, 4, 5 or 6 R₁₀;

Each of L₄ at each occurrence is independently selected from absent, (CR₅R₆)ₘ, C(═O), O, NR₈, S, S(═O) or S(═O)₂;

Each of R₄ at each occurrence is independently selected from

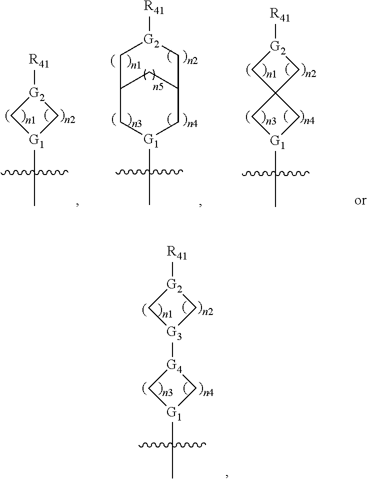

each of which at each occurrence is independently optionally substituted by 1, 2, 3, 4, 5 or 6 R₄₂;

Each of G₁, G₂, G₃ and G₄ at each occurrence is independently selected from N or CR₅;

Each of n1, n2, n3, n4, n5 at each occurrence is independently selected from 0, 1, 2, 3, 4, 5 or 6, provided that n1 and n2 is not 0 at the same time, n3 and n4 is not 0 at the same time;

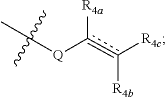

Each of R₄₁ at each occurrence is independently selected from R₄

Each of Q at each occurrence is independently selected from C(═O), NR₈C(═O), S(═O)₂ or NR₈S(═O)₂; [symbol] is selected from ═ or [symbol] ;

Each of R₄ₐ, R_{4b} and R_{4c} at each occurrence is independently selected from hydrogen, halogen, oxo, —C₁₋₆ alkyl, —C₁₋₆alkylene-(halo)₁₋₃, heteroC₁₋₆alkyl, —CN, —OR₈, —C₁₋₆alkylene-(OR₈)₁₋₃, —NR₈R₉, —C₁₋₆alkylene-NR₈R₉, —C(═O)R₈, —C(═O)OR₈, —OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —S(O)₂NR₈R₉ or —C₃₋₁₀carbocyclic; each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉; or R_{4b} and R_{4c} with the carbon which they both attach to form a ring selected from C₃₋₁₀carbocyclic or 3-10 membered heterocyclic, each of heterocyclic at each occurrence contains 1, 2 or 3 heteroatoms selected from N, O, S, SO or S(O)₂ and each of carbocyclic or heterocyclic may be substituted by 1, 2, 3, 4, 5 or 6 substituents selected from halogen, —C₁₋₆ alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉ when [symbol] is selected from ═; or

Each of R₄ₐ is absent and one of R_{4b} and R_{4c} is absent, another of R_{4b} and R_{4c} is selected from hydrogen, halogen, oxo, —C₁₋₆alkyl, —C₁₋₆alkylene-(halo)₁₋₃, heteroC₁₋₆alkyl, —CN, —OR₈, —C₁₋₆alkylene-(OR₈)₁₋₃, —NR₈R₉, —C₁₋₆alkylene-NR₈R₉, —C(═O)R₈, —C(═O)OR₈, —OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —S(O)₂NR₈R₉ or —C₃₋₁₀carbocyclic; each of which is independently optionally substituted by 1, 2, 3, 4, 5 or 6 substituents selected from halogen, —C₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉ when [symbol] is selected from [symbol] ;

Each of R₄₂ is selected from halogen, oxo, —C₁₋₆alkyl, —C₁₋₆alkylene-(halo)₁₋₃, heteroC₁₋₆alkyl, —C₂₋₆alkenyl, —C₂₋₆alkynyl, —OR₈, —C₁₋₆alkylene-(OR₈)₁₋₃, —NR₈R₉, —C₁₋₆alkylene-NR₈R₉, —CN, —C₁₋₆alkylene-CN, —C(═O)R₈, —C(═O)OR₈, —OC(═O)R₈, —C(═O)NR₈R₉, —C₁₋₆alkylene-C(═O)NR₈R₉, —NR₈C(═O)R₈, —C₁₋₆alkylene-NR₈C(═O)R₈ or —S(O)₂NR₈R₉; each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of R₅ and R₆ at each occurrence is independently selected from hydrogen, halogen, —C₁₋₆alkyl, —C₂₋₆alkenyl, —C₂₋₆alkynyl, oxo, —OR₈, —NR₈R₉, —CN, —C(═O)R₈, —C(═O)OR₈, —OC(═O)R₈, —C(═O)NR₈R₉, —NR₈C(═O)R₈, —S(O)₂NR₈R₉ or —C₃₋₁₀carbocyclic, each of heterocyclic and heteroaryl at each occurrence independently contains 1, 2, 3 or 4 heteroatoms selected from N, O, S, S═O or S(═O)₂; each of which at each occurrence is independently optionally substituted by 1, 2, 3, 4, 5 or 6 substituents selected from halogen, oxo, —C₁₋₆alkyl, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of R₇ at each occurrence is independently selected from halogen, —C₁₋₆alkyl, —C₁₋₆alkylene-(halo)₁₋₃, heteroC₁₋₆alkyl, —C₂₋₆alkenyl, —C₂₋₆alkynyl, oxo, —OR₈, —C₁₋₆alkylene-(OR₈)₁₋₃, —O—C₁₋₆alkylene-(halo)₁₋₃, —NR₈R₉, —C₁₋₆alkylene-NR₈R₉, —CN, —C(═O)R₈, —C(═O)OR₈, —OC(═O)R₈, —C(═O)NR₈R₉, —NR₈C(═O)R₈, —S(O)₂NR₈R₉, —C₆₋₁₀aryl, 5-10 membered heteroaryl, 3-10 membered heterocyclic or —C₃₋₁₀carbocyclic, each of heterocyclic and heteroaryl at each occurrence independently contains 1, 2, 3 or 4 heteroatoms selected from N, O, S, S═O or S(═O)₂; each of which at each occurrence is independently optionally substituted by 1, 2, 3, 4, 5 or 6 substituents selected from halogen, oxo, —C₁₋₆alkyl, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of R₈ and R₉ at each occurrence is independently selected from hydrogen or —C₁₋₆alkyl, each of which is independently optionally substituted by 1, 2, 3, 4, 5 or 6 R₁₀; or

R₈ and R₉ together with the N atom which they both attach form a 3-10 membered heterocyclic ring, the 3-10 membered heterocyclic ring may further contain 1, 2, 3 or 4 heteroatoms selected from N, O, S, S(═O) or S(═O)₂, and the 3-10 membered heterocyclic ring is independently optionally substituted by 1, 2, 3, 4, 5 or 6 R₁₀;

Each of R₁₀ at each occurrence is independently selected from halogen, oxo, —C₁₋₆alkyl, —C₁₋₆alkylene-(halo)₁₋₃, heteroC₁₋₆alkyl, —CN, —OH, —OC₁₋₆alkyl, —C₁₋₆alkylene-(OH)₁₋₃, —C₁₋₆alkylene-(OC₁₋₆alkyl)₁₋₃, —NH₂, —NHC₁₋₆alkyl, —N(C₁₋₆alkyl)₂, —C₁₋₆alkylene-NH₂, —C₁₋₆alkylene-NHC₁₋₆alkyl, —C₁₋₆alkylene-N(C₁₋₆alkyl)₂, —C(═O)C₁₋₆alkyl, —C(═O)OC₁₋₆alkyl, —OC(═O)C₁₋₆alkyl, —C(═O)NH₂, —C(═O)NHC₁₋₆alkyl, —C(═O)N(C₁₋₆alkyl)₂, —NHC(═O)C₁₋₆alkyl, —N(C₁₋₆alkyl)C(═O)C₁₋₆alkyl, —S(O)₂NH₂, —S(O)₂NH(CH₃), —S(O)₂NHC₁₋₆alkyl, —S(O)₂N(C₁₋₆alkyl) or —C₃₋₆carbocyclic;

m is selected from 0, 1, 2, 3, 4, 5 or 6;

r is selected from 0, 1, 2, 3, 4, 5 or 6;

s is selected from 0, 1, 2, 3, 4, 5 or 6;

p is selected from 0, 1, 2, 3, 4, 5 or 6;

q is selected from 0, 1, 2, 3, 4, 5 or 6.

In some embodiments, each of L₁ at each occurrence is independently selected from absent or (CR₅R₆)ₘ.

In some embodiments, each of L₁ at each occurrence is independently selected from absent.

In some embodiments, each of R₁ at each occurrence is independently selected from hydrogen, —C₁₋₃alkyl, —C₂₋₃alkenyl, —C₂₋₃alkynyl, —C₆₋₁₀aryl, —C₁₋₃alkylene-C₁₋₆aryl, 5-10 membered heteroaryl, —C₁₋₃alkylene-(5-10 membered heteroaryl), 3-6 membered heterocyclic, —C₁₋₃alkylene-(3-6 membered heterocyclic), —C₃₋₆carbocyclic or —C₁₋₃alkylene-C₃₋₆carbocyclic, each of heterocyclic and heteroaryl at each occurrence independently contains 1, 2, 3 or 4 heteroatoms selected from N, O, or S; each of which at each occurrence is independently optionally substituted by 1, 2, 3, 4, 5 or 6 R₁₁ or 1, 2, 3, 4, 5 or 6 R₁₂.

In some embodiments, each of R₁ at each occurrence is independently selected from hydrogen, methyl, ethyl, propyl, isopropyl, ethenyl, propenyl, isopropenyl, ethynyl, propynyl, isopropynyl, phenyl, naphthyl, -methylene-C₆₋₁₀aryl, -ethylene-C₆₋₁₀aryl, -propylene-C₆₋₁₀aryl, -isopropylene-C₆₋₁₀aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -isopropylene-(5-10 membered heteroaryl), 3 membered heterocyclic, 4 membered heterocyclic, 5 membered heterocyclic, 6 membered heterocyclic, -methylene-(3-6 membered heterocyclic), -ethylene-(3-6 membered heterocyclic), -propylene-(3-6 membered heterocyclic), -isopropylene-(3-6 membered heterocyclic), 3 membered carbocyclic, 4 membered carbocyclic, 5 membered carbocyclic, 6 membered carbocyclic, -methylene-C₃₋₆carbocyclic, -ethylene-C₃₋₆carbocyclic, -propylene-C₃₋₆carbocyclic or -isopropylene-C₃₋₆carbocyclic, each of heterocyclic and heteroaryl at each occurrence independently contains 1, 2, 3 or 4 heteroatoms selected from N, O, or S; each of which at each occurrence is independently optionally substituted by 1, 2, 3, 4, 5 or 6 R₁₁ or 1, 2, 3, 4, 5 or 6 R₁₂.

In some embodiments, each of R₁ at each occurrence is independently selected from phenyl, naphthyl, 5 membered heteroaryl, 6 membered heteroaryl, 7 membered heteroaryl, 8 membered heteroaryl, 9 membered heteroaryl or 10 membered heteroaryl, each of heteroaryl at each occurrence independently contains 1, 2, 3 or 4 heteroatoms selected from N, O, or S; each of which at each occurrence is independently optionally substituted by 1, 2, 3, 4, 5 or 6 R₁₁ or 1, 2, 3, 4, 5 or 6 R₁₂.

In some embodiments, each of R₁ at each occurrence is independently selected from phenyl or naphthyl, each of which at each occurrence is independently optionally substituted by 1, 2, 3, 4, 5 or 6 R₁ or 1, 2, 3, 4, 5 or 6 R₁₂.

In some embodiments, each of R₁ at each occurrence is independently selected from phenyl, naphthyl, pyridyl, indolyl, indazolyl, indolizinyl, benzo[d]thiazole, benzo[d]isothiazole, quinolyl, isoquinolyl or phthalazinyl, each of which at each occurrence is independently optionally substituted by 1, 2, 3, 4, 5 or 6 R or 1, 2, 3, 4, 5 or 6 R₁₂.

In some embodiments, each of R₁₁ at each occurrence is independently selected from —C₆₋₁₀aryl, —C₁₋₃alkylene-C₁₋₆aryl, 5-10 membered heteroaryl, —C₁₋₃alkylene-(5-10 membered heteroaryl), 3-6 membered heterocyclic, —C₁₋₃alkylene-(3-6 membered heterocyclic), —C₃₋₆carbocyclic or —C₁₋₃alkylene-C₃₋₆carbocyclic; each of which is independently optionally substituted by 1, 2, 3, 4, 5 or 6 R₁₂.

In some embodiments, each of R₁ at each occurrence is independently selected from phenyl, naphthyl, -methylene-C₆₋₁₀aryl, -ethylene-C₆₋₁₀aryl, -propylene-C₆₋₁₀aryl, -isopropylene-C₆₋₁₀aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -isopropylene-(5-10 membered heteroaryl), 3 membered heterocyclic, 4 membered heterocyclic, 5 membered heterocyclic, 6 membered heterocyclic, -methylene-(3-6 membered heterocyclic), -ethylene-(3-6 membered heterocyclic), -propylene-(3-6 membered heterocyclic), -isopropylene-(3-6 membered heterocyclic), 3 membered carbocyclic, 4 membered carbocyclic, 5 membered carbocyclic, 6 membered carbocyclic, -methylene-C₃₋₆carbocyclic, -ethylene-C₃₋₆carbocyclic, -propylene-C₃₋₆carbocyclic or -isopropylene-C₃₋₆carbocyclic, each of heterocyclic and heteroaryl at each occurrence independently contains 1, 2, 3 or 4 heteroatoms selected from N, O, or S; each of which at each occurrence is independently optionally substituted by 1, 2, 3, 4, 5 or 6 R₁₂.

In some embodiments, each of R₁₁ at each occurrence is independently selected from phenyl, naphthyl, 5 membered heteroaryl, 6 membered heteroaryl, 7 membered heteroaryl, 8 membered heteroaryl, 9 membered heteroaryl or 10 membered heteroaryl, each of heteroaryl at each occurrence independently contains 1, 2, 3 or 4 heteroatoms selected from N, O, or S; each of which at each occurrence is independently optionally substituted by 1, 2, 3, 4, 5 or 6 R₁₂.

In some embodiments, each of R₁ is selected from:

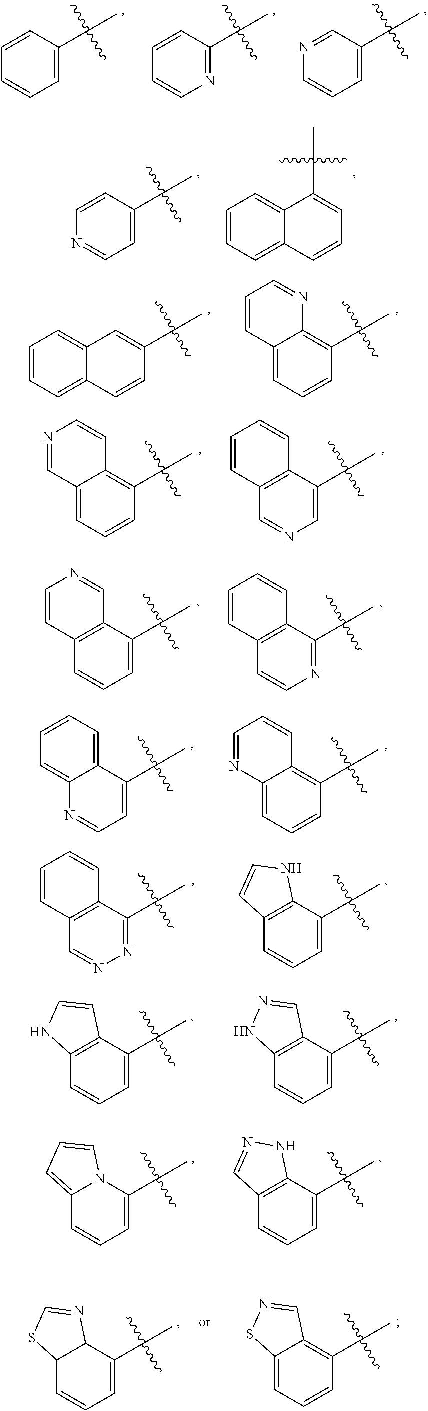

Each of which at each occurrence is independently optionally substituted by 1, 2, 3, 4, 5 or 6 R₁₂.

In some embodiments, each of R₁ is selected from:

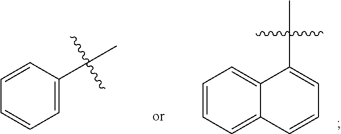 [SMILES: *c1ccccc1 ; *c1cccc2ccccc12]

Each of which at each occurrence is independently optionally substituted by 1, 2, 3, 4, 5 or 6 R₁₂.

In some embodiments, each of R₁₂ at each occurrence is independently selected from halogen, oxo, —C₁₋₃alkyl, —C₁₋₃alkylene-(halo)₁₋₃, heteroC₁₋₃alkyl, —CN, —OR₈, —C₁₋₃alkylene-(OR₈)₁₋₃, —O—C₁₋₃alkylene-(halo)₁₋₃, —SR₈, —S—C₁₋₃alkylene-(halo)₁₋₃, —NR₈R₉, —C₁₋₃alkylene-NR₈R₉, —C(═O)R₈, —C(═O)OR₈, —OC(═O)R₈, —C(═O)NR₈R₉, —NR₈C(═O)R₈, —S(O)₂NR₈R₉ or —C₃₋₆carbocyclic; each of which is independently optionally substituted by 1, 2, 3, 4, 5 or 6 substituents selected from halogen, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉;

Each of (R₈ and R₉) in R₁₂ at each occurrence is independently selected from hydrogen or —C₁₋₃alkyl.

In some embodiments, each of R₁₂ at each occurrence is independently selected from —F, —Cl, —Br, oxo, methyl, ethyl, propyl, isopropyl, -methylene-(halo)₁₋₃, -ethylene-(halo)₁₋₃-propylene-(halo)₁₋₃, heteromethyl, heteroethyl, heteropropyl, —CN, —OR₈, -methylene-(OR₈)₁₋₃, -ethylene-(OR₈)₁₋₃, -propylene-(OR₈)₁₋₃, —O— methylene-(halo)₁₋₃, —O-ethylene-(halo)₁₋₃, —O-propylene-(halo)₁₋₃, —SR₈, —S-methylene-(halo)₁₃, —S-ethylene-(halo)₁₃, —S-propylene-(halo)₁₋₃, —NR₈R₉, -methylene-NR₈R₉, -ethylene-NR₈R₉, -propylene-NR₈R₉, —C(═O)R₈, —C(═O)OR₈, —OC(═O)R₈, —C(═O)NR₈R₉, —NR₈C(═O)R₈, —S(O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉;

Each of (R₈ and R₉) in R₁₂ at each occurrence is independently selected from hydrogen, methyl, ethyl, propyl or isopropyl.

In some embodiments, each of R₁₂ at each occurrence is independently selected from —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OCH₂CH₂F, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SCH₂F, —SCHF₂, —SCF₃, —SCH₂CH₂F, —SCH₂CHF₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each of R₁₂ at each occurrence is independently selected from —F, —Cl, —OH, —NH₂, —CN, methyl, ethyl, propyl, isopropyl, methoxy, ethoxy, propoxy, isopropoxy, —CF₃, —OCF₃, —OCH₂OCH₃, —NH(CH₃), —N(CH₃)₂, —COCH₃, —COCF₃, —OCOCH₃, —OCOCF₃, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OH, —CH₂CH₂OH, —CH₂CH₂CH₂OH, —CH₂OCH₃, —CH₂OCH₃, —CH₂CH₂OCH₃, —CH₂CH₂CH₂OCH₃ or 3 membered carbocyclic.

In some embodiments, each of R₁ is selected from:

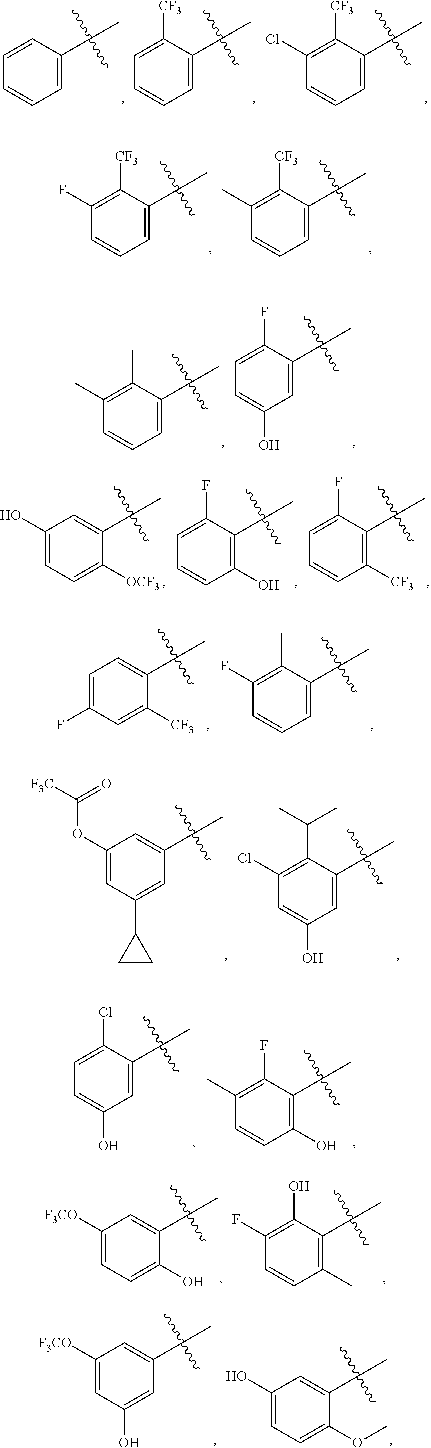

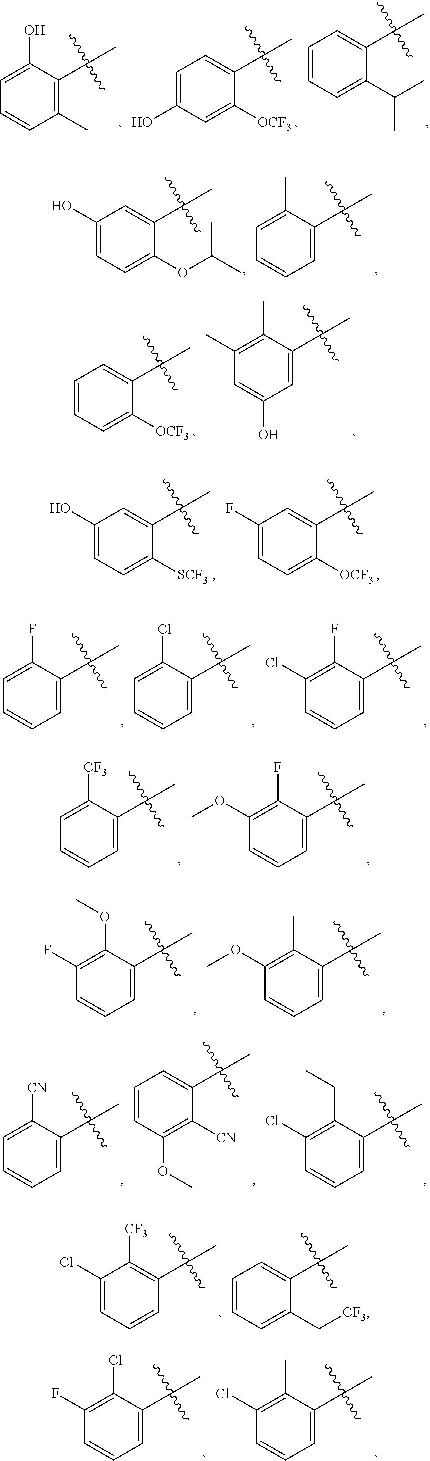

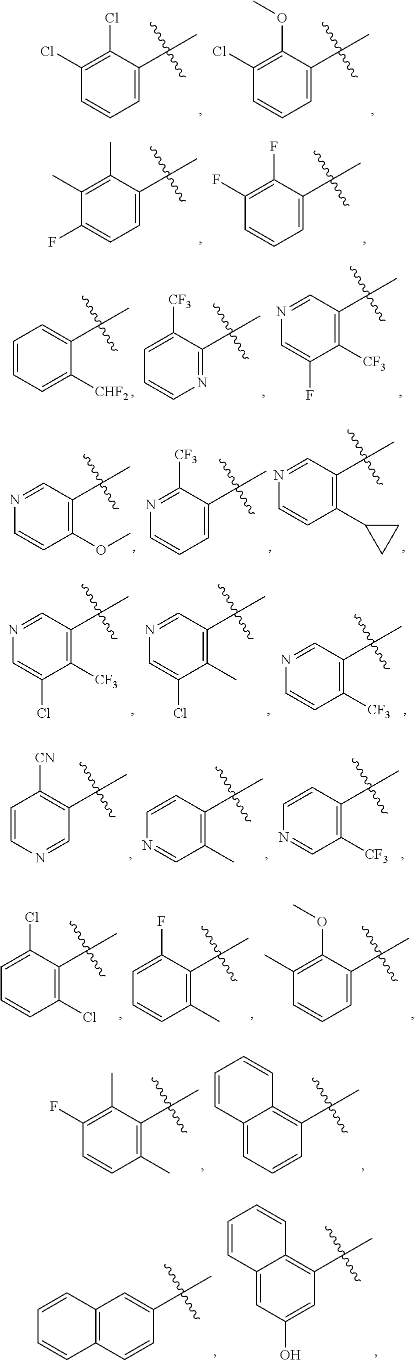

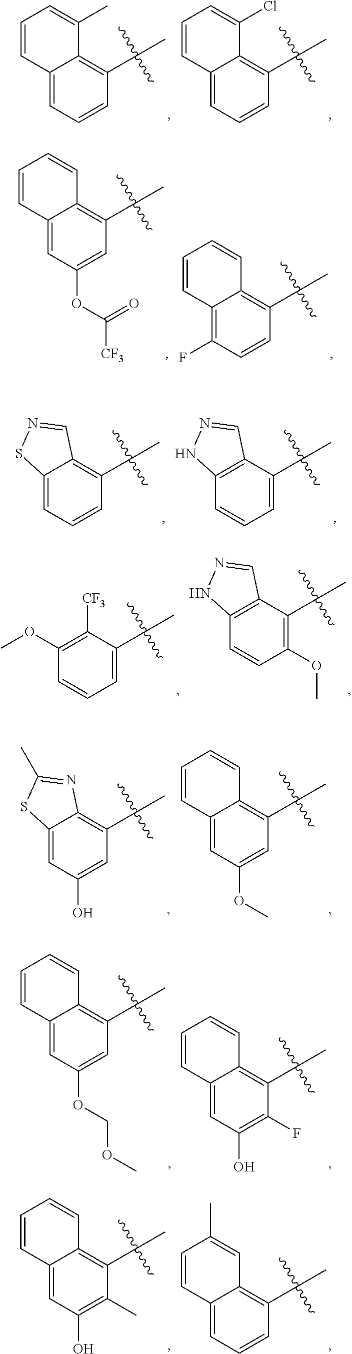

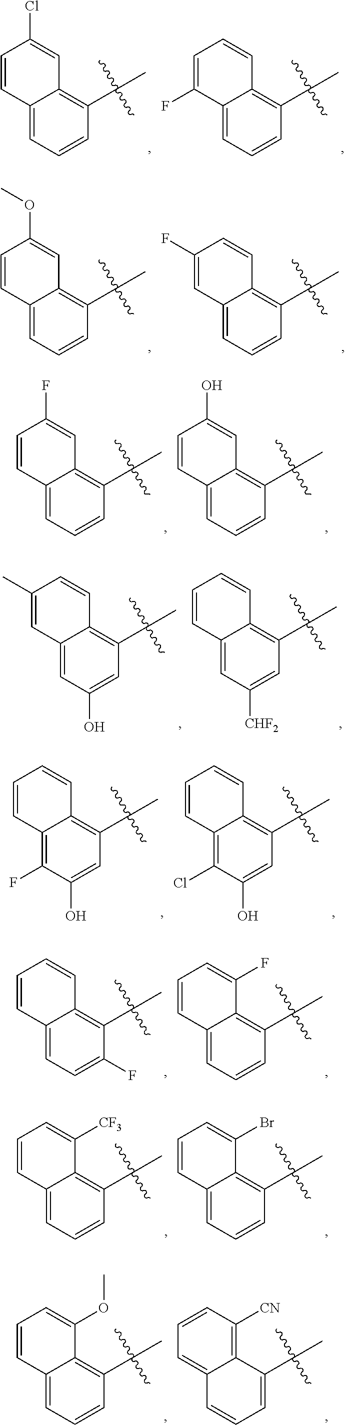

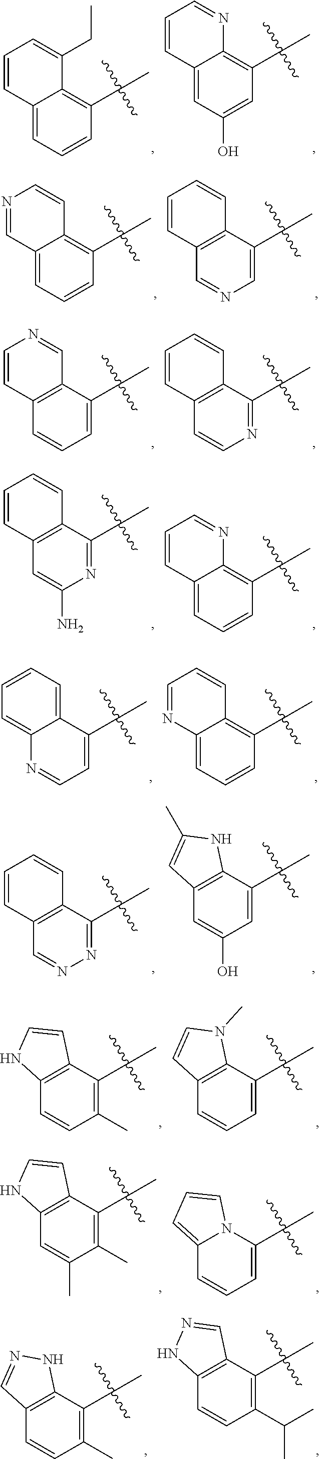

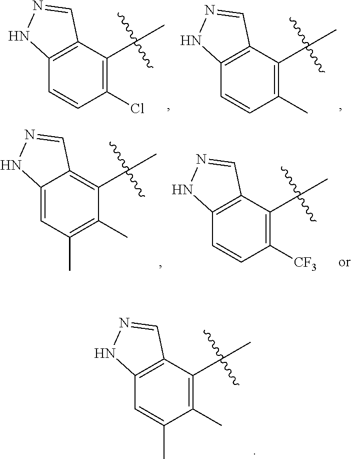

In some embodiments, each of R₁ is selected from:

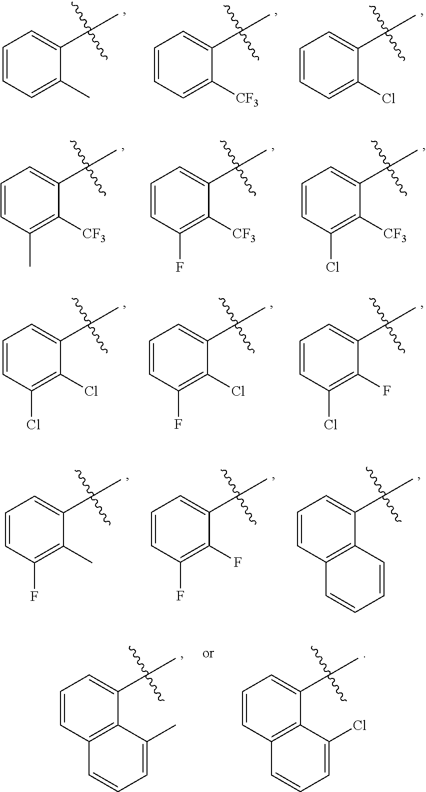

In some embodiments, each of R₁ is selected from:

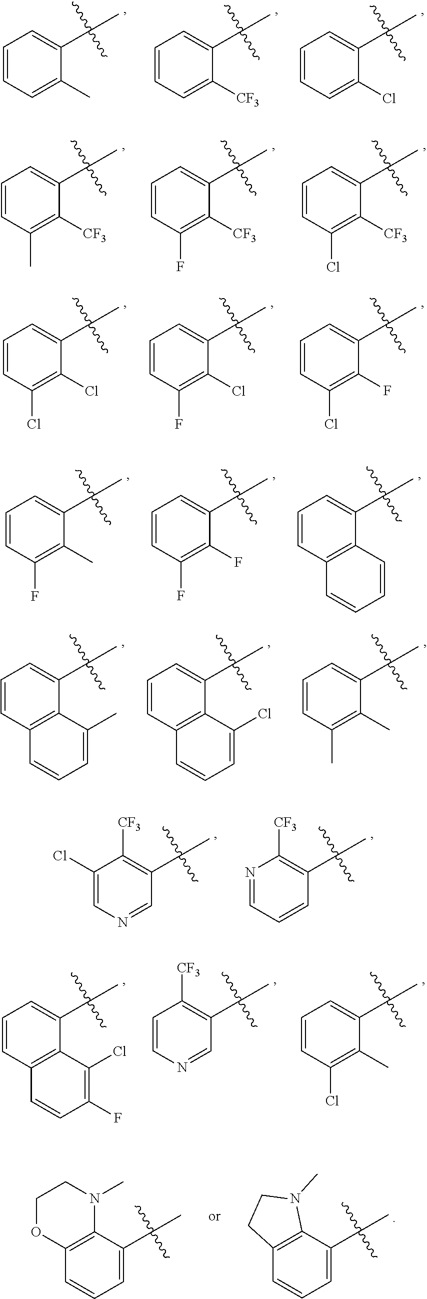

In some embodiments, each of R₂ at each occurrence is independently selected from —F, —Cl, —Br, oxo, —C₁₋₃alkyl, —C₁₋₃alkylene-(halo)₁₋₃, heteroC₁₋₃alkyl, —CN, —OR₈, —C₁₋₃alkylene-(OR₈)₁₋₃, —NR₈R₉, —C₁₋₃alkylene-NR₈R₉, —C(═O)R₈, —C(═O)OR₈, —OC(═O)R₈, —C(═O)NR₈R₉, —NR₈C(═O)R₈, —S(O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of (R₈ and R₉) in R₂ at each occurrence is independently selected from hydrogen or —C₁₋₃alkyl.

In some embodiments, each of R₂ at each occurrence is independently selected from —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, methyl, ethyl, propyl, isopropyl, methoxy, ethoxy, propoxy, isopropoxy, oxo, —OH, —NH₂, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each of R₂ at each occurrence is independently selected from —F, —Cl, oxo, methyl, ethyl, propyl, isopropyl, —CH₂F, —CHF₂, —CF₃, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃ or —NHCH(CH₃)₂.

In some embodiments, r is selected from 0, 1 or 2.

In some embodiments, r is selected from 0.

In some embodiments, each of L₃ at each occurrence is independently selected from 0, NR₈ or S;

Each of R₈ in L₃ at each occurrence is independently selected from hydrogen or —C₁₋₃alkyl.

In some embodiments, each of L₃ at each occurrence is independently selected from O, NH, N(CH₃), N(CH₂CH₃), N(CH₂CH₂CH₃), NCH(CH₃)₂ or S.

In some embodiments, each of L₃ at each occurrence is independently selected from O, NH or S.

In some embodiments, each of ring A is a C₃₋₆ carbocyclic ring, and the

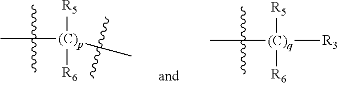

may attach to the same carbon atom or different atom of the ring A.

In some embodiments, each of ring A is a 3 membered carbocyclic ring, 4 membered carbocyclic ring, 5 membered carbocyclic ring or 6 membered carbocyclic ring, and the

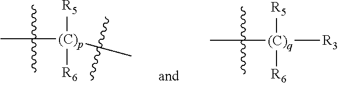

may attach to the same carbon atom or different atom of the ring A.

In some embodiments, each of R₃ at each occurrence is independently selected from —OR₈, —NR₈R₉, —SR₈, —S(═O)R₈, —S(═O)₂R₈ or 3-8 membered heterocyclic, each of heterocyclic at each occurrence independently contains 1, 2, 3 or 4 heteroatoms selected from N, O or S, each of which at each occurrence is independently optionally substituted by 1, 2, 3, 4, 5 or 6 R₁₀;

Each of (R₈ and R₉) in R₃ at each occurrence is independently selected from hydrogen or —C₁₋₃alkyl; or

R₈ and R₉ together with the N atom which they both attach form a 3-8 membered heterocyclic ring, the 3-8 membered heterocyclic ring may further contain 1, 2, 3 or 4 heteroatoms selected from N, O or S, and the 3-8 membered heterocyclic ring is independently optionally substituted by 1, 2, 3, 4, 5 or 6 R₁₀.

In some embodiments, each of R₃ at each occurrence is independently selected from —OR₈, —NR₈R₉, —SR₈, —S(═O)R₈, —S(═O)₂R₈ or 3-6 membered heterocyclic, each of heterocyclic at each occurrence independently contains 1, 2, 3 or 4 heteroatoms selected from N, O or S, each of which at each occurrence is independently optionally substituted by 1, 2, 3, 4, 5 or 6 R₁₀;

Each of (R₈ and R₉) in R₃ at each occurrence is independently selected from hydrogen or —C₁₋₃alkyl; or

R₈ and R₉ together with the N atom which they both attach form a 3-6 membered heterocyclic ring, the 3-6 membered heterocyclic ring may further contain 1, 2, 3 or 4 heteroatoms selected from N, O or S, and the 3-6 membered heterocyclic ring is independently optionally substituted by 1, 2, 3, 4, 5 or 6 R₁₀.

In some embodiments, each of R₃ at each occurrence is independently selected from —NR₈R₉ or 3-6 membered heterocyclic, each of heterocyclic at each occurrence independently contains 1 heteroatom selected from N, each of which at each occurrence is independently optionally substituted by 1, 2, 3, 4, 5 or 6 R₁₀;

Each of (R₈ and R₉) in R₃ at each occurrence is independently selected from hydrogen, methyl, ethyl, propyl or isopropyl; or

(R₈ and R₉) in R₃ together with the N atom which they both attach form a 3-6 membered heterocyclic ring, the 3-6 membered heterocyclic ring may further contain 1 heteroatom selected from N, and the 3-6 membered heterocyclic ring is independently optionally substituted by 1, 2, 3, 4, 5 or 6 R₁₀.

In some embodiments, each of R₃ at each occurrence is independently selected from —NH₂, —N(CH₃)₂, —N(CH₃)(CH₂CH₃), —N(CH₂CH₃)₂,

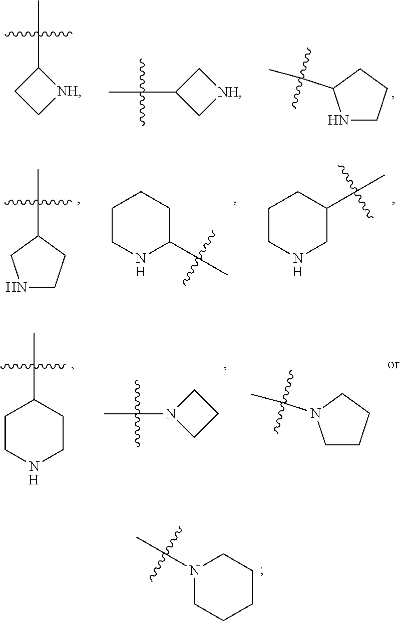

each of which is independently optionally substituted by 1, 2, 3, 4, 5 or 6 R₁₀.

In some embodiments, each of R₃ at each occurrence is independently selected from —NH₂, —N(CH₃)₂, —N(CH₃)(CH₂CH₃), —N(CH₂CH₃)₂,

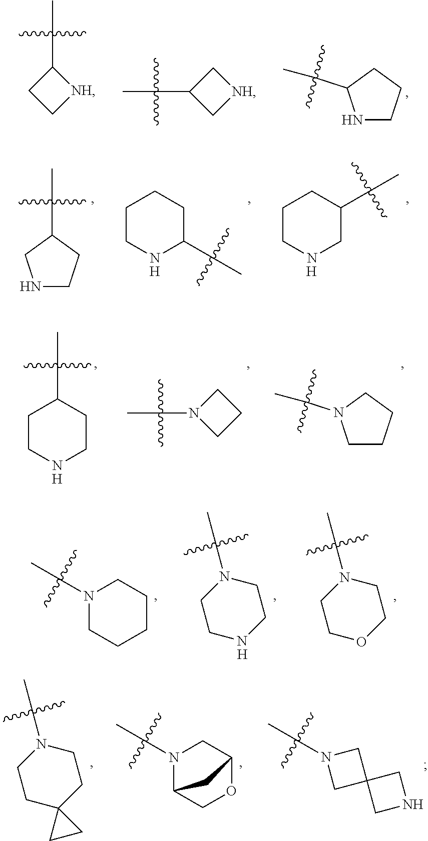

each of which is independently optionally substituted by 1, 2, 3, 4, 5 or 6 R₁₀.

In some embodiments, each of R₁₀ at each occurrence is independently selected from —F, —Cl, —Br, oxo, —C₁₋₃alkyl, —C₁₋₃alkylene-(halo)₁₋₃, heteroC₁₋₃alkyl, —CN, —OH, —OC₁₋₃alkyl, —C₁₋₃alkylene-(OH)₁₋₃, —C₁₋₃alkylene-(OC₁₋₃alkyl)₁₋₃, —NH₂, —NHC₁₋₃alkyl, —N(C₁₋₃alkyl)₂, —C₁₋₃alkylene-NH₂, —C₁₋₃alkylene-NHC₁₋₃alkyl, —C₁₋₃alkylene-N(C₁₋₃alkyl)₂, —C(═O)C₁₋₃alkyl, —C(═O)OC₁₋₃alkyl, —OC(═O)C₁₋₃alkyl, —C(═O)NH₂, —C(═O)NHC₁₋₃alkyl, —C(═O)N(C₁₋₃alkyl)₂, —NHC(═O)C₁₋₃alkyl, —N(C₁₋₃alkyl)C(═O)C₁₋₃alkyl, —S(O)₂NH₂, —S(O)₂NH(CH₃), —S(O)₂NHC₁₋₃alkyl, —S(O)₂N(C₁₋₃alkyl) or 3 membered carbocyclic, 4 membered carbocyclic, 5 membered carbocyclic or 6 membered carbocyclic.

In some embodiments, each of R₁₀ at each occurrence is independently selected from —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CH₂CF₃, —CH₂CH₃, —CH₂CH₂CH₃, —CH₂CH₂CH₂CH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic.

In some embodiments, each of R₁₀ at each occurrence is independently selected from —F, —CH₃, —CH(CH₃)₂, —OH or —OCH₃.

In some embodiments, each of R₃ is selected from:

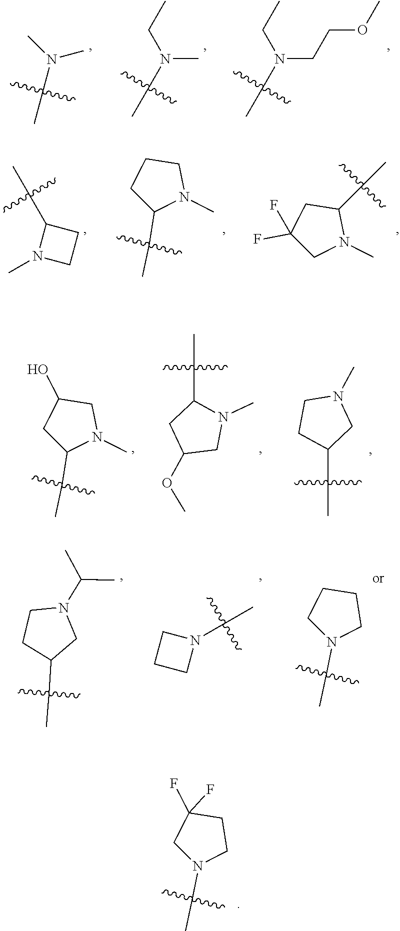

In some embodiments, each of R₃ is selected from:

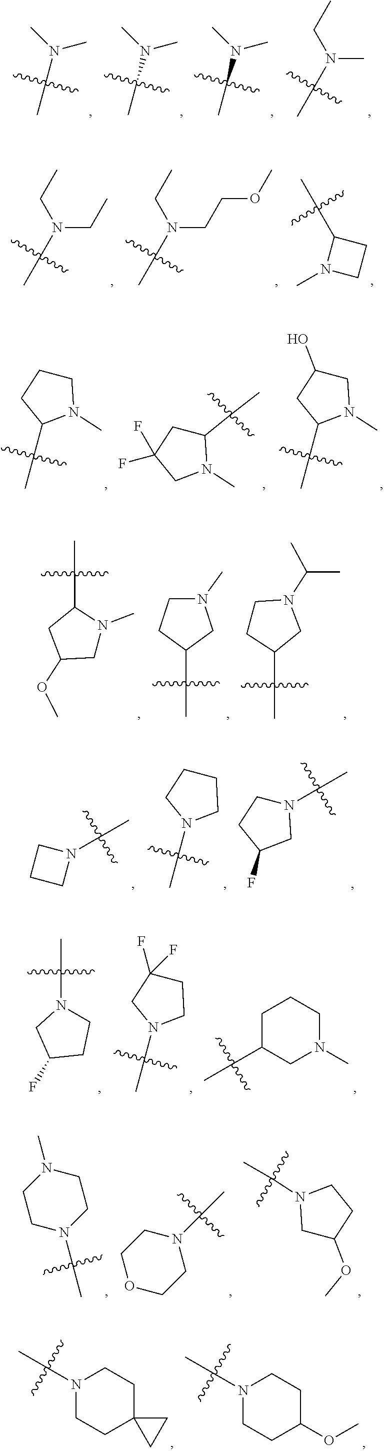

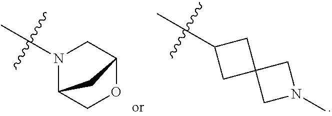

In some embodiments, each of R₅ and R₆ at each occurrence is independently selected from hydrogen, —F, —Cl, —Br, —C₁₋₃alkyl, —C₂₋₃alkenyl, —C₂₋₃alkynyl, oxo, —OR₈, —NR₈R₉, —CN, —C(═O)R₈, —C(═O)OR₈, —OC(═O)R₈, —C(═O)NR₈R₉, —NR₈C(═O)R₈, —S(O)₂NR₈R₉ or —C₃₋₆carbocyclic; each of which at each occurrence is independently optionally substituted by 1, 2, 3, 4, 5 or 6 substituents selected from halogen, oxo, —C₁₋₃alkyl, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉;

Each of (R₈ and R₉) in (R₅ or R₆) is independently selected from hydrogen or —C₁₋₃alkyl.

In some embodiments, each of R₅ and R₆ at each occurrence is independently selected from hydrogen, —F, —Cl, —Br, methyl, ethyl, propyl, isopropyl, ethenyl, propenyl, isopropenyl, ethynyl, propynyl, oxo, —OH, —OCH₃, —OCH₂CH₃, —OCH₂CH₂CH₃, —OCH(CH₃)₂, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, oxo, methyl, ethyl, propyl, isopropyl, —OH, —OCH₃, —OCH₂CH₃, —OCH₂CH₂CH₃, —OCH(CH₃)₂, —NH₂, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each of R₅ and R₆ at each occurrence is independently selected from hydrogen, —F or methyl.

In some embodiments, p is selected from 0, 1, 2 or 3.

In some embodiments, p is selected from 0, 1 or 2.

In some embodiments, p is selected from 0 or 1.

In some embodiments, p is selected from 0.

In some embodiments, p is selected from 1.

In some embodiments, q is selected from 0, 1, 2 or 3.

In some embodiments, q is selected from 0, 1 or 2.

In some embodiments, q is selected from 0 or 1.

In some embodiments, q is selected from 0.

In some embodiments, q is selected from 1.

In some embodiments, each of R₇ at each occurrence is independently selected from —F, —Cl, —Br, —C₁₋₃alkyl, —C₁₋₃alkylene-(halo)₁₋₃, heteroC₁₋₃alkyl, —C₂₋₃alkenyl, —C₂₋₃alkynyl, oxo, —OR₈, —C₁₋₃alkylene-(OR₈)₁₋₃, —O—C₁-3alkylene-(halo)₁₃, —NR₈R₉, —C₁₋₃alkylene-NR₈R₉, —CN, —C(═O)R₈, —C(═O)OR₈, —OC(═O)R₈, —C(═O)NR₈R₉, —NR₈C(═O)R₈, —S(O)₂NR₈R₉, —C₆₋₁₀aryl, 5-10 membered heteroaryl, 3-6 membered heterocyclic or —C₃₋₆carbocyclic, each of heterocyclic and heteroaryl at each occurrence independently contains 1, 2, 3 or 4 heteroatoms selected from N, O or S; each of which at each occurrence is independently optionally substituted by 1, 2, 3, 4, 5 or 6 substituents selected from —F, —Cl, —Br, oxo, —C₁₋₃alkyl, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉;

Each of (R₈ and R₉) in R₇ at each occurrence is independently selected from hydrogen or —C₁₋₃alkyl; or

(R₈ and R₉) in R₇ together with the N atom which they both attach form a 3-6 membered heterocyclic ring, the 3-6 membered heterocyclic ring may further contain 1, 2, 3 or 4 heteroatoms selected from N, O or S.

In some embodiments, each of R₇ at each occurrence is independently selected from —F, —Cl, —Br, methyl, ethyl, propyl, isopropyl, -methylene-(halo)₁₋₃, -ethylene-(halo)₁₋₃, -propylene-(halo)₁₋₃, heteromethyl, heteroethyl, heteropropyl, ethenyl, propenyl, ethynyl, propynyl, oxo, —OR₈, -methylene-(OR₈)₁₋₃, -ethylene-(OR₈)₁₋₃, -propylene-(OR₈)₁₋₃, —O-methylene-(halo)₁₋₃, —O-ethylene-(halo)₁₋₃, —O-propylene-(halo)₁₋₃, —NR₈R₉, -methylene-NR₈R₉, -ethylene-NR₈R₉, -propylene-NR₈R₉, —CN, —C(═O)R₈, —C(═O)OR₈, —OC(═O)R₈, —C(═O)NR₈R₉, —NR₈C(═O)R₈, —S(O)₂NR₈R₉, phenyl, naphthyl, 5 membered heteroaryl, 6 membered heteroaryl, 7 membered heteroaryl, 8 membered heteroaryl, 9 membered heteroaryl, 10 membered heteroaryl, 3 membered heterocyclic, 4 membered heterocyclic, 5 membered heterocyclic, 6 membered heterocyclic, 3 membered carbocyclic, 4 membered carbocyclic, 5 membered carbocyclic or 6 membered carbocyclic, each of heterocyclic and heteroaryl at each occurrence independently contains 1, 2, 3 or 4 heteroatoms selected from N, O or S; each of which at each occurrence is independently optionally substituted by 1, 2, 3, 4, 5 or 6 substituents selected from —F, —Cl, —Br, oxo, methyl, ethyl, propyl, isopropyl, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of (R₈ and R₉) in R₇ at each occurrence is independently selected from hydrogen, methyl, ethyl, propyl or isopropyl; or

(R₈ and R₉) in R₇ together with the N atom which they both attach form

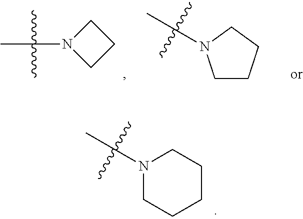 [SMILES: *N1CCC1 ; *N1CCCC1 ; *N1CCCCC1]

In some embodiments, each of R₇ at each occurrence is independently selected from —F, —Cl, —Br, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂CH₃, ethenyl, propenyl, ethynyl, propynyl, oxo, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OCH₂CH₂F, —OCH₂CHF₂, —OCH₂CF₃, —OCH₂CH₂CH₂F, —OCH₂CH₂CHF₂, —OCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —CN, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃) —C(═O)N(CH₃)₂,

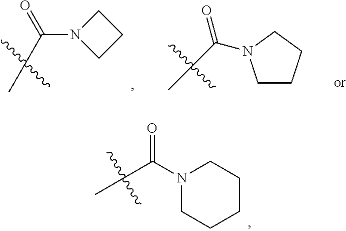 [SMILES: *C(=O)N1CCC1 ; *C(=O)N1CCCC1 ; *C(=O)N1CCCCC1]

—NHC(═O)CH₃, —N(CH₃)C(═O)CH₃, —S(O)₂NH₂, —S(O)₂NH(CH₃), —S(O)₂N(CH₃)₂, phenyl, naphthyl, 5 membered heteroaryl, 6 membered heteroaryl, 7 membered heteroaryl, 8 membered heteroaryl, 9 membered heteroaryl, 10 membered heteroaryl, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, oxo, methyl, ethyl, propyl, isopropyl, methoxy, ethoxy, propoxy, isopropoxy, —OH, —NH₂, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each of R₇ at each occurrence is independently selected from —F, —CH₃, —CH₂CH₂OCH₃, —CF₃₁—OCF₃, —CN,

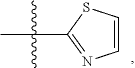 [SMILES: *c1nccs1]

—C(═O)OCH₂CH₃, —C(═O)N(CH₃)₂,

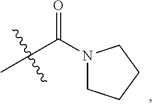 [SMILES: *C(=O)N1CCCC1]

—C(O)NHCH₃, —NHC(═O)CH₃ or —S(═O)₂N(CH₃)₂.

In some embodiments,

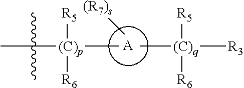

is selected from:

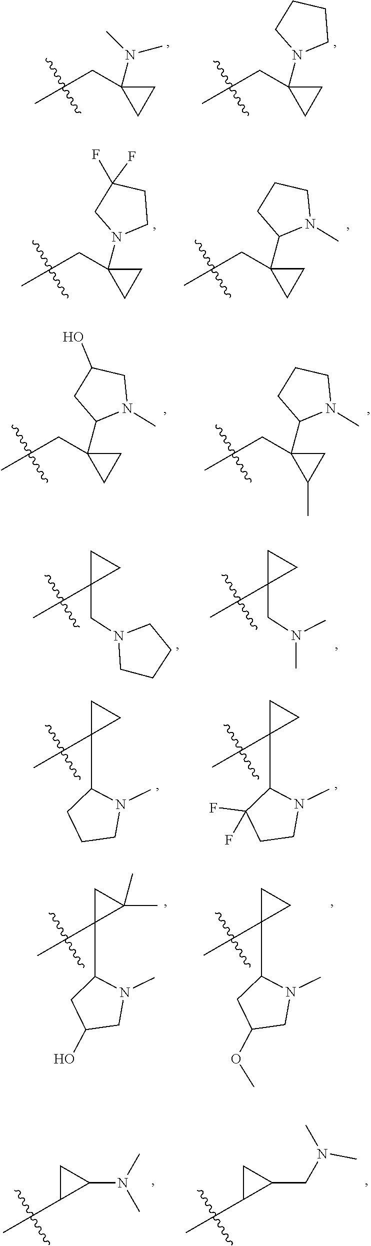

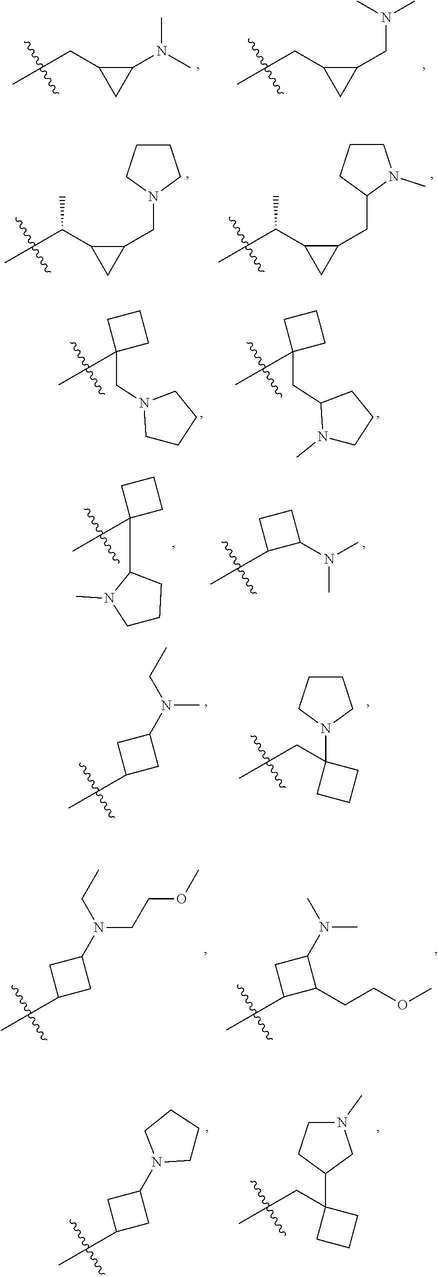

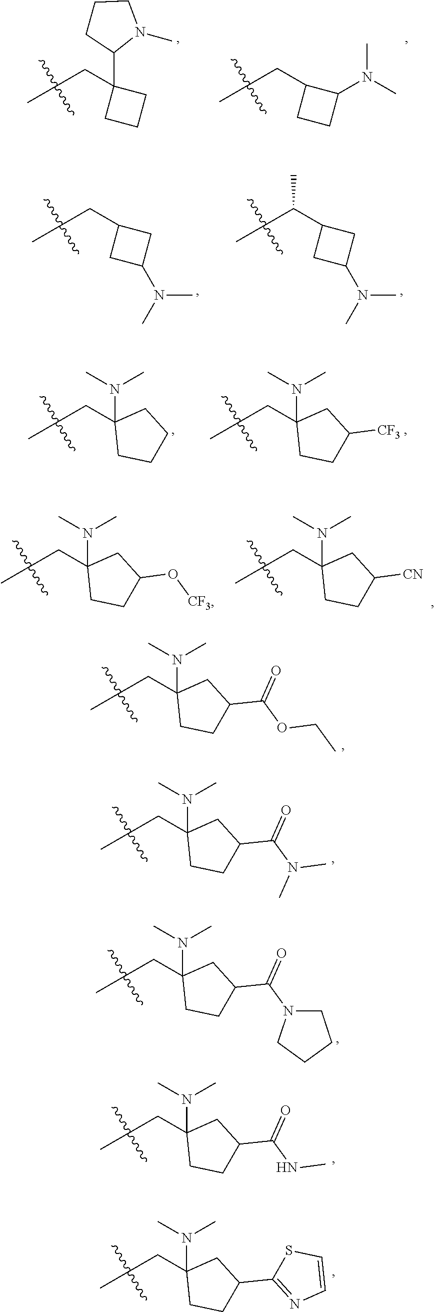

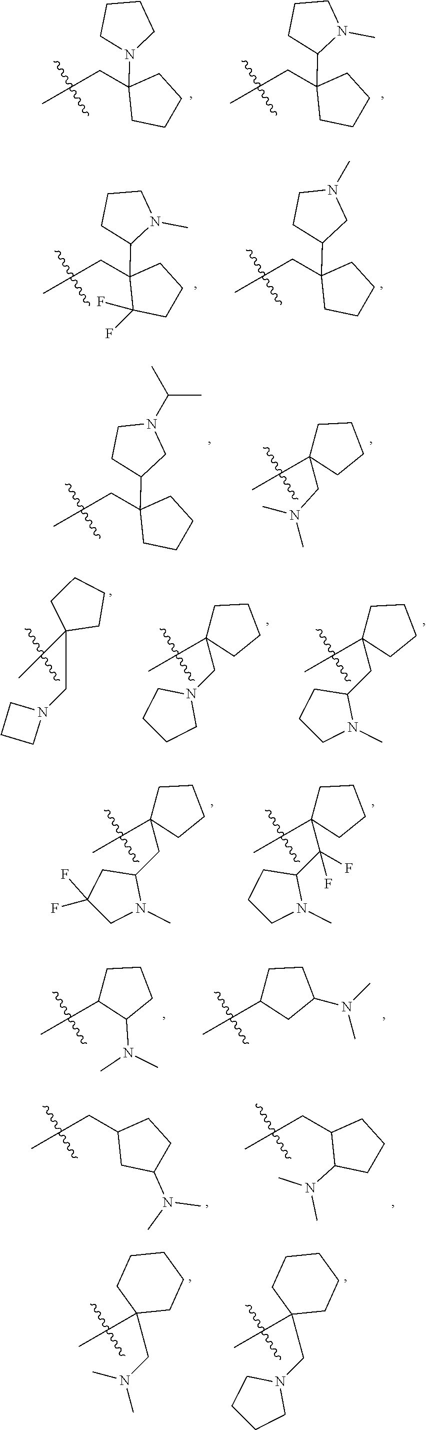

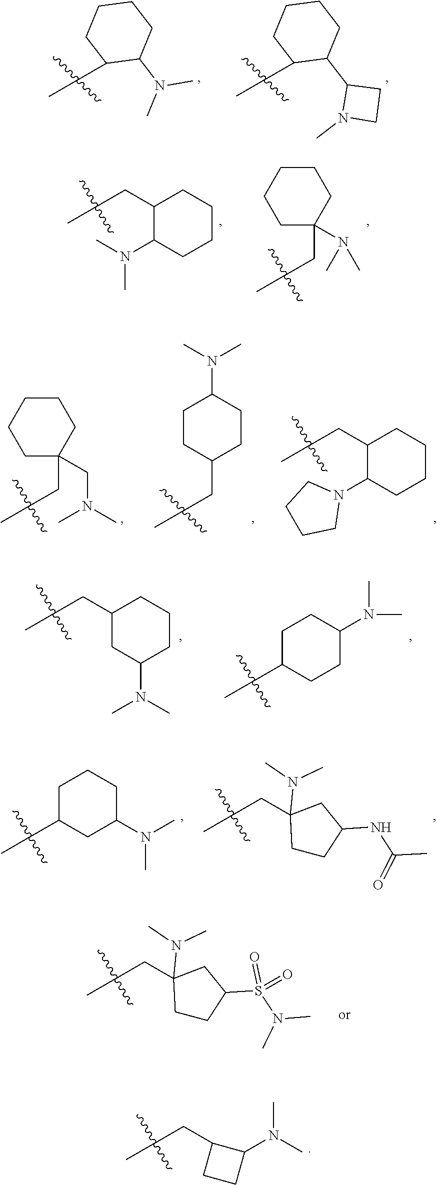

In some embodiments,

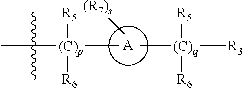

is selected from:

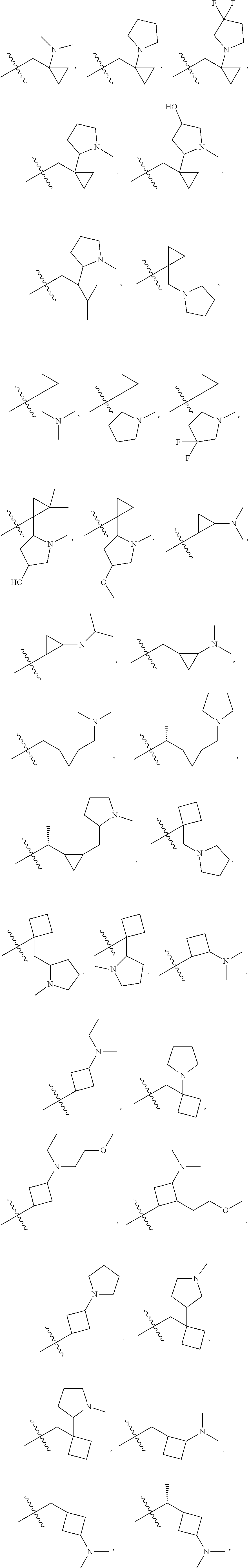

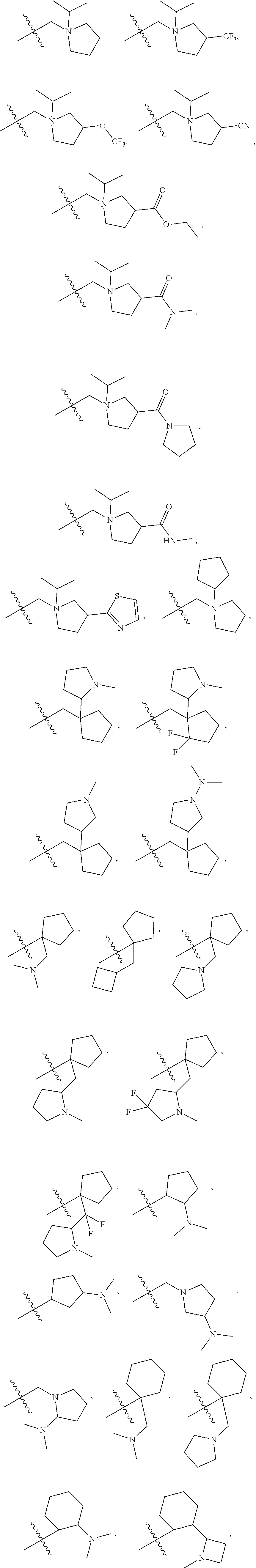

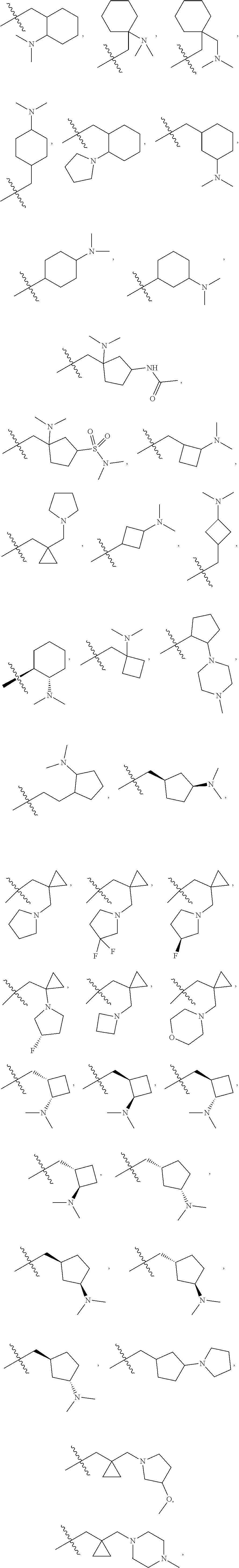

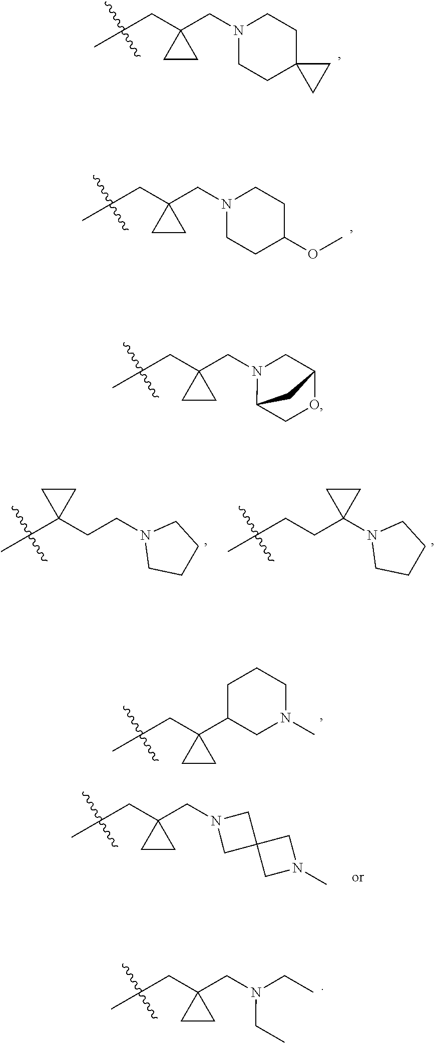

In some embodiments,

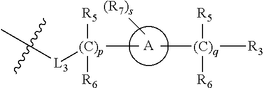

is selected from:

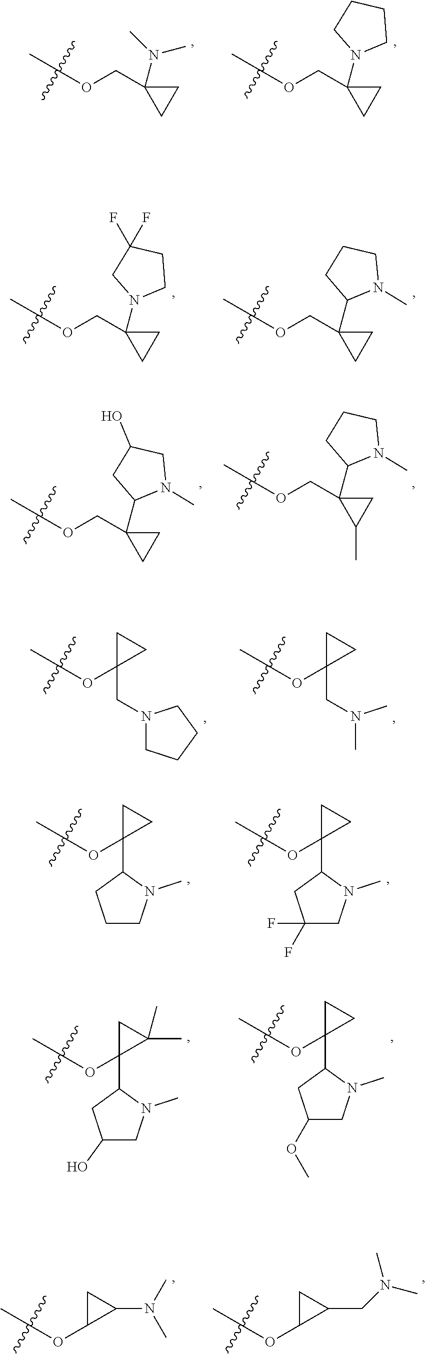

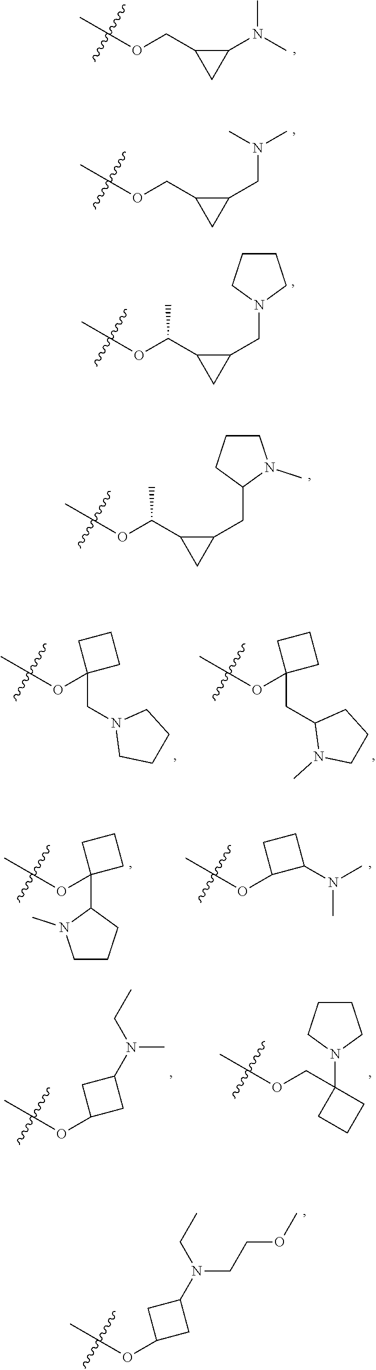

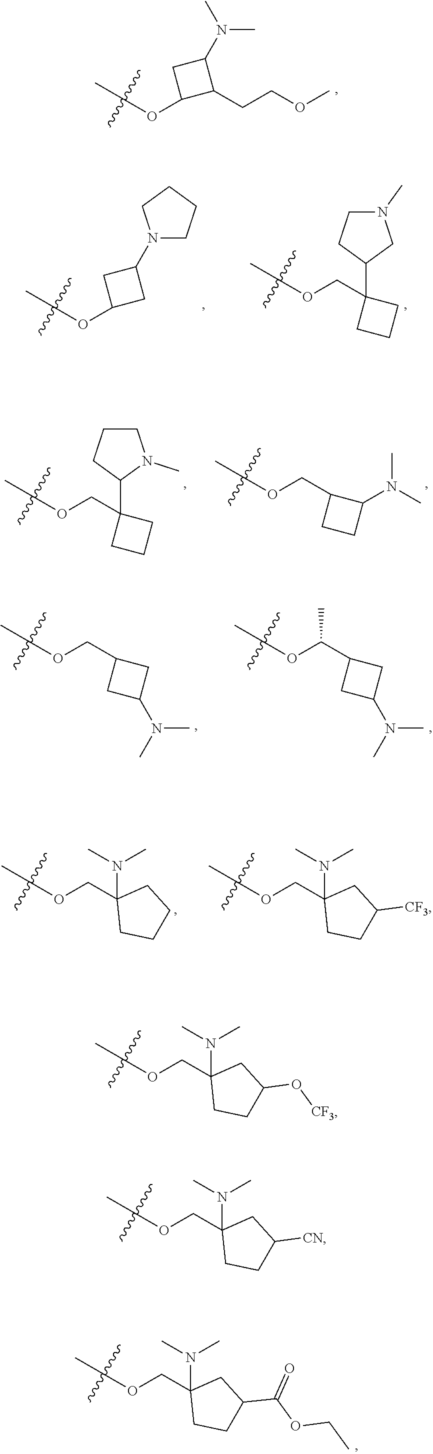

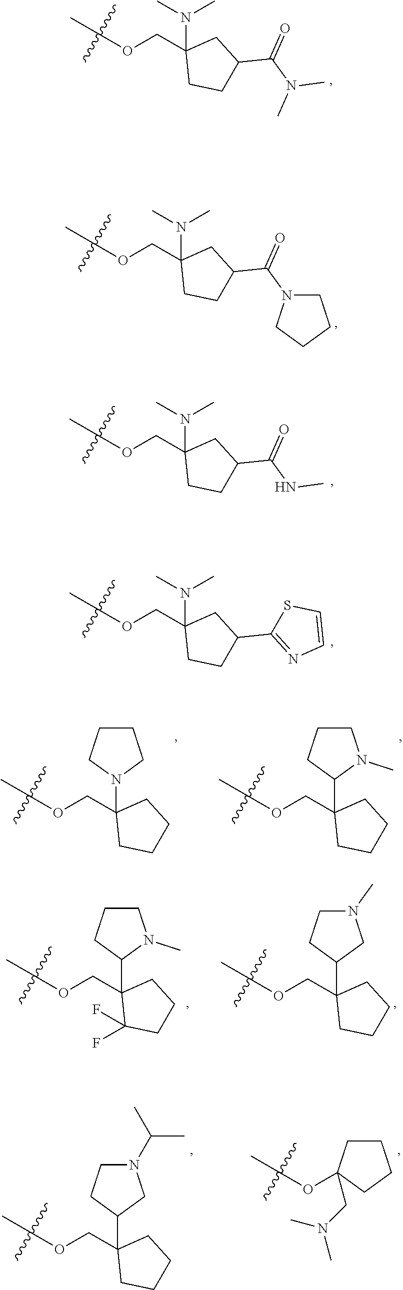

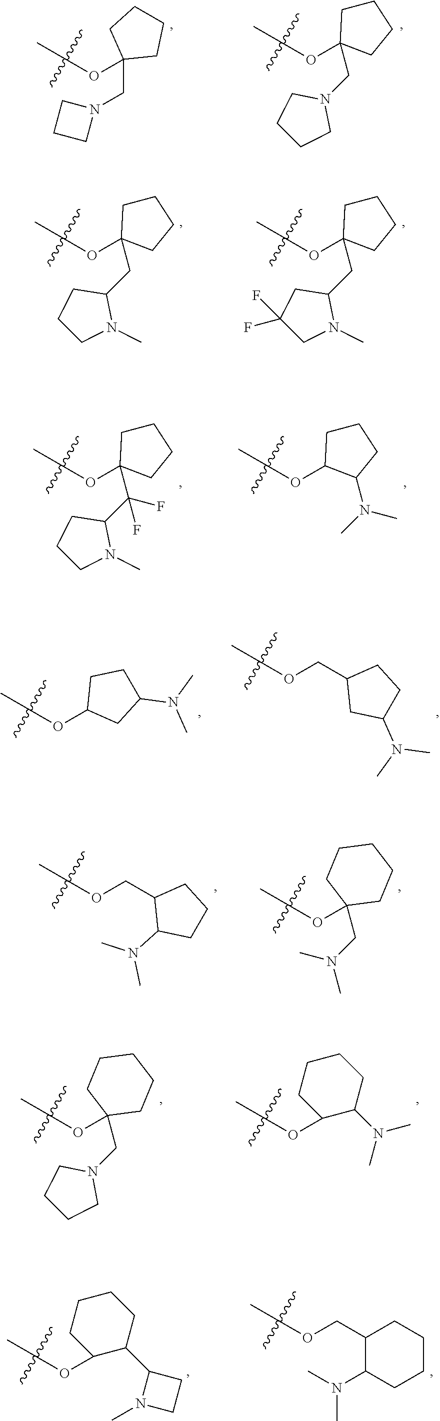

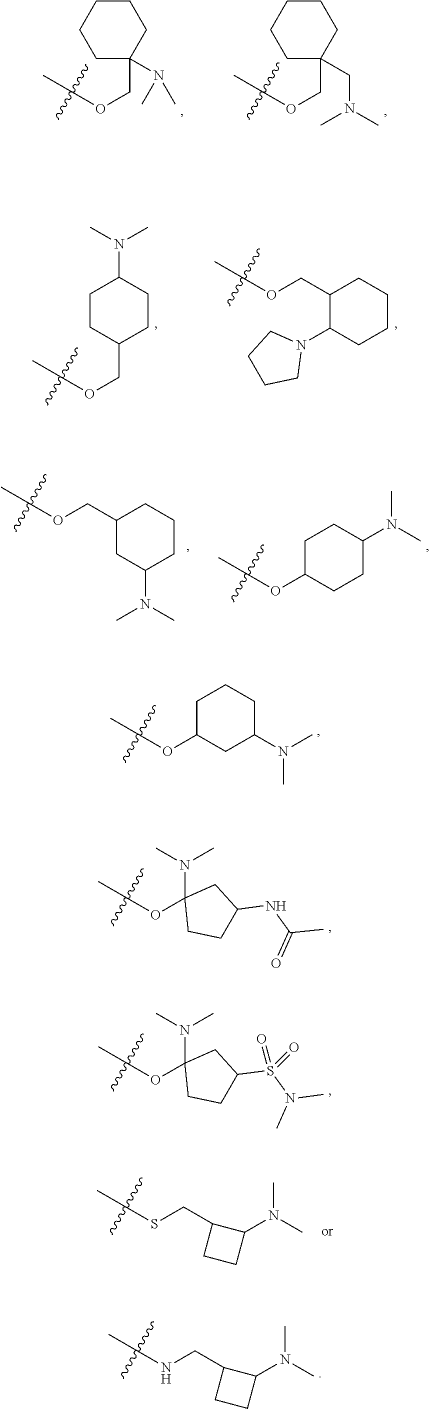

In some embodiments,

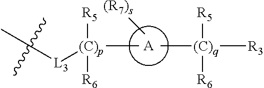

is selected from:

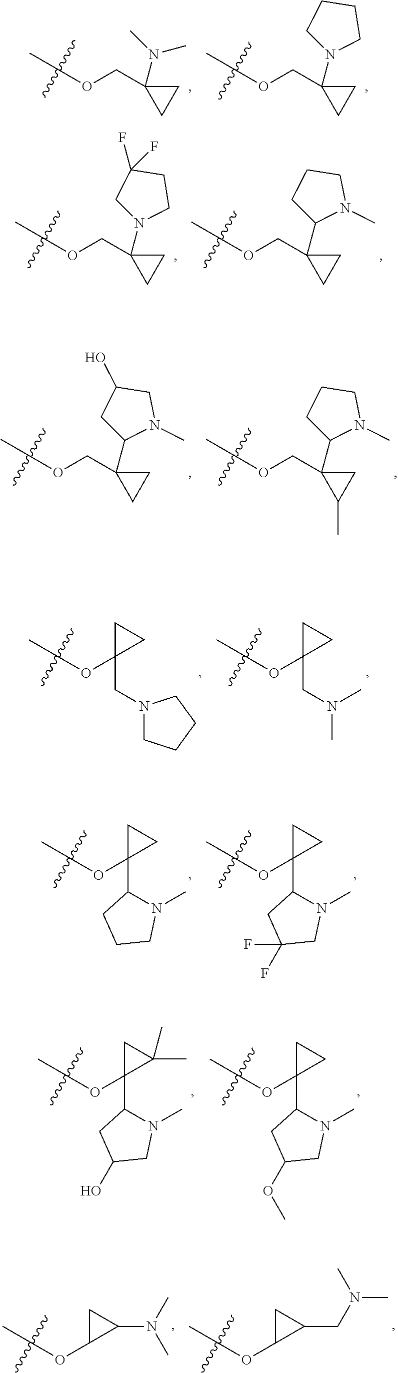

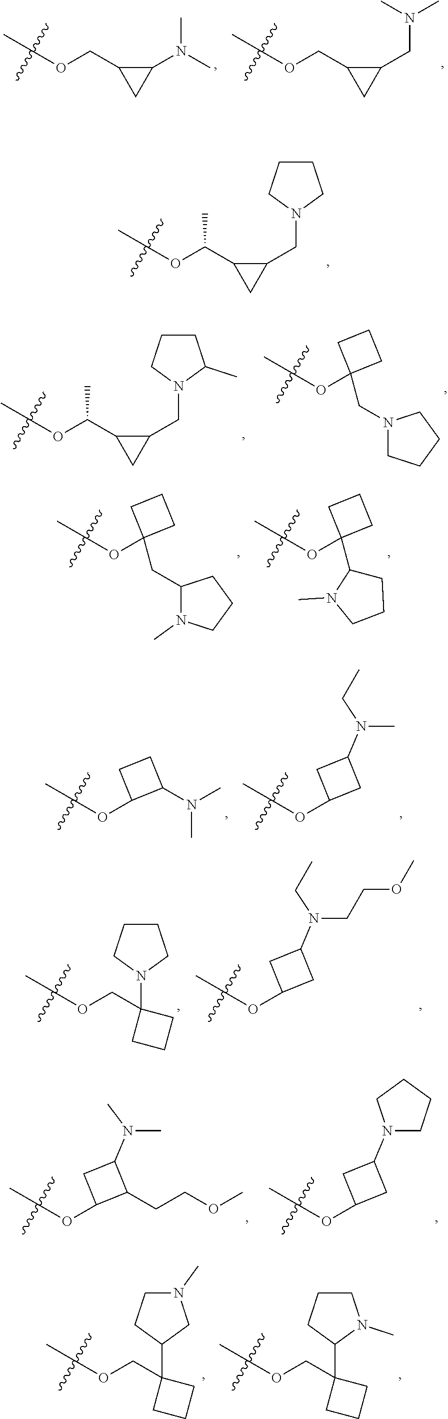

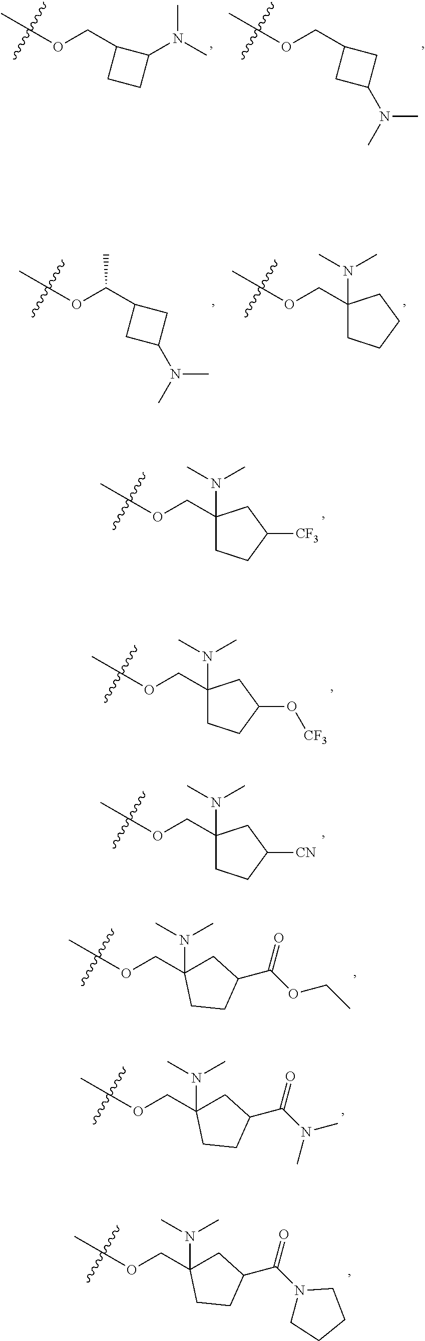

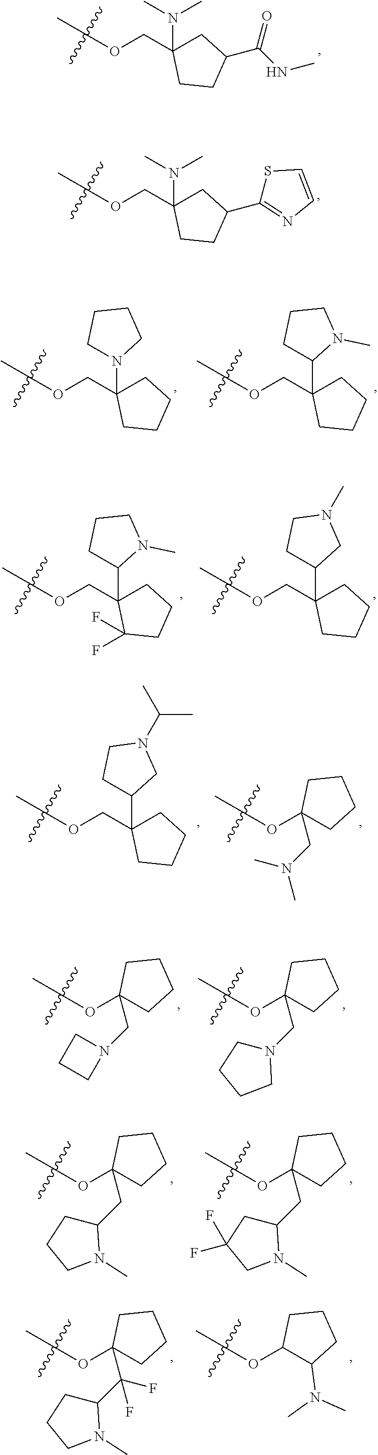

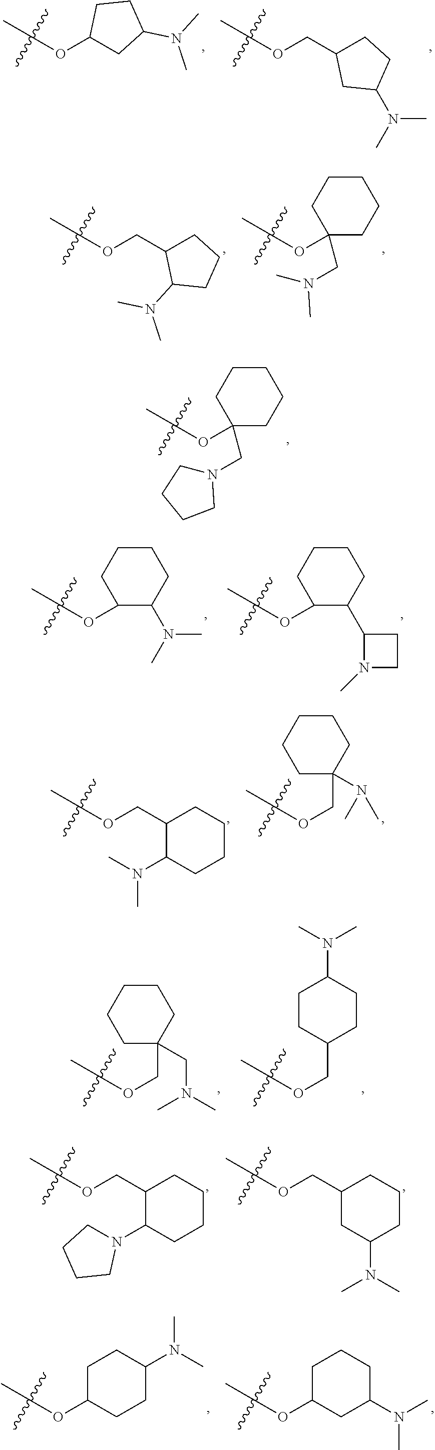

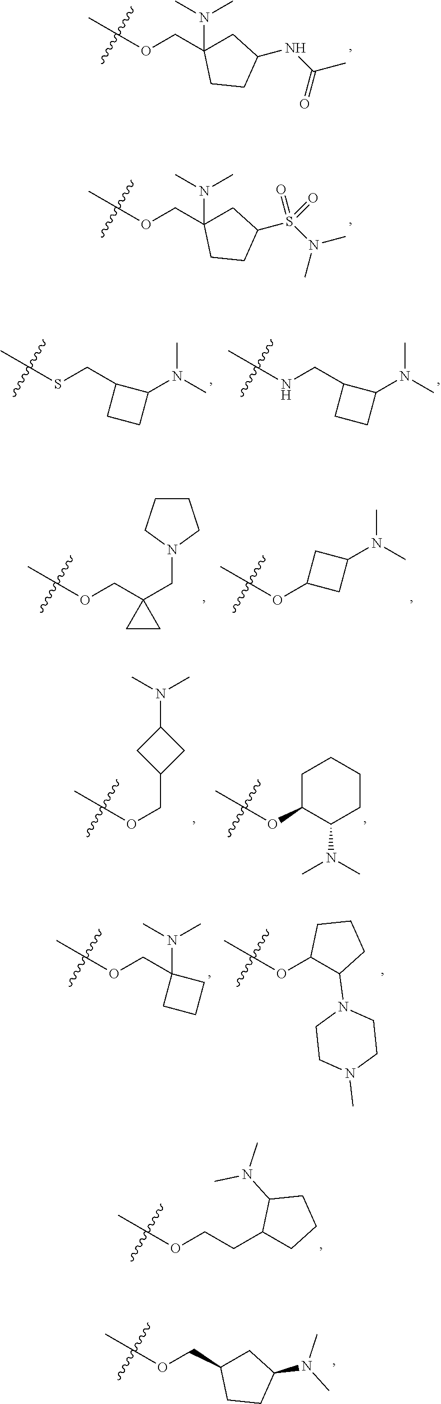

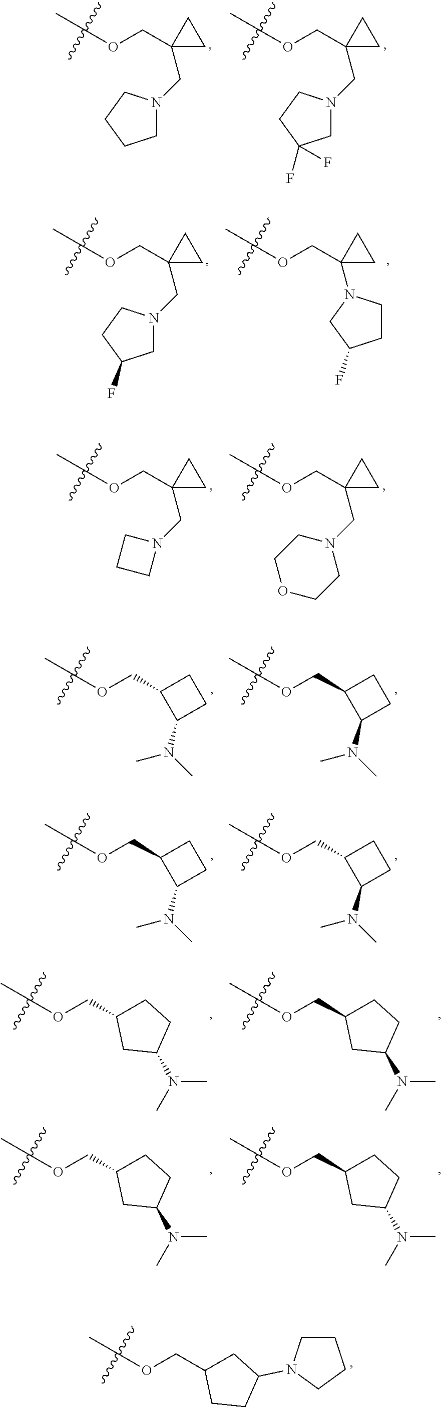

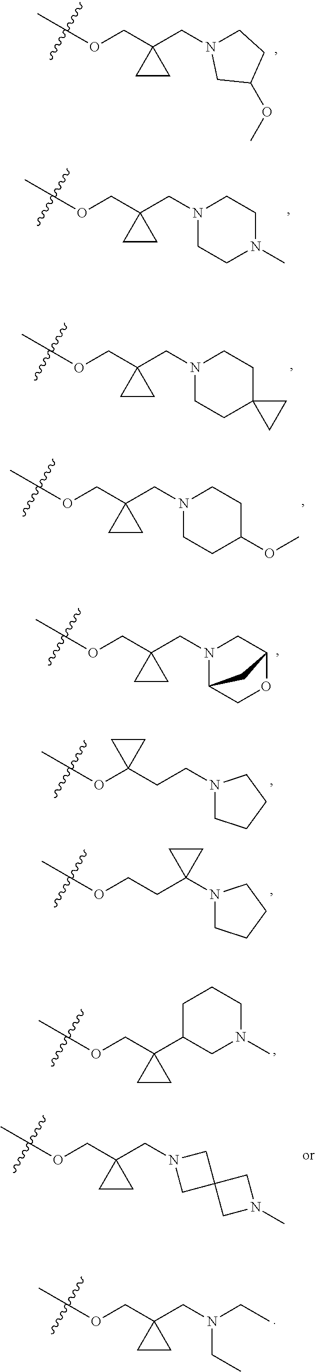

In some embodiments, each of L₄ at each occurrence is independently selected from absent or (CR₅R₆)ₘ.

In some embodiments, each of L₄ at each occurrence is independently selected from absent.

In some embodiments, each of R₄ at each occurrence is independently selected from

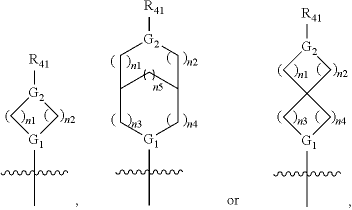

at each occurrence is independently optionally substituted by 1, 2, 3, 4, 5 or 6 R₄₂.

In some embodiments, each of R* at each occurrence is independently selected from

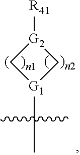

each of which at each occurrence is independently optionally substituted by 1, 2, 3, 4, 5 or 6 R₄₂.

In some embodiments, each of G₁ and G₂ at each occurrence is independently selected from N.

In some embodiments, each of n1 and n2 at each occurrence is independently selected from 1, 2 or 3.

In some embodiments, each of R₄ at each occurrence is independently selected from

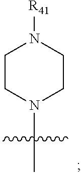

each of which at each occurrence is independently optionally substituted by 1, 2, 3, 4, 5 or 6 R₄₂.

In some embodiments, each of R₄ at each occurrence is independently selected from

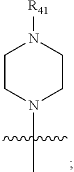

each of which at each occurrence is independently optionally substituted by 1, 2, 3, 4, 5 or 6 R₄₂, and L₄ is selected from absent.

In some embodiments, each of R₄₁ at each occurrence is independently selected from

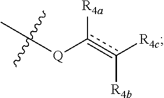

Each of Q at each occurrence is independently selected from C(═O) or S(═O)₂.

In some embodiments, each of R₄₁ at each occurrence is independently selected from

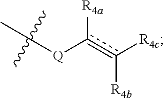

Each of Q at each occurrence is independently selected from or C(═O).

In some embodiments, each of R₄ₐ, R_{4b} and R_{4c} at each occurrence is independently selected from hydrogen, —F, —Cl, —Br, oxo, —C₁₋₃alkyl, —C₁₋₃alkylene-(halo)-3, heteroC₁₋₃alkyl, —CN, —OR₈, —C₁₋₃alkylene-(OR₈)₁₋₃, —NR₈R₉, —C₁₋₃alkylene-NR₈R₉, —C(═O)R₈, —C(═O)OR₈, —OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(O)₂NR₈R₉ or —C₃₋₆carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉; or R_{4b} and R_{4c} with the carbon which they both attach to form a ring selected from C₃₋₆ carbocyclic or 3-6 membered heterocyclic, each of 3-6 membered heterocyclic at each occurrence contains 1, 2 or 3 heteroatoms selected from N, O or S, and each of C₃₋₆ carbocyclic or 3-6 membered heterocyclic may be optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉ when [symbol] is selected from ═;

Each of R₄ₐ is absent and one of R_{4b} and R_{4c} is absent, another of R_{4b} and R_{4c} is selected from hydrogen, —F, —Cl, —Br, oxo, —C₁₋₃alkyl, —C₁₋₃alkylene-(halo)₁₋₃, heteroC₁₋₃alkyl, —CN, —OR₈, —C₁₋₃alkylene-(OR₈)₁₋₃, —NR₈R₉, —C₁₋₃alkylene-NR₈R₉, —C(═O)R₈, —C(═O)OR₈, —OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(O)₂NR₈R₉ or —C₃₋₆carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉ when [symbol] is selected from [symbol] ;

Each of R₈ and R₉ in R₄ₐ, R_{4b} or R_{4c} at each occurrence is independently selected from hydrogen or —C₁-3alkyl, or

R₈ and R₉ in R₄ₐ, R_{4b} or R_{4c} together with the N atom which they both attach form a 3-6 membered heterocyclic ring, the 3-6 membered heterocyclic ring may further contain 1, 2, 3 or 4 heteroatoms selected from N, O or S.

In some embodiments, each of R₄ₐ, R_{4b} and R_{4c} at each occurrence is independently selected from hydrogen, —F, —Cl, oxo, methyl, ethyl, propyl, isopropyl, -methylene-(halo)₁₋₃, -ethylene-(halo)₁₋₃, -propylene-(halo)₁₋₃, heteroethyl, heteropropthyl, —CN, —OR₈, -methylene-(OR₈)₁₋₃, -ethylene-(OR₈)₁₋₃, -propylene-(OR₈)₁₋₃, —NR₈R₉, -methylene-NR₈R₉, -ethylene-NR₈R₉, -propylene-NR₈R₉, —C(═O)R₈, —C(═O)OR₈, —OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈, —S(O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉; or R_{4b} and R_{4c} with the carbon which they both attach to form a ring selected from 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic, each of 3-6 membered heterocyclic at each occurrence contains 1 or 2 heteroatoms selected from N or O, and each of 3-6 membered carbocyclic or 3-6 membered heterocyclic may be optionally substituted by 1, 2, 3, 4, 5 or 6 substituents selected from —F, —Cl, methyl, ethyl, propyl, isopropyl, methoxy, ethoxy, propoxy, isopropyl, oxo, —OR₈, —NR₈R₉, —CN, —C(═O)R₈, —C(═O)OR₈, —OC(═O)R₈, —C(═O)NR₈R₉, —NR₈C(═O)R₈ or —S(O)₂NR₈R₉ when [symbol] is selected from ═;

Each of R₄ₐ is absent and one of R_{4b} and R_{4c} is absent, another of R_{4b} and R_{4c} is selected from hydrogen, —F, —Cl, oxo, methyl, ethyl, propyl, isopropyl, -methylene-(halo)₁₋₃, -ethylene-(halo)₁₋₃, -propylene-(halo)₁₋₃, heteroethyl, heteropropthyl, —CN, —OR₈, -methylene-(OR₈)₁₋₃, -ethylene-(OR₈)₁₋₃, -propylene-(OR₈)₁₋₃, —NR₈R₉, -methylene-NR₈R₉, -ethylene-NR₈R₉, -propylene-NR₈R₉, —C(═O)R₈, —C(═O)OR₈, —OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈, —S(O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉ when [symbol] is selected from [symbol] ;

Each of (R₈ and R₉) in (R₄ₐ, R_{4b} or R_{4c}) at each occurrence is independently selected from hydrogen, methyl, ethyl, propyl or isopropyl; or

(R₈ and R₉) in (R₄ₐ, R_{4b} or R_{4c}) together with the N atom which they both attach form

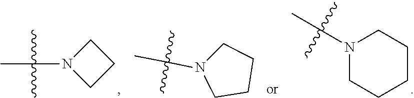 [SMILES: *N1CCC1 ; *N1CCCC1 ; *N1CCCCC1]

In some embodiments, each of R₄ₐ, R_{4b} and R_{4c} at each occurrence is independently selected from hydrogen, —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂,

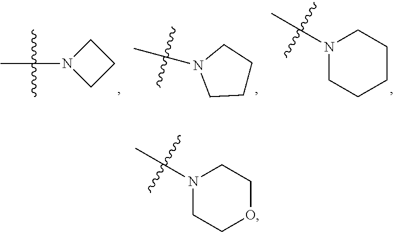 [SMILES: *N1CCC1 ; *N1CCCC1 ; *N1CCCCC1 ; *N1CCOCC1]

—CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂,

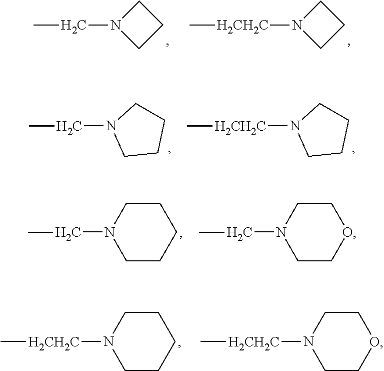

—C(═O)CH₃, —C(═O)OCH₃, —C(═O)OCH₂CH₃, —C(═O)OCH₂CH₂CH₃, —OC(═O)CH₃, —C(═O)NH₂, —C(═)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂; or R_{4b} and R_{4c} with the carbon which they both attach to form a ring selected from 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic, each of 3-6 membered heterocyclic at each occurrence contains 1 or 2 heteroatoms selected from N or O, and each of 3-6 membered carbocyclic or 3-6 membered heterocyclic may be optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂ when [symbol] is selected from ═; or

Each of R₄ₐ is absent and one of R_{4b} and R_{4c} is absent, another of R_{4b} and R_{4c} is selected from hydrogen, —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂,

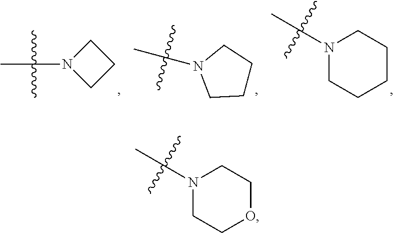 [SMILES: *N1CCC1 ; *N1CCCC1 ; *N1CCCCC1 ; *N1CCOCC1]

—CH₂NH₂, —CH₂CH₂NH₂, —CH₂CH₂CH₂NH₂, —CH₂N(CH₃)₂,

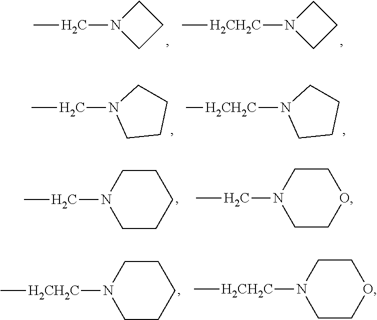

—C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂ when [symbol] is selected from [symbol] .

In some embodiments, each of R₄ₐ, R_{4b} and R_{4c} at each occurrence is independently selected from hydrogen, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂CH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂,

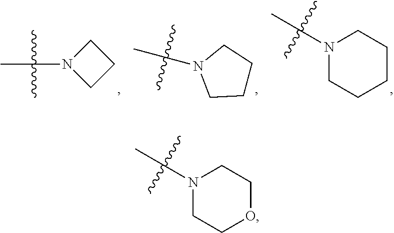 [SMILES: *N1CCC1 ; *N1CCCC1 ; *N1CCCCC1 ; *N1CCOCC1]

CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂,

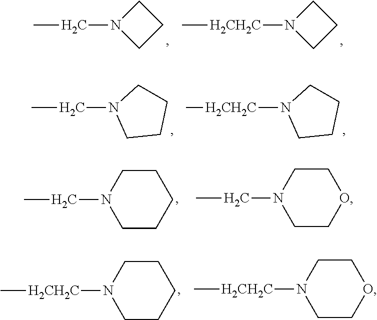

—C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic, or R_{4b} and R_{4c} with the carbon which they both attach to form a ring selected from 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic, each of 3-6 membered heterocyclic at each occurrence contains 1 or 2 heteroatoms selected from N or O when [symbol] is selected from ═;

Each of R₄ₐ is absent and one of R_{4b} and R_{4c} is absent, another of R_{4b} and R_{4c} is selected from hydrogen, —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃—NHCH(CH₃)₂, —N(CH₂, —N(CH₃)CH₂CH₃, —N(CH₃)CH₂CH₂CH₃, —N(CH₃)CH(CH₃)₂,

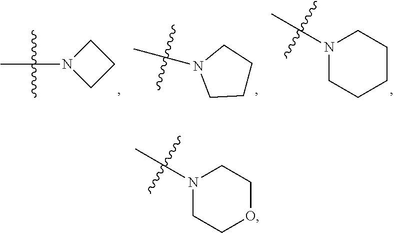 [SMILES: *N1CCC1 ; *N1CCCC1 ; *N1CCCCC1 ; *N1CCOCC1]

—CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂,

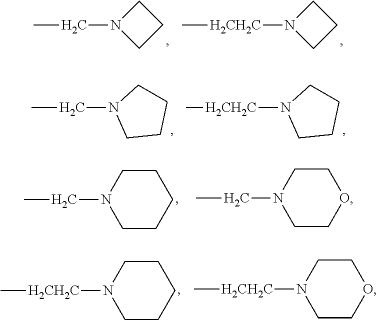

—C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃), —S(O)₂N(CH₃)₂, 3 membered carbocyclic, 4 membered carbocyclic, 5 membered carbocyclic or 6 membered carbocyclic when [symbol] is selected from [symbol] .

In some embodiments, each of R₄ₐ, R_{4b} and R_{4c} at each occurrence is independently selected from hydrogen, —F, methyl,

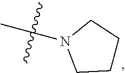 [SMILES: *N1CCCC1]

—CH₂N(CH₃)₂,

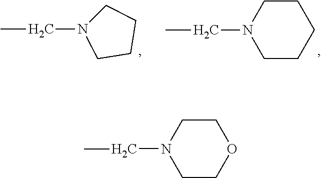

when [symbol] is selected from ═;

Each of R₄ₐ is absent and one of R_{4b} and R_{4c} is absent, another of R_{4b} and R_{4c} is selected from hydrogen or methyl when [symbol] is selected from [symbol] .

In some embodiments, each of R₄ₐ, R_{4b}, R_{4b} and R_{4c} at each occurrence is independently selected from hydrogen, —F, —CH₂F, methyl,

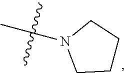 [SMILES: *N1CCCC1]

—CH₂N(CH₃)₂,

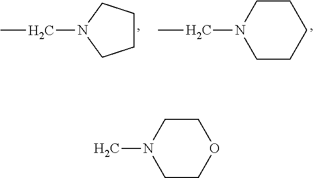

when [symbol] is selected from ═;

Each of R₄ₐ is absent and one of R_{4b} and R_{4c} is absent, another of R_{4b} and R_{4c} is selected from hydrogen or methyl when [symbol] is selected from [symbol] .

In some embodiments, R₄₁ is selected from

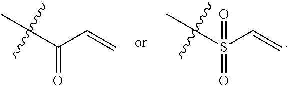

In some embodiments, R₄₁ is selected from

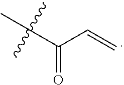 [SMILES: *C(=O)C=C]

In some embodiments, each of R₄₂ is selected from —F, —Cl, —Br, oxo, —C₁₋₃alkyl, —C₁₋₃alkylene-(halo)₁₋₃, heteroC₁₋₃alkyl, —C₂₋₃alkenyl, —C₂₋₃alkynyl, —OR₈, —C₁₋₃alkylene-(OR₈)₁₋₃, —NR₈R₉, —C₁₋₃alkylene-NR₈R₉, —CN, —C₁₋₃alkylene-CN, —C(═O)R₈, —C(═O)OR₈, —OC(═O)R₈, —C(═O)NR₈R₉, —C₁₋₃alkylene-C(═O)NR₈R₉, —NR₈C(═O)R₈, —C₁₋₃alkylene-NR₈C(═O)R₈ or —S(O)₂NR₈R₉; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of (R₈ and R₉) in R₄₂ at each occurrence is independently selected from hydrogen or —C₁₋₃alkyl.

In some embodiments, each of R₄₂ is selected from —F, —Cl, oxo, methyl, ethyl, propyl, isopropyl, -methylene-(halo)₁₋₃, -ethylene-(halo)₁₋₃, -propylene-(halo)₁₋₃, heteroethyl, heteropropyl, ethenyl, propenyl, ethynyl, propynyl, —OR₈, -methylene-(OR₈)₁₋₃, -ethylene-(OR₈)₁₋₃, -propylene-(OR₈)₁₋₃, —NR₈R₉, -methylene-NR₈R₉, -ethkylene-NR₈R₉, -propylene-NR₈R₉, —CN, -methylene-CN, -ethylene-CN, -propyl-CN, —C(═O)R₈, —C(═O)OR₈, —OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈ or —S(O)₂NR₈R₉; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈, or —S(O)₂NR₈R₉;

Each of (R₈ and R₉) in R₄₂ at each occurrence is independently selected from hydrogen, methyl, ethyl, propyl or isopropyl.

In some embodiments, each of R₄₂ is selected from —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, ethenyl, propenyl, ethynyl, propynyl, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —CN, —CH₂CN, —CH₂CH₂CN, —CH₂CH₂CN, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃) or —S(O)₂N(CH₃)₂; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃) —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each of R₄₂ is selected from —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂CH₃, —CH₂CH₂CH₂CH₃, ethenyl, propenyl, ethynyl, propynyl, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —CN, —CH₂CN, —CH₂CH₂CN, —CH₂CH₂CN, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(O)₂NH₂, —S(O)₂NH(CH₃) or —S(O)₂N(CH₃)₂.

In some embodiments, each of R₄₂ is selected from methyl, —C₂CH₂F, —CH₂CHF₂, —CH₂CF₃, —CH₂OCH₃—CH₂CH₂OCH₃, propynyl, —CH₂OH, —CH₂CH₂OH, —CN, —CH₂CN or —C(═O)N(CH₃)₂.

In some embodiments, each of R₄ is independently selected from:

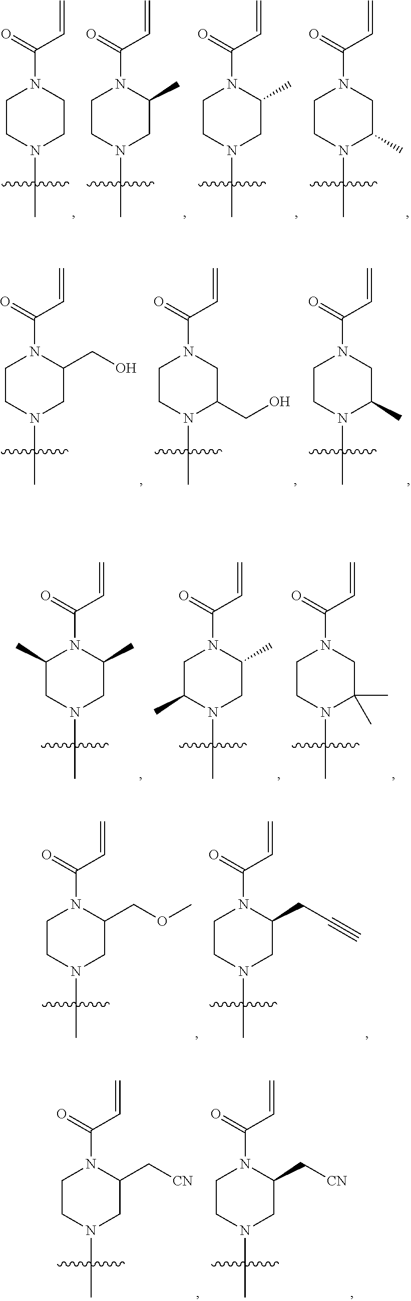

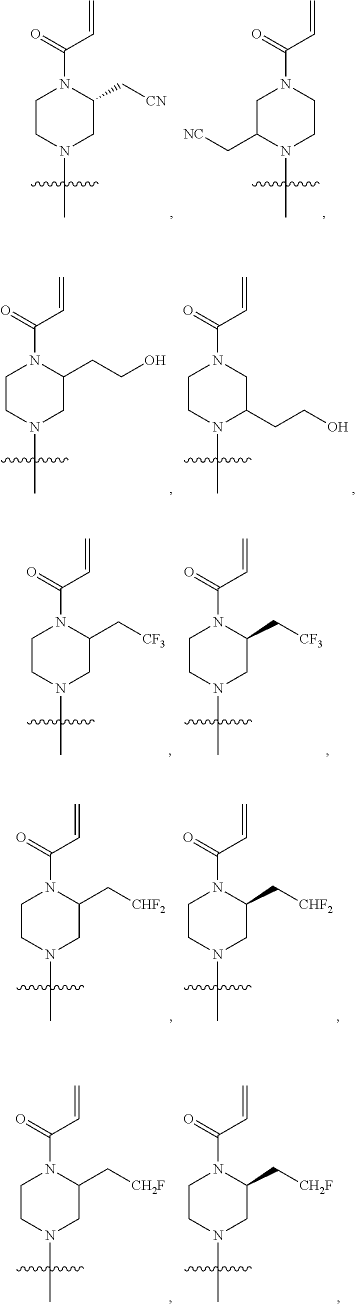

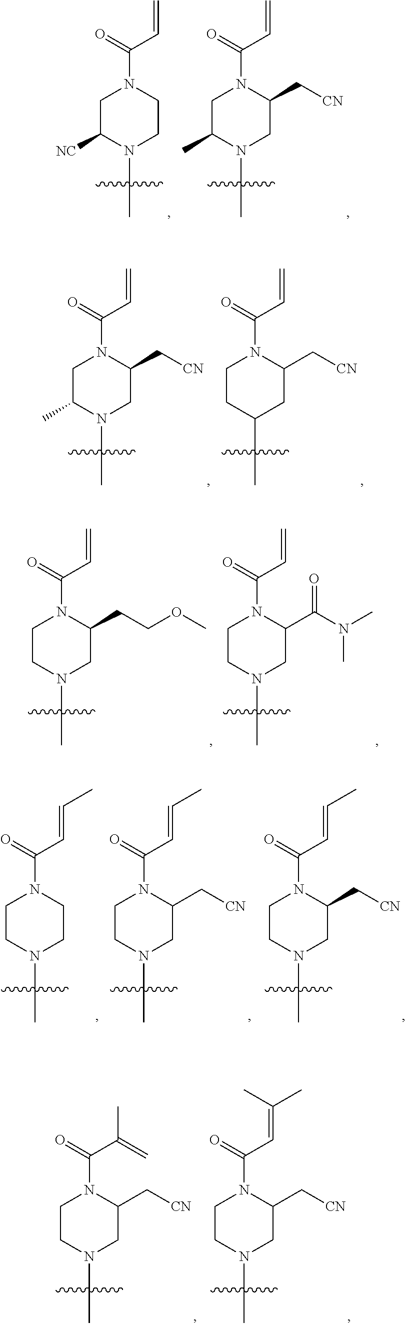

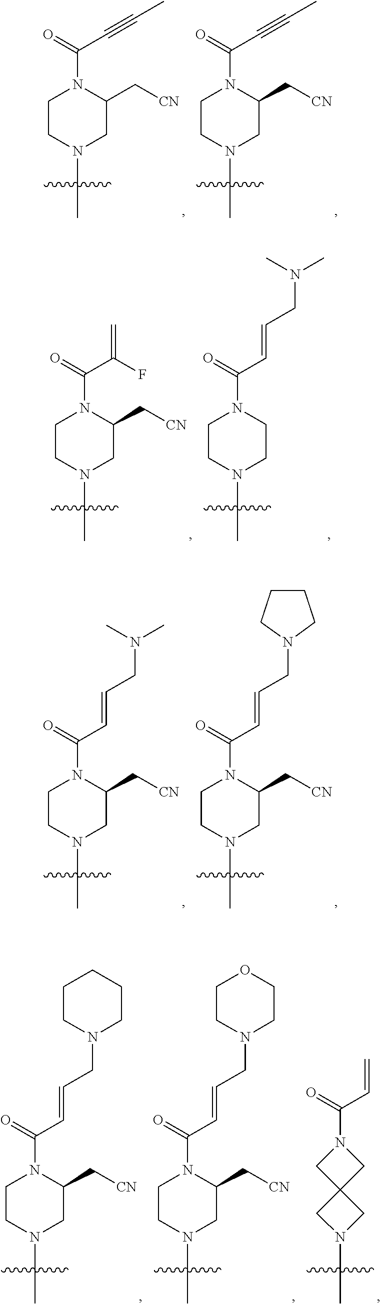

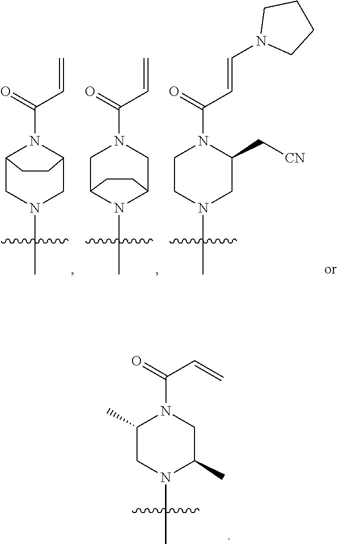

In some embodiments, each of R₄ is independently selected from:

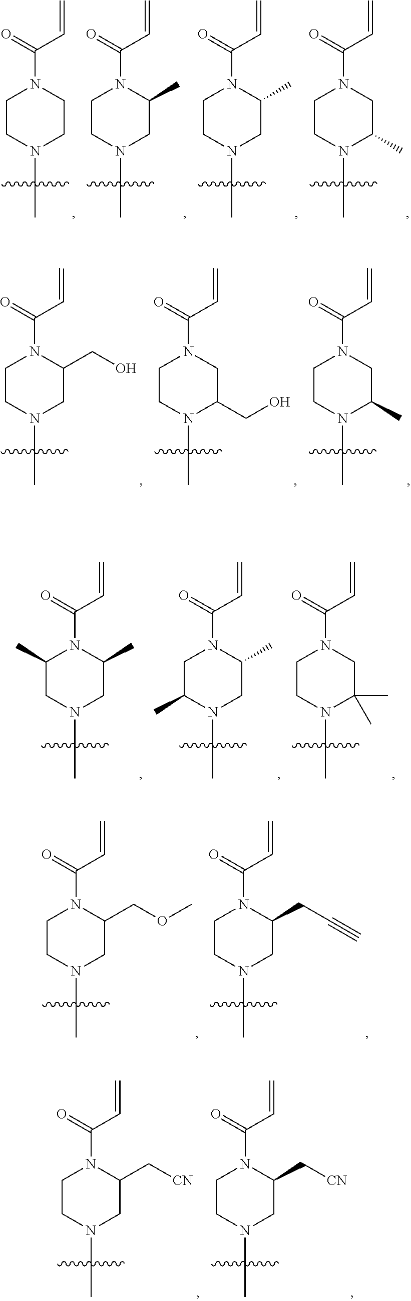

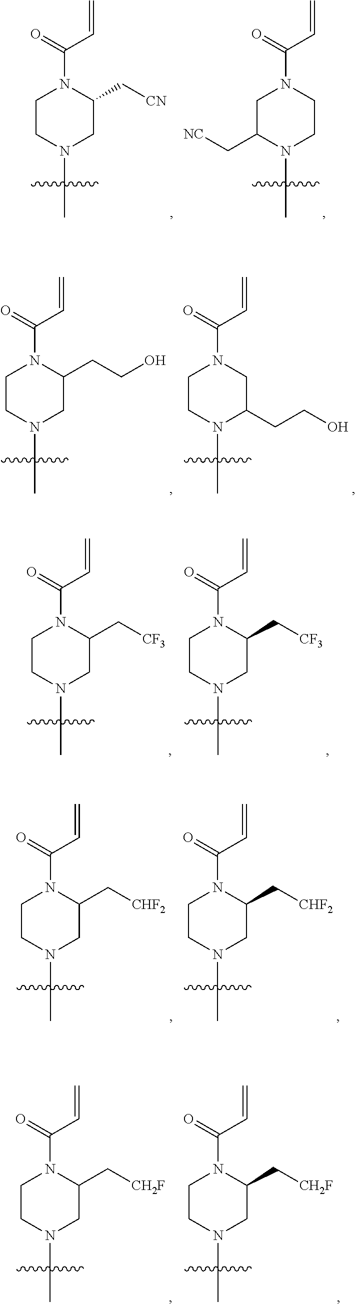

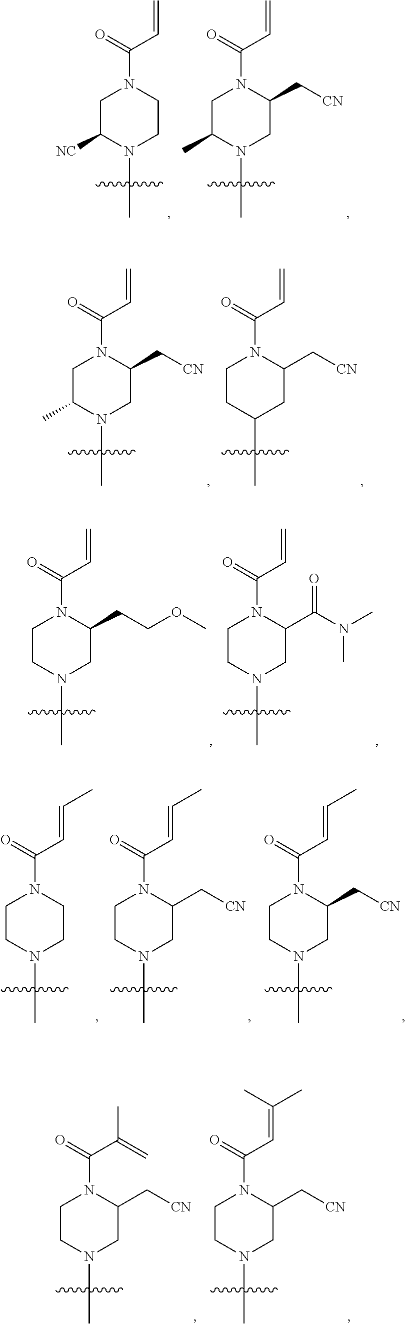

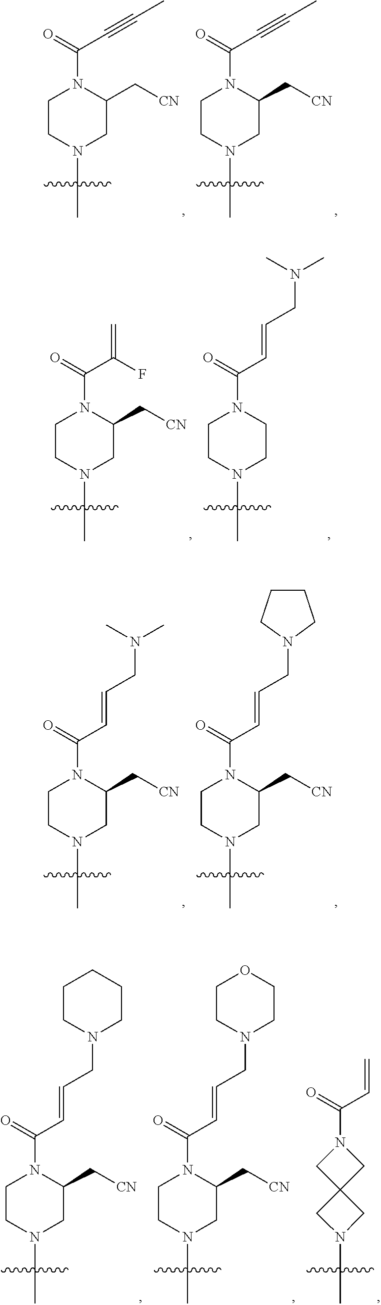

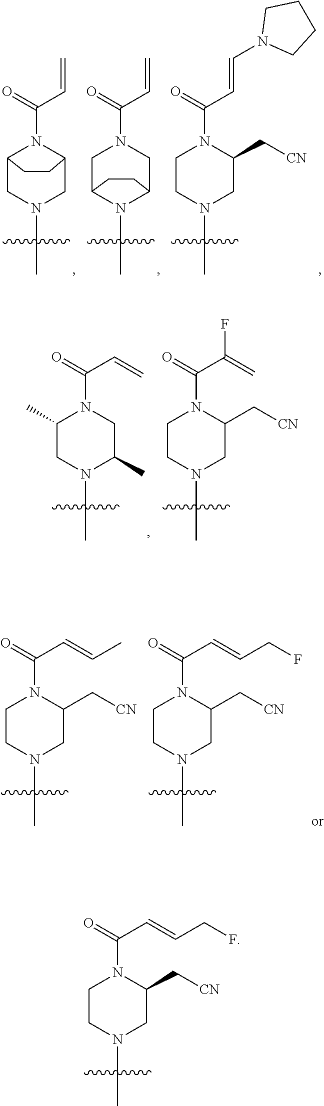

In some embodiments, the compound is selected from:

| | |
|---|---|
| 1. | 2-(1-acryloyl-4-(2-(((1r,4r)-4-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 2. | 2-(1-acryloyl-4-(2-((1-((dimethylamino)methyl)cyclopropyl)methoxy)-7-(2-methyl-1-oxo-1,2- |
| | dihydroisoquinolin-8-yl)-5,6,7,8-tetrahydropyrido[3,4-d]pyrimidin-4-yl)piperazin-2-yl)acetonitrile; |
| 3. | 2-(1-acryloyl-4-(2-((1-((dimethylamino)methyl)cyclopropyl)methoxy)-7-(5-methyl-1H-indazol-4- |
| | yl)-5,6,7,8-tetrahydropyrido[3,4-d]pyrimidin-4-yl)piperazin-2-yl)acetonitrile; |
| 4. | 2-(1-acryloyl-4-(7-(naphthalen-1-yl)-2-((1-(pyrrolidin-1-ylmethyl)cyclopropyl)methoxy)-5,6,7,8- |
| | tetrahydropyrido[3,4-d]pyrimidin-4-yl)piperazin-2-yl)acetonitrile; |
| 5. | 2-(1-acryloyl-4-(2-((3-(dimethylamino)cyclohexyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 6. | 2-(1-acryloyl-4-(2-((3-(dimethylamino)cyclohexyl)methoxy)-7-(2-(trifluoromethyl)phenyl)-5,6,7,8- |
| | tetrahydropyrido[3,4-d]pyrimidin-4-yl)piperazin-2-yl)acetonitrile; |
| 7. | 2-(1-acryloyl-4-(2-(3-(dimethylamino)cyclobutoxy)-7-(naphthalen-1-yl)-5,6,7,8- |
| | tetrahydropyrido[3,4-d]pyrimidin-4-yl)piperazin-2-yl)acetonitrile; |
| 8. | 2-(1-acryloyl-4-(2-((3-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 9. | 2-(1-acryloyl-4-(2-((1-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 10. | 2-(1-acryloyl-4-(2-((3-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 11. | 2-(1-acryloyl-4-(2-((2-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 12. | 2-(1-acryloyl-4-(2-((1-(dimethylamino)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 13. | 2-(1-acryloyl-4-(2-((3-(dimethylamino)cyclopentyl)oxy)-7-(naphthalen-1-yl)-5,6,7,8- |
| | tetrahydropyrido[3,4-d]pyrimidin-4-yl)piperazin-2-yl)acetonitrile; |
| 14. | 2-(1-acryloyl-4-(2-((2-(dimethylamino)cyclopentyl)methoxy)-7-(naphthalen-1-yl)-5,6,7,8- |
| | tetrahydropyrido[3,4-d]pyrimidin-4-yl)piperazin-2-yl)acetonitrile; |
| 15. | 2-(1-acryloyl-4-(2-((3-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 16. | 2-(1-acryloyl-4-(2-((2-(4-methylpiperazin-1-yl)cyclopentyl)oxy)-7-(naphthalen-1-yl)-5,6,7,8- |
| | tetrahydropyrido[3,4-d]pyrimidin-4-yl)piperazin-2-yl)acetonitrile; |
| 17. | 2-(1-acryloyl-4-(2-((3-(dimethylamino)cyclopentyl)methoxy)-7-(naphthalen-1-yl)-5,6,7,8- |
| | tetrahydropyrido[3,4-d]pyrimidin-4-yl)piperazin-2-yl)acetonitrile; |
| 18. | 2-(1-acryloyl-4-(2-((2-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 19. | 2-(1-acryloyl-4-(2-((2-((dimethylamino)methyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 20. | 2-(1-acryloyl-4-(2-((1-(dimethylamino)cyclopropyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 21. | 2-(1-acryloyl-4-(2-(2-(2-(dimethylamino)cyclopentyl)ethoxy)-7-(naphthalen-1-yl)-5,6,7,8- |
| | tetrahydropyrido[3,4-d]pyrimidin-4-yl)piperazin-2-yl)acetonitrile; |
| 22. | 2-(1-acryloyl-4-(2-(((1R,3S)-3-(dimethylamino)cyclopentyl)methoxy)-7-(8-methylnaphthalen-1- |
| | yl)-5,6,7,8-tetrahydropyrido[3,4-d]pyrimidin-4-yl)piperazin-2-yl)acetonitrile; |
| 23. | 2-(1-acryloyl-4-(2-((2-(dimethylamino)cyclopentyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 24. | 2-(1-acryloyl-4-(2-((3-(dimethylamino)cyclopentyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 25. | 2-(1-acryloyl-4-(7-(2,3-dichlorophenyl)-2-((2-(dimethylamino)cyclopentyl)methoxy)-5,6,7,8- |
| | tetrahydropyrido[3,4-d]pyrimidin-4-yl)piperazin-2-yl)acetonitrile; |
| 26. | 2-(1-acryloyl-4-(2-((2-(dimethylamino)cyclopentyl)methoxy)-7-(3-fluoro-2- |
| | (trifluoromethyl)phenyl)-5,6,7,8-tetrahydropyrido[3,4-d]pyrimidin-4-yl)piperazin-2-yl)acetonitrile; |
| 27. | 2-(1-acryloyl-4-(2-((2-(dimethylamino)cyclopentyl)methoxy)-7-(2-(trifluoromethyl)phenyl)- |
| | 5,6,7,8-tetrahydropyrido[3,4-d]pyrimidin-4-yl)piperazin-2-yl)acetonitrile; |
| 28. | 2-(1-acryloyl-4-(2-((2-(dimethylamino)cyclopentyl)methoxy)-7-(2,3-dimethylphenyl)-5,6,7,8- |
| | tetrahydropyrido[3,4-d]pyrimidin-4-yl)piperazin-2-yl)acetonitrile; |
| 29. | 2-(1-acryloyl-4-(7-(2-chloro-3-fluorophenyl)-2-((3-(dimethylamino)cyclopentyl)methoxy)-5,6,7,8- |
| | tetrahydropyrido[3,4-d]pyrimidin-4-yl)piperazin-2-yl)acetonitrile; |
| 30. | 2-(1-acryloyl-4-(2-((3-(dimethylamino)cyclopentyl)methoxy)-7-(4-(trifluoromethyl)26aphthal-3- |
| | yl)-5,6,7,8-tetrahydropyrido[3,4-d]pyrimidin-4-yl)piperazin-2-yl)acetonitrile; |
| 31. | 2-(1-acryloyl-4-(7-(2-chlorophenyl)-2-((3-(dimethylamino)cyclopentyl)methoxy)-5,6,7,8- |
| | tetrahydropyrido[3,4-d]pyrimidin-4-yl)piperazin-2-yl)acetonitrile; |
| 32. | 2-(1-acryloyl-4-(7-(naphthalen-1-yl)-2-((1-(pyrrolidin-1-yl)cyclopropyl)methoxy)-5,6,7,8- |
| | tetrahydropyrido[3,4-d]pyrimidin-4-yl)piperazin-2-yl)acetonitrile; |
| 33. | 2-(1-acryloyl-4-(7-(26aphthalene-1-yl)-2-((3-(pyrrolidin-1-yl)cyclopentyl)methoxy)-5,6,7,8- |
| | tetrahydropyrido[3,4-d]pyrimidin-4-yl)piperazin-2-yl)acetonitrile; |
| 34. | 2-(1-acryloyl-4-(7-(3-fluoro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 35. | 2-(1-acryloyl-4-(7-(2,3-dichlorophenyl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 36. | 2-(1-acryloyl-4-(2-((1-(pyrrolidin-1-ylmethyl)cyclopropyl)methoxy)-7-(2-(trifluoromethyl)pyridin- |
| | 3-yl)-5,6,7,8-tetrahydropyrido[3,4-d]pyrimidin-4-yl)piperazin-2-yl)acetonitrile; |
| 37. | 2-(1-acryloyl-4-(2-((1-((dimethylamino)methyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 38. | 2-(1-acryloyl-4-(7-(3-chloro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 39. | 2-(1-acryloyl-4-(2-((1-(azetidin-1-ylmethyl)cyclopropyl)methoxy)-7-(3-chloro-2- |
| | (trifluoromethyl)phenyl)-5,6,7,8-tetrahydropyrido[3,4-d]pyrimidin-4-yl)piperazin-2-yl)acetonitrile; |
| 40. | 2-(1-acryloyl-4-(7-(3-chloro-2-(trifluoromethyl)phenyl)-2-((1- |
| | (morpholinomethyl)cyclopropyl)methoxy)-5,6,7,8-tetrahydropyrido[3,4-d]pyrimidin-4- |
| | yl)piperazin-2-yl)acetonitrile; |
| 41. | 2-(1-acryloyl-4-(2-((1-(azetidin-1-ylmethyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 42. | 2-(1-acryloyl-4-(2-((1-(azetidin-1-ylmethyl)cyclopropyl)methoxy)-7-(8-chloronaphthalen-1-yl)- |
| | 5,6,7,8-tetrahydropyrido[3,4-d]pyrimidin-4-yl)piperazin-2-yl)acetonitrile; |
| 43. | 2-(1-acryloyl-4-(2-((1-(azetidin-1-ylmethyl)cyclopropyl)methoxy)-7-(8-methylnaphthalen-1-yl)- |
| | 5,6,7,8-tetrahydropyrido[3,4-d]pyrimidin-4-yl)piperazin-2-yl)acetonitrile; |
| 44. | 2-(1-acryloyl-4-(7-(naphthalen-1-yl)-2-((1-(pyrrolidin-1-ylmethyl)cyclobutyl)methoxy)-5,6,7,8- |
| | tetrahydropyrido[3,4-d]pyrimidin-4-yl)piperazin-2-yl)acetonitrile; |
| 45. | 2-(1-acryloyl-4-(7-(8-methylnaphthalen-1-yl)-2-((1-(pyrrolidin-1-ylmethyl)cyclobutyl)methoxy)- |
| | 5,6,7,8-tetrahydropyrido[3,4-d]pyrimidin-4-yl)piperazin-2-yl)acetonitrile; |
| 46. | 2-(1-acryloyl-4-(7-(8-chloronaphthalen-1-yl)-2-((1- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 47. | 2-(1-acryloyl-4-(2-((1-((dimethylamino)methyl)cyclopropyl)methoxy)-7-(8-methylnaphthalen-1- |
| | yl)-5,6,7,8-tetrahydropyrido[3,4-d]pyrimidin-4-yl)piperazin-2-yl)acetonitrile; |
| 48. | 2-(1-acryloyl-4-(2-((1-((dimethylamino)methyl)cyclopropyl)methoxy)-7-(2,3-dimethylphenyl)- |
| | 5,6,7,8-tetrahydropyrido[3,4-d]pyrimidin-4-yl)piperazin-2-yl)acetonitrile; |
| 49. | 2-(1-acryloyl-4-(2-((1-(((R)-3-fluoropyrrolidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 50. | 2-(1-acryloyl-4-(7-(benzo[b]thiophen-7-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 51. | 2-(1-acryloyl-4-(7-(benzo[b]thiophen-4-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 52. | 2-(1-acryloyl-4-(7-(benzo[d]thiazol-4-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 53. | 2-(1-acryloyl-4-(7-(2,3-dihydrobenzofuran-7-yl)-2-((1- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 54. | 2-(1-acryloyl-4-(7-(benzo[d]thiazol-7-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 55. | 2-(1-acryloyl-4-(7-(2-amino-6-fluorophenyl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 56. | 2-(1-acryloyl-4-(7-(8-methylnaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 57. | 2-(1-acryloyl-4-(2-(1-(1-((dimethylamino)methyl)cyclopropyl)ethoxy)-7-(8-methylnaphthalen-1- |
| | yl)-5,6,7,8-tetrahydropyrido[3,4-d]pyrimidin-4-yl)piperazin-2-yl)acetonitrile; |
| 58. | 2-(1-acryloyl-4-(7-(8-methylnaphthalen-1-yl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 59. | 2-(1-acryloyl-4-(2-((1-((3-(dimethylamino)azetidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 60. | 2-(1-acryloyl-4-(2-(((1S,2S)-2-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 61. | 2-(1-acryloyl-4-(7-(8-chloronaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 62. | 2-(1-acryloyl-4-(2-((1-((3-fluoroazetidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 63. | 2-(1-acryloyl-4-(7-(8-methylnaphthalen-1-yl)-2-((1-(4-methylpiperazine-1- |
| | carbonyl)cyclopropyl)methoxy)-5,6,7,8-tetrahydropyrido[3,4-d]pyrimidin-4-yl)piperazin-2- |
| | yl)acetonitrile; |
| 64. | 2-(1-acryloyl-4-(2-((1-(((2-(dimethylamino)ethyl)(methyl)amino)methyl)cyclopropyl)methoxy)-7- |
| | (8-methylnaphthalen-1-yl)-5,6,7,8-tetrahydropyrido[3,4-d]pyrimidin-4-yl)piperazin-2- |
| | yl)acetonitrile; |
| 65. | 2-(1-acryloyl-4-(2-((1-(((S)-3-(dimethylamino)pyrrolidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 66. | 2-(4-(2-((1-((4-acetylpiperazin-1-yl)methyl)cyclopropyl)methoxy)-7-(8-methylnaphthalen-1-yl)- |
| | 5,6,7,8-tetrahydropyrido[3,4-d]pyrimidin-4-yl)-1-acryloylpiperazin-2-yl)acetonitrile; |
| 67. | (S)-2-(1-acryloyl-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 68. | (S,E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-fluorobut-2-enoyl)piperazin-2-yl)acetonitrile; |
| 69. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-hydroxybut-2-enoyl)piperazin-2- |
| | yl)acetonitrile; |
| 70. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-methoxybut-2-enoyl)piperazin-2- |
| | yl)acetonitrile; |
| 71. | 2-(1-(2-fluoroacryloyl)-4-(7-(8-methylnaphthalen-1-yl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 72. | 2-(1-(2-fluoroacryloyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 73. | 2-(4-(2-((1-(azetidin-1-ylmethyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 74. | (E)-2-(1-(but-2-enoyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 75. | 2-(4-(2-((1-((3-(dimethylamino)azetidin-1-yl)methyl)cyclopropyl)methoxy)-7-(8- |
| | methylnaphthalen-1-yl)-5,6,7,8-tetrahydropyrido[3,4-d]pyrimidin-4-yl)-1-(2- |
| | fluoroacryloyl)piperazin-2-yl)acetonitrile; |
| 76. | 2-(4-(2-((1-((diethylamino)methyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 77. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-fluorobut-2-enoyl)piperazin-2-yl)acetonitrile; |
| 78. | 2-(4-(2-((1-((dimethylamino)methyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 79. | 2-(4-(7-(8-chloronaphthalen-1-yl)-2-((1-((dimethylamino)methyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 80. | 2-(4-(2-((1-((dimethylamino)methyl)cyclopropyl)methoxy)-7-(2,3-dimethylphenyl)-5,6,7,8- |
| | tetrahydropyrido[3,4-d]pyrimidin-4-yl)-1-(2-fluoroacryloyl)piperazin-2-yl)acetonitrile; |
| 81. | 2-(4-(2-((1-((3,3-difluoropyrrolidin-1-yl)methyl)cyclopropyl)methoxy)-7-(8-methylnaphthalen-1- |
| | yl)-5,6,7,8-tetrahydropyrido[3,4-d]pyrimidin-4-yl)-1-(2-fluoroacryloyl)piperazin-2-yl)acetonitrile; |
| 82. | (E)-2-(1-(but-2-enoyl)-4-(2-((1-((dimethylamino)methyl)cyclopropyl)methoxy)-7-(2,3- |
| | dimethylphenyl)-5,6,7,8-tetrahydropyrido[3,4-d]pyrimidin-4-yl)piperazin-2-yl)acetonitrile; |
| 83. | (S)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(2-fluoroacryloyl)piperazin-2-yl)acetonitrile; |
| 84. | (S,E)-2-(4-(7-(8-chloronaphthalen-1-yl)-2-((1-((dimethylamino)methyl)cyclopropyl)methoxy)- |
| | 5,6,7,8-tetrahydropyrido[3,4-d]pyrimidin-4-yl)-1-(4-fluorobut-2-enoyl)piperazin-2-yl)acetonitrile; |
| 85. | (E)-2-(1-(4-(dimethylamino)but-2-enoyl)-4-(2-((1-((dimethylamino)methyl)cyclopropyl)methoxy)- |
| | 7-(8-methylnaphthalen-1-yl)-5,6,7,8-tetrahydropyrido[3,4-d]pyrimidin-4-yl)piperazin-2- |
| | yl)acetonitrile; |
| 86. | (E)-2-(1-(4-(dimethylamino)but-2-enoyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile. |
| 87. | (S)-2-(1-acryloyl-4-(7-(3-fluoro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 88. | (S)-2-(1-acryloyl-4-(7-(2,3-dichlorophenyl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 89. | (S)-2-(1-acryloyl-4-(7-(3-chloro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 90. | 2-(1-acryloyl-4-(7-(5-chloro-4-(trifluoromethyl)pyridin-3-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 91. | (S)-2-(1-acryloyl-4-(7-(5-chloro-4-(trifluoromethyl)pyridin-3-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 92. | (S)-2-(1-(2-fluoroacryloyl)-4-(2-((1-(pyrrolidin-1-ylmethyl)cyclopropyl)methoxy)-7-(2- |
| | (trifluoromethyl)pyridin-3-yl)-5,6,7,8-tetrahydropyrido[3,4-d]pyrimidin-4-yl)piperazin-2- |
| | yl)acetonitrile; |
| 93. | (S)-2-(1-(2-fluoroacryloyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 94. | 2-(4-(7-(8-chloronaphthalen-1-yl)-2-((1-(pyrrolidin-1-ylmethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 95. | (S)-2-(4-(7-(8-chloronaphthalen-1-yl)-2-((1-(pyrrolidin-1-ylmethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 96. | 2-(1-acryloyl-4-(2-((1-((3,3-difluoropyrrolidin-1-yl)methyl)cyclopropyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 97. | 2-(1-acryloyl-4-(7-(3-chloro-2-(trifluoromethyl)phenyl)-2-((1-(((S)-3-fluoropyrrolidin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 98. | (S)-2-(4-(7-(8-chloronaphthalen-1-yl)-2-((1-(pyrrolidin-1-yl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 99. | (S)-2-(4-(7-(8-chloro-7-fluoronaphthalen-1-yl)-2-((1-(pyrrolidin-1-yl)cyclopropyl)methoxy)- |
| | 5,6,7,8-tetrahydropyrido[3,4-d]pyrimidin-4-yl)-1-(2-fluoroacryloyl)piperazin-2-yl)acetonitrile; |
| 100. | (S)-2-(1-acryloyl-4-(7-(3-methyl-2-(trifluoromethyl)phenyl)-2-((1-(pyrrolidin-1- |
| | yl)cyclopropyl)methoxy)-5,6,7,8-tetrahydropyrido[3,4-d]pyrimidin-4-yl)piperazin-2-yl)acetonitrile; |
| 101. | (S)-2-(1-acryloyl-4-(7-(3-chloro-2-(trifluoromethyl)phenyl)-2-((1-(pyrrolidin-1- |
| | yl)cyclopropyl)methoxy)-5,6,7,8-tetrahydropyrido[3,4-d]pyrimidin-4-yl)piperazin-2-yl)acetonitrile; |
| 102. | 2-((S)-1-acryloyl-4-(7-(2,3-dichlorophenyl)-2-((1-((S)-3-fluoropyrrolidin-1- |
| | yl)cyclopropyl)methoxy)-5,6,7,8-tetrahydropyrido[3,4-d]pyrimidin-4-yl)piperazin-2-yl)acetonitrile; |
| 103. | 2-(1-acryloyl-4-(2-((1-(azetidin-1-ylmethyl)cyclopropyl)methoxy)-7-(2,3-dichlorophenyl)-5,6,7,8- |
| | tetrahydropyrido[3,4-d]pyrimidin-4-yl)piperazin-2-yl)acetonitrile; |
| 104. | (S)-2-(1-acryloyl-4-(2-((1-(azetidin-1-ylmethyl)cyclopropyl)methoxy)-7-(3-chloro-2- |
| | (trifluoromethyl)phenyl)-5,6,7,8-tetrahydropyrido[3,4-d]pyrimidin-4-yl)piperazin-2-yl)acetonitrile; |
| 105. | (S)-2-(4-(2-((1-(azetidin-1-ylmethyl)cyclopropyl)methoxy)-7-(3-chloro-2-(trifluoromethyl)phenyl)- |
| | 5,6,7,8-tetrahydropyrido[3,4-d]pyrimidin-4-yl)-1-(2-fluoroacryloyl)piperazin-2-yl)acetonitrile; |
| 106. | (S)-2-(4-(2-((1-(azetidin-1-ylmethyl)cyclopropyl)methoxy)-7-(8-chloronaphthalen-1-yl)-5,6,7,8- |
| | tetrahydropyrido[3,4-d]pyrimidin-4-yl)-1-(2-fluoroacryloyl)piperazin-2-yl)acetonitrile; |
| 107. | (S)-2-(4-(2-((1-(azetidin-1-ylmethyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 108. | (S)-2-(1-acryloyl-4-(7-(8-methylnaphthalen-1-yl)-2-((1-(morpholinomethyl)cyclopropyl)methoxy)- |
| | 5,6,7,8-tetrahydropyrido[3,4-d]pyrimidin-4-yl)piperazin-2-yl)acetonitrile; |
| 109. | (S)-2-(4-(7-(8-chloronaphthalen-1-yl)-2-((1-(morpholinomethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 110. | 2-((S)-1-acryloyl-4-(7-(8-chloronaphthalen-1-yl)-2-(((1R,2S)-2- |
| | (dimethylamino)cyclobutyl)methoxy)-5,6,7,8-tetrahydropyrido[3,4-d]pyrimidin-4-yl)piperazin-2- |
| | yl)acetonitrile; |
| 111. | 2-((S)-4-(7-(8-chloronaphthalen-1-yl)-2-(((1S,2R)-2-(dimethylamino)cyclobutyl)methoxy)-5,6,7,8- |
| | tetrahydropyrido[3,4-d]pyrimidin-4-yl)-1-(2-fluoroacryloyl)piperazin-2-yl)acetonitrile; |
| 112. | 2-((S)-1-((E)-but-2-enoyl)-4-(7-(8-chloronaphthalen-1-yl)-2-(((1S,2S)-2- |
| | (dimethylamino)cyclobutyl)methoxy)-5,6,7,8-tetrahydropyrido[3,4-d]pyrimidin-4-yl)piperazin-2- |
| | yl)acetonitrile; |
| 113. | 2-((S)-4-(7-(8-chloronaphthalen-1-yl)-2-(((1R,2R)-2-(dimethylamino)cyclobutyl)methoxy)-5,6,7,8- |
| | tetrahydropyrido[3,4-d]pyrimidin-4-yl)-1-((E)-4-fluorobut-2-enoyl)piperazin-2-yl)acetonitrile; |
| 114. | 2-((S)-4-(7-(8-chloronaphthalen-1-yl)-2-(((1R,3S)-3-(dimethylamino)cyclopentyl)methoxy)- |
| | 5,6,7,8-tetrahydropyrido[3,4-d]pyrimidin-4-yl)-1-(2-fluoroacryloyl)piperazin-2-yl)acetonitrile; |
| 115. | 2-((S)-1-((E)-but-2-enoyl)-4-(7-(3-chloro-2-(trifluoromethyl)phenyl)-2-(((1S,3R)-3- |
| | (dimethylamino)cyclopentyl)methoxy)-5,6,7,8-tetrahydropyrido[3,4-d]pyrimidin-4-yl)piperazin-2- |
| | yl)acetonitrile; |
| 116. | 2-((S)-1-acryloyl-4-(2-(((1R,3R)-3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 117. | 2-((S)-4-(7-(2,3-dichlorophenyl)-2-(((1S,3S)-3-(dimethylamino)cyclopentyl)methoxy)-5,6,7,8- |
| | tetrahydropyrido[3,4-d]pyrimidin-4-yl)-1-((E)-4-fluorobut-2-enoyl)piperazin-2-yl)acetonitrile; |
| 118. | 2-((S)-1-acryloyl-4-(2-(((1R,3S)-3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 119. | 2-((S)-1-acryloyl-4-(2-(((1R,3R)-3-(dimethylamino)cyclopentyl)methoxy)-7-(4- |
| | (trifluoromethyl)pyridin-3-yl)-5,6,7,8-tetrahydropyrido[3,4-d]pyrimidin-4-yl)piperazin-2- |
| | yl)acetonitrile; |
| 120. | 2-((S)-4-(7-(5-chloro-4-(trifluoromethyl)pyridin-3-yl)-2-(((1S,3S)-3- |
| | (dimethylamino)cyclopentyl)methoxy)-5,6,7,8-tetrahydropyrido[3,4-d]pyrimidin-4-yl)-1-(2- |
| | fluoroacryloyl)piperazin-2-yl)acetonitrile; |
| 121. | 2-((S)-1-acryloyl-4-(7-(3-chloro-2-(trifluoromethyl)phenyl)-2-(((1S,3R)-3- |
| | (dimethylamino)cyclopentyl)methoxy)-5,6,7,8-tetrahydropyrido[3,4-d]pyrimidin-4-yl)piperazin-2- |
| | yl)acetonitrile; |
| 122. | 2-((S)-1-acryloyl-4-(7-(2,3-dichlorophenyl)-2-(((1R,3R)-3-(dimethylamino)cyclopentyl)methoxy)- |
| | 5,6,7,8-tetrahydropyrido[3,4-d]pyrimidin-4-yl)piperazin-2-yl)acetonitrile; |
| 123. | 2-((S)-1-acryloyl-4-(2-(((1R,3R)-3-(dimethylamino)cyclopentyl)methoxy)-7-(8-methylnaphthalen- |
| | 1-yl)-5,6,7,8-tetrahydropyrido[3,4-d]pyrimidin-4-yl)piperazin-2-yl)acetonitrile; |
| 124. | 2-((S)-4-(7-(8-chloronaphthalen-1-yl)-2-(((1S,3R)-3-(dimethylamino)cyclopentyl)methoxy)- |
| | 5,6,7,8-tetrahydropyrido[3,4-d]pyrimidin-4-yl)-1-(2-fluoroacryloyl)piperazin-2-yl)acetonitrile; |
| 125. | 2-(4-(7-(3-chloro-2-(trifluoromethyl)phenyl)-2-((1-((3-methoxypyrrolidin-1- |
| | yl)methyl)cyclopropyl)methoxy)-5,6,7,8-tetrahydropyrido[3,4-d]pyrimidin-4-yl)-1-(2- |
| | fluoroacryloyl)piperazin-2-yl)acetonitrile; |
| 126. | (E)-2-(1-(but-2-enoyl)-4-(7-(3-chloro-2-(trifluoromethyl)phenyl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 127. | 2-(4-(2-((1-((6-azaspiro[2.5]octan-6-yl)methyl)cyclopropyl)methoxy)-7-(3-chloro-2- |
| | methylphenyl)-5,6,7,8-tetrahydropyrido[3,4-d]pyrimidin-4-yl)-1-acryloylpiperazin-2- |
| | yl)acetonitrile; |
| 128. | 2-(1-acryloyl-4-(2-((1-((4-methoxypiperidin-1-yl)methyl)cyclopropyl)methoxy)-7-(4-methyl-3,4- |
| | dihydro-2H-benzo[b][1,4]oxazin-5-yl)-5,6,7,8-tetrahydropyrido[3,4-d]pyrimidin-4-yl)piperazin-2- |
| | yl)acetonitrile; |
| 129. | 2-(1-acryloyl-4-(2-((1-((4-methoxypiperidin-1-yl)methyl)cyclopropyl)methoxy)-7-(1- |
| | methylindolin-7-yl)-5,6,7,8-tetrahydropyrido[3,4-d]pyrimidin-4-yl)piperazin-2-yl)acetonitrile; |
| 130. | 2-(4-(2-((1-(((1R,4R)-2-oxa-5-azabicyclo[2.2.1]heptan-5-yl)methyl)cyclopropyl)methoxy)-7-(8- |
| | chloronaphthalen-1-yl)-5,6,7,8-tetrahydropyrido[3,4-d]pyrimidin-4-yl)-1-acryloylpiperazin-2- |
| | yl)acetonitrile; |
| 131. | 2-(1-acryloyl-4-(7-(3-chloro-2-(trifluoromethyl)phenyl)-2-(1-(2-(pyrrolidin-1- |
| | yl)ethyl)cyclopropoxy)-5,6,7,8-tetrahydropyrido[3,4-d]pyrimidin-4-yl)piperazin-2-yl)acetonitrile; |
| 132. | 2-(1-acryloyl-4-(7-(3-methyl-2-(trifluoromethyl)phenyl)-2-(2-(1-(pyrrolidin-1- |
| | yl)cyclopropyl)ethoxy)-5,6,7,8-tetrahydropyrido[3,4-d]pyrimidin-4-yl)piperazin-2-yl)acetonitrile; |
| 133. | 2-(1-acryloyl-4-(7-(3-chloro-2-(trifluoromethyl)phenyl)-2-((1-(1-methylpiperidin-3- |
| | yl)cyclopropyl)methoxy)-5,6,7,8-tetrahydropyrido[3,4-d]pyrimidin-4-yl)piperazin-2-yl)acetonitrile; |
| 134. | (E)-2-(1-(but-2-enoyl)-4-(7-(3-chloro-2-(trifluoromethyl)phenyl)-2-((1-((6-methyl-2,6- |
| | diazaspiro[3.3]heptan-2-yl)methyl)cyclopropyl)methoxy)-5,6,7,8-tetrahydropyrido[3,4- |
| | d]pyrimidin-4-yl)piperazin-2-yl)acetonitrile; |
| 135. | 2-(1-acryloyl-4-(7-(3-chloro-2-(trifluoromethyl)phenyl)-2-((1- |
| | ((diethylamino)methyl)cyclopropyl)methoxy)-5,6,7,8-tetrahydropyrido[3,4-d]pyrimidin-4- |
| | yl)piperazin-2-yl)acetonitrile; |
| 136. | 2-(1-acryloyl-4-(7-(8-chloronaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 137. | (S)-2-(1-acryloyl-4-(7-(8-chloronaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 138. | (S)-2-(1-acryloyl-4-(2-((1-(pyrrolidin-1-yl)cyclopropyl)methoxy)-7-(2-(trifluoromethyl)phenyl)- |
| | 5,6,7,8-tetrahydropyrido[3,4-d]pyrimidin-4-yl)piperazin-2-yl)acetonitrile; |
| 139. | (S)-2-(1-acryloyl-4-(2-((1-(3,3-difluoropyrrolidin-1-yl)cyclopropyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 140. | 2-((2S)-1-acryloyl-4-(7-(3-fluoro-2-(trifluoromethyl)phenyl)-2-((1-(1-methylpyrrolidin-2- |
| | yl)cyclopropyl)methoxy)-5,6,7,8-tetrahydropyrido[3,4-d]pyrimidin-4-yl)piperazin-2-yl)acetonitrile; |
| 141. | 2-((2S)-1-acryloyl-4-(7-(3-chloro-2-(trifluoromethyl)phenyl)-2-((1-(4-hydroxy-1-methylpyrrolidin- |
| | 2-yl)cyclopropyl)methoxy)-5,6,7,8-tetrahydropyrido[3,4-d]pyrimidin-4-yl)piperazin-2- |
| | yl)acetonitrile; |
| 142. | 2-((2S)-1-acryloyl-4-(7-(2,3-dichlorophenyl)-2-((2-methyl-1-(1-methylpyrrolidin-2- |
| | yl)cyclopropyl)methoxy)-5,6,7,8-tetrahydropyrido[3,4-d]pyrimidin-4-yl)piperazin-2-yl)acetonitrile; |
| 143. | 2-(1-acryloyl-4-(7-(2,3-dichlorophenyl)-2-(1-(pyrrolidin-1-ylmethyl)cyclopropoxy)-5,6,7,8- |
| | tetrahydropyrido[3,4-d]pyrimidin-4-yl)piperazin-2-yl)acetonitrile; |
| 144. | 2-(1-acryloyl-4-(2-(1-((dimethylamino)methyl)cyclopropoxy)-7-(naphthalen-1-yl)-5,6,7,8- |
| | tetrahydropyrido[3,4-d]pyrimidin-4-yl)piperazin-2-yl)acetonitrile; |
| 145. | 2-(1-acryloyl-4-(7-(8-methylnaphthalen-1-yl)-2-(1-(1-methylpyrrolidin-2-yl)cyclopropoxy)-5,6,7,8- |
| | tetrahydropyrido[3,4-d]pyrimidin-4-yl)piperazin-2-yl)acetonitrile; |
| 146. | 2-((2S)-4-(7-(8-chloronaphthalen-1-yl)-2-(1-(4,4-difluoro-1-methylpyrrolidin-2-yl)cyclopropoxy)- |
| | 5,6,7,8-tetrahydropyrido[3,4-d]pyrimidin-4-yl)-1-(2-fluoroacryloyl)piperazin-2-yl)acetonitrile; |
| 147. | 2-((2S)-1-acryloyl-4-(7-(2-chlorophenyl)-2-(1-(4-hydroxy-1-methylpyrrolidin-2-yl)-2,2- |
| | dimethylcyclopropoxy)-5,6,7,8-tetrahydropyrido[3,4-d]pyrimidin-4-yl)piperazin-2-yl)acetonitrile; |
| 148. | 2-((2S)-4-(7-(2-chloro-3-fluorophenyl)-2-(1-(4-methoxy-1-methylpyrrolidin-2-yl)cyclopropoxy)- |
| | 5,6,7,8-tetrahydropyrido[3,4-d]pyrimidin-4-yl)-1-((E)-4-(dimethylamino)but-2-enoyl)piperazin-2- |
| | yl)acetonitrile; |
| 149. | 2-((2S)-4-(2-(2-(dimethylamino)cyclopropoxy)-7-(naphthalen-1-yl)-5,6,7,8-tetrahydropyrido[3,4- |
| | d]pyrimidin-4-yl)-1-(2-fluoroacryloyl)piperazin-2-yl)acetonitrile; |
| 150. | 2-((2S)-1-((E)-but-2-enoyl)-4-(2-(2-((dimethylamino)methyl)cyclopropoxy)-7-(8- |
| | methylnaphthalen-1-yl)-5,6,7,8-tetrahydropyrido[3,4-d]pyrimidin-4-yl)piperazin-2-yl)acetonitrile; |
| 151. | 2-((2S)-1-acryloyl-4-(2-((2-(dimethylamino)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 152. | 2-((2S)-1-acryloyl-4-(7-(3-chloro-2-fluorophenyl)-2-((2- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 153. | 2-((2S)-1-acryloyl-4-(2-((1R)-1-(2-(pyrrolidin-1-ylmethyl)cyclopropyl)ethoxy)-7-(o-tolyl)-5,6,7,8- |
| | tetrahydropyrido[3,4-d]pyrimidin-4-yl)piperazin-2-yl)acetonitrile; |
| 154. | 2-((2S)-1-acryloyl-4-(7-(3-fluoro-2-methylphenyl)-2-((1R)-1-(2-((1-methylpyrrolidin-2- |
| | yl)methyl)cyclopropyl)ethoxy)-5,6,7,8-tetrahydropyrido[3,4-d]pyrimidin-4-yl)piperazin-2- |
| | yl)acetonitrile; |
| 155. | (S)-2-(1-acryloyl-4-(7-(2,3-dichlorophenyl)-2-(1-(pyrrolidin-1-ylmethyl)cyclobutoxy)-5,6,7,8- |
| | tetrahydropyrido[3,4-d]pyrimidin-4-yl)piperazin-2-yl)acetonitrile; |
| 156. | 2-((2S)-1-acryloyl-4-(7-(3-chloro-2-(trifluoromethyl)phenyl)-2-(1-((1-methylpyrrolidin-2- |
| | yl)methyl)cyclobutoxy)-5,6,7,8-tetrahydropyrido[3,4-d]pyrimidin-4-yl)piperazin-2-yl)acetonitrile; |
| 157. | 2-((2S)-1-acryloyl-4-(7-(3-chloro-2-(trifluoromethyl)phenyl)-2-(1-(1-methylpyrrolidin-2- |
| | yl)cyclobutoxy)-5,6,7,8-tetrahydropyrido[3,4-d]pyrimidin-4-yl)piperazin-2-yl)acetonitrile; |
| 158. | 2-((2S)-1-acryloyl-4-(2-(2-(dimethylamino)cyclobutoxy)-7-(2-(trifluoromethyl)phenyl)-5,6,7,8- |
| | tetrahydropyrido[3,4-d]pyrimidin-4-yl)piperazin-2-yl)acetonitrile; |
| 159. | (S)-2-(1-acryloyl-4-(2-(3-(ethyl(methyl)amino)cyclobutoxy)-7-(naphthalen-1-yl)-5,6,7,8- |
| | tetrahydropyrido[3,4-d]pyrimidin-4-yl)piperazin-2-yl)acetonitrile; |
| 160. | (S)-2-(1-acryloyl-4-(2-(3-(ethyl(2-methoxyethyl)amino)cyclobutoxy)-7-(naphthalen-1-yl)-5,6,7,8- |
| | tetrahydropyrido[3,4-d]pyrimidin-4-yl)piperazin-2-yl)acetonitrile; |
| 161. | 2-((2S)-1-acryloyl-4-(2-(3-(dimethylamino)-2-(2-methoxyethyl)cyclobutoxy)-7-(naphthalen-1-yl)- |
| | 5,6,7,8-tetrahydropyrido[3,4-d]pyrimidin-4-yl)piperazin-2-yl)acetonitrile; |
| 162. | (S)-2-(1-acryloyl-4-(7-(naphthalen-1-yl)-2-(3-(pyrrolidin-1-yl)cyclobutoxy)-5,6,7,8- |
| | tetrahydropyrido[3,4-d]pyrimidin-4-yl)piperazin-2-yl)acetonitrile; |
| 163. | (S)-2-(1-acryloyl-4-(7-(2-chlorophenyl)-2-((1-(pyrrolidin-1-yl)cyclobutyl)methoxy)-5,6,7,8- |
| | tetrahydropyrido[3,4-d]pyrimidin-4-yl)piperazin-2-yl)acetonitrile; |
| 164. | 2-((2S)-1-acryloyl-4-(2-((1-(1-methylpyrrolidin-3-yl)cyclobutyl)methoxy)-7-(2- |
| | (trifluoromethyl)phenyl)-5,6,7,8-tetrahydropyrido[3,4-d]pyrimidin-4-yl)piperazin-2-yl)acetonitrile; |
| 165. | 2-((2S)-1-acryloyl-4-(2-((1-(1-methylpyrrolidin-2-yl)cyclobutyl)methoxy)-7-(2- |
| | (trifluoromethyl)phenyl)-5,6,7,8-tetrahydropyrido[3,4-d]pyrimidin-4-yl)piperazin-2-yl)acetonitrile; |
| 166. | 2-((2S)-1-acryloyl-4-(2-((2-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 167. | (S)-2-(1-acryloyl-4-(7-(3-chloro-2-(trifluoromethyl)phenyl)-2-((3- |
| | (dimethylamino)cyclobutyl)methoxy)-5,6,7,8-tetrahydropyrido[3,4-d]pyrimidin-4-yl)piperazin-2- |
| | yl)acetonitrile; |
| 168. | 2-((S)-1-acryloyl-4-(2-((R)-1-(3-(dimethylamino)cyclobutyl)ethoxy)-7-(3-fluoro-2- |
| | (trifluoromethyl)phenyl)-5,6,7,8-tetrahydropyrido[3,4-d]pyrimidin-4-yl)piperazin-2-yl)acetonitrile; |
| 169. | (S)-2-(1-acryloyl-4-(7-(2-chlorophenyl)-2-((1-(dimethylamino)cyclopentyl)methoxy)-5,6,7,8- |
| | tetrahydropyrido[3,4-d]pyrimidin-4-yl)piperazin-2-yl)acetonitrile; |
| 170. | 2-((2S)-1-acryloyl-4-(7-(2-chlorophenyl)-2-((1-(dimethylamino)-3- |
| | (trifluoromethyl)cyclopentyl)methoxy)-5,6,7,8-tetrahydropyrido[3,4-d]pyrimidin-4-yl)piperazin-2- |
| | yl)acetonitrile; |
| 171. | 2-((2S)-1-acryloyl-4-(7-(2-chlorophenyl)-2-((1-(dimethylamino)-3- |
| | (trifluoromethoxy)cyclopentyl)methoxy)-5,6,7,8-tetrahydropyrido[3,4-d]pyrimidin-4-yl)piperazin- |
| | 2-yl)acetonitrile; |
| 172. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentane-1-carbonitrile; |
| 173. | ethyl 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentane-1-carboxylate; |
| 174. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N,N-dimethylcyclopentane- |
| | 1-carboxamide; |
| 175. | 2-((2S)-1-acryloyl-4-(7-(2-chlorophenyl)-2-((1-(dimethylamino)-3-(pyrrolidine-1- |
| | carbonyl)cyclopentyl)methoxy)-5,6,7,8-tetrahydropyrido[3,4-d]pyrimidin-4-yl)piperazin-2- |
| | yl)acetonitrile; |
| 176. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N-methylcyclopentane-1- |
| | carboxamide; |
| 177. | 2-((2S)-1-acryloyl-4-(7-(2-chlorophenyl)-2-((1-(dimethylamino)-3-(thiazol-2- |
| | yl)cyclopentyl)methoxy)-5,6,7,8-tetrahydropyrido[3,4-d]pyrimidin-4-yl)piperazin-2-yl)acetonitrile; |
| 178. | (S)-2-(1-acryloyl-4-(7-(2,3-difluorophenyl)-2-((1-(pyrrolidin-1-yl)cyclopentyl)methoxy)-5,6,7,8- |
| | tetrahydropyrido[3,4-d]pyrimidin-4-yl)piperazin-2-yl)acetonitrile; |
| 179. | 2-((2S)-1-acryloyl-4-(2-((1-(1-methylpyrrolidin-2-yl)cyclopentyl)methoxy)-7-(naphthalen-1-yl)- |
| | 5,6,7,8-tetrahydropyrido[3,4-d]pyrimidin-4-yl)piperazin-2-yl)acetonitrile; |
| 180. | 2-((2S)-1-acryloyl-4-(7-(2-chloro-3-fluorophenyl)-2-((2,2-difluoro-1-(1-methylpyrrolidin-2- |
| | yl)cyclopentyl)methoxy)-5,6,7,8-tetrahydropyrido[3,4-d]pyrimidin-4-yl)piperazin-2-yl)acetonitrile; |
| 181. | 2-((2S)-1-acryloyl-4-(7-(2-chloro-3-fluorophenyl)-2-((1-(1-methylpyrrolidin-3- |
| | yl)cyclopentyl)methoxy)-5,6,7,8-tetrahydropyrido[3,4-d]pyrimidin-4-yl)piperazin-2-yl)acetonitrile; |
| 182. | 2-((2S)-1-acryloyl-4-(7-(2,3-dichlorophenyl)-2-((1-(1-isopropylpyrrolidin-3- |
| | yl)cyclopentyl)methoxy)-5,6,7,8-tetrahydropyrido[3,4-d]pyrimidin-4-yl)piperazin-2-yl)acetonitrile; |
| 183. | (S)-2-(1-acryloyl-4-(7-(2-chlorophenyl)-2-((1-((dimethylamino)methyl)cyclopentyl)oxy)-5,6,7,8- |
| | tetrahydropyrido[3,4-d]pyrimidin-4-yl)piperazin-2-yl)acetonitrile; |
| 184. | (S)-2-(1-acryloyl-4-(2-((1-(azetidin-1-ylmethyl)cyclopentyl)oxy)-7-(2-(trifluoromethyl)phenyl)- |
| | 5,6,7,8-tetrahydropyrido[3,4-d]pyrimidin-4-yl)piperazin-2-yl)acetonitrile; |
| 185. | (S)-2-(1-acryloyl-4-(7-(3-fluoro-2-(trifluoromethyl)phenyl)-2-((1-(pyrrolidin-1- |
| | ylmethyl)cyclopentyl)oxy)-5,6,7,8-tetrahydropyrido[3,4-d]pyrimidin-4-yl)piperazin-2- |
| | yl)acetonitrile; |
| 186. | 2-((2S)-1-acryloyl-4-(7-(3-chloro-2-(trifluoromethyl)phenyl)-2-((1-((1-methylpyrrolidin-2- |
| | yl)methyl)cyclopentyl)oxy)-5,6,7,8-tetrahydropyrido[3,4-d]pyrimidin-4-yl)piperazin-2- |
| | yl)acetonitrile; |
| 187. | 2-((2S)-1-acryloyl-4-(2-((1-((4,4-difluoro-1-methylpyrrolidin-2-yl)methyl)cyclopentyl)oxy)-7- |
| | (naphthalen-1-yl)-5,6,7,8-tetrahydropyrido[3,4-d]pyrimidin-4-yl)piperazin-2-yl)acetonitrile; |
| 188. | 2-((2S)-1-acryloyl-4-(2-((1-(difluoro(1-methylpyrrolidin-2-yl)methyl)cyclopentyl)oxy)-7-(3- |
| | methyl-2-(trifluoromethyl)phenyl)-5,6,7,8-tetrahydropyrido[3,4-d]pyrimidin-4-yl)piperazin-2- |
| | yl)acetonitrile; |
| 189. | 2-((2S)-1-acryloyl-4-(2-((2-(dimethylamino)cyclopentyl)oxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 190. | 2-((2S)-1-acryloyl-4-(2-((3-(dimethylamino)cyclopentyl)oxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 191. | 2-((2S)-1-acryloyl-4-(2-((3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 192. | 2-((2S)-1-acryloyl-4-(2-((2-(aimetnylamino)cyclopentyl)methoxy)-7-(3-metnyl-2- |
| | (trifluoromethyl)phenyl)-5,6,7,8-tetrahydropyrido[3,4-d]pyrimidin-4-yl)piperazin-2-yl)acetonitrile; |
| 193. | (S)-2-(1-acryloyl-4-(7-(2-chlorophenyl)-2-((1-((dimethylamino)methyl)cyclohexyl)oxy)-5,6,7,8- |
| | tetrahydropyrido[3,4-d]pyrimidin-4-yl)piperazin-2-yl)acetonitrile; |
| 194. | (S)-2-(1-acryloyl-4-(2-((1-(pyrrolidin-1-ylmethyl)cyclohexyl)oxy)-7-(2-(trifluoromethyl)phenyl)- |
| | 5,6,7,8-tetrahydropyrido[3,4-d]pyrimidin-4-yl)piperazin-2-yl)acetonitrile; |
| 195. | 2-((2S)-1-acryloyl-4-(7-(2-chloro-3-fluorophenyl)-2-((2-(dimethylamino)cyclohexyl)oxy)-5,6,7,8- |
| | tetrahydropyrido[3,4-d]pyrimidin-4-yl)piperazin-2-yl)acetonitrile; |
| 196. | 2-((2S)-1-acryloyl-4-(7-(2,3-dichlorophenyl)-2-((2-(1-methylazetidin-2-yl)cyclohexyl)oxy)-5,6,7,8- |
| | tetrahydropyrido[3,4-d]pyrimidin-4-yl)piperazin-2-yl)acetonitrile; |
| 197. | 2-((2S)-1-acryloyl-4-(7-(3-chloro-2-(trifluoromethyl)phenyl)-2-((2- |
| | (dimethylamino)cyclohexyl)methoxy)-5,6,7,8-tetrahydropyrido[3,4-d]pyrimidin-4-yl)piperazin-2- |
| | yl)acetonitrile; |
| 198. | 2-((2S)-1-acryloyl-4-(2-((2-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 199. | (S)-2-(1-acryloyl-4-(7-(2-chlorophenyl)-2-((1-(dimethylamino)cyclohexyl)methoxy)-5,6,7,8- |
| | tetrahydropyrido[3,4-d]pyrimidin-4-yl)piperazin-2-yl)acetonitrile; |
| 200. | (S)-2-(1-acryloyl-4-(7-(2,3-difluorophenyl)-2-((1-((dimethylamino)methyl)cyclohexyl)methoxy)- |
| | 5,6,7,8-tetrahydropyrido[3,4-d]pyrimidin-4-yl)piperazin-2-yl)acetonitrile; |
| 201. | (S)-2-(1-acryloyl-4-(2-((4-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 202. | 2-((2S)-1-acryloyl-4-(7-(3-chloro-2-(trifluoromethyl)phenyl)-2-((2-(pyrrolidin-1- |
| | yl)cyclohexyl)methoxy)-5,6,7,8-tetrahydropyrido[3,4-d]pyrimidin-4-yl)piperazin-2-yl)acetonitrile; |
| 203. | 2-((2S)-1-acryloyl-4-(7-(3-chloro-2-(trifluoromethyl)phenyl)-2-((3- |
| | (dimethylamino)cyclohexyl)methoxy)-5,6,7,8-tetrahydropyrido[3,4-d]pyrimidin-4-yl)piperazin-2- |
| | yl)acetonitrile; |
| 204. | 2-((2S)-1-acryloyl-4-(7-(3-chloro-2-(trifluoromethyl)phenyl)-2-((3- |
| | (dimethylamino)cyclohexyl)oxy)-5,6,7,8-tetrahydropyrido[3,4-d]pyrimidin-4-yl)piperazin-2- |
| | yl)acetonitrile; |
| 205. | N-(3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentyl)acetamide; |
| 206. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N,N-dimethylcyclopentane- |
| | 1-sulfonamide; |
| 207. | 2-((2S)-1-acryloyl-4-(2-(((2-(dimethylamino)cyclobutyl)methyl)thio)-7-(naphthalen-1-yl)-5,6,7,8- |
| | tetrahydropyrido[3,4-d]pyrimidin-4-yl)piperazin-2-yl)acetonitrile; or |
| 208. | 2-((2S)-1-acryloyl-4-(2-(((2-(dimethylamino)cyclobutyl)methyl)amino)-7-(naphthalen-1-yl)- |
| | 5,6,7,8-tetrahydropyrido[3,4-d]pyrimidin-4-yl)piperazin-2-yl)acetonitrile. |

In some embodiments, the compound is 2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

In some embodiments, the compound is (S,E)-2-(4-(2-((1-((dimethylamino)methyl)cyclopropyl) methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile.

In some embodiments, the compound is (S)-2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

In some embodiments, the compound is (S)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(2-fluoroacryloyl)piperazin-2-yl)acetonitrile.

In another aspect, there is provided a pharmaceutical composition comprising at least one compound of formula (I), a pharmaceutically acceptable salt thereof or stereoisomer thereof of the present invention, and at least one pharmaceutically acceptable excipient. In some embodiments, the said compound in a weight ratio to the said excipient within the range from about 0.0001 to about 10. In some embodiments, the said compound in a weight ratio to the said excipient within the range from about 0.01 to about 0.8. In some embodiments, the said compound in a weight ratio to the said excipient within the range from about 0.02 to about 0.2. In some embodiments, the said compound in a weight ratio to the said excipient within the range from about 0.05 to about 0.15. In some embodiments, the compound is 2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile. In some embodiments, the compound is (S,E)-2-(4-(2-((1-((dimethylamino)methyl)cyclopropyl) methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile. In some embodiments, the compound is (S)-2-(1-acryloyl-4-(7-(8-methyl naphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile. In some embodiments, the compound is (S)-2-(4-(2-((1-((dimethylamino) methyl)cyclopropyl)methoxy)-7-(8-methylnaphthalen-1-yl)-5,6,7,8-tetrahydropyrido[3,4-d]pyrimidin-4-yl)-1-(2-fluoroacryloyl)piperazin-2-yl)acetonitrile.

In another aspect, there is provided use of the compound of formula I, a pharmaceutically acceptable salt thereof or stereoisomer thereof of the present invention; or the pharmaceutical composition of the present invention for the manufacture of a medicament for the treatment of diseases or conditions related to KRAS mutant protein. In some embodiments, the diseases or conditions related to KRAS mutant protein is the diseases or conditions related to KRAS G12C mutant protein. In some embodiments, the diseases or conditions related to KRAS mutant protein is cancer related to KRAS G12C mutant protein. In some embodiments, the cancer is selected from blood cancer, pancreatic cancer, colon cancer, rectal cancer, colorectal cancer or lung cancer. In some embodiments, the blood cancer is selected from acute myeloid leukemia or acute lymphocytic leukemia; the lung cancer is selected from non-small cell lung cancer or small cell lung cancer.

In another aspect, there is provided a method of treating a subject having a diseases or conditions related to KRAS mutant protein, said method comprising administering to the subject a therapeutically effective amount of at least one compound of formula (I), a pharmaceutically acceptable salt thereof or stereoisomer thereof of the present invention; or the pharmaceutical composition of the present invention. In some embodiments, the diseases or conditions related to KRAS mutant protein is the diseases or conditions related to KRAS G12C mutant protein. In some embodiments, the diseases or conditions related to KRAS mutant protein is cancer related to KRAS G12C mutant protein. In some embodiments, the cancer is selected from blood cancer, pancreatic cancer, colon cancer, rectal cancer, colorectal cancer or lung cancer. In some embodiments, the blood cancer is selected from acute myeloid leukemia or acute lymphocytic leukemia; the lung cancer is selected from non-small cell lung cancer or small cell lung cancer. In some embodiments, the compound is 2-(1-acryloyl-4-(7-(8-methylnaphthalen-1-yl)-2-((1-(pyrrolidin-1-ylmethyl)cyclopropyl) methoxy)-5,6,7,8-tetrahydropyrido[3,4-d]pyrimidin-4-yl)piperazin-2-yl)acetonitrile. In some embodiments, the compound is (S,E)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile. In some embodiments, the compound is (S)-2-(1-acryloyl-4-(7-(8-methylnaphthalen-1-yl)-2-((1-(pyrrolidin-1-ylmethyl) cyclopropyl)methoxy)-5,6,7,8-tetrahydropyrido[3,4-d]pyrimidin-4-yl)piperazin-2-yl)acetonitrile. In some embodiments, the compound is (S)-2-(4-(2-((1-((dimethylamino)methyl)cyclopropyl)methoxy)-7-(8-methyl naphthalen-1-yl)-5,6,7,8-tetrahydropyrido[3,4-d]pyrimidin-4-yl)-1-(2-fluoroacryloyl)piperazin-2-yl)acetonitrile.

### Definition

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, alkyl radicals include methyl, ethyl, propyl, isopropyl, cyclopropyl, n-butyl, isobutyl, sec-butyl, t-butyl, cyclobutyl, n-pentyl, 3-(2-methyl) butyl, 2-pentyl, 2-methylbutyl, neopentyl, cyclcopentyl, n-hexyl, 2-hexyl, 2-methylpentyl and cyclohexyl. Similarly, C₁-6, as in C₁₋₆alkyl is defined to identify the group as having 1, 2, 3, 4, 5 or 6 carbon atoms in a linear or branched arrangement.

The term “alkylene” means a difunctional group obtained by removal of a hydrogen atom from an alkyl group that is defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, heptenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The two adjacent substituents of the aryl can form a C₃₋₆ carbocyclic or C₃₋₆ heterocyclic ring defined in the present invention. The preferred aryls are mono cyclic or bicyclic 6-10 membered aromatic ring systems. Phenyl and naphthyl are preferred aryls. The most preferred aryl is phenyl.

The term “heterocyclic”, as used herein, unless otherwise indicated, refers to unsubstituted and substituted mono or polycyclic non-aromatic ring system containing one or more heteroatoms. Preferred heteroatoms include N, O, and S, including N-oxides, sulfur oxides, and dioxides. Preferably the ring is three to eight membered and is either fully saturated or has one or more degrees of unsaturation. Multiple degrees of substitution, preferably one, two or three, are included within the present definition.

Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. The two adjacent substituents of the heteroaryl can form a C₃₋₆ carbocyclic or C₃₋₆ heterocyclic ring defined in the present invention. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “carbocyclic” refers to a substituted or unsubstituted monocyclic, bicyclic or polycyclic non-aromatic saturated ring, which optionally includes an alkylene linker through which the cycloalkyl may be attached. Exemplary “cycloalkyl” groups includes but not limited to, cyclopropyl, cyclobutyl, cyclopentyl, cyclohexyl and so on.

The term “oxo” refers to oxygen atom together with the attached carbon atom.forms the group

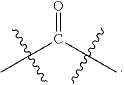 [SMILES: *C(*)=O]

The term “carboxyl” refers to the group C(O)OH.

The term “—C₁₋₆alkyleneC₆₋₁₀aryl” refers to the —C₁₋₆alkyl as defined above substituted by C₆₋₁₀aryl as defined above.

The term “—C₁₋₆alkylene-(5-10 membered heteroaryl)” refers to the —C₁₋₆alkyl as defined above substituted by 5-10 membered heteroaryl as defined above.

The term “—C₁₋₆alkylene-(3-10 membered heterocyclic)” refers to the —C₁₋₆alkyl as defined above substituted by 3-10 membered heterocyclic as defined above.

The term “—C₁₋₆alkylene-C₃₋₁₀carbocyclic” refers to the —C₁₋₆alkyl as defined above substituted by C₃₋₁₀carbocyclic as defined above.

The term “—C₁₋₆alkylene-(halo)₁₋₃” refers to the —C₁₋₆alkyl as defined above substituted by 1, 2 or 3 halogen as defined above.

The term “heteroC₁₋₆alkyl” refers to the C₁₋₆alkyl as defined above wherein one or more carbon atoms in the chain are replaced by a heteroatom selected from 0, S or N.

The term “—C₁₋₆alkylene-(OR₈)₁₋₃” refers to the —C₁₋₆alkyl as defined above substituted by 1, 2 or 3 OR₈, wherein R₈ is defined as above, preferred R₈ is selected from hydrogen, methyl, ethyl or propyl.

The term “—O—C₁₋₆alkylene-(halo)₁₋₃” refers to the oxygen ethers of —C₁₋₆alkylene-(halo)₁₋₃ as defined above.

The term “—S—C₁₋₆alkylene-(halo)₁₃” refers to the S ethers of —C₁₋₆alkylene-(halo)₁₋₃ as defined above.

The term “—C₁₋₆alkylene-NR₈R₉” refers to the —C₁₋₆alkyl as defined above substituted by —NR₈R₉, wherein the R₈ and R₉ is defined as above.

The term “−C₆alkylene-C(═O)NR₈R₉” refers to the —C₁₋₆alkyl as defined above substituted by —C(═O)NR₈R₉, wherein the R₈ and R₉ is defined as above.

The term “−C₆alkylene-NR₈C(═O)R₈” refers to the —C₁₋₆alkyl as defined above substituted by —NR₈C(═O)R₈.

The term “—C₁₋₆alkylene-CN” refers to the —C₁₋₆alkyl as defined above substituted by —CN.

The term“composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The invention includes all possible stereoisomers of the compound.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H(hydrogen), ²H(deuterium) and ³H(tritium). They are also commonly denoted as D for deuterium and T for tritium. In the application, CD₃ denotes a methyl group wherein all of the hydrogen atom are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

When a tautomer of the compound of Formula (I) exists, the present invention includes any possible tautomers and pharmaceutically acceptable salts thereof, and mixtures thereof, except where specifically stated otherwise.

When the compound of Formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds of Formula (I) are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt, of Formula I. The compounds of Formula I or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

### BRIEF DESCRIPTION OF THE FIGURES

FIG. 1 is a graph showing the tumor volume of mice varies with the number of days after cell inoculation in the experiment of using compound 56, compound 68, compound 83, and MRTX849 to inhibit tumor growth.

FIG. 2 shows the body weight of mice varies with the number of days after cell inoculation in the experiment of using compound 56, compound 68, compound 83, and MRTX849 to inhibit tumor growth.

FIG. 3 is a graph showing the tumor volume of mice varies with the number of days after cell inoculation in the experiment of using compound 67 and MRTX849 to inhibit tumor growth.

FIG. 4 is a graph showing the body weight of mice with the number of days after cell inoculation in the experiment of using compound 67 and MRTX849 to inhibit tumor growth.

### METHODS OF PREPARATION

Reaction Schemes

Compound (I) may be prepared from commercially available reagents using the synthetic methods and reaction schemes described herein, or using other reagents and conventional methods well known to those skilled in the art.

For instance, compounds in the present invention maybe prepared according to the general reaction schemes.

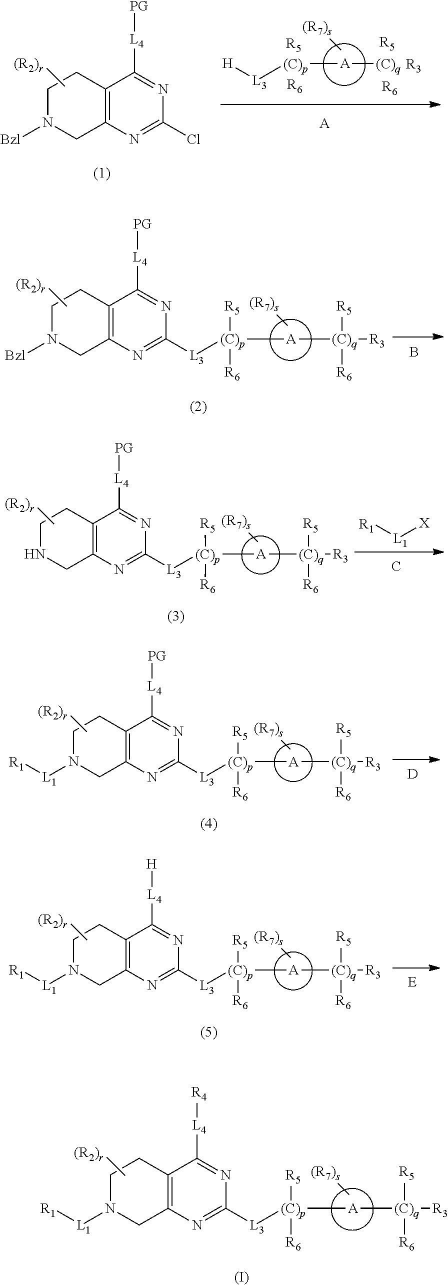

In step A, the substituent

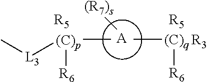

can be introduced by substitution of the chlorine by a nucleophile, for example trans-4-(dimethylamino)cyclohexanol in a solvent such as THF to provide compound (2); alternatively

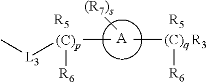

an be introduced by palladium coupling with compound (1), for example in the presence of a palladium catalyst such as Pd₂(dba)₃/BINAP in a solvent such as toluene with a base such as cesium carbonate or sodium tert-butoxide to provide compound (2).

In step B, benzyl group is removed using conditions known in the art, for example hydrogenolysis by Pd/C in the presence of H₂ in a polar solvent such as methanol to provide compound (3).

In step C, the substituent “-L₁-R₁” is introduced with palladium coupling, using a suitable functionalized aryl or heteroaryl system in the presence of a palladium catalyst such as Pd₂(dba)₃/Ruphos in a solvent such as toluene with a base such as cesium carbonate to provide compound (4).

In step D, the protecting group of L₄ is removed using conditions known in the art, for example with trifluoroacetic acid in the solvent such as dichloromethane to provide compound (5).

In step E, R₄ is introduced to provide a compound of formula (I), for example by treating with an acid chloride having the formula Cl—C(O)—CR═CR or an anhydride having the formula CR═CR—C(O)—O—C(O)—CR═CR in the presence of a solvent such as dichloromethane with a base such as Hunig base to provide a compound of formula (I).

In some cases, R₁ and

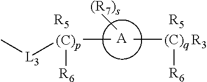

may also contain protecting groups, which can be removed at a subsequent step in the synthetic sequence.

### EXAMPLES

The following Examples are provided to better illustrate the present invention. All parts and percentages are by weight and all temperatures are degrees Celsius, unless explicitly stated otherwise. The following abbreviations have been used in the examples:

| | |
|---|---|
| MeOH | Methanol |
| DCE | 1,2-dichloroethane |
| Hex | hexane |
| THF | tetrahydrofuran |
| DMF | N,N-dimethylformamide |
| DCM | Dichloromethane |
| EtOAc | ethyl acetate |
| ACN | acetonitrile |
| TEA | Triethylamine |
| DIEA | N-ethyl-N-isopropylpropan-2-amine |
| Pd₂(dba)₃ | Tris(dibenzylideneacetone)dipalladium(0) |
| BINAP | Racemic-2,2′-Bis(diphenylphosphino)-1,1′-binaphthyl |
| RuPhos | Dicyclohexyl(2′,6′-diisopropoxy-[1,1′-biphenyl]-2- |
| | yl)phosphine |
| BOP | Benzotriazol-1-yloxytris(dimethylamino)phosphonium |
| | Hexafluorophosphate |
| HATU | 2-(7-Azabenzotriazol-1-yl)-N,N,N′,N′-tetramethyluronium |
| | hexafluorophosphate |
| DBU | 1,8-Diazabicyclo[5.4.0]undec-7-ene |
| NaBH₄ | Sodium borohydride |
| TFA | Trifluoroacetic acid |
| NaOMe | sodium methanolate |
| (Boc)₂O | di-tert-butyl dicarbonate |
| t-BuONa | sodium 2-methylpropan-2-olate |
| m-CPBA | 3-chlorobenzoperoxoic acid |
| K₂CO₃ | potassium carbonate |
| Na₂SO₄ | sodium sulfate |
| min | minute(s) |
| h | hour(s) |
| Pre-TLC | Preparative thin layer chromatography |

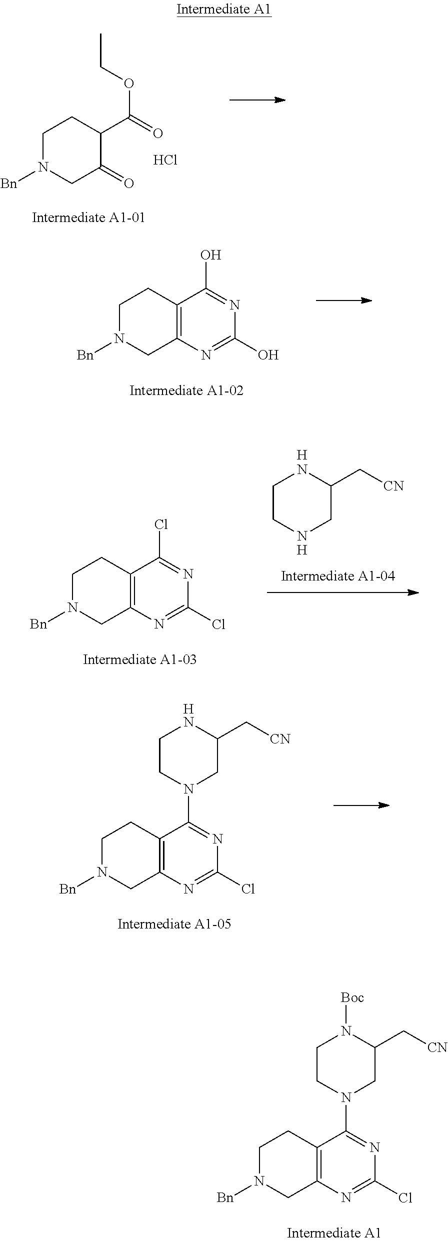

Following procedures of WO2017201161 A1, Intermediate A1 was prepared from Ethyl N-benzyl-3-oxo-4-piperidinecarboxylate hydrochloride in 4 steps.

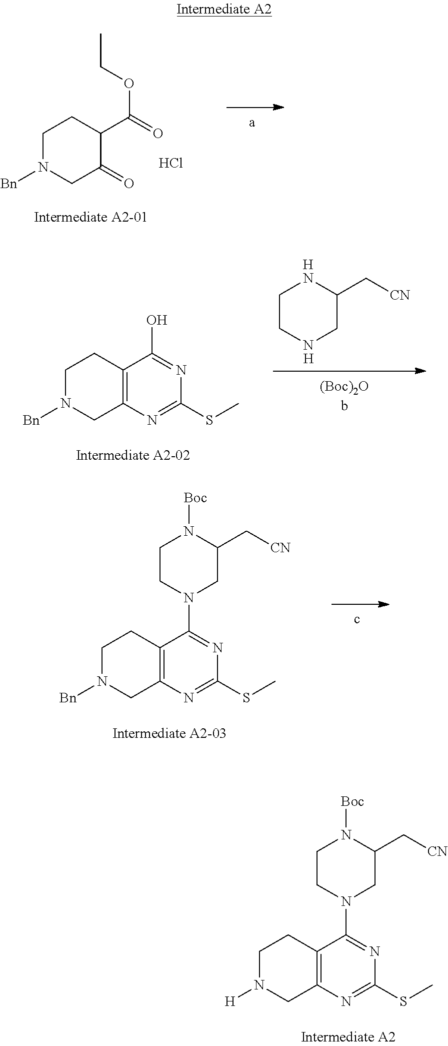

Step a: To a suspension of Intermediate A2-01 (59.46 g, 0.20 mol) and 2-methylisothiourea (57.21 g, 0.30 mol, 0.5 H₂SO₄) in MeOH (800 mL) was added NaOMe (54.22 g, 1.00 mol) at room temperature. The reaction mixture was stirred at room temperature for 26 hours. The mixture was concentrated under reduced pressure. The residue was suspended in water (500 mL) and acidified by con.HCl until pH=3-4. The suspension was filtered and the filter cake was dried under vacuum to give Intermediate A2-02 (56.00 g, 0.19 mol) as a light brown solid. MS: m/z 288 [M+H]⁺.

Step b: To a solution of Intermediate A2-02 (4.04 g, 14.06 mmol), 2-(piperazin-2-yl)acetonitrile (4.10 g, 20.70 mmol, 2HCl) and BOP (8.00 g, 18.09 mmol) in DMF (80 mL) was added DIEA (9.10 g, 70.41 mmol). The mixture was warmed to 70° C. and stirred for overnight. To the reaction mixture was added (Boc)₂O (9.30 g, 42.61 mmol) and stirred the mixture at room temperature for 4 hours. Water was added to the mixture and the mixture was extracted with EtOAc twice. The combined organic phase was washed with brine, dried over anhydrous Na₂SO₄, filtered and concentrated under reduced pressure. The residue was purified by silica gel chromatography (Hex:EtOAc from 50:1 to 2:1) to give Intermediate A2-03 (4.00 g, 8.09 mmol) as a yellow solid. MS: m/z 495 [M+H]⁺.

Step c: To a solution of Intermediate A2-03 (4.00 g, 8.09 mmol) and DIEA (3.15 g, 24.37 mmol) in DCE (100 mL) was added 1-chloroethyl carbonochloridate (2.90 g, 20.28 mmol) at 0° C. The mixture was stirred at room temperature for 4 hours. The reaction mixture was concentrated under reduced pressure. The residue was dissolved in MeOH (120 mL) and the mixture was stirred reflux for 1.5 hours. The reaction mixture was concentrated under reduced pressure. The residue was purified by silica gel chromatography (DCM:MeOH from 50:1 to 10:1) to give Intermediate A2 (3.15 g, 7.79 mmol, 96.3% yield) as a pink solid. MS: m/z 405 [M+H]⁺.

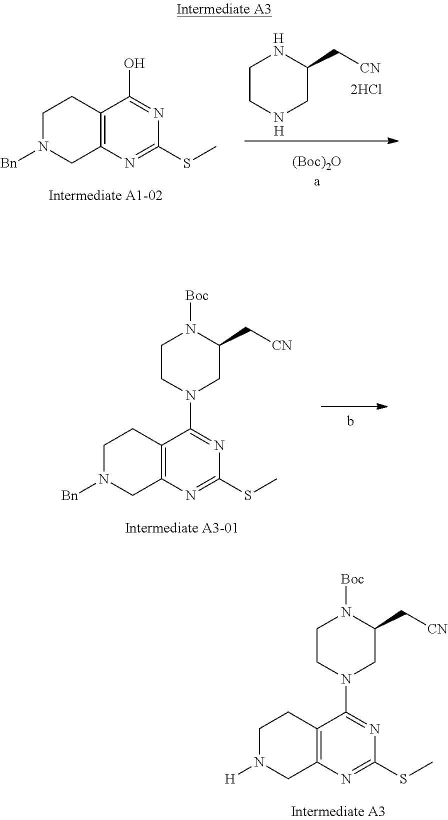

Step a: To a solution of Intermediate A1-02 (43.89 g, 152.73 mmol), (S)-2-(piperazin-2-yl)acetonitrile (32.63 g, 164.72 mmol, 2HCl) and BOP (89.37 g, 202.07 mmol) in DMF (80 mL) was added DBU (134.82 g, 885.60 mmol). The mixture was warmed to RT and stirred for 40 min. To the reaction mixture was added (Boc)₂O (68.84 g, 315.42 mmol) and stirred the mixture at 40° C. for overnight. Water was added to the mixture and the mixture was extracted with EtOAc twice. The combined organic phase was washed with brine, dried over anhydrous Na₂SO₄, filtered and concentrated under reduced pressure. The residue was purified by silica gel chromatography (Hex:EtOAc from 50:1 to 2:1) to give Intermediate A3-01 (43.59 g, 88.12 mmol) as a yellow solid. MS: m/z 495 [M+H]⁺.

Step b: To a solution of Intermediate A3-01 (36.60 g, 73.99 mmol) and DIEA (44.27 g, 342.54 mmol) in DCE (250 mL) was added 1-chloroethyl carbonochloridate (39.54 g, 276.56 mmol) at 0° C. The mixture was stirred at room temperature for 4 hours. The reaction mixture was concentrated under reduced pressure. The residue was dissolved in MeOH (250 mL) and the mixture was stirred reflux for 1.5 hours. The residue was dispersed with EA (200 mL) and extracted with 10% Citric acid solution (150 mL*2). The aqueous layer was adjusted to pH 12 with K₂CO₃ solid, extracted with EA (200 mL*2), dried over anhydrous Na₂SO₄, filtered and concentrated under reduced pressure to give Intermediate A3 (27.82 g). MS: m/z 405 [M+H]⁺.

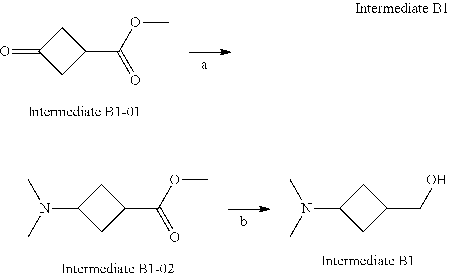

Step a: A solution of Intermediate B1-01 (2.11 g, 16.47 mmol), dimethylamine (2 M solution in THF, 20 mL, 40 mmol) in EA (20 mL) was stirred at RT for 1.25 h. Sodium triacetoxyborohydride (9.85 g, 46.28 mmol) was added, and stirred for 24 h at RT. The reaction mixture was filtered and concentrated under reduced pressure.

The residue was purified by silica chromatography (eluting with DCM:MeOH=10:1, v/v) to give Intermediate B1-02 (2.21 g). MS: m/z 158 [M+1]⁺.

Step b: To a 0° C. solution of Intermediate B1-02 (2.01 g, 12.79 mmol) in THF (10 mL) was added Lithium Aluminum Hydride (1.01 g, 14.85 mmol), stirred for 1.5 h at RT. The reaction mixture was quenched with ice water (20 mL), and filtered. The filtrate was saturated with K₂CO₃ solid, extracted with EA (3×20 mL), dried over anhydrous Na₂SO₄, filtered and concentrated under reduced pressure. The residue was purified by silica chromatography (eluting with DCM:MeOH=20:1, v/v) to give Intermediate B1 (653 mg). MS: m/z 130 [M+1]⁺.

The following intermediates were synthesized using the above procedure or modifications procedure with the corresponding starting materials.

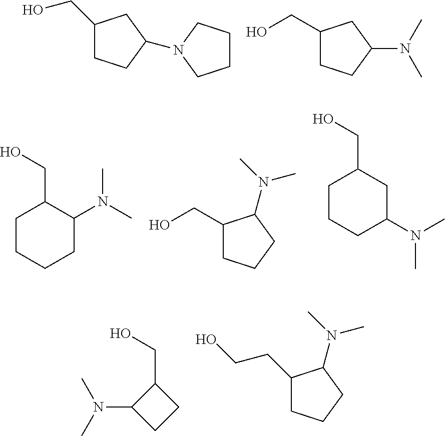

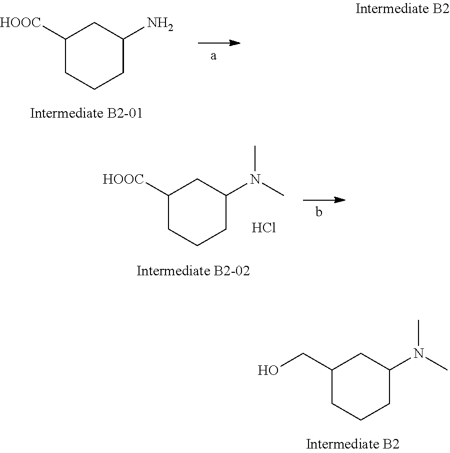

Step a: A solution of Intermediate B2-01 (1.29 g, 8.98 mmol), formalin (3.7 mL) and formic acid (6 mL) in 50 mL tube sealing was stirred at 110° C. for 19 h. After cooling to RT, concentrated hydrochloric acid (3 mL) was added, and concentrated under reduced pressure. The residue was dispersed with isopropyl alcohol (5 mL) and diethyl ether (10 mL), and placed in refrigerator for 1.5 h. The supernatant was discarded, and the sank residue was washed with Hex (2×5 mL). The residue was dried under vacuum to give Intermediate B2-02 (1328 mg). MS: m/z 172 [M+1]⁺

Step b: To a solution of Lithium Aluminum Hydride (1M solution in THF, 31 mL, 31 mmol) was added Intermediate B2-02 (1.18 g, 5.68), stirred for 2.5 h at RT. The reaction mixture was quenched with water (1.2 mL), sodium hydroxide aqueous solution (4 mol/L, 1.2 mL) and water (3.6 mL) and filtered. The filtrate was concentrated under reduced pressure. The residue was purified by silica chromatography (eluting with DCM:MeOH=4:1, v/v) to give Intermediate B2 (180 mg). MS: m/z 158 [M+1]⁺

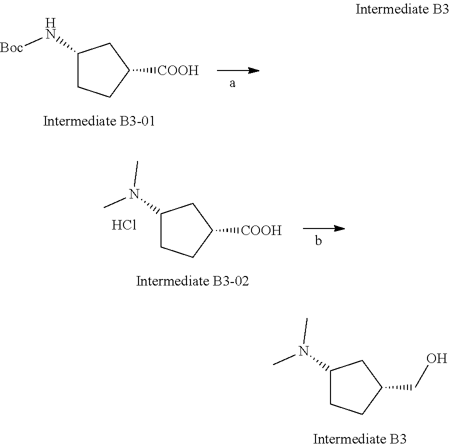

Step a: A solution of Intermediate B3-01 (1.00 g, 4.36 mmol) and formic acid (6 mL) in 48 mL tube sealing was stirred at 75° C. for 1.5 h. Formalin (5 mL) was added, and stirred at 110° C. for 4 h. After cooling to RT, concentrated hydrochloric acid (5 mL) was added, and concentrated under reduced pressure. The residue was dispersed with isopropyl alcohol (5 mL) and diethyl ether (10 mL), and placed in refrigerator for 1.5 h. The supernatant was discarded, and the sank residue was washed with Hex (2×5 mL). The residue was dried under vacuum to give Intermediate B3-02 (760 mg). MS: m/z 158 [M+1]⁺

Step b: To a 0° C. solution of Intermediate B3-02 (760 mg, 3.92 mmol) in THF (10 mL) was added Lithium Aluminum Hydride (364 mg, 9.59 mmol), stirred for 3 h at RT. The reaction mixture was quenched with ice water (0.8 mL), and filtered. The filtrate was concentrated under reduced pressure. The residue was purified by silica chromatography (eluting with DCM:MeOH=100:1-10:1, v/v) to give Intermediate B3 (358 mg). MS: m/z 144 [M+1]⁺

The following intermediates were synthesized using the above procedure or modifications procedure with the corresponding starting materials.

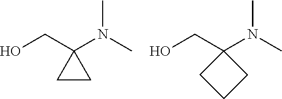

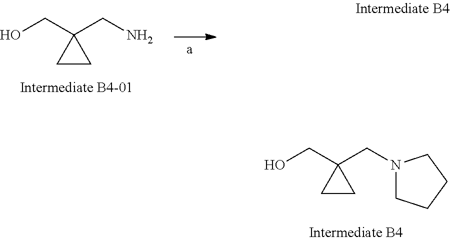 [SMILES: NCC1(CO)CC1 ; OCC1(CN2CCCC2)CC1]

Step a: A suspension of Intermediate B4-01 (300 mg, 2.97 mmol), 1,4-dibromobutane (672 mg, 3.11 mmol) and K₂CO₃ (1.07 g, 7.75 mmol) in ACN (15 mL) was stirred at RT for 15 h. The reaction mixture was filtered and concentrated under reduced pressure. The residue was purified by silica chromatography (eluting with DCM:MeOH=10:1, v/v) to give Intermediate B4 (183 mg). MS: m/z 156 [M+1]⁺.

The following intermediates were synthesized using the above procedure or modifications procedure with the corresponding starting materials.

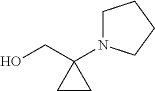

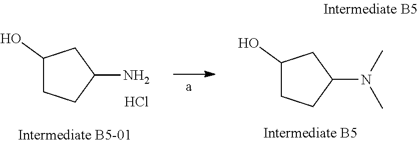

Step a: A solution of Intermediate B5-01 (192 mg, 1.40 mmol), formalin (0.6 mL) and formic acid (1 mL) in 50 mL tube sealing was stirred at 110° C. for 19 h. After cooling to RT, concentrated hydrochloric acid (0.5 mL) and formic acid (5 mL) were added, and concentrated under reduced pressure. The residue was dispersed with MeOH (10 mL) and sodium bicarbonate (3.12 g), and then the mixture was filtered. The collected filtrate was concentrated under reduced pressure and purified by silica chromatography (eluting with DCM:MeOH=4:1, v/v) to give Intermediate B5 (161 mg). MS: m/z 130 [M+1]⁺

The following intermediates were synthesized using the above procedure or modifications procedure with the corresponding starting materials.

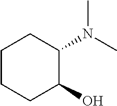

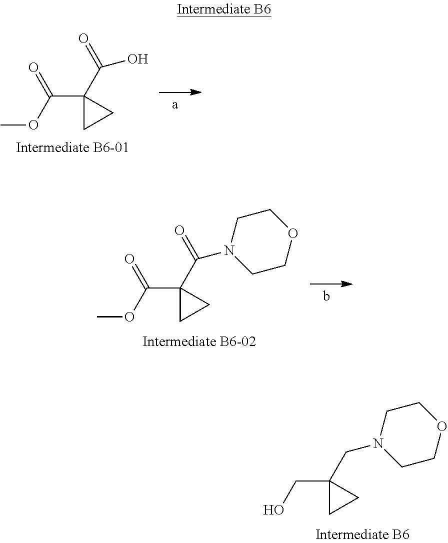

Following procedures of WO2009093012 A1, Intermediate B6 was prepared from 1-(methoxycarbonyl)cyclopropane-1-carboxylic acid in two steps.

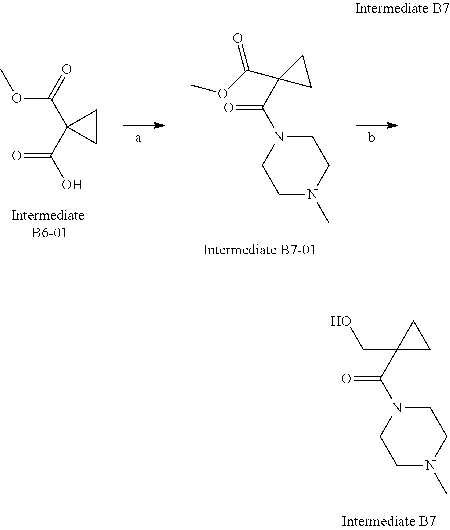

Step a: To a solution of Intermediate B6-01 (6.34 g, 43.99 mmol) in DCM (50 mL) were added dropwise DIEA (3 drops) and oxalyl chloride (7.71 g, 60.74 mmol). The mixture was stirred at RT for 1.5 h and concentrated under reduced pressure. The residue was added dropwise to a solution of 1-methylpiperazine (5.86 g, 58.51 mmol) and TEA (9.30 g, 91.91 mmol) in DCM (100 mL). The reaction mixture was stirred for 1.5 h and quenched with water (50 mL). The organic layer was collected, dried over anhydrous Na₂SO₄, filtered and concentrated under reduced pressure. The residue was purified by silica chromatography (eluting with DCM:MeOH=100/1-30/1, v/v) to give Intermediate B7-01 (9.61 g). MS: m/z 227 [M+1]⁺.

Step b: To a solution of Intermediate B7-01 (2.94 g, 12.99 mmol) in MeOH (30 mL) were added CaCl₂ (1.73 g) and NaBH₄ (2.96 g, 78.24 mmol). NaBH₄ (1.04 g) was added to the reaction mixture after stirring for 3 h. The mixture was stirred for another 20 h and quenched with water (5 mL), filtered. The collected filtrate was concentrated under reduced pressure. The residue was purified by silica chromatography (eluting with DCM:MeOH=50/1-10/1, v/v) to give Intermediate B7 (0.99 g). MS: m/z 199 [M+1]⁺.

The following intermediates were synthesized using the above procedure or modifications procedure (Intermediate B6/Intermediate B7) with the corresponding starting materials.

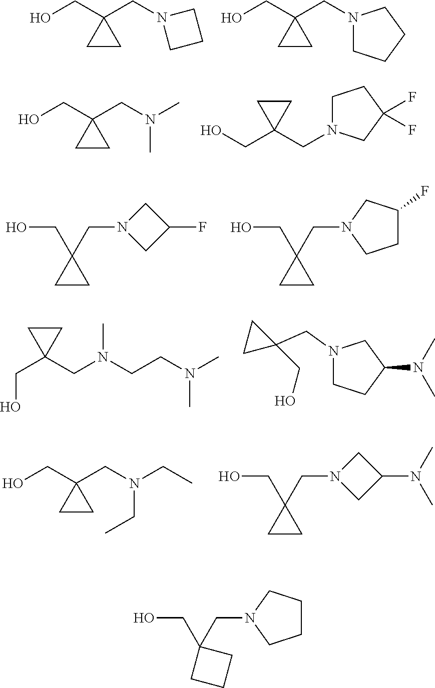

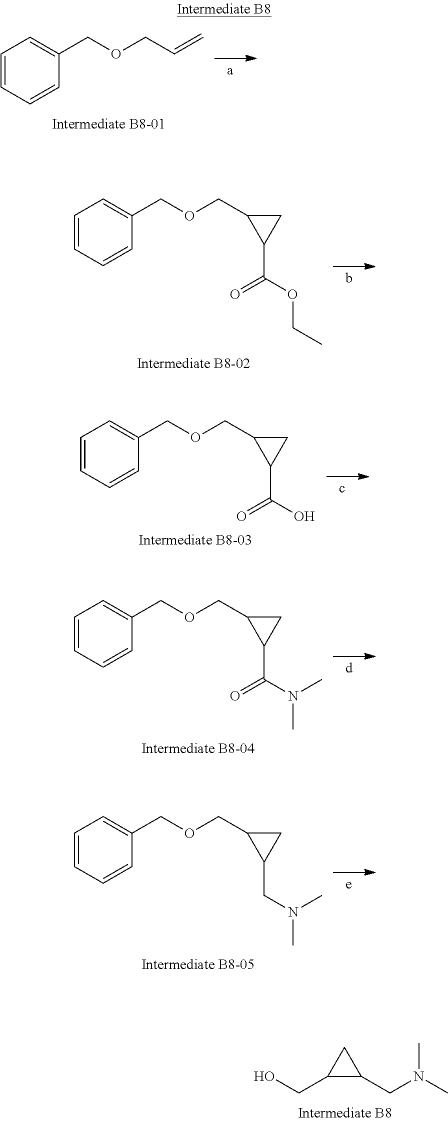

Step a: To a solution of Intermediate B8-01 (5.18 g, 38.60 mmol) and Rhodium(II) acetate dimer (78 mg, 0.18 mmol) in 15 mL Et₂O was added dropwise ethyl diazoacetate (4.78 g, 41.89 mmol) at 25° C.-30° C. The mixture was stirred for 22 h and filtered. The filtrate was concentrated under reduced pressure and purified by silica chromatography (eluting with EtOAc:Hex=1:25, v/v) to give Intermediate B8-02 (2060 mg).

Step b: A solution of Intermediate B8-02 (2286 mg, 9.76 mmol) and NaOH (3.22 g, 80.50 mmol) in EtOH (25 mL) and water (25 mL) was stirred at 70° C. for 2 h. The reaction mixture was adjusted pH to 7 by concentrated HCl and concentrated under reduced pressure. The residue was dispersed in EtOAc and citric acid solution. The organic layer was collected and washed with brine, dried over anhydrous Na₂SO₄, filtered and concentrated under reduced pressure. The residue was purified by silica chromatography (eluting with Hex:EtOAc=4:1, v/v) to give Intermediate B8-03 (1196 mg). MS: m/z 191 [M−1]⁺

Step c: To a solution of Intermediate B8-03 (645 mg, 3.13 mmol), HATU (1310 mg, 3.45 mmol) and dimethylamine hydrochloride in (445 mg, 5.46 mmol) in DCM (50 mL) was added dropwise DIEA 1243 mg, 9.62 mmol), stirred for 2 h at RT. The reaction mixture was concentrated under reduced pressure and purified by silica chromatography (eluting with MeOH:DCM=1:40, v/v) to give Intermediate B8-04 (685 mg). MS: m/z 220 [M+1]⁺.

Step d: To a solution of Lithium Aluminum Hydride (12 mL, 1 mol/L) in THF (8 mL) was added Intermediate B8-04 (760 mg, 3.92 mmol), stirred for 2 h at 40° C. The reaction mixture was quenched with ice water, and filtered. The filtrate was concentrated under reduced pressure. The residue was purified by silica chromatography (eluting with DCM:MeOH=8:1, v/v) to give Intermediate B8-05 (576 mg). MS: m/z 206 [M+1]⁺.

Step e: To a solution of Intermediate B8-05 (264 ng, 1.20 mmol) in MeOH (25 mL) was added Pd—C (10%, 0.53 g). The reaction mixture was degassed under vacuum and purged with H₂ 3 times, and stirred at 45° C. for 2.5 h. The mixture was filtered. The filtrate was concentrated under reduced pressure to give Intermediate B8 (153 mg). MS: m/z 116 [M+1]⁺.

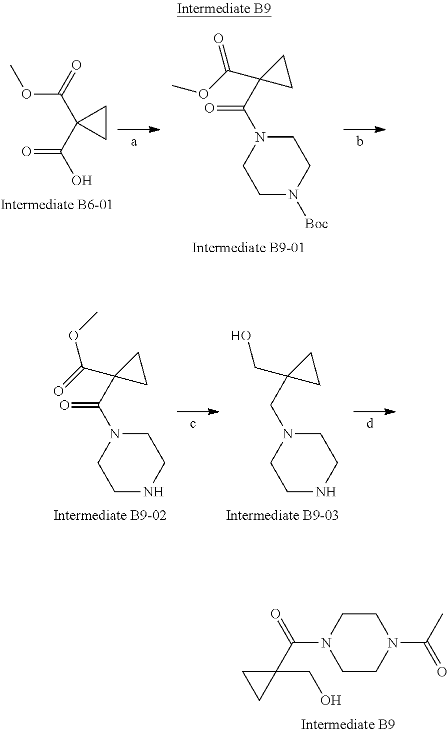

Step a: To a solution of Intermediate B6-01 (7.61 g, 52.80 mmol) in DCM (50 mL) were added dropwise DIEA (3 drops) and oxalyl chloride (8.89 g, 70.04 mmol). The mixture was stirred at 25° C. for 2 h and concentrated under reduced pressure. The residue was added dropwise to a solution of tert-Butyl 1-piperazinecarboxylate (12.60 g, 67.65 mmol) and TEA (10.66 g, 105.35 mmol) in DCM (70 mL). The reaction mixture was stirred for 0.5 h and quenched with water (50 mL). The organic layer was collected, dried over anhydrous Na₂SO₄, filtered and concentrated under reduced pressure. The residue was purified by silica chromatography (eluting with DCM:MeOH=50:1, v/v) to give Intermediate B9-01 (16.69 g). MS: m/z 313 [M+1]⁺.

Step b: To a solution of Intermediate B9-01 (16.69 g, 53.43 mmol) in EtOAc (30 mL) were added HCl (4 mol/L in EtOAc, 85 mL). The reaction mixture was stirred for 3 h and concentrated under reduced pressure. The residue was suspended in EtOAc (100 mL). The mixture was adjusted pH to 12 by TEA and filtered. The filtrate was concentrated under reduced pressure to give Intermediate B9-02 (12.02 g). MS: m/z 213 [M+1]⁺.

Step c: To a solution of Intermediate B9-02 (12.02 g, 56.63 mmol) in THF (150 mL) were added LiAlH₄ (4.34 g, 114.36 mmol) in portions below −20° C. LiAlH₄ (1.05 g) was added to the reaction mixture in portions after stirring for 3 h. The mixture was stirred for another 3 h and quenched with water (5 mL), NaOH solution (15%, 5 mL) and water (15 mL), filtered. The collected filtrate was concentrated under reduced pressure. The residue was purified by silica chromatography (eluting with DCM:MeOH=10/1-3/1, v/v) to give Intermediate B9-03 (2.39 g). MS: m/z 171 [M+1]⁺.

Step d: To a solution of Intermediate B9-03 (1.42 g, 8.34 mmol) in DCM (15 mL) were added dropwise a solution of acetyl chloride (447 mg, 5.69 mmol) in DCM (1 mL) below −70° C. The reaction mixture was stirred for 20 min. TEA was added to the mixture to adjust pH to 12. The mixture was filtered and the collected filtrate was concentrated under reduced pressure. The residue was suspended in EA (15 mL), and the mixture was filtered. The collected filtrate was concentrated under reduced pressure. The residue was purified by silica chromatography (eluting with DCM:MeOH=50/1, v/v) to give Intermediate B9 (1.13 g). MS: m/z 227 [M+1]

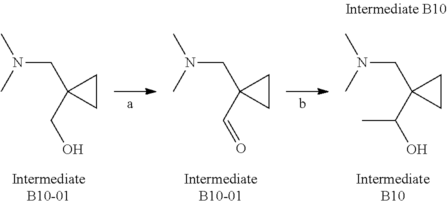

Step a: To a solution of Intermediate B10-01 (1.50 g, 11.61 mmol) in DCM (30 mL) were added Dess-Martin periodinane (7.40 g, 17.45 mmol) in portions. DCM (100 mL) and potassium carbonate solution (40%, 20 mL) were added to the mixture after stirring at RT for 17 h. The mixture was filtered and the filtrate was separated. The organic layer was concentrated under reduced pressure. The residue was purified by silica chromatography (eluting with DCM:MeOH=20:1, v/v) to give Intermediate B10-02 (0.64 g). MS: m/z 128 [M+1]⁺.

Step b: To a solution of Intermediate B10-02 (0.64 g, 5.03 mmol) in THF (10 mL) were added dropwise methyllithium (1 mol/L in THF, 5 mL) below −50° C. After stirring for 19 h, the mixture was concentrated under reduced pressure. The residue was purified by silica chromatography (eluting with DCM:MeOH=20:1, v/v) to give Intermediate B10 (408 mg). MS: m/z 144 [M+1]⁺.

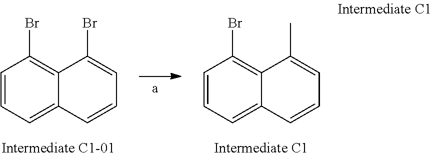

Following procedures of Journal of Organic Chemistry (2016), 81(22), 10791-10801, Intermediate C1 was prepared from 1,8-dibromonaphthalene in one step.

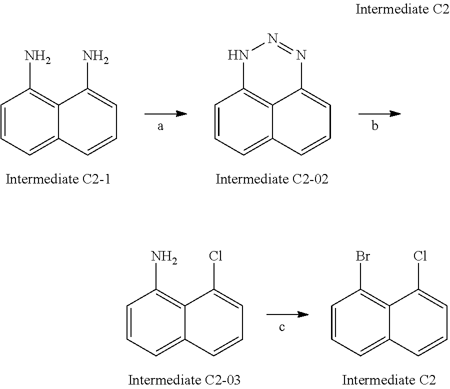 [SMILES: Nc1cccc2cccc(N)c12 ; c1cc2c3c(cccc3c1)NN=N2 ; Nc1cccc2cccc(Cl)c12 ; Clc1cccc2cccc(Br)c12]

Following procedures of US20180072723 A1, Intermediate C2 was prepared from naphthalene-1,8-diamine in 3 steps.

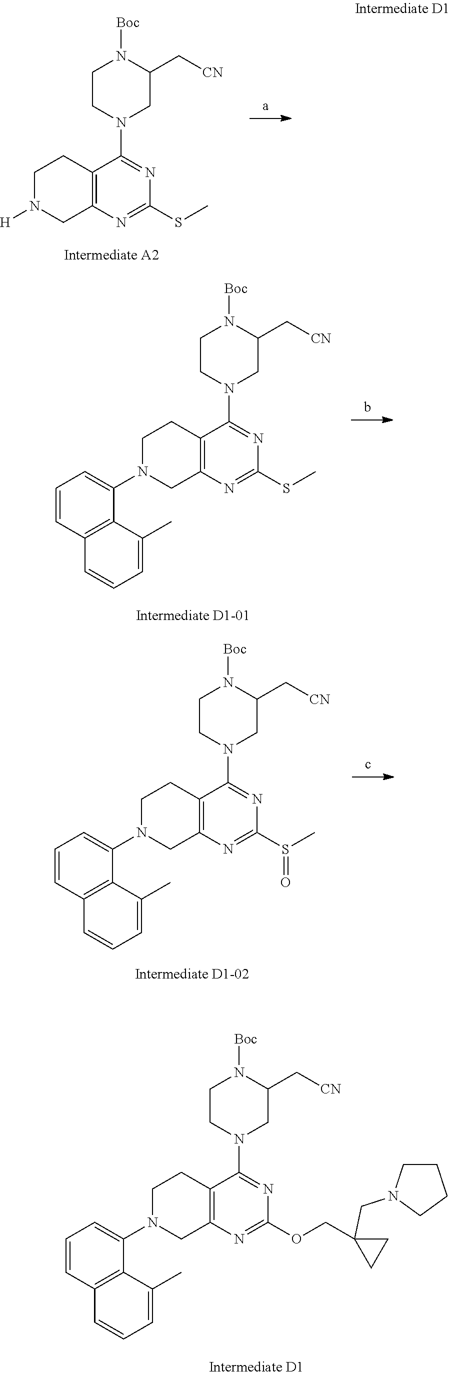 [SMILES: CSc1nc2c(c(N3CCN(C(=O)OC(C)(C)C)C(CC#N)C3)n1)CCNC2 ; CSc1nc2c(c(N3CCN(C(=O)OC(C)(C)C)C(CC#N)C3)n1)CCN(c1cccc3cccc(C)c13)C2 ; Cc1cccc2cccc(N3CCc4c(nc(S(C)=O)nc4N4CCN(C(=O)OC(C)(C)C)C(CC#N)C4)C3)c12 ; Cc1cccc2cccc(N3CCc4c(nc(OCC5(CN6CCCC6)CC5)nc4N4CCN(C(=O)OC(C)(C)C)C(CC#N)C4)C3)c12]

Step a: To a solution of Intermediate A2 (3.14 g, 7.76 mmol), Intermediate C1 (2.11 g, 9.54 mmol) and Cs₂CO₃ (7.72 g, 23.69 mmol) in toluene (50 mL) was added RuPhos (316 mg, 0.70 mmol) and Pd₂(dba)₃ (307 mg, 0.34 mmol), the suspension was degassed under vacuum and purged with nitrogen three times. The reaction mixture was stirred at 100° C. for 24 hour. Upon completion, the resulting mixture was filtered. The filtrate was concentrated under reduced pressure. The residue was purified by silica chromatography (eluting with Hex:EA=10:1, v/v) to give Intermediate D1-01 (2.42 g) as a brown solid. MS: m/z 545 [M+H]⁺.

Step b: To a solution of Intermediate D1-01 (2.42 g, 4.44 mmol) in DCM (5 mL) was added m-CPBA (965 mg, 4.75 mmol, 85% purity) at 0° C. and the mixture stirred at 0° C. for 45 minutes. DCM (30 mL) was added to the reaction mixture, and then quenched by saturated Na₂S₂O₃ (20 mL) and separated. The collected organic layer was washed with saturated NaHCO₃ (20 mL), dried over anhydrous Na₂SO₄, filtered and concentrated under reduced pressure. The residue was purified by silica chromatography (eluting with DCM:MeOH=80:1-20:1, v/v) to give Intermediate D1-02 (2.19 g) as a yellow solid. MS: m/z 561 [M+H]⁺.

Step c: To a solution of Intermediate B4 (507 mg, 3.27 mmol) in dry THF (6 mL) was added t-BuONa (626 mg, 6.54 mmol) under nitrogen at −20° C. and stirred for 30 minutes. Then the solution of Intermediate D1-02 (610 mg, 1.09 mmol) in dry THF (4 mL) was added to the reaction mixture and stirred at −20° C. for 20 minutes. Upon completion, the mixture was quenched by water (20 mL) and extracted with EtOAc (20 mL*2). The combined organic layers were concentrated under reduced pressure. The residue was purified by silica chromatography (eluting with DCM:MeOH=30:1, v/v) to give Intermediate D1 (597 mg) as a red oil. MS: m/z 652 [M+H]⁺.

The following intermediates were synthesized using the above procedure or modifications procedure with the corresponding starting materials or intermediates.

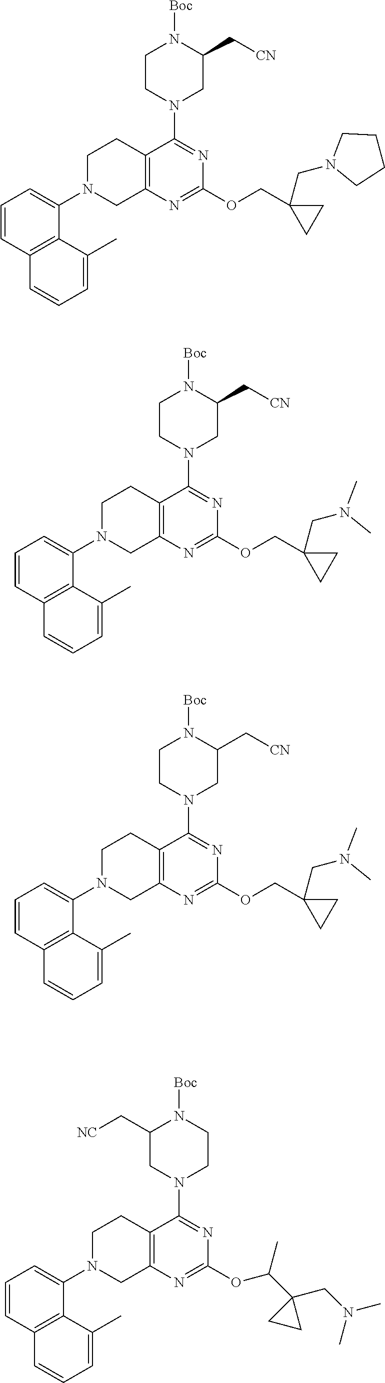 [SMILES: Cc1cccc2cccc(N3CCc4c(nc(OCC5(CN6CCCC6)CC5)nc4N4CCN(C(=O)OC(C)(C)C)[C@@H](CC#N)C4)C3)c12 ; Cc1cccc2cccc(N3CCc4c(nc(OCC5(CN(C)C)CC5)nc4N4CCN(C(=O)OC(C)(C)C)[C@@H](CC#N)C4)C3)c12 ; Cc1cccc2cccc(N3CCc4c(nc(OCC5(CN(C)C)CC5)nc4N4CCN(C(=O)OC(C)(C)C)C(CC#N)C4)C3)c12 ; Cc1cccc2cccc(N3CCc4c(nc(OC(C)C5(CN(C)C)CC5)nc4N4CCN(C(=O)OC(C)(C)C)C(CC#N)C4)C3)c12]

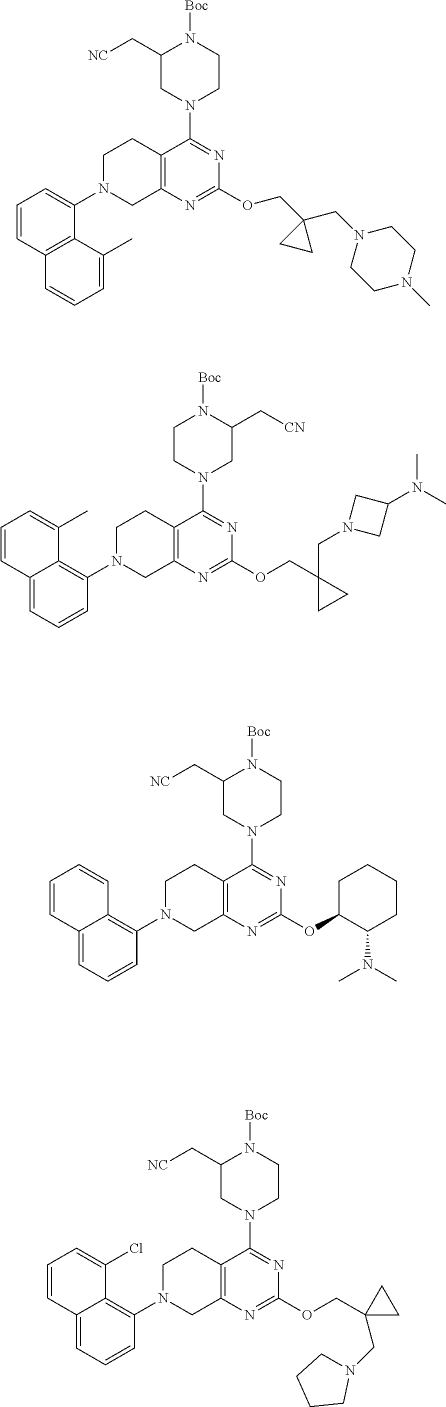 [SMILES: Cc1cccc2cccc(N3CCc4c(nc(OCC5(CN6CCN(C)CC6)CC5)nc4N4CCN(C(=O)OC(C)(C)C)C(CC#N)C4)C3)c12 ; Cc1cccc2cccc(N3CCc4c(nc(OCC5(CN6CC(N(C)C)C6)CC5)nc4N4CCN(C(=O)OC(C)(C)C)C(CC#N)C4)C3)c12 ; CN(C)[C@H]1CCCC[C@@H]1Oc1nc2c(c(N3CCN(C(=O)OC(C)(C)C)C(CC#N)C3)n1)CCN(c1cccc3ccccc13)C2 ; CC(C)(C)OC(=O)N1CCN(c2nc(OCC3(CN4CCCC4)CC3)nc3c2CCN(c2cccc4cccc(Cl)c24)C3)CC1CC#N]

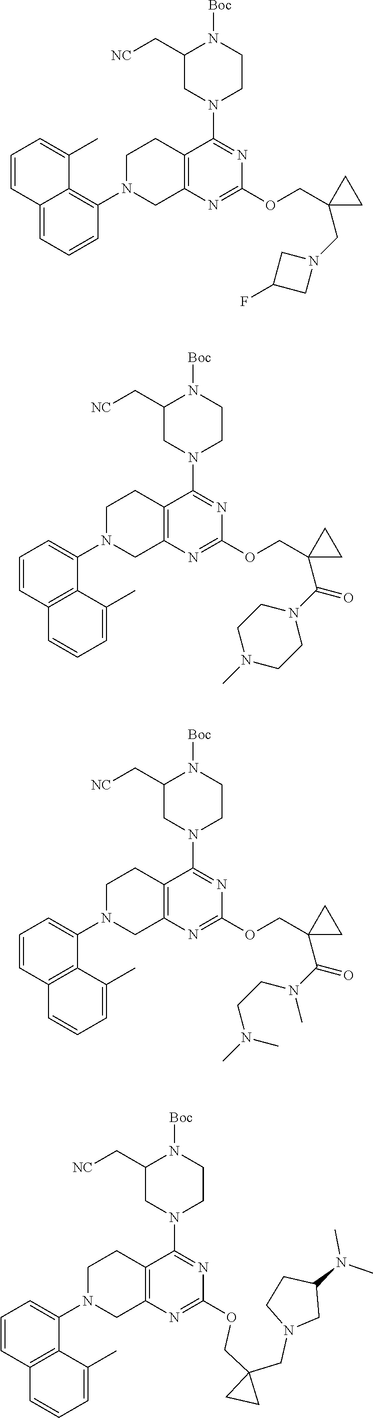

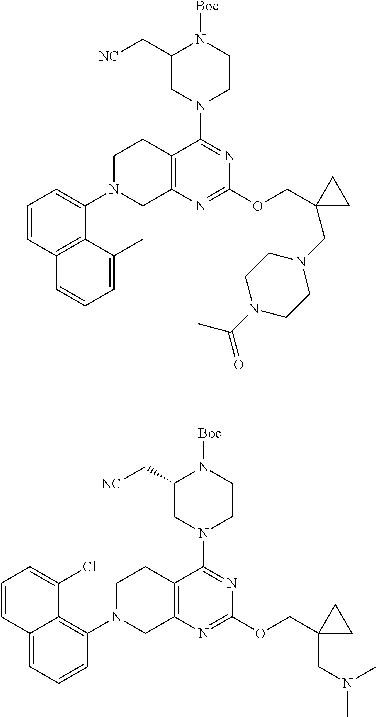 [SMILES: CC(=O)N1CCN(CC2(COc3nc4c(c(N5CCN(C(=O)OC(C)(C)C)C(CC#N)C5)n3)CCN(c3cccc5cccc(C)c35)C4)CC2)CC1 ; CN(C)CC1(COc2nc3c(c(N4CCN(C(=O)OC(C)(C)C)[C@@H](CC#N)C4)n2)CCN(c2cccc4cccc(Cl)c24)C3)CC1]

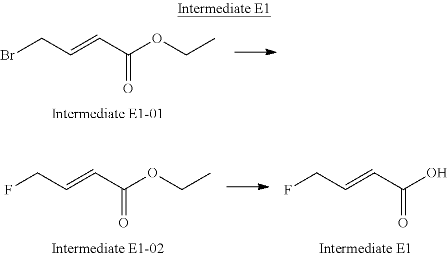

Following procedures of WO2019099524, Intermediate E1 was prepared from ethyl(E)-4-bromobut-2-enoate in 2 steps.

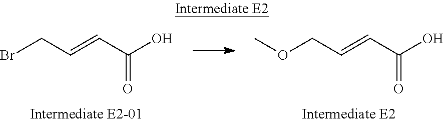

Following procedures of WO2013010869 A1, Intermediate E2 was prepared from (E)-4-bromobut-2-enoic acid in 1 step.

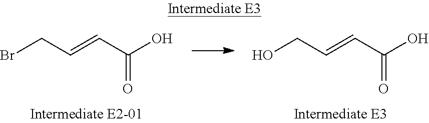 [SMILES: O=C(O)/C=C/CBr ; O=C(O)/C=C/CO]

Following procedures of WO2019099524, Intermediate E3 was prepared from (E)-4-bromobut-2-enoic acid in 1 step.

### Example 1

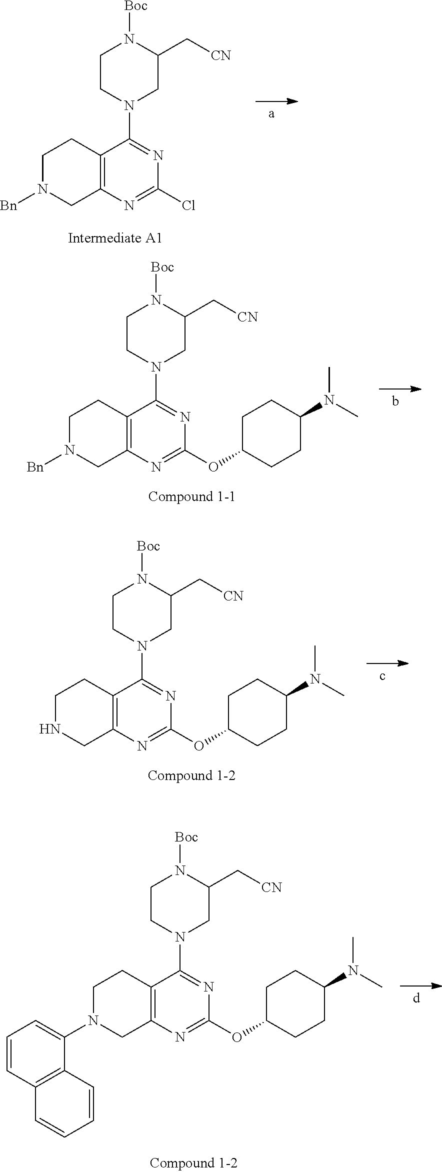 [SMILES: CC(C)(C)OC(=O)N1CCN(c2nc(Cl)nc3c2CCN(Cc2ccccc2)C3)CC1CC#N ; CN(C)[C@H]1CC[C@H](Oc2nc3c(c(N4CCN(C(=O)OC(C)(C)C)C(CC#N)C4)n2)CCN(Cc2ccccc2)C3)CC1 ; CN(C)[C@H]1CC[C@H](Oc2nc3c(c(N4CCN(C(=O)OC(C)(C)C)C(CC#N)C4)n2)CCNC3)CC1 ; CN(C)[C@H]1CC[C@H](Oc2nc3c(c(N4CCN(C(=O)OC(C)(C)C)C(CC#N)C4)n2)CCN(c2cccc4ccccc24)C3)CC1]

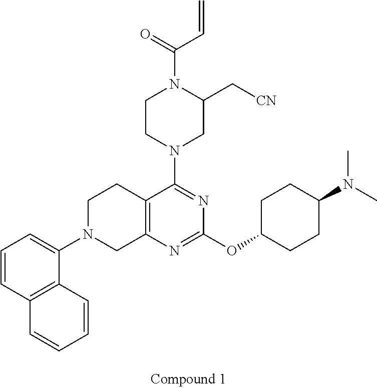 [SMILES: C=CC(=O)N1CCN(c2nc(O[C@H]3CC[C@H](N(C)C)CC3)nc3c2CCN(c2cccc4ccccc24)C3)CC1CC#N]

Step a: To a mixture of Intermediate A1 (300 mg, 0.62 mmol), (1r,4r)-4-(dimethylamino)cyclohexan-1-ol (140 mg, 0.98 mmol) and sodium tert-butoxide (170 mg, 1.77 mmol) in toluene (10 mL) was added BINAP (65 mg, 0.10 mmol) and Pd₂(dba)₃ (55 mg, 0.06 mmol) and the mixture was purged with nitrogen followed by stirring at 95° C. for 2.5 h. The mixture was diluted with ethyl acetate (30 mL) and water (40 mL) and the organic layer was separated. The organic layer was washed with brine (40 mL) and dried over anhydrous Na₂SO₄, filtered and concentrated under reduced pressure. The residue was purified by Pre-TLC (DCM:MeOH=9:1) to give Compound 1-1 (239 mg, 0.41 mmol). MS: m/z 590 [M+1]⁺.

Step b: To a solution of Compound 1-1 (229 mg, 0.39 mmol) in MeOH (10 mL) was added Pd/C (375 mg, 10%). The suspension was degassed under reduced pressure and purged with H₂ three times. The mixture was stirred under H₂ at 60° C. for 1 h. The mixture was filtered and the filter cake was washed with MeOH (20 mL). The filtrate was concentrated under reduced pressure to give Compound 1-2 (193 mg, 0.39 mmol). MS: m/z 500 [M+1]⁺.

Step c: To a solution of Compound 1-2 (193 mg, 0.39 mmol), 1-bromonaphthalene (140 mg, 0.68 mmol) and Cs₂CO₃ (315 mg, 0.97 mmol) in toluene (10 mL) was added RuPhos (53 mg, 0.11 mmol) and Pd₂(dba)₃ (37 mg, 0.04 mmol) and the mixture was purged with nitrogen followed by stirring at 100° C. for 6 h. The mixture was filtered and concentrated under reduced pressure. The residue was purified by Pre-TLC (DCM:MeOH=9; 1) to give Compound 1-3 (141 mg, 0.23 mmol). MS: m/z 626 [M+1]⁺.

Step d: To a solution of Compound 1-3 (141 mg, 0.23 mmol) in DCM (3 mL) was added TFA (1.2 mL). The reaction mixture was stirred at 25° C. for 0.5 h. Upon completion, the reaction mixture was concentrated under reduced pressure. The residue was dissolved in DCM (5 mL). To the solution was added TEA (0.5 mL) and acryloyl chloride (69 mg, 0.76 mmol) and the mixture was stirred at 25° C. for 10 minutes. The reaction mixture was diluted with DCM (40 mL) and water (30 mL) and the organic layer was separated. The organic layer was washed with brine (30 mL) and dried over anhydrous Na₂SO₄, filtered and concentrated under reduced pressure.

The residue was purified by Pre-TLC (DCM:MeOH=9:1) to give Compound 1 (68 mg). MS: m/z 580 [M+1]⁺. ¹H NMR (400 MHz, MeOH-d4) δ 8.31-8.18 (m, 1H), 7.93-7.82 (m, 1H), 7.62 (d, 1H), 7.57-7.48 (m, 2H), 7.44 (t, 1H), 7.21 (d, 1H), 6.84 (s, 1H), 6.31 (d, 1H), 5.86 (d, 1H), 5.16-4.96 (m, 2H), 4.33-4.03 (m, 4H), 3.65 (s, 1H), 3.44 (s, 2H), 3.40-3.21 (m, 3H), 3.13-2.95 (m, 3H), 2.88 (s, 6H), 2.38 (s, 2H), 2.18 (d, 2H), 1.80 (d, 2H), 1.72-1.53 (m, 2H), 1.43-1.22 (m, 2H).

The following Compounds were synthesized using the above procedure or modification procedure using the corresponding Intermediate.

| Com- | | | |
|---|---|---|---|
| pound | Chemical Name | Structure | MS: (M + H )⁺/¹HNMR |
| 2. | 2-(1-acryloyl-4-(2-((1- ((dimethylamino)methyl) cyclopropyl)methoxy)-7- (2-methyl-1-oxo-1,2- dihydroisoquinolin-8-yl)- 5,6,7,8- tetrahydropyrido[3,4- d]pyrimidin-4- yl)piperazin-2- yl)acetonitrile | 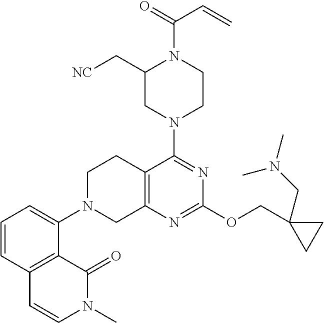 [SMILES: C=CC(=O)N1CCN(c2nc(OCC3(CN(C)C)CC3)nc3c2CCN(c2cccc4ccn(C)c(=O)c24)C3)CC1CC#N] | MS: 597 ¹H NMR (400 MHz, MeOD) δ 7.59-7.44 (m, 1H), 7.26 (d, 1H), 7.15 (d, 1H), 7.07 (d, 1H), 6.72 (s, 1H), 6.50 (d, 1H), 6.19 (d, 1H), 5.73 (d, 1H), 4.97 (s, 1H), 4.54- 4.37 (m, 1H), 4.36-3.86 (m, 5H), 3.61-3.38 (m, 4H), 3.19- 2.81 (m, 8H), 2.81-2.58 (m, 6H), 1.35-1.02 (m, 3H), 0.86- 0.55 (m, 4H). |
| 3. | 2-(1-acryloyl-4-(2-((1- ((dimethylamino)methyl) cyclopropyl)methoxy)-7- (5-methyl-1H-indazol-4- yl)-5,6,7,8- tetrahydropyrido[3,4- d]pyrimidin-4- yl)piperazin-2- yl)acetonitrile | 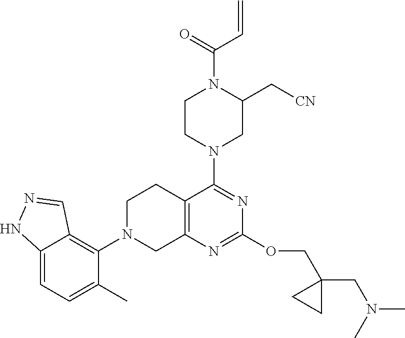 [SMILES: C=CC(=O)N1CCN(c2nc(OCC3(CN(C)C)CC3)nc3c2CCN(c2c(C)ccc4[nH]ncc24)C3)CC1CC#N] | MS: 571 |
| 4. | 2-(1-acryloyl-4-(7- (naphthalen-1-yl)-2-((1- (pyrrolidin-1- ylmethyl)cyclopropyl)meth- oxy)-5,6,7,8- tetrahydropyrido[3,4- d]pyrimidin-4- yl)piperazin-2- yl)acetonitrile | 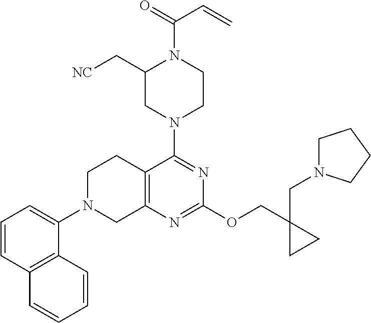 [SMILES: C=CC(=O)N1CCN(c2nc(OCC3(CN4CCCC4)CC3)nc3c2CCN(c2cccc4ccccc24)C3)CC1CC#N] | MS: 592 ¹H NMR (400 MHz, CDCl₃) δ 8.13 (d, 1H), 7.78 (d, 1H), 7.43 (ddd, 4H), 7.08 (d, 1H), 6.51 (s, 1H), 6.32 (d, 1H), 5.75 (d, 1H), 5.01 (s, 1H), 4.33 (dd, 6H), 3.90 (s, 2H), 3.57 (s, 1H), 3.47-3.00 (m, 6H), 2.82 (s, 5H), 2.24 (s, 2H), 1.96 (s, 2H), 1.21 (d, 2H), 0.89 (s, 2H), 0.79 (s, 2H). |
| 5. | 2-(1-acryloyl-4-(2-((3- (dimethylamino)cyclohex- yl)methoxy)-7-(8- methylnaphthalen-1-yl)- 5,6,7,8- tetrahydropyrido[3,4- d]pyrimidin-4- yl)piperazin-2- yl)acetonitrile | 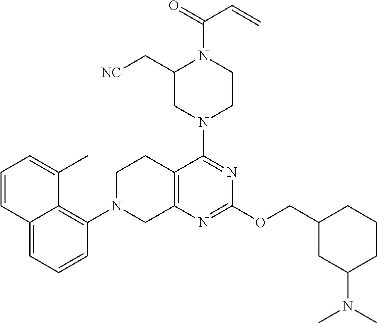 [SMILES: C=CC(=O)N1CCN(c2nc(OCC3CCCC(N(C)C)C3)nc3c2CCN(c2cccc4cccc(C)c24)C3)CC1CC#N] | MS: 608 ¹H NMR (400 MHz, DMSO) δ 7.76 (d, 1H), 7.70 (d, 1H), 7.46 (dd, 1H), 7.35 (dd, 2H), 7.27 (d, 1H), 6.86 (s, 1H), 6.19 (d, 1H), 5.78 (d, 1H), 4.01 (dd, 6H), 3.80-3.62 (m, 2H), 3.20-3.04 (m, 5H), 2.86 (s, 4H), 2.64 (s, 7H), 2.19-1.97 (m, 3H), 1.92- 1.67 (m, 3H), 1.23 (dd, 26.6 Hz, 6H) |
| 6. | 2-(1-acryloyl-4-(2-((3- (dimethylamino)cyclohex- yl)methoxy)-7-(2- (trifluoromethyl)phenyl)- 5,6,7,8- tetrahydropyrido[3,4- d]pyrimidin-4- yl)piperazin-2- yl)acetonitrile | 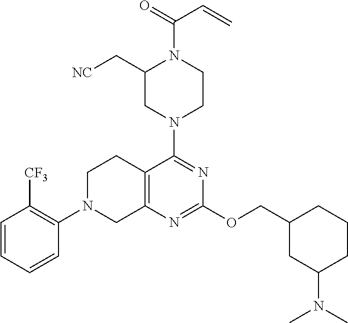 [SMILES: C=CC(=O)N1CCN(c2nc(OCC3CCCC(N(C)C)C3)nc3c2CCN(c2ccccc2C(F)(F)F)C3)CC1CC#N] | MS: 612 ¹H NMR (400 MHz, DMSO) δ 7.80-7.60 (m, 3H), 7.40 (t, 1H), 6.86 (s, 1H), 6.19 (d, 1H), 5.78 (d, 1H), 4.88 (d, 1H), 4.08 (dd, 3H), 4.02 (s, 4H), 3.06 (ddd, 7H), 2.78 (s, 2H), 2.66 (s, 6H), 2.16 (d, 1H), 2.04 (s, 1H), 1.86 (d, 2H), 1.73 (d, 1H), 1.41-1.16 (m, 4H), 0.99 (d, 1H). |
| 7. | 2-(1-acryloyl-4-(2-(3- (dimethylamino)cyclobu- toxy)-7-(naphthalen-1-yl)- 5,6,7,8- tetrahydropyrido[3,4- d]pyrimidin-4- yl)piperazin-2- yl)acetonitrile | 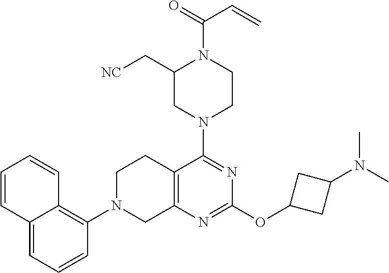 [SMILES: C=CC(=O)N1CCN(c2nc(OC3CC(N(C)C)C3)nc3c2CCN(c2cccc4ccccc24)C3)CC1CC#N] | MS: 552 |
| 8. | 2-(1-acryloyl-4-(2-((3- (dimethylamino)cyclobu- tyl)methoxy)-7- (naphthalen-1-yl)-5,6,7,8- tetrahydropyrido[3,4- d]pyrimidin-4- yl)piperazin-2- yl)acetonitrile | 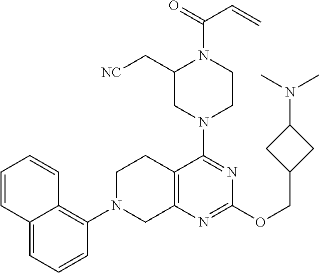 [SMILES: C=CC(=O)N1CCN(c2nc(OCC3CC(N(C)C)C3)nc3c2CCN(c2cccc4ccccc24)C3)CC1CC#N] | MS: 566 ¹H NMR (400 MHz, MeOD) δ 8.21-8.02 (m, 1H), 7.84-7.66 (m, 1H), 7.52 (d, 1H), 7.46-7.24 (m, 3H), 7.11 (d, 1H), 6.74 (s, 1H), 6.19 (d, 1H), 5.74 (d, 1H), 4.99 (s, 1H), 4.56-4.37 (m, 1H), 4.38-3.90 (m, 5H), 3.69-3.43 (m, 2H), 3.43-3.07 (m, 4H), 3.05-2.68 (m, 6H), 2.64-2.24 (m, 8H), 2.08-1.90 (m, 2H). |
| 9. | 2-(1-acryloyl-4-(2-((1- (dimethylamino)cyclobu- tyl)methoxy)-7- (naphthalen-1-yl)-5,6,7,8- tetrahydropyrido[3,4- d]pyrimidin-4- yl)piperazin-2- yl)acetonitrile | 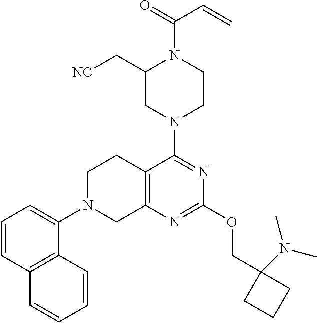 [SMILES: C=CC(=O)N1CCN(c2nc(OCC3(N(C)C)CCC3)nc3c2CCN(c2cccc4ccccc24)C3)CC1CC#N] | MS: 566 ¹H NMR (400 MHz, MeOD) δ 8.33-8.19 (m, 1H), 7.96-7.82 (m, 1H), 7.71-7.59 (m, 1H), 7.57-7.36 (m, 3H), 7.23 (d, 1H), 6.84 (s, 1H), 6.32 (d, 1H), 5.84 (d, 1H), 5.10 (s, 1H), 4.58 (s, 1H), 4.42 (d, 1H), 4.33-4.06 (m, 4H), 3.77-3.35 (m, 4H), 3.21-2.90 (m, 4H), 2.84 (s, 6H), 2.54-2.23 (m, 4H), 2.10-1.92 (m, 2H), 1.43-1.22 (m, 2H). |
| 10. | 2-(1-acryloyl-4-(2-((3- (dimethylamino)cyclohex- yl)oxy)-7-(naphthalen-1- yl)-5,6,7,8- tetrahydropyrido[3,4- d]pyrimidin-4- yl)piperazin-2- yl)acetonitrile | 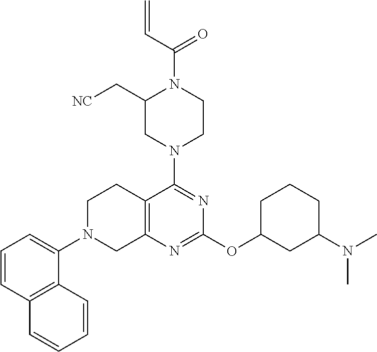 [SMILES: C=CC(=O)N1CCN(c2nc(OC3CCCC(N(C)C)C3)nc3c2CCN(c2cccc4ccccc24)C3)CC1CC#N] | MS: 578 ¹H NMR (400 MHz, MeOD) δ 8.20-8.06 (m, 1H), 7.82-7.71 (m, 1H), 7.51 (d, 1H), 7.46-7.27 (m, 3H), 7.10 (d, 1H), 6.72 (s, 1H), 6.20 (d, 1H), 5.74 (d, 1H), 4.98 (s, 1H), 4.22-3.89 (m, 4H), 3.53 (s, 1H), 3.44-3.27 (m, 2H), 3.27-2.78 (m, 7H), 2.74-2.58 (m, 5H), 2.53-2.35 (m, 1H), 2.10 (d, 1H), 2.02-1.82 (m, 2H), 1.64-1.30 (m, 4H), 1.27-1.09 (m, 3H). |
| 11. | 2-(1-acryloyl-4-(2-((2- (dimethylamino)cyclohex- yl)oxy)-7-(naphthalen-1- yl)-5,6,7,8- tetrahydropyrido[3,4- d]pyrimidin-4- yl)piperazin-2- yl)acetonitrile | 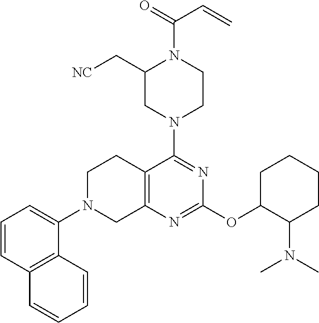 [SMILES: C=CC(=O)N1CCN(c2nc(OC3CCCCC3N(C)C)nc3c2CCN(c2cccc4ccccc24)C3)CC1CC#N] | MS: 580 |
| 12. | 2-(1-acryloyl-4-(2-((1- (dimethylamino)cyclopro- pyl)methoxy)-7- (naphthalen-1-yl)-5,6,7,8- tetrahydropyrido[3,4- d]pyrimidin-4- yl)piperazin-2- yl)acetonitrile | 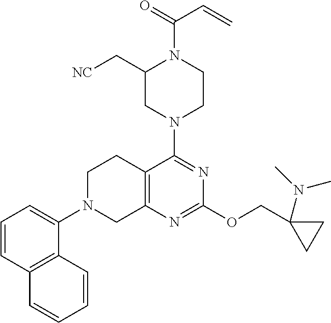 [SMILES: C=CC(=O)N1CCN(c2nc(OCC3(N(C)C)CC3)nc3c2CCN(c2cccc4ccccc24)C3)CC1CC#N] | MS: 552 |
| 13. | 2-(1-acryloyl-4-(2-((3- (dimethylamino)cyclopen- tyl)oxy)-7-(naphthalen-1- yl)-5,6,7,8- tetrahydropyrido[3,4- d]pyrimidin-4- yl)piperazin-2- yl)acetonitrile | 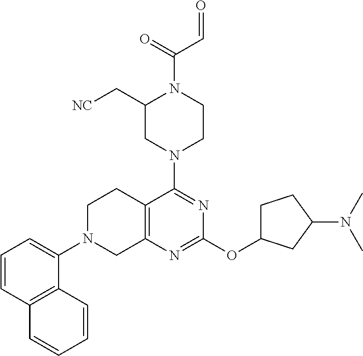 [SMILES: CN(C)C1CCC(Oc2nc3c(c(N4CCN(C(=O)C=O)C(CC#N)C4)n2)CCN(c2cccc4ccccc24)C3)C1] | MS: 566 |
| 14. | 2-(1-acryloyl-4-(2-((2- (dimethylamino)cyclopen- tyl)methoxy)-7- (naphthalen-1-yl)-5,6,7,8- tetrahydropyrido[3,4- d]pyrimidin-4- yl)piperazin-2- yl)acetonitrile | 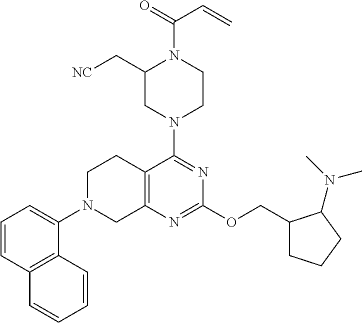 [SMILES: C=CC(=O)N1CCN(c2nc(OCC3CCCC3N(C)C)nc3c2CCN(c2cccc4ccccc24)C3)CC1CC#N] | MS: 580 ¹H NMR (400 MHz, DMSO) δ 8.38-8.10 (m, 1H), 8.06-7.86 (m, 1H), 7.65 (d, 1H), 7.61-7.40 (m, 3H), 7.23 (d, 1H), 6.88 (s, 1H), 6.20 (d, 1H), 5.79 (d, 1H), 4.88 (d, 1H), 4.69-4.24 (m, 2H), 4.10 (d, 5H), 3.66-3.37 (m, 2H), 3.21 (s, 3H), 3.00 (d, 6H), 2.74 (d, 2H), 2.26 (d, 4H), 1.72 (d, 5H), 1.20 (d, 1H). |
| 15. | 2-(1-acryloyl-4-(2-((3- (dimethylamino)cyclohex- yl)methoxy)-7- (naphthalen-1-yl)-5,6,7,8- tetrahydropyrido[3,4- d]pyrimidin-4- yl)piperazin-2- yl)acetonitrile | 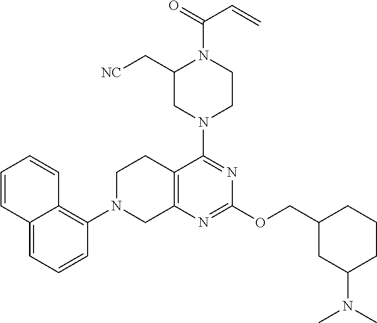 [SMILES: C=CC(=O)N1CCN(c2nc(OCC3CCCC(N(C)C)C3)nc3c2CCN(c2cccc4ccccc24)C3)CC1CC#N] | MS: 594, ¹H NMR (400 MHz, MeOD) δ 8.34-8.17 (m, 1H), 7.96-7.84 (m, 1H), 7.63 (d, 1H), 7.58-7.37 (m, 3H), 7.22 (d, 1H), 6.83 (s, 1H), 6.31 (d, 1H), 5.85 (d, 1H), 5.10 (s, 1H), 4.40-4.01 (m, 6H), 3.66 (s, 1H), 3.44 (s, 2H), 3.34-3.22 (m, 2H), 3.20-2.92 (m, 4H), 2.87 (s, 6H), 2.28 (d, 1H), 2.18-1.96 (m, 3H), 1.90 (d, 1H), 1.59-1.07 (m, 6H). |
| 16. | 2-(1-acryloyl-4-(2-((2-(4- methylpiperazin-1- yl)cyclopentyl)oxy)-7- (naphthalen-1-yl)-5,6,7,8- tetrahydropyrido[3,4- d]pyrimidin-4- yl)piperazin-2- yl)acetonitrile | 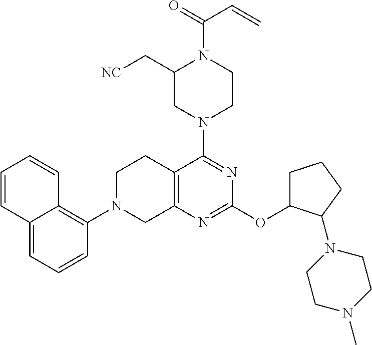 [SMILES: C=CC(=O)N1CCN(c2nc(OC3CCCC3N3CCN(C)CC3)nc3c2CCN(c2cccc4ccccc24)C3)CC1CC#N] | MS: 621 |
| 17. | 2-(1-acryloyl-4-(2-((3- (dimethylamino)cyclopen- tyl)methoxy)-7- (naphthalen-1-yl)-5,6,7,8- tetrahydropyrido[3,4- d]pyrimidin-4- yl)piperazin-2- yl)acetonitrile | 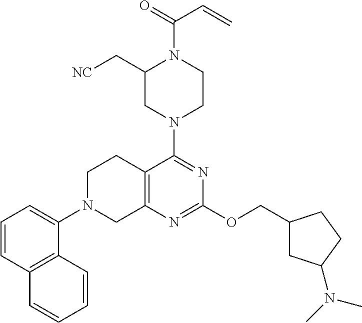 [SMILES: C=CC(=O)N1CCN(c2nc(OCC3CCC(N(C)C)C3)nc3c2CCN(c2cccc4ccccc24)C3)CC1CC#N] | MS: 580 ¹H NMR (400 MHz, MeOD) δ 8.24 (d, 1H), 7.87 (d, 1H), 7.61 (d, 1H), 7.56-7.47 (m, 2H), 7.44 (t, 1H), 7.21 (d, 1H), 6.82 (s, 1H), 6.30 (d, 1H), 5.85 (d, 1H), 4.38- 4.07 (m, 7H), 3.77-3.57 (m, 1H), 3.52-3.40 (m, 1H), 3.12- 2.83 (m, 4H), 2.72-2.57 (m, 1H), 2.52-2.40 (m, 1H), 2.31 (s, 6H), 2.23-2.10 (m, 1H), 2.05- 1.82 (m, 2H), 1.81-1.67 (m, 1H), 1.66-1.40 (m, 3H), 1.39- 1.17 (m, 2H), 1.05-0.95 (m, 1H). |
| 18. | 2-(1-acryloyl-4-(2-((2- (dimethylamino)cyclohex- yl)methoxy)-7- (naphthalen-1-yl)-5,6,7,8- tetrahydropyrido[3,4- d]pyrimidin-4- yl)piperazin-2- yl)acetonitrile | 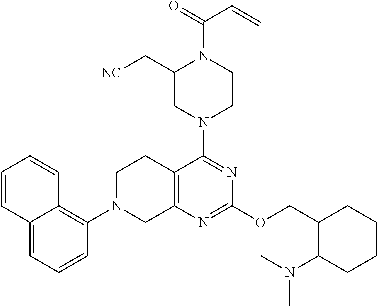 [SMILES: C=CC(=O)N1CCN(c2nc(OCC3CCCCC3N(C)C)nc3c2CCN(c2cccc4ccccc24)C3)CC1CC#N] | MS: 594 ¹H NMR (400 MHz, MeOD) δ 8.21-8.06 (m, 1H), 7.85-7.69 (m, 1H), 7.52 (d, 1H), 7.46-7.24 (m, 3H), 7.10 (d, 1H), 6.72 (s, lH), 6.20 (d, 1H), 5.74 (d, 1H), 4.99 (s, 1H), 4.57-4.40 (m, 2H), 4.22 (d, 1H), 4.16-3.90 (m, 3H), 3.53 (s, 2H), 3.33 (s, 2H), 3.27- 3.05 (m, 2H), 2.87 (d, 5H), 2.75 (s, 6H), 2.16-1.75 (m, 4H), 1.68- 1.28 (m, 6H). |
| 19. | 2-(1-acryloyl-4-(2-((2- ((dimethylamino)methyl) cyclopropyl)methoxy)-7- (naphthalen-1-yl)-5,6,7,8- tetrahydropyrido[3,4- d]pyrimidin-4- yl)piperazin-2- yl)acetonitrile | 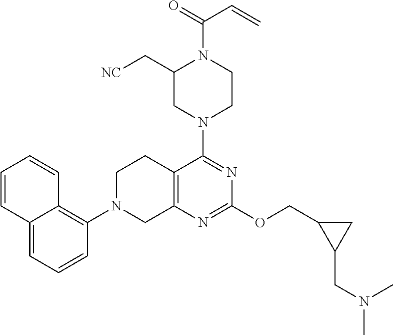 [SMILES: C=CC(=O)N1CCN(c2nc(OCC3CC3CN(C)C)nc3c2CCN(c2cccc4ccccc24)C3)CC1CC#N] | MS: 566 |
| 20. | 2-(1-acryloyl-4-(2-((1- (dimethylamino)cyclopro- pyl)methoxy)-7-(8- methylnaphthalen-1-yl)- 5,6,7,8- tetrahydropyrido[3,4- d]pyrimidin-4- yl)piperazin-2- yl)acetonitrile | 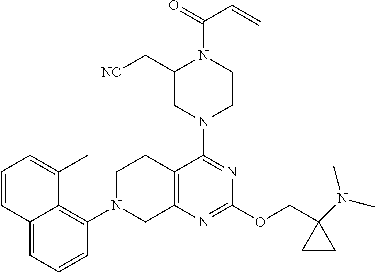 [SMILES: C=CC(=O)N1CCN(c2nc(OCC3(N(C)C)CC3)nc3c2CCN(c2cccc4cccc(C)c24)C3)CC1CC#N] | MS: 566 ¹H NMR (400 MHz, MeOD) δ 7.67-7.48 (m, 2H), 7.39-7.26 (m, 1H), 7.26-7.08 (m, 3H), 6.70 (s, 1H), 6.19 (d, 1H), 5.00 (s, 1H), 4.47 (s, 1H), 4.33 (s, 2H), 4.21 (d, 1H), 4.14-3.89 (m, 3H), 3.69-3.49 (m, 1H), 3.48-3.27 (m, 2H), 3.18-2.90 (m, 4H), 2.86-2.75 (m, 4H), 2.63 (d, 1H), 2.38 (d, 6H), 2.32-2.19 (m, 1H), 0.90-0.54 (m, 4H). |
| 21. | 2-(1-acryloyl-4-(2-(2-(2- (dimethylamino)cyclopen- tyl)ethoxy)-7- (naphthalen-1-yl)-5,6,7,8- tetrahydropyrido[3,4- d]pyrimidin-4- yl)piperazin-2- yl)acetonitrile | 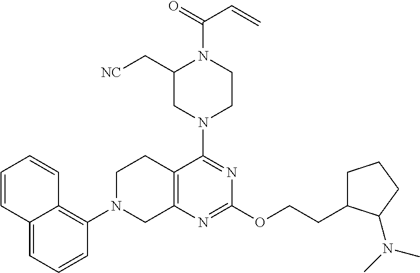 [SMILES: C=CC(=O)N1CCN(c2nc(OCCC3CCCC3N(C)C)nc3c2CCN(c2cccc4ccccc24)C3)CC1CC#N] | MS: 594 ¹H NMR (400 MHz, MeOD) δ 8.13 (d, 1H), 7.84-7.67 (m, 1H), 7.58-7.48 (m, 1H), 7.47-7.24 (m, 3H), 7.10 (d, 1H), 6.72 (s, 1H), 6.19 (d, 1H), 5.74 (d, 1H), 4.99 (s, 1H), 4.52-4.36 (m, 1H), 4.29 (s, 1H), 4.19 (s, 1H), 4.13- 3.87 (m, 3H), 3.42-3.26 (m, 2H), 3.17-2.77 (m, 6H), 2.70- 2.52 (m, 4H), 2.43-2.29 (m, 1H), 2.14-1.90 (m, 3H), 1.89- 1.61 (m, 5H), 1.61-1.34 (m, 3H), 0.86-0.72 (m, 2H). |
| 22. | 2-(1-acryloyl-4-(2- (((1R,3S)-3- (dimethylamino)cyclopen- tyl)methoxy)-7-(8- methylnaphthalen-1-yl)- 5,6,7,8- tetahydropyrido[3,4- d]pyrimidin-4- yl)piperazin-2- yl)acetonitrile | 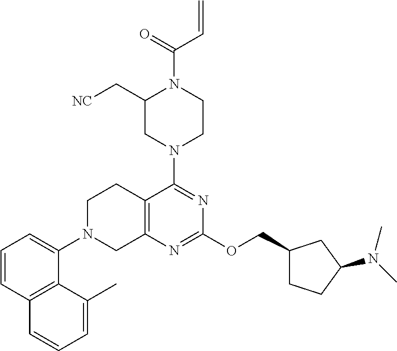 [SMILES: C=CC(=O)N1CCN(c2nc(OC[C@@H]3CC[C@H](N(C)C)C3)nc3c2CCN(c2cccc4cccc(C)c24)C3)CC1CC#N] | MS: 594 ¹H NMR (400 MHz, CDCl₃) δ 7.75-7.55 (m, 2H), 7.45-7.17 (m, 4H), 6.55 (s, 1H), 6.32 (d, 1H), 5.75 (d, 1H), 4.19 (dd, 4H), 3.36 (d, 3H), 3.11-3.00 (m, 5H), 2.84 (s, 3H), 2.72 (s, 7H), 1.32 (dd, 5H), 1.25 (s, 5H), 0.80 (d, 2H) |
| 23. | 2-(1-acryloyl-4-(2-((2- (dimethylamino)cyclopen- tyl)methoxy)-7-(8- methylnaphthalen-1-yl)- 5,6,7,8- tetrahydropyrido[3,4- d]pyrimidin-4- yl)piperazin-2- yl)acetonitrile | 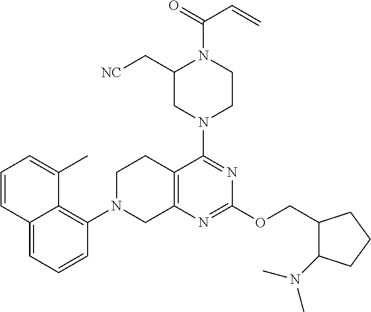 [SMILES: C=CC(=O)N1CCN(c2nc(OCC3CCCC3N(C)C)nc3c2CCN(c2cccc4cccc(C)c24)C3)CC1CC#N] | MS: 594 ¹H NMR (400 MHz, CDCl₃) δ 7.70-7.52 (m, 2H), 7.31 (ddd, 3H), 7.19-7.14 (m, 1H), 6.56 (s, 1H), 6.31 (d, 1H), 5.72 (d, 1H), 4.95 (s, 1H), 4.70 (s, 2H), 4.34-3.91 (m, 4H), 3.80-3.57 (m, 2H), 3.43 (s, 2H), 3.23 (s, 2H), 3.10 (s, 2H), 2.85 (s, 5H), 2.74 (d, 5H), 2.56 (d, 1H), 2.23- 1.88 (m, 4H), 1.72 (s, 2H), 1.29- 1.13 (m, 2H). |
| 24. | 2-(1-acryloyl-4-(2-((3- (dimethylamino)cyclopen- tyl)methoxy)-7-(8- methylnaphthalen-1-yl)- 5,6,7,8- tetrahydropyrido[3,4- d]pyrimidin-4- yl)piperazin-2- yl)acetonitrile | 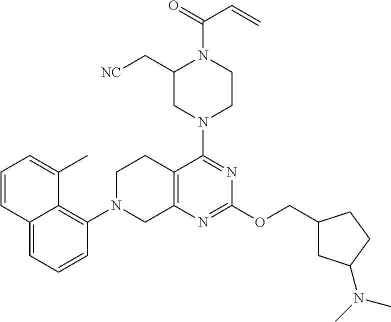 [SMILES: C=CC(=O)N1CCN(c2nc(OCC3CCC(N(C)C)C3)nc3c2CCN(c2cccc4cccc(C)c24)C3)CC1CC#N] | MS: 594 ¹H NMR (400 MHz, MeOD) δ 7.71 (dd, 4.7 Hz, 2H), 7.48-7.41 (m, 1H), 7.38-7.32 (m, 2H), 7.31-7.23 (m, 1H), 6.91-6.73 (m, 1H), 6.31 (d, 1H), 5.85 (d, 1H), 5.15-4.93 (m, 1H), 4.68- 4.37 (m, 4H), 4.25-4.04 (m, 2H), 3.95-3.80 (m, 2H), 3.72- 3.47 (m, 4H), 3.28-3.17 (m, 2H), 3.11-3.03 (m, 1H), 2.93 (d, 3H), 2.90 (s, 6H), 2.79-2.68 (m, 1H), 2.66-2.49 (m, 1H), 2.46- 2.35 (m, 1H), 2.27-2.15 (m, 1H), 2.06-1.50 (m, 5H). |
| 25. | 2-(1-acryloyl-4-(7-(2,3- dichlorophenyl)-2-((2- (dimethylamino)cyclopen- tyl)methoxy)-5,6,7,8- tetrahydropyrido[3,4- d]pyrimidin-4- yl)piperazin-2- yl)acetonitrile | 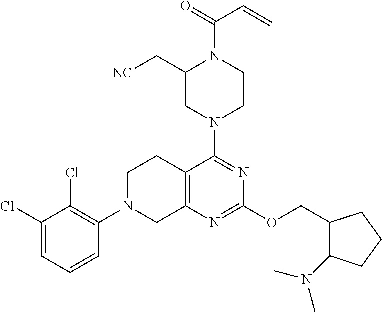 [SMILES: C=CC(=O)N1CCN(c2nc(OCC3CCCC3N(C)C)nc3c2CCN(c2cccc(Cl)c2Cl)C3)CC1CC#N] | MS: 598 ¹H NMR (400 MHz, CDCl₃) δ 7.12 (q, 2H), 6.93 (d, 1H), 6.54 (s, 1H), 6.30 (d, 1H), 5.72 (d, 1H), 4.96 (s, 1H), 4.74 (s, 2H), 4.34- 3.78 (m, 5H), 3.33 (dd, 5H), 3.11 (s, 1H), 2.86 (d, 7H), 2.71 (s, 2H), 2.13 (s, 3H), 1.94 (s, 1H), 1.74 (s, 2H), 1.19 (s, 2H) |
| 26. | 2-(1-acryloyl-4-(2-((2- (dimethylamino)cyclopen- tyl)methoxy)-7-(3-fluoro- 2- (trifluoromethyl)phenyl)- 5,6,7,8- tetrahydropyrido[3,4- d]pyrimidin-4- yl)piperazin-2- yl)acetonitrile | 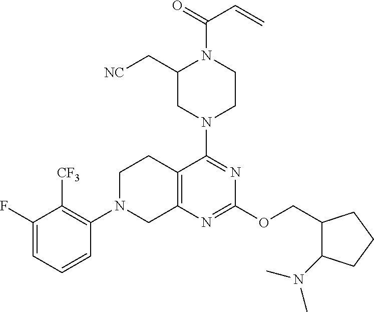 [SMILES: C=CC(=O)N1CCN(c2nc(OCC3CCCC3N(C)C)nc3c2CCN(c2cccc(F)c2C(F)(F)F)C3)CC1CC#N] | MS: 616 ¹H NMR (400 MHz, CDDCl₃) δ 7.49 (dd, 1H), 7.07 (d, 1H), 7.02- 6.90 (m, 1H), 6.65 (s, 1H), 6.39 (d, 1H), 5.81 (d, 1H), 5.05 (s, 1H), 4.84 (s, 2H), 4.25 (dd, 2H), 4.12 (s, 3H), 3.37 (dd, 5H), 3.18 (s, 1H), 2.94 (d, 7H), 2.80 (s, 2H), 2.18 (dd, 4H), 1.83 (s, 2H), 1.35- 1.21 (m, 2H) |
| 27. | 2-(1-acryloyl-4-(2-((2- (dimethylamino)cylcopen- tyl)methoxy)-7-(2- (trifluoromethyl)phenyl)- 5,6,7,8- tetrahydropyrido[3,4- d]pyrimidin-4- yl)piperazin-2- yl)acetonitrile | 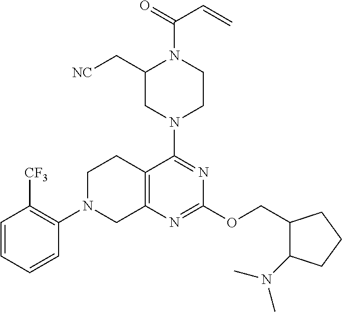 [SMILES: C=CC(=O)N1CCN(c2nc(OCC3CCCC3N(C)C)nc3c2CCN(c2ccccc2C(F)(F)F)C3)CC1CC#N] | MS: 598 ¹H NMR (400 MHz, MeOD) δ 7.70-7.52 (m, 2H), 7.52-7.43 (m, 1H), 7.35-7.20 (m, 1H), 6.71 (s, 1H), 6.19 (d, 1H), 5.80- 5.66 (m, 1H), 5.00 (s, 1H), 4.44- 4.30 (m, 1H), 4.25-3.97 (m, 3H), 3.90 (s, 2H), 3.53 (s, 1H), 3.16-2.99 (m, 3H), 2.91 (s, 2H), 2.77 (s, 2H), 2.59 (d, 3H), 2.40 (s, 6H), 2.02-1.41 (m, 7H). |
| 28. | 2-(1-acryloyl-4-(2-((2- (dimethylamino)cyclopen- tyl)methoxy)-7-(2,3- dimethylphenyl)-5,6,7,8- tetrahydropyrido[3,4- d]pyrimidin-4- yl)piperazin-2- yl)acetonitrile | 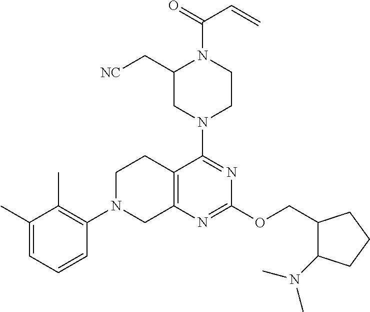 [SMILES: C=CC(=O)N1CCN(c2nc(OCC3CCCC3N(C)C)nc3c2CCN(c2cccc(C)c2C)C3)CC1CC#N] | MS: 558 |
| 29. | 2-(1-acryloyl-4-(7-(2- chloro-3-fluorophenyl)-2- ((3- (dimethylamino)cyclopen- tyl)methoxy)-5,6,7,8- tetrahydropyrido[3,4- d]pyrimidin-4- yl)piperazin-2- yl)acetonitrile | 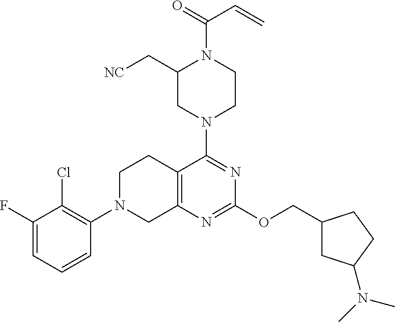 [SMILES: C=CC(=O)N1CCN(c2nc(OCC3CCC(N(C)C)C3)nc3c2CCN(c2cccc(F)c2Cl)C3)CC1CC#N] | MS: 582 |
| 30. | 2-(1-acryloyl-4-(2-((3- (dimethylamino)cyclopen- tyl)methoxy)-7-(4- (trifluoromethyl)61aphthal- 3-yl)-5,6,7,8- tetrahydropyrido[3,4- d]pyrimidin-4- yl)piperazin-2- yl)acetonitrile | 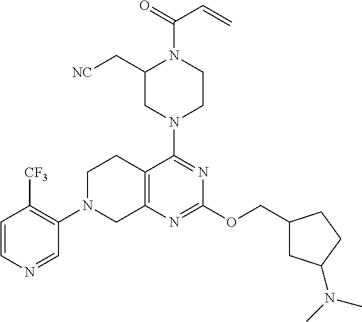 [SMILES: C=CC(=O)N1CCN(c2nc(OCC3CCC(N(C)C)C3)nc3c2CCN(c2cnccc2C(F)(F)F)C3)CC1CC#N] | MS: 599 |
| 31. | 2-(1-acryloyl-4-(7-(2- chlorophenyl)-2-((3- (dimethylamino)cyclopen- tyl)methoxy)-5,6,7,8- tetrahydropyrido[3,4- d]pyrimidin-4- yl)piperazin-2- yl)acetonitrile | 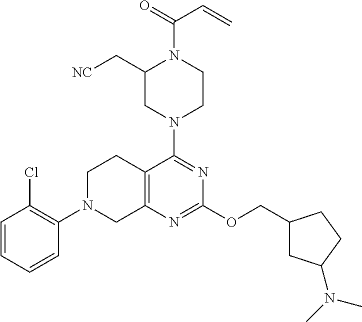 [SMILES: C=CC(=O)N1CCN(c2nc(OCC3CCC(N(C)C)C3)nc3c2CCN(c2ccccc2Cl)C3)CC1CC#N] | MS: 564 |
| 32. | 2-(1-acryloyl-4-(7- (naphthalen-1-yl)-2-((1- (pyrrolidin-1- yl)cyclopropyl)methoxy)- 5,6,7,8- tetrahydropyrido[3,4- d]pyrimidin-4- yl)piperazin-2- yl)acetonitrile | 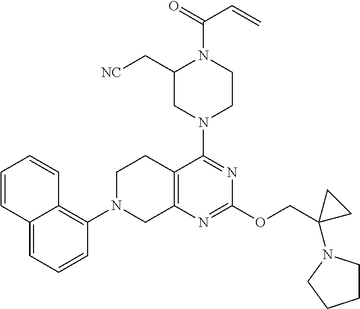 [SMILES: C=CC(=O)N1CCN(c2nc(OCC3(N4CCCC4)CC3)nc3c2CCN(c2cccc4ccccc24)C3)CC1CC#N] | MS: 578 |
| 33. | 2-(1-acryloyl-4-(7- (62aphthalene-1-yl)-2- ((3-(pyrrolidin-1- yl)cyclopentyl)methoxy)- 5,6,7,8- tetrahydropyrido[3,4- d]pyrimidin-4- yl)piperazin-2- yl)acetonitrile | 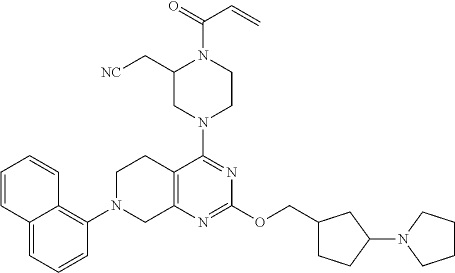 [SMILES: C=CC(=O)N1CCN(c2nc(OCC3CCC(N4CCCC4)C3)nc3c2CCN(c2cccc4ccccc24)C3)CC1CC#N] | MS: 606 ¹H NMR (400 MHz, MeOD) δ 8.25 (d, 1H), 7.89 (d, 1H), 7.76- 7.39 (m, 4H), 7.22 (d, 1H), 6.91 (d, 1H), 6.31 (d, 1H), 5.86 (d, 1H), 5.11 (s, 1H), 4.58 (s, 1H), 4.45-4.01 (m, 5H), 3.78-3.59 (m, 2H), 3.45 (s, 2H), 3.28-3.10 (m, 7H), 3.13-2.89 (m, 3H), 2.63-2.33 (m, 2H), 2.32-1.54 (m, 8H), 1.02-0.81 (m, 2H). |
| 34. | 2-(1-acryloyl-4-(7-(3- fluoro-2- (trifluoromethyl)phenyl)- 2-((1-(pyrrolidin-1- ylmethyl)cyclopropyl)meth- oxy)-5,6,7,8- tetrahydropyrido[3,4- d]pyrimidin-4- yl)piperazin-2- yl)acetonitrile | 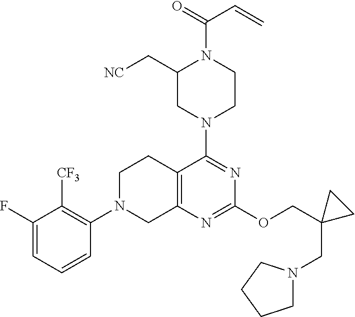 [SMILES: C=CC(=O)N1CCN(c2nc(OCC3(CN4CCCC4)CC3)nc3c2CCN(c2cccc(F)c2C(F)(F)F)C3)CC1CC#N] | MS: 628 |
| 35. | 2-(1-acryloyl-4-(7-(2,3- dichlorophenyl)-2-((1- (pyrrolidin-1- ylmethyl)cyclopropyl)meth- oxy)-5,6,7,8- tetrahydropyrido[3,4- d]pyrimidin-4- yl)piperazin-2- yl)acetonitrile | 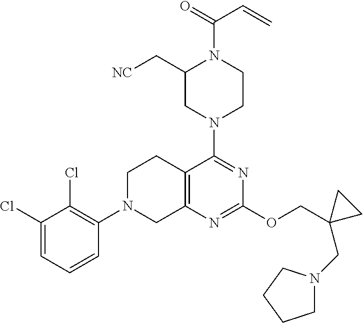 [SMILES: C=CC(=O)N1CCN(c2nc(OCC3(CN4CCCC4)CC3)nc3c2CCN(c2cccc(Cl)c2Cl)C3)CC1CC#N] | MS: 610 ¹H NMR (400 MHz, MeOD) δ 7.26-7.16 (m, 2H), 7.14-7.03 (m, 1H), 6.73 (s, 1H), 6.18 (d, 1H), 5.73 (d, 1H), 4.98 (s, 1H), 4.45 (s, 1H), 4.29-3.85 (m, 6H), 3.60-3.41 (m, 1H), 3.36-3.01 (m, 3H), 2.96-2.75 (m, 3H), 2.58 (d, 5H), 2.27-2.05 (m, 1H), 1.79-1.66 (m, 3H), 1.30-1.08 (m, 2H), 0.94-0.69 (m, 1H), 0.58 (s, 2H), 0.48 (s, 2H). |
| 36. | 2-(1-acryloyl-4-(2-((1- (pyrrolidin-1- ylmethyl)cyclopropyl)meth- oxy)-7-(2- (trifluoromethyl)pyridin- 3-yl)-5,6,7,8- tetrahydropyrido[3,4- d]pyrimidin-4- yl)piperazin-2- yl)acetonitrile | 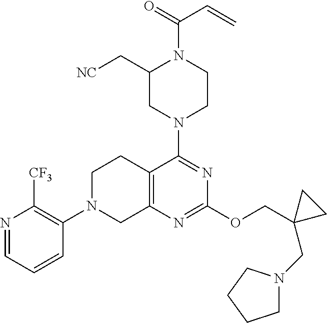 [SMILES: C=CC(=O)N1CCN(c2nc(OCC3(CN4CCCC4)CC3)nc3c2CCN(c2cccnc2C(F)(F)F)C3)CC1CC#N] | MS: 611 |
| 37. | 2-(1-acryloyl-4-(2-((1- ((dimethylamino)methyl) cyclopropyl)methoxy)-7- (naphthalen-1-yl)-5,6,7,8- tetrahydropyrido[3,4- d]pyrimidin-4- yl)piperazin-2- yl)acetonitrile | 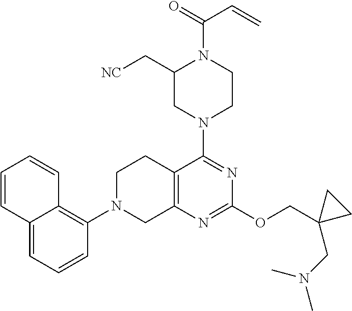 [SMILES: C=CC(=O)N1CCN(c2nc(OCC3(CN(C)C)CC3)nc3c2CCN(c2cccc4ccccc24)C3)CC1CC#N] | MS: 566 ¹H NMR (400 MHz, MeOD) δ 8.14 (d, J = 3.4 Hz, 1H), 7.77 (d, J = 5.1 Hz, 1H), 7.52 (d, J = 8.1 Hz, 1H), 7.43-7.37 (m, 2H), 7.34 (d, J = 7.7 Hz, 1H), 7.10 (d, J = 7.3 Hz, 1H), 6.73 (s, 1H), 6.22 (s, 1H), 5.74 (d, J = 10.0 Hz, 1H), 4.15 (d, J = 46.2 Hz, 6H), 2.92 (s, 6H), 2.77-2.55 (m, 7H), 1.19 (s, 6H), 0.78 (m, 2H), 0.64 (m, 2H). |
| 38. | 2-(1-acryloyl-4-(7-(3- chloro-2- (trifluoromethyl)phenyl)- 2-((1-(pyrrolidin-1- ylmethyl)cyclopropyl)meth- oxy)-5,6,7,8- tetrahydropyrido[3,4- d]pyrimidin-4- yl)piperazin-2- yl)acetonitrile | 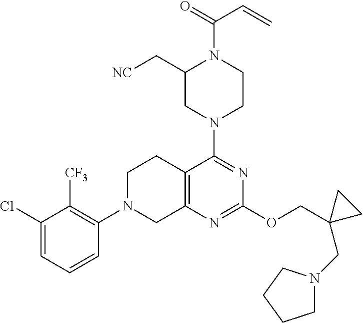 [SMILES: C=CC(=O)N1CCN(c2nc(OCC3(CN4CCCC4)CC3)nc3c2CCN(c2cccc(Cl)c2C(F)(F)F)C3)CC1CC#N] | MS: 644 |
| 39. | 2-(1-acryloyl-4-(2-((1- (azetidin-1- ylmethyl)cyclopropyl)meth- oxy)-7-(3-chloro-2- (trifluoromethyl)phenyl)- 5,6,7,8- tetrahydropyrido[3,4- d]pyrimidin-4- yl)piperazin-2- yl)acetonitrile | 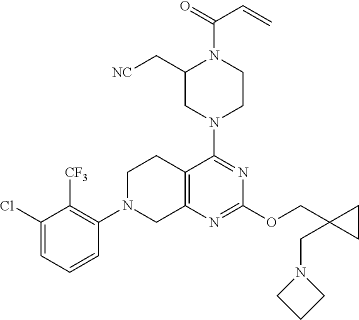 [SMILES: C=CC(=O)N1CCN(c2nc(OCC3(CN4CCC4)CC3)nc3c2CCN(c2cccc(Cl)c2C(F)(F)F)C3)CC1CC#N] | MS: 630 ¹H NMR (400 MHz, MeOD) δ 7.45 (t, J = 8.1 Hz, 1H), 7.28 (dt, J = 19.1, 9.6 Hz, 2H), 6.71 (s, 1H), 6.19 (d, J = 16.9 Hz, 1H), 5.73 (d, J = 10.6 Hz, 1H), 4.13 (s, 5H), 3.95 (s, 4H), 3.11 (q, J = 7.3 Hz, 6H), 2.96-2.61 (m, 5H), 1.21 (dd, J = 12.6, 5.3 Hz, 5H), 0.72 (m, 4H). |
| 40. | 2-(1-acryloyl-4-(7-(3- chloro-2- (trifluoromethyl)phenyl)- 2-((1- (morpholinomethyl)cyclo- propyl)methoxy)-5,6,7,8- tetrahydropyrido[3,4- d]pyrimidin-4- yl)piperazin-2- yl)acetonitrile | 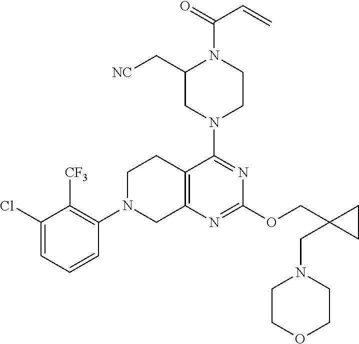 [SMILES: C=CC(=O)N1CCN(c2nc(OCC3(CN4CCOCC4)CC3)nc3c2CCN(c2cccc(Cl)c2C(F)(F)F)C3)CC1CC#N] | MS: 660 |
| 41. | 2-(1-acryloyl-4-(2-((1- (azetidin-1- ylmethyl)cyclopropyl)meth- oxy)-7-(naphthalen-1- yl)-5,6,7,8- tetrahydropyrido[3,4- d]pyrimidin-4- yl)piperazin-2- yl)acetonitrile | 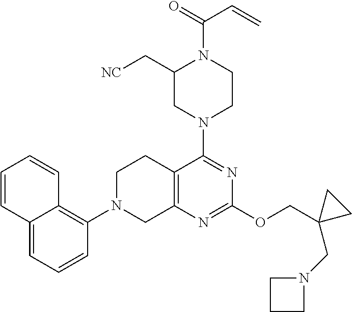 [SMILES: C=CC(=O)N1CCN(c2nc(OCC3(CN4CCC4)CC3)nc3c2CCN(c2cccc4ccccc24)C3)CC1CC#N] | MS: 578 ¹H NMR (400 MHz, MeOD) δ 8.29-8.23 (m, 1H), 7.94-7.87 (m, 1H), 7.63 (s, 1H), 7.55-7.50 (m, 2H), 7.46 (s, 1H), 7.23 (d, J = 7.4 Hz, 1H), 6.85 (s, 1H), 6.31 (d, J = 16.6 Hz, 1H), 5.86 (d, J = 10.6 Hz, 1H), 4.24 (d, J = 13.7 Hz, 8H), 3.20 (dt, J = 22.4, 11.2 Hz, 3H), 3.13-2.90 (m, 4H), 2.50 (s, 2H), 1.32 (dd, J = 15.3, 7.8 Hz, 8H), 0.83 (s, 4H). |
| 42. | 2-(1-acryloyl-4-(2-((1- (azetidin-1- ylmethyl)cyclopropyl)meth- oxy)-7-(8- chloronaphthalen-1-yl)- 5,67,8- tetrahydropyrido[3,4- d]pyrimidin-4- yl)piperazin-2- yl)acetonitrile | 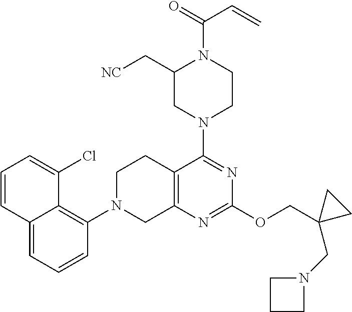 [SMILES: C=CC(=O)N1CCN(c2nc(OCC3(CN4CCC4)CC3)nc3c2CCN(c2cccc4cccc(Cl)c24)C3)CC1CC#N] | MS: 612 |
| 43. | 2-(1-acryloyl-4-(2-((1- (azetidin-1- ylmethyl)cyclopropyl)meth- oxy)-7-(8- methylnaphthalen-1-yl)- 5,6,7,8- tetrahydropyrido[3,4- d]pyrimidin-4- yl)piperazin-2- yl)acetonitrile | 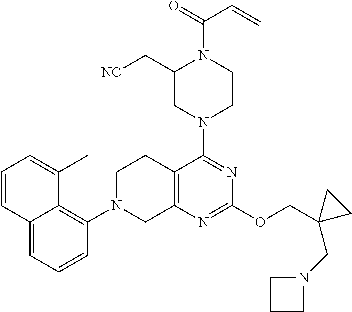 [SMILES: C=CC(=O)N1CCN(c2nc(OCC3(CN4CCC4)CC3)nc3c2CCN(c2cccc4cccc(C)c24)C3)CC1CC#N] | MS: 592 |
| 44. | 2-(1-acryloyl-4-(7- (naphthalen-1-yl)-2-((1- (pyrrolidin-1- ylmethyl)cyclobutyl)meth- oxy)-5,6,7,8- tetrahydropyrido[3,4- d]pyrimidin-4- yl)piperazin-2- yl)acetonitrile | 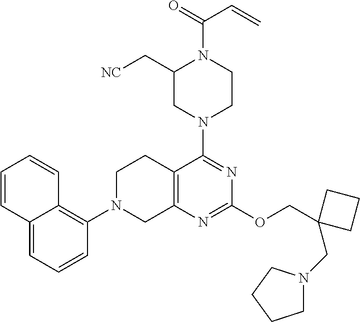 [SMILES: C=CC(=O)N1CCN(c2nc(OCC3(CN4CCCC4)CCC3)nc3c2CCN(c2cccc4ccccc24)C3)CC1CC#N] | MS: 606 ¹H NMR (400 MHz, MeOD) δ 8.32-8.22 (m, 1H), 7.93-7.86 (m, 1H), 7.65 (d, J = 8.2 Hz, 1H), 7.58-7.40 (m, 3H), 7.24 (d, J = 7.4 Hz, 1H), 6.97-6.77 (m, 1H), 6.33 (d, J = 16.7 Hz, 1H), 5.87 (d, J = 10.6 Hz, 1H), 4.58 (s, 2H), 4.46-4.32 (m, 1H), 4.30-4.02 (m, 4H), 3.79-3.38 (m, 4H), 3.30-2.94 (m, 10H), 2.09 (s, 2H), 1.49-1.12 (m, 10H). |
| 45. | 2-(1-acryloyl-4-(7-(8- methylnaphthalen-1-yl)- 2-((1-(pyrrolidin-1- ylmethyl)cyclobutyl)meth- oxy)-5,6,7,8- tetrahydropyrido[3,4- d]pyrimidin-4- yl)piperazin-2- yl)acetonitrile | 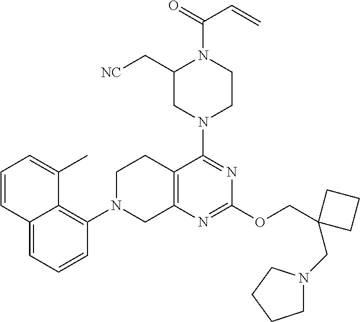 [SMILES: C=CC(=O)N1CCN(c2nc(OCC3(CN4CCCC4)CCC3)nc3c2CCN(c2cccc4cccc(C)c24)C3)CC1CC#N] | MS: 620 ¹H NMR (400 MHz, MeOD) δ 7.37-7.64 (m, 2H), 7.47-7.22 (m, 4H), 7.06-6.66 (m, 1H), 6.30 (d, J = 6.3 Hz, 1H), 5.84 (d, J = 10.8 Hz, 1H), 4.62-4.51 (m, 2H), 4.33-4.23 (m, 1H), 4.20- 3.99 (m, 2H), 3.83-3.44 (m, 3H), 3.25-3.06 (m, 6H), 2.92 (s, 3H), 2.84-2.66 (m, 2H), 2.12- 1.91 (m, 13H), 1.31-1.27 (m, 4H). |
| 46. | 2-(1-acryloyl-4-(7-(8- chloronaphthalen-1-yl)-2- ((1- ((dimethylamino)methyl) cyclopropyl)methoxy)- 5,6,7,8- tetrahydropyrido[3,4- d]pyrimidin-4- yl)piperazin-2- yl)acetonitrile | 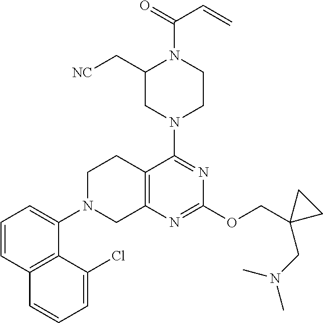 [SMILES: C=CC(=O)N1CCN(c2nc(OCC3(CN(C)C)CC3)nc3c2CCN(c2cccc4cccc(Cl)c24)C3)CC1CC#N] | MS: 600 ¹H NMR (400 MHz, MeOD) δ 7.89 (d, J = 8.1 Hz, 1H), 7.74 (d, J = 8.0 Hz, 1H), 7.63-7.51 (m, 2H), 7.48-7.33 (m, 2H), 7.05- 6.77 (m, 1H), 6.35 (d, J = 16.9 Hz, 1H), 5.90 (d, J = 10.6 Hz, 1H), 4.49-4.08 (m, 6H), 3.92- 3.43 (m, 4H), 3.29-3.12 (m, 3H), 3.04-2.90 (m, 1H), 2.86- 2.66 (m, 1H), 2.66-2.44 (m, 2H), 2.32 (s, 6H), 1.47-1.27 (m, 2H), 0.82-0.66 (m, 2H), 0.65- 0.45 (m, 2H). |
| 47. | 2-(1-acryloyl-4-(2-((1- ((dimethylamino)methyl) cyclopropyl)methoxy)-7- (8-methylnaphthalen-1- yl)-5,6,7,8- tetrahydropyrido[3,4- d]pyrimidin-4- yl)piperazin-2- yl)acetonitrile | 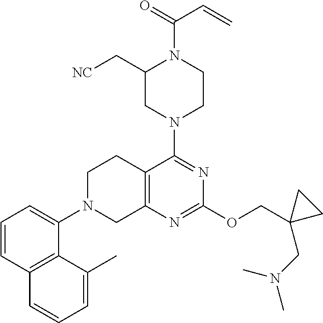 [SMILES: C=CC(=O)N1CCN(c2nc(OCC3(CN(C)C)CC3)nc3c2CCN(c2cccc4cccc(C)c24)C3)CC1CC#N] | MS: 580 ¹H NMR (400 MHz, MeOD) δ 7.82-7.65 (m, 2H), 7.49-7.40 (m, 1H), 7.39-7.23 (m, 3H), 7.01-6.75 (m, 1H), 6.32 (d, J = 16.4 Hz, 1H), 5.86 (d, J = 10.9 Hz, 1H), 4.45-3.91 (m, 6H), 3.73 (t, J= 17.5 Hz, 1H), 3.63- 3.42 (m, 2H), 3.33-3.03 (m, 5H), 2.94 (s, 3H), 2.92-2.82 (m, 1H), 2.82-2.64 (m, 1H), 2.59- 2.42 (m, 2H), 2.24 (s, 6H), 1.40- 1.25 (m, 1H), 0.78-0.63 (m, 2H), 0.59-0.40 (m, 2H). |
| 48. | 2-(1-acryloyl-4-(2-((1- ((dimethylamino)methyl) cyclopropyl)methoxy)-7- (2,3-dimethylphenyl)- 5,6,7,8- tetrahydropyrido[3,4- d]pyrimidin-4- yl)piperazin-2- yl)acetonitrile | 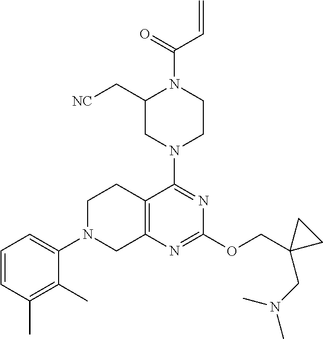 [SMILES: C=CC(=O)N1CCN(c2nc(OCC3(CN(C)C)CC3)nc3c2CCN(c2cccc(C)c2C)C3)CC1CC#N] | MS: 544 ¹H NMR (400 MHz, MeOD) δ 7.10 (t, 1H), 7.03-6.94 (m, 2H), 6.93-6.77 (m, 1H), 6.32 (d, 1H), 5.86 (d, 1H), 4.31-4.13 (m, 4H), 3.95 (s, 2H), 3.34-3.08 (m, 7H), 3.07-2.80 (m, 4H), 2.45 (s, 2H), 2.32 (m, 12H), 0.71 (t, 2H), 0.53 (t, 2H). |
| 49. | 2-(1-acryloyl-4-(2-((1- (((R)-3-fluoropyrrolidin- 1- yl)methyl)cyclopropyl)meth- oxy)-7-(8- methylnaphthalen-1-yl)- 5,6,7,8- tetrahydropyrido[3,4- d]pyrimidin-4- yl)piperazin-2- yl)acetonitrile | 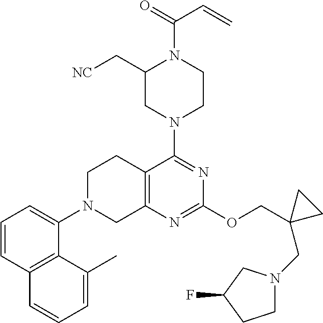 [SMILES: C=CC(=O)N1CCN(c2nc(OCC3(CN4CC[C@@H](F)C4)CC3)nc3c2CCN(c2cccc4cccc(C)c24)C3)CC1CC#N] | MS: 624 ¹H NMR (400 MHz, MeOD) δ 7.70 (m, 2H), 7.52-7.39 (m, 1H), 7.39-7.23 (m, 3H), 7.08- 6.71 (m, 1H), 6.32 (d, 1H), 5.86 (d, 1H), 4.43-4.04 (m, 6H), 3.85- 3.37 (m, 4H), 3.30-2.97 (m, 6H), 2.94 (s, 3H), 2.91-2.63 (m, 4H), 2.56 (d, 2H), 2.23-1.97 (m, 2H), 1.32 (s, 2H), 0.75-0.43 (m, 4H). |
| 50. | 2-(1-acryloyl-4-(7- (benzo[b]thiophen-7-yl)- 2-((1- ((dimethylamino)methyl) cyclopropyl)methoxy)- 5,6,7,8- tetrahydropyrido[3,4- d]pyrimidin-4- yl)piperazin-2- yl)acetonitrile | 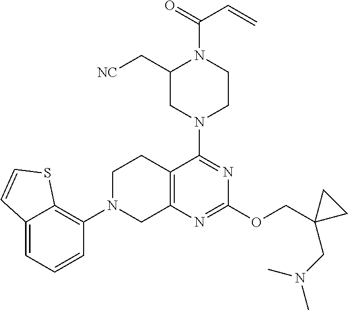 [SMILES: C=CC(=O)N1CCN(c2nc(OCC3(CN(C)C)CC3)nc3c2CCN(c2cccc4ccsc24)C3)CC1CC#N] | MS: 572 |
| 51. | 2-(1-acryloyl-4-(7- (benzo[b]thiophen-4-yl)- 2-((1- ((dimethylamino)methyl) cyclopropyl)methoxy)- 5,6,7,8- tetrahydropyrido[3,4- d]pyrimidin-4- yl)piperazin-2- yl)acetonitrile | 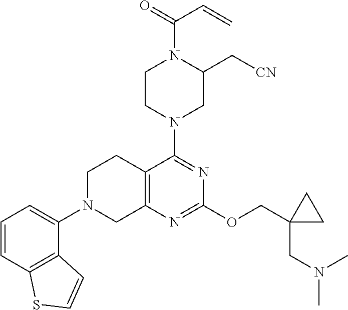 [SMILES: C=CC(=O)N1CCN(c2nc(OCC3(CN(C)C)CC3)nc3c2CCN(c2cccc4sccc24)C3)CC1CC#N] | MS: 572 ¹H NMR (400 MHz, MeOD) δ 7.70-7.52 (m, 3H), 7.34 (t, 1H), 7.03 (d, 1H), 6.94-6.77 (m, 1H), 6.32 (d, 1H), 5.87 (d, 1H), 4.46- 4.32 (m, 3H), 4.32-4.11 (m, 4H), 3.70-3.39 (m, 4H), 3.24-3.11 (m, 5H), 3.08-2.94 (m, 9H), 0.99-0.88 (m, 4H). |
| 52. | 2-(1-acryloyl-4-(7- (benzo[d]thiazol-4-yl)-2- ((1- ((dimethylamino)methyl) cyclopropyl)methoxy)- 5,6,7,8- tetrahydropyrido[3,4- d]pyrimidin-4- yl)piperazin-2- yl)acetonitrile | 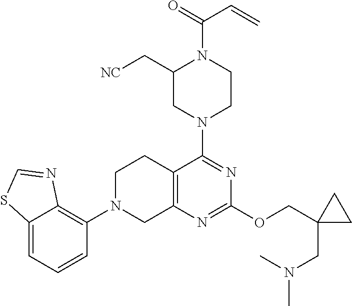 [SMILES: C=CC(=O)N1CCN(c2nc(OCC3(CN(C)C)CC3)nc3c2CCN(c2cccc4scnc24)C3)CC1CC#N] | MS: 573 |
| 53. | 2-(1-acryloyl-4-(7-(2,3- dihydrobenzofuran-7-yl)- 2-((1- ((dimethylamino)methyl) cyclopropyl)methoxy)- 5,6,7,8- tetrahydropyrido[3,4- d]pyrimidin-4- yl)piperazin-2- yl)acetonitrile | 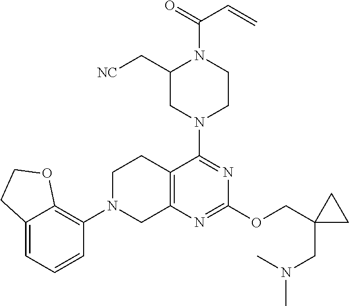 [SMILES: C=CC(=O)N1CCN(c2nc(OCC3(CN(C)C)CC3)nc3c2CCN(c2cccc4c2OCC4)C3)CC1CC#N] | MS: 558 ¹H NMR (400 MHz, MeOD) δ 6.93 (d, 1H), 6.89-6.76 (m, 3H), 6.31 (d, 1H), 5.86 (d, 1H), 4.61 (t, 2H), 4.29-4.10 (m, 7H), 3.31- 3.08 (m, 7H), 3.06-2.79 (m, 5H), 2.74-2.42 (m, 8H), 0.82- 0.57 (m, 4H). |
| 54. | 2-(1-acryloyl-4-(7- (benzo[d]thiazol-7-yl)-2- ((1- ((dimethylamino)methyl) cyclopropyl)methoxy)- 5,6,7,8- tetrahydropyrido[3,4- d]pyrimidin-4- yl)piperazin-2- yl)acetonitrile | 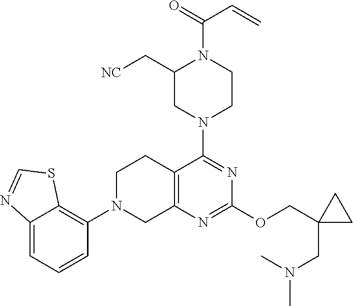 [SMILES: C=CC(=O)N1CCN(c2nc(OCC3(CN(C)C)CC3)nc3c2CCN(c2cccc4ncsc24)C3)CC1CC#N] | MS: 573 ¹H NMR (400 MHz, MeOD) δ 9.15 (s, 1H), 7.67 (d, 1H), 7.42 (t, 1H), 7.08 (d, 1H), 6.99-6.75 (m, 1H), 6.31 (d, 1H), 5.86 (d, 1H), 4.47 (m, 2H), 4.34-4.11 (m, 4H), 3.93-3.67 (m, 2H), 3.41-3.27 (m, 4H), 3.26-2.92 (m, 5H), 2.51-2.24 (m, 8H), 0.80-0.49 (m, 4H). |
| 55. | 2-(1-acryloyl-4-(7-(2- amino-6-fluorophenyl)-2- ((1- ((dimethylamino)methyl) cyclopropyl)methoxy)- 5,6,7,8- tetrahydropyrido[3,4- d]pyrimidin-4- yl)piperazin-2- yl)acetonitrile | 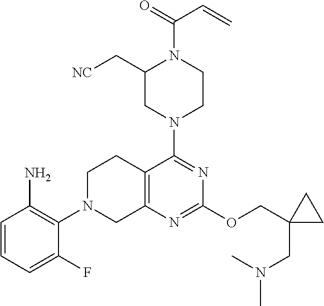 [SMILES: C=CC(=O)N1CCN(c2nc(OCC3(CN(C)C)CC3)nc3c2CCN(c2c(N)cccc2F)C3)CC1CC#N] | MS: 549 |

### Example 56

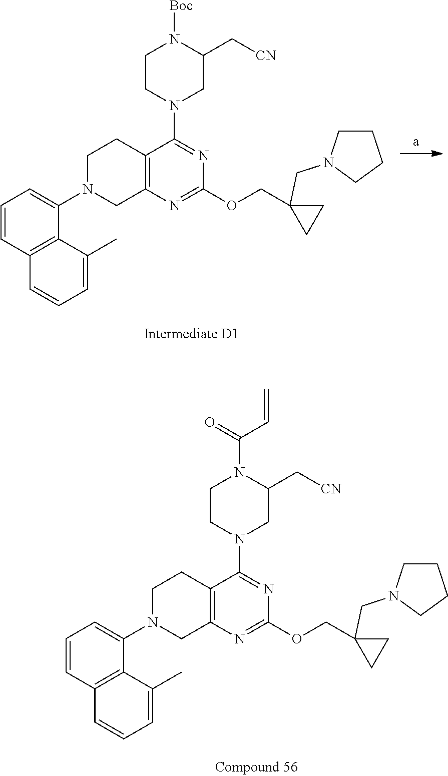 [SMILES: Cc1cccc2cccc(N3CCc4c(nc(OCC5(CN6CCCC6)CC5)nc4N4CCN(C(=O)OC(C)(C)C)C(CC#N)C4)C3)c12 ; C=CC(=O)N1CCN(c2nc(OCC3(CN4CCCC4)CC3)nc3c2CCN(c2cccc4cccc(C)c24)C3)CC1CC#N]

Step a: To a solution of Intermediate D1 (87 mg, 0.13 mmol) in DCM (10 mL) was added TFA (1 mL). The reaction mixture was stirred at 25° C. for 3 h. Upon completion, the reaction mixture was concentrated under reduced pressure. The residue was dissolved in DCM (10 mL). To the solution was added TEA (0.5 mL) and acryloyl chloride (46 mg, 0.51 mmol), then the mixture was stirred at 25° C. for 40 minutes. Upon completion, the mixture was concentrated under reduced pressure. The residue was purified by Pre-TLC (DCM:MeOH=10:1) to give Compound 56 (34 mg) as a yellow solid. MS: m/z 606[M+H]⁺, ¹H NMR (400 MHz, DMSO-d6) δ 7.76 (d, 1H), 7.73-7.66 (m, 1H), 7.52-7.41 (m, 1H), 7.41-7.24 (m, 3H), 6.94-6.77 (m, 1H), 6.19 (d, 1H), 5.78 (d, 1H), 4.10 (d, 2H), 4.08-3.79 (m, 4H), 3.79-3.55 (m, 2H), 3.42 (d, 2H), 3.29-3.16 (m, 2H), 3.16-3.02 (m, 4H), 2.98-2.89 (m, 1H), 2.86 (s, 3H), 2.81-2.57 (m, 2H), 2.47-2.24 (m, 4H), 1.81-1.49 (m, 4H), 0.70-0.22 (m, 4H).

The following Compounds were synthesized using the above procedure or modification procedure using the corresponding Intermediate.

| Compound | Chemical name | Structure | MS: (M + H)⁺/¹HNMR |
|---|---|---|---|
| 57 | 2-(1-acryloyl-4-(2-(1-(1- ((dimethylamino)methyl)cy- clopropyl)ethoxy)-7-(8- methylnaphthalen-1-yl)- 5,6,7,8- tetrahydropyrido[3,4- d]pyrimidin-4-yl)piperazin- 2-yl)acetonitrile | 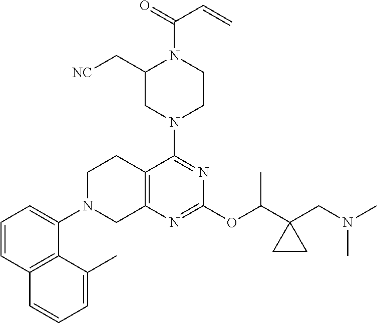 [SMILES: C=CC(=O)N1CCN(c2nc(OC(C)C3(CN(C)C)CC3)nc3c2CCN(c2cccc4cccc(C)c24)C3)CC1CC#N] | MS: 594 ¹H NMR (400 MHz, MeOD) δ 7.80-7.58 (m, 2H), 7.43 (m, 1H), 7.37-7.19 (m, 3H), 6.83 (s, 1H), 6.38-6.22 (m, 1H), 5.85 (d, 1H), 5.22-5.01 (m, 1H), 4.59 (s, 1H), 4.40-3.97 (m, 4H), 3.80-3.62 (m, 2H), 3.65-3.41 (m, 2H), 3.29-3.06 (m, 4H), 2.93 (m, 4H), 2.84- 2.65 (m, 2H), 2.61- 2.09 (m, 7H), 1.46- 1.27 (m, 3H), 0.77 (d, 2H), 0.43 (s, 2H). |
| 58 | 2-(1-acryloyl-4-(7-(8- methylnaphthalen-1-yl)-2- ((1-((4-methylpiperazin-1- yl)methyl)cyclopropyl)meth- oxy)-5,6,7,8- tetrahydropyrido[3,4- d]pyrimidin-4-yl)piperazin- 2-yl)acetonitrile | 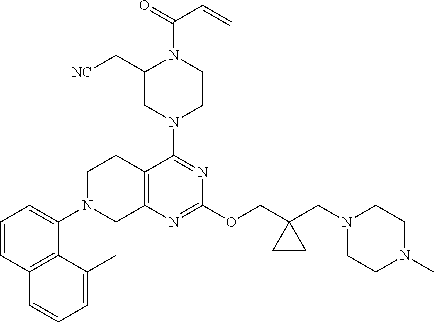 [SMILES: C=CC(=O)N1CCN(c2nc(OCC3(CN4CCN(C)CC4)CC3)nc3c2CCN(c2cccc4cccc(C)c24)C3)CC1CC#N] | MS: 635 ¹H NMR (400 MHz, MeOD) δ 7.78-7.60 (m, 2H), 7.49-7.39 (m, 1H), 7.37-7.18 (m, 3H), 6.85 (s, 1H), 6.31 (d, 1H), 5.91-5.76 (m, 1H), 5.08 (s, 1H), 4.58 (s, 1H), 4.39-4.00 (m, 5H), 3.89-3.65 (m, 2H), 3.63-3.42 (m, 2H), 3.28-2.97 (m, 6H), 2.97-2.82 (m, 4H), 2.80-2.35 (m, 9H), 2.28 (d, 3H), 0.67 (s, 2H), 0.50 (d, 2H). |
| 59 | 2-(1-acryloyl-4-(2-((1-((3- (dimethylamino)azetidin-1- yl)methyl)cyclopropyl)meth- oxy)-7-(8- methylnaphthalen-1-yl)- 5,6,7,8- tetrahydropyrido[3,4- d]pyrimidin-4-yl)piperazin- 2-yl)acetonitrile | 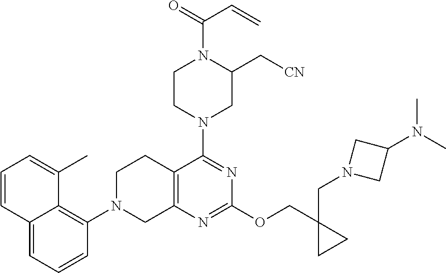 [SMILES: C=CC(=O)N1CCN(c2nc(OCC3(CN4CC(N(C)C)C4)CC3)nc3c2CCN(c2cccc4cccc(C)c24)C3)CC1CC#N] | MS: 635 |
| 60 | 2-(1-acryloyl-4-(2- (((1S,2S)-2- (dimethylamino)cyclohexyl) oxy)-7-(naphthalen-1-yl)- 5,6,7,8- tetrahydropyrido[3,4- d]pyrimidin-4-yl)piperazin- 2-yl)acetonitrile | 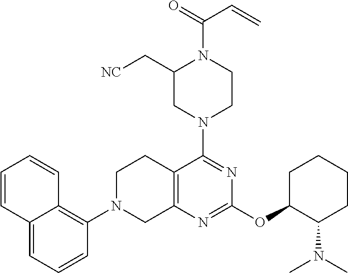 [SMILES: C=CC(=O)N1CCN(c2nc(O[C@H]3CCCC[C@@H]3N(C)C)nc3c2CCN(c2cccc4ccccc24)C3)CC1CC#N] | MS: 580 ¹H NMR (400 MHz, MeOD) δ 8.31-8.19 (m, 1H), 7.98-7.82 (m, 1H), 7.71-7.59 (m, 1H), 7.59-7.38 (m, 3H), 7.29-7.17 (m, 1H), 6.84 (d, 1H), 6.31 (d, 1H), 5.86 (d, 1H), 5.09 (s, 1H), 4.59 (s, 1H), 4.40-3.99 (m, 5H), 3.76-3.36 (m, 4H), 3.20-2.88 (m, 4H), 2.79 (s, 6H), 2.39 (s, 1H), 2.22-2.08 (m, 1H), 1.99-1.75 (m, 2H), 1.74-1.57 (m, |
| | | | 1H), 1.59-1.20 (m, |
| | | | 5H). |
| 61 | 2-(1-acryloyl-4-(7-(8- chloronaphthalen-1-yl)-2- ((1-(pyrrolidin-1- ylmethyl)cyclopropy)meth- oxy)-5,6,7,8- tetrahydropyrido[3,4- d]pyrimidin-4-yl)piperazin- 2-yl)acetonitrile | 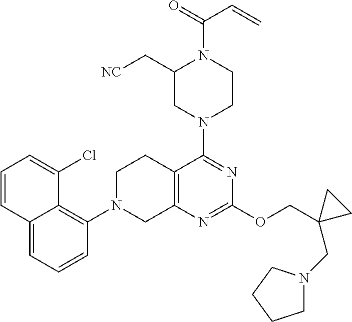 [SMILES: C=CC(=O)N1CCN(c2nc(OCC3(CN4CCCC4)CC3)nc3c2CCN(c2cccc4cccc(Cl)c24)C3)CC1CC#N] | MS: 626 ¹H NMR (400 MHz, MeOD) δ 7.75 (d, J = 8.1 Hz, 1H), 7.59 (s, 1H), 7.45 (d, J = 7.5 Hz, 2H), 7.30 (d, J = 7.8 Hz, 2H), 6.74 (s, 1H), 6.21 (d, J = 16.8 Hz, 1H), 5.75 (d, J = 10.9 Hz, 1H), 4.99 (s, 1H), 4.39 (d, J = 84.3 Hz, 1H), 4.35-4.11 (m, 4H), 4.17-3.83 (m, 3H), 3.76-3.56 (m, 2H), 3.52 (s, 1H), 3.41 (s, 1H), 3.13 (s, 4H), 2.63 (s, 6H), 1.76 (s, 4H), 0.61 (s, 2H), 0.48 (s, 2H). |
| 62 | 2-(1-acryloyl-4-(2-((1-((3- fluoroazetidin-1- yl)methyl)cyclopropyl)meth- oxy)-7-(8- methylnaphthalen-1-yl)- 5,6,7,8- tetrahydropyrido[3,4- d]pyrimidin-4-yl)piperazin- 2-yl)acetonitrile | 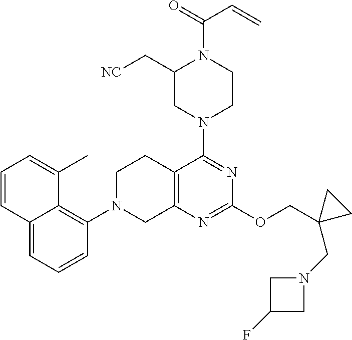 [SMILES: C=CC(=O)N1CCN(c2nc(OCC3(CN4CC(F)C4)CC3)nc3c2CCN(c2cccc4cccc(C)c24)C3)CC1CC#N] | MS: 610 ¹H NMR (400 MHz, MeOD) δ 7.58 (d, J = 8.0 Hz, 1H), 7.53 (s, 1H), 7.30 (d, J = 5.5 Hz, 1H), 7.20 (d, J = 7.7 Hz, 2H), 7.15 (s, 1H), 6.73 (s, 1H), 6.19 (d, J = 16.4 Hz, 1H), 5.73 (d, J = 10.4 Hz, 1H), 4.99 (d, J = 57.6 Hz, 2H), 4.16- 4.01 (m, 3H), 3.97 (d, J = 10.2 Hz, 2H), 3.68- 3.52 (m, 3H), 3.48- 3.30 (m, 2H), 3.19- 2.94 (m, 7H), 2.81 (s, 4H), 2.59 (dd, J = 30.3, 15.3 Hz, 2H), 2.51 (d, J = 12.7 Hz, 2H), 0.48 (d, J = 3.5 Hz, 2H), 0.41 (s, 2H). |
| 63 | 2-(1-acryloyl-4-(7-(8- methylnaphthalen-1-yl)-2- ((1-(4-methylpiperazine-1- carbonyl)cyclopropyl)meth- oxy)-5,6,7,8- tetrahydropyrido[3,4- d]pyimidin-4-yl)piperazin- 2-yl)acetonitrile | 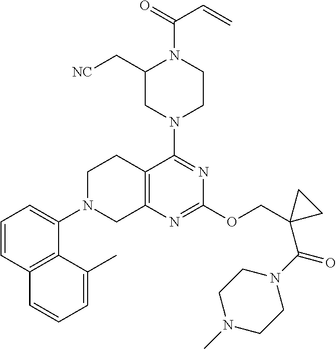 [SMILES: C=CC(=O)N1CCN(c2nc(OCC3(C(=O)N4CCN(C)CC4)CC3)nc3c2CCN(c2cccc4cccc(C)c24)C3)CC1CC#N] | MS: 649 |
| 64 | 2-(1-acryloyl-4-(2-((1-(((2- (dimethylamino)ethyl)(meth- yl)amino)methyl)cyclopro- pyl)methoxy)7-(8- methylnaphthalen-1-yl)- 5,6,7,8- tetrahydropyrido[3,4- d]pyrimidin-4-yl)piperazin- 2-yl)acetonitrile | 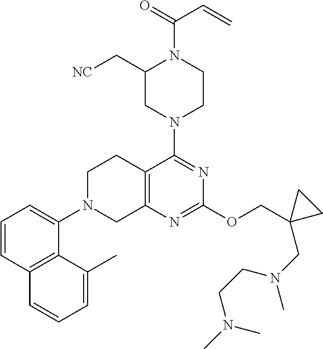 [SMILES: C=CC(=O)N1CCN(c2nc(OCC3(CN(C)CCN(C)C)CC3)nc3c2CCN(c2cccc4cccc(C)c24)C3)CC1CC#N] | MS: 637 ¹H NMR (400 MHz, MeOD) δ 7.79-7.63 (m, 2H), 7.48-7.40 (m, 1H), 7.39-7.19 (m, 3H), 6.97-6.74 (m, 1H), 6.32 (d, J = 16.6 Hz, 1H), 5.86 (d, J = 10.5 Hz, 1H), 4.46- 3.93 (m, 6H), 3.76- 3.51 (m, 4H), 3.30- 3.05 (m, 5H), 2.94 (s, 3H), 2.92-2.85 (m, 1H), 2.83-2.65 (m, 1H), 2.64-2.53 (m, 4H), 2.53-2.38 (m, 2H), 2.37-2.24 (m, 8H), 1.47-1.27 (m, 1H), 0.74-0.62 (m, 2H), 0.58-0.43 (m, 2H). |
| 65 | 2-(1-acryloyl-4-(2-((1- (((S)-3- (dimethylamino)pyrrolidin- 1- yl)methyl)cyclopropyl)meth- oxy)-7-(8- methylnaphthalen-1-yl)- 5,6,7,8- tetrahydropyrido[3,4- d]pyrimidin-4-yl)piperazin- 2-yl)acetonitrile | 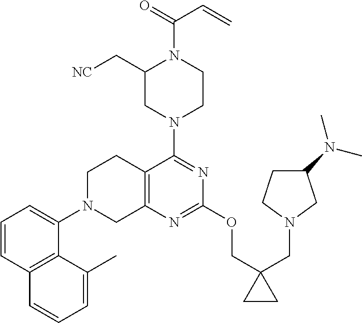 | MS: 649 ¹H NMR (400 MHz, MeOD) δ 7.78-7.62 (m, 2H), 7.48-7.40 (m, 1H), 7.38-7.21 (m, 3H), 6.96-6.76 (m, 1H), 6.32 (d, J = 16.5 Hz, 1H), 5.86 (d, J = 10.4 Hz, 1H), 4.43- 4.02 (m, 6H), 3.86- 3.64 (m, 2H), 3.61- 3.41 (m, 2H), 3.30- 3.05 (m, 5H), 2.94 (s, 3H), 2.91-2.86 (m, 1H), 2.81-2.72 (m, 2H), 2.70-2.43 (m, 4H), 2.25 (s, 6H), 2.12- 1.93 (m, 1H), 1.85- 1.69 (m, 1H), 1.41- 1.25 (m, 2H), 0.74- 0.62 (m, 2H), 0.57- |
| | | | 0.48 (m, 2H). |
| 66 | 2-(4-(2-((1-((4- acetylpiperazin-1- yl)methyl)cyclopropyl)meth- oxy)-7-(8- methylnaphthalen-1-yl)- 5,6,7,8- tetrahydropyrido[3,4- d]pyrimidin-4-yl)-1- acryloylpiperazin-2- yl)acetontrile | 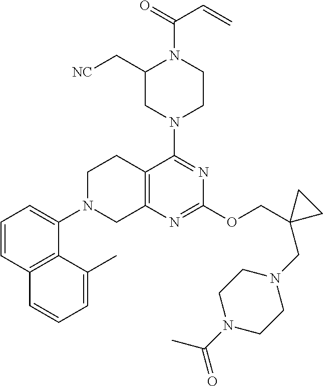 [SMILES: C=CC(=O)N1CCN(c2nc(OCC3(CN4CCN(C(C)=O)CC4)CC3)nc3c2CCN(c2cccc4cccc(C)c24)C3)CC1CC#N] | MS: 663 ¹H NMR (400 MHz, MeOD) δ 7.75-7.63 (m, 2H), 7.48-7.40 (m, 1H), 7.39-7.22 (m, 3H), 6.99-6.75 (m, 1H), 6.32 (d, J = 17.0 Hz, 1H), 5.86 (d, J = 10.3 Hz, 1H), 4.44- 4.03 (m, 6H), 3.80- 3.65 (m, 1H), 3.64- 3.42 (m, 6H), 3.34- 3.07 (m, 6H), 2.94 (s, 3H), 2.93-2.86 (m, 1H), 2.83-2.66 (m, 1H), 2.61-2.37 (m, 6H), 2.10 (s, 3H), 0.77- 0.62 (m, 2H), 0.55- 0.42 (m, 2H). |
| 67 | (S)-2-(1-acryloyl-4-(7-(8- methylnaphthalen-1-yl)-2- ((1-(pyrrolidin-1- ylmethyl)cyclopropyl)meth- oxy)-5,6,7,8- tetrahydropyrido[3,4- d]pyrimidin-4-yl)piperazin- 2-yl)acetonitrile | 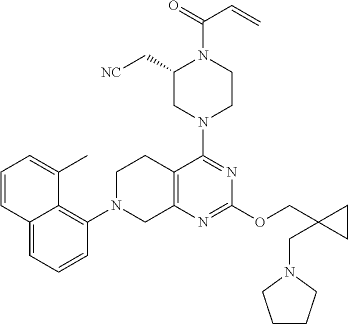 [SMILES: C=CC(=O)N1CCN(c2nc(OCC3(CN4CCCC4)CC3)nc3c2CCN(c2cccc4cccc(C)c24)C3)C[C@@H]1CC#N] | MS: 606 ¹H NMR (400 MHz, MeOD) δ 7.74-7.65 (m, 2H), 7.48-7.39 (m, 1H), 7.38-7.25 (m, 3H), 6.99-6.76 (m, 1H), 6.32 (d, 1H), 5.86 (d, 1H), 4.39-4.05 (m, 6H), 3.72 (t, 1H), 3.62- 3.43 (m, 2H), 3.30- 3.05 (m, 5H), 3.03- 2.88 (m, 4H), 2.85- 2.49 (m, 8H), 1.91- 1.68 (m, 4H), 0.74- 0.48 (m, 4H). |

### Example 68

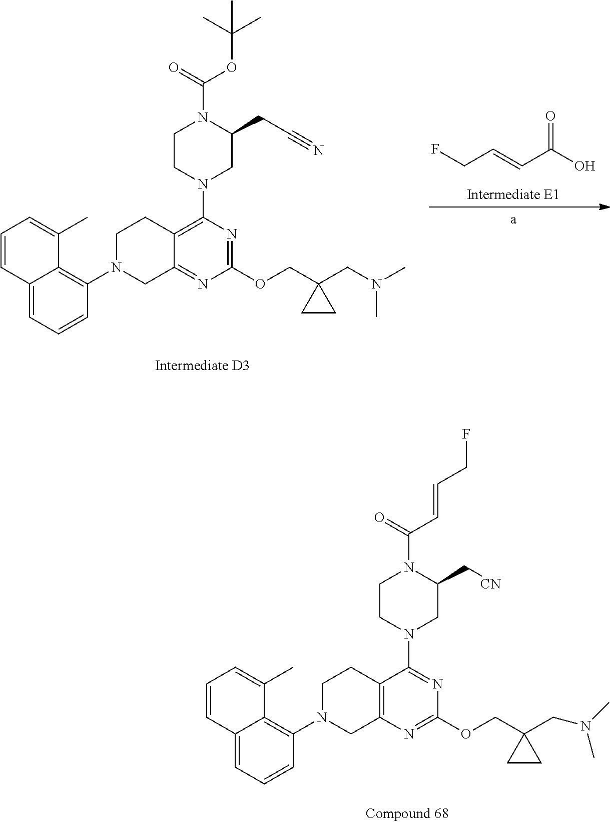

Step a: To a solution of intermediate D3 (0.97 g, 5 mmol) in DCM (1 mL was added TFA (3.5 mL). The reaction mixture was stirred at 25° C. for 1 hour. Upon completion, the reaction mixture was concentrated under reduced pressure. The residue was dissolved in DCM (10 mL). To the solution was added DIEA (4.0 mL), then Intermediate E1 (803 mg, 7.72 mmol) and HATU (2.99 g, 7.86 mmol) were added, and the mixture content was stirred at 25° C. for 0.5 hour. The reaction mixture was concentrated under reduced pressure, the residue was purified by silica gel (50-30% DCM in MeOH) to provide crude. The crude was further purified by Pre-HPLC (column: Daisogel-C18-10 um-100 A; mobile phase: [water (20 mmol NH₄HCO₃)-ACN]; B %: 30%-60%, 30 min) to give Compound 68 (196 mg, 0.32 mmol, 20.78% yield) as an off-white solid. MS: m/z 612 [M+H]+. ¹H NMR (400 MHz, MeOD) δ 7.83-7.62 (m, 2H), 7.55-7.17 (m, 4H), 7.09-6.48 (m, 2H), 5.41-5.01 (m, 2H), 4.48-3.91 (m, 6H), 3.88-3.43 (m, 4H), 3.28-3.04 (m, 4H), 2.94 (s, 3H), 2.88-2.59 (m, 2H), 2.60-2.39 (m, 2H), 2.32 (s, 6H), 1.41-1.16 (m, 1H), 0.83-0.65 (m, 2H), 0.61-0.35 (m, 2H).

The following compounds were synthesized using the above procedure or modification procedure using the corresponding intermediates.

| Compound | Chemical name | Structure | MS: (M + H)⁺/¹HNMR |
|---|---|---|---|
| 69 | (E)-2-(4-(2-((1- ((dimethylamino)methyl)cy- clopropyl)methoxy)-7- (8-methylnaphthalen-1- yl)-5,6,7,8- tetrahydropyrido[3,4- d]pyrimidin-4-yl)-1-(4- hydroxybut-2- enoyl)piperazin-2- yl)acetonitrile | 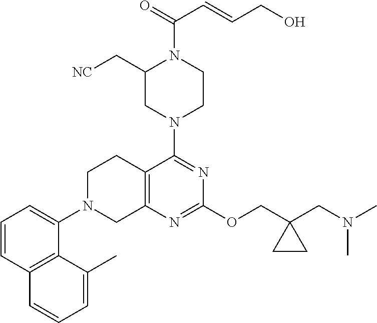 [SMILES: Cc1cccc2cccc(N3CCc4c(nc(OCC5(CN(C)C)CC5)nc4N4CCN(C(=O)/C=C/CO)C(CC#N)C4)C3)c12] | MS: 610 ¹H NMR (400 MHz, MeOD) δ 7.74-7.65 (m, 2H), 7.48-7.39 (m, 1H), 7.38-7.25 (m, 3H), 6.99- 6.76 (m, 1H), 6.32 (d, 1H), 5.86 (d, 1H), 4.39- 4.05 (m, 6H), 3.72 (t, 1H), 3.62-3.43 (m, 2H), 3.30- 3.05 (m, 5H), 3.03- 2.88 (m, 4H), 2.85-2.49 (m, 8H), 1.91-1.68 (m, 4H), 0.74-0.48 (m, 4H). |
| 70 | (E)-2-(4-(2-((1- ((dimethylamino)methyl)cy- clopropyl)methoxy)-7- (8-methylnaphthalen-1- yl)-5,6,7,8- tetrahydropyrido[3,4- d]pyrimidin-4-yl)-1-(4- methoxybut-2- enoyl)piperazin-2- yl)acetonitrile | 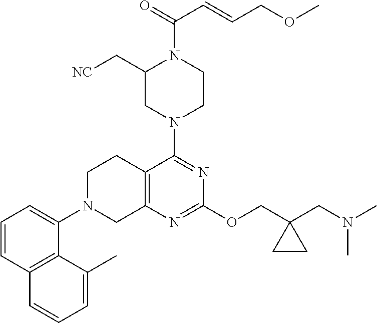 [SMILES: COC/C=C/C(=O)N1CCN(c2nc(OCC3(CN(C)C)CC3)nc3c2CCN(c2cccc4cccc(C)c24)C3)CC1CC#N] | MS: 624 ¹H NMR (400 MHz, MeOD) δ 7.83-7.66 (m, 2H), 7.50-7.43 (m, 1H), 7.41-7.33 (m, 2H), 7.32- 7.25 (m, 1H), 6.99- 6.83 (m, 1H), 6.82-6.60 (m, 1H), 5.15-4.98 (m, 1H), 4.66-4.50 (m, 1H), 4.49-4.28 (m, 2H), 4.26- 4.02 (m, 2H), 3.91- 3.79 (m, 2H), 3.64-3.20 (m, 10H), 3.17-2.64 (m, 15H), 1.07-0.80 (m, 4H). |
| 71 | 2-(1-(2-fluoroacryloyl)-4- (7-(8-methylnaphthalen-1- yl)-2-((1-((4- methylpiperazin-1- yl)methyl)cyclopropyl)meth- oxy)-5,6,7,8- tetrahydropyrido[3,4- d]pyrimidin-4- yl)piperazin-2- yl)acetonitrile | 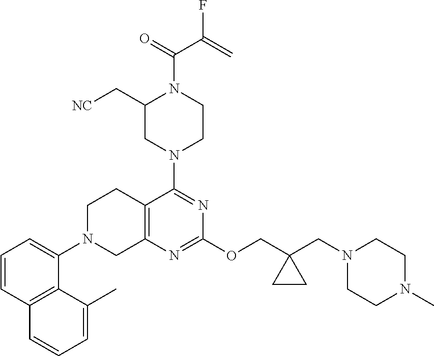 [SMILES: C=C(F)C(=O)N1CCN(c2nc(OCC3(CN4CCN(C)CC4)CC3)nc3c2CCN(c2cccc4cccc(C)c24)C3)CC1CC#N] | MS: 653 |
| 72 | 2-(1-(2-fluoroacryloyl)-4- (7-(8-methylnaphthalen-1- yl)-2-((1-(pyrrolidin-1- ylmethyl)cyclopropyl)meth- oxy)-5,6,7,8- tetrahydropyrido[3,4- d]pyrimidin-4- yl)piperazin-2- yl)acetonitrile | 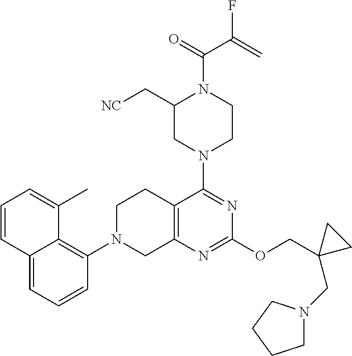 [SMILES: C=C(F)C(=O)N1CCN(c2nc(OCC3(CN4CCCC4)CC3)nc3c2CCN(c2cccc4cccc(C)c24)C3)CC1CC#N] | MS: 624 ¹H NMR (400 MHz, MeOD) δ 7.58 (d, J = 8.0 Hz, 1H), 7.54 (d, J = 7.9 Hz, 1H), 7.35-7.26 (m, 1H), 7.20 (dd, J = 13.4, 5.8 Hz, 2H), 7.14 (d, J = 7.1 Hz, 1H), 6.70 (s, 1H), 6.19 (d, J = 16.8 Hz, 1H), 5.74 (s, 1H), 4.95 (s, 1H), 4.31-4.03 (m, 4H), 4.05- 3.90 (m, 2H), 3.59 (m, 2H), 3.42 (dd, J = 15.2, 9.9 Hz, 2H), 3.17-2.92 (m, 4H), 2.79 (d, J = 10.8 Hz, 4H), 2.59 (d, J = 16.1 Hz, 6H), 1.73 (s, 4H), 0.58 (d, J = 2.4 Hz, 2H), 0.45 (s, 2H). |
| 73 | 2-(4-(2-((1-(azetidin-1- ylmethyl)cyclopropyl)meth- oxy)-7-(8- methylnaphthalen-1-yl)- 5,6,7,8- tetrahydropyrido[3,4- d]pyrimidin-4-yl)-1-(2- fluoroacryloyl)piperazin- 2-yl)acetonitrile | 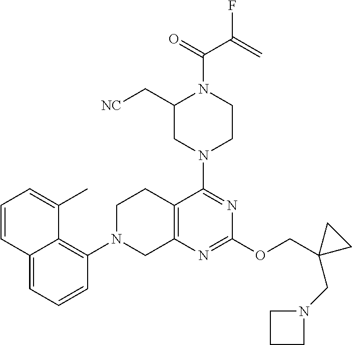 [SMILES: C=C(F)C(=O)N1CCN(c2nc(OCC3(CN4CCC4)CC3)nc3c2CCN(c2cccc4cccc(C)c24)C3)CC1CC#N] | MS: 610 ¹H NMR (400 MHz, MeOD) δ 7.57 (dd, J = 16.3, 8.2 Hz, 2H), 7.36- 7.27 (m, 1H), 7.18 (dt, J = 17.1, 7.2 Hz, 3H), 5.31- 5.13 (m, 2H), 4.10 (d, J = 8.2 Hz, 3H), 4.08-3.90 (m, 6H), 3.70-3.52 (m, 2H), 3.43 (d, J = 7.4 Hz, 2H), 3.17-2.94 (m, 6H), 2.80 (s, 3H), 2.39-2.24 (m, 2H), 1.26 (dd, J = 6.9, 4.6 Hz, 2H), 1.19 (d, J = 3.1 Hz, 2H), 0.67 (s, 4H). |
| 74 | (E)-2-(1-(but-2-enoyl)-4- (7-(8-methylnaphthalen-1- yl)-2-((1-(pyrrolidin-1- ylmethyl)cyclopropyl)meth- oxy)-5,6,7,8- tetrahydropyrido[3,4- d]pyrimidin-4- yl)piperazin-2- yl)acetonitrile | 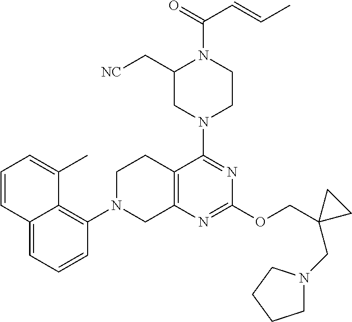 [SMILES: C/C=C/C(=O)N1CCN(c2nc(OCC3(CN4CCCC4)CC3)nc3c2CCN(c2cccc4cccc(C)c24)C3)CC1CC#N] | MS: 620 ¹H NMR (400 MHz, MeOD) δ 7.61 (d, J = 8.1 Hz, 1H), 7.57 (d, J = 8.0 Hz, 1H), 7.33 (dd, J = 13.4, 7.6 Hz, 1H), 7.23 (d, J = 7.7 Hz, 2H), 7.17 (s, 1H), 5.88 (td, J = 16.5, 6.4 Hz, 1H), 5.26-5.08 (m, 2H), 4.95 (d, J = 19.5 Hz, 1H), 4.51 (d, J = 30.8 Hz, 1H), 4.19 (s, 2H), 4.14- 3.94 (m, 3H), 3.62 (t, J = 19.1 Hz, 2H), 3.51-3.31 (m, 2H), 3.13 (s, 4H), 2.83 (s, 3H), 2.78 (dd, J = 7.4, 3.6 Hz, 1H), 2.63 (d, J = 11.4 Hz, 7H), 1.76 (s, 4H), 1.22 (s, 2H), 0.61 (s, 2H), 0.48 (s, 2H). |
| 75 | 2-(4-(2-((1-((3- (dimethylamino)azetidin- 1- yl)methyl)cyclopropyl)meth- oxy)-7-(8- methylnaphthalen-1-yl)- 5,6,7,8- tetrahydropyrido[3,4- d]pyrimidin-4-yl)-1-(2- fluoroacryloyl)piperazin- 2-yl)acetonitrile | 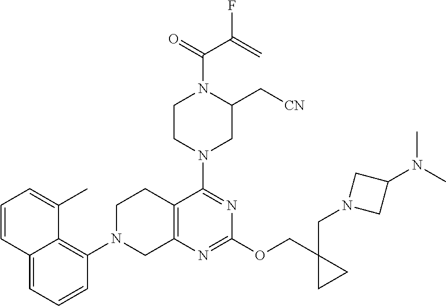 [SMILES: C=C(F)C(=O)N1CCN(c2nc(OCC3(CN4CC(N(C)C)C4)CC3)nc3c2CCN(c2cccc4cccc(C)c24)C3)CC1CC#N] | MS: 653 ¹H NMR (400 MHz, MeOD) δ 7.71 (d, J = 8.1 Hz, 1H), 7.65 (s, 1H), 7.43 (s, 1H), 7.32 (d, J = 7.7 Hz, 2H), 7.27 (s, 1H), 5.43- 5.26 (m, 2H), 4.20 (d, J = 9.3 Hz, 4H), 4.08 (s, 2H), 3.72 (s, 4H), 3.60- 3.42 (m, 2H), 3.22 (d, J = 4.1 Hz, 4H), 3.10 (s, 3H), 3.05-2.97 (m, 2H), 2.92 (s, 3H), 2.70 (s, 3H), 2.14 (s, 6H), 0.64 (d, J = 3.0 Hz, 2H), 0.57 (s, 2H). |
| 76 | 2-(4-(2-((1- ((diethylamino)methyl)cy- clopropyl)methoxy)-7-(8- methylnaphthalen-1-yl)- 5,6,7,8- tetrahydropyrido[3,4- d]pyrimidin-4-yl)-1-(2- fluoroacryloyl)piperazin- 2-yl)acetonitrile | 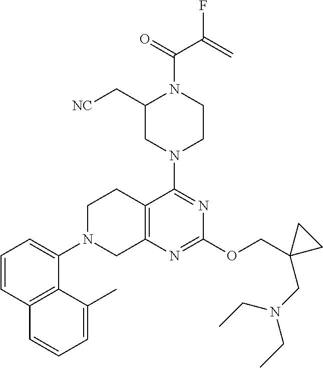 [SMILES: C=C(F)C(=O)N1CCN(c2nc(OCC3(CN(CC)CC)CC3)nc3c2CCN(c2cccc4cccc(C)c24)C3)CC1CC#N] | MS: 626 ¹H NMR (400 MHz, MeOD) δ 7.72 (dd, J = 16.7, 7.9 Hz, 2H), 7.50- 7.43 (m, 1H), 7.41-7.26 (m, 3H), 5.48-5.30 (m, 2H), 4.43-4.09 (m, 6H), 3.75 (dd, J = 17.7, 13.9 Hz, 1H), 3.63-3.47 (m, 2H), 3.34-3.06 (m, 5H), 2.95 (s, 3H), 2.84-2.51 (m, 8H), 1.44-1.31 (m, 1H), 1.07 (t, J = 6.7 Hz, 6H), 0.76-0.64 (m, 2H), 0.61-0.49 (m, 2H). |
| 77 | (E)-2-(4-(2-((1- ((dimethylamino)methyl)cy- clopropyl)methoxy)-7- (8-methylnaphthalen-1- yl)-5,6,7,8- tetrahydropyrido[3,4- d]pyrimidin-4-yl)-1-(4- fluorobut-2- enoyl)piperazin-2- yl)acetonitrile | 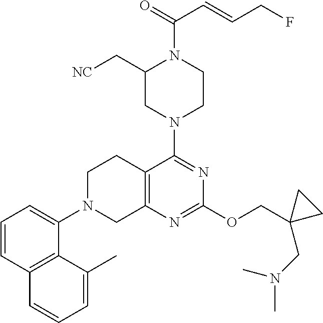 [SMILES: Cc1cccc2cccc(N3CCc4c(nc(OCC5(CN(C)C)CC5)nc4N4CCN(C(=O)/C=C/CF)C(CC#N)C4)C3)c12] | MS: 612 ¹H NMR (400 MHz, MeOD) δ 7.76-7.65 (m, 2H), 7.47-7.41 (m, 1H), 7.38-7.24 (m, 3H), 7.02- 6.72 (m, 2H), 5.23- 5.07 (m, 2H), 4.31-4.04 (m, 5H), 3.87-3.66 (m, 2H), 3.62-3.42 (m, 2H), 3.30-3.07 (m, 6H), 2.94 (s, 3H), 2.92-2.85 (m, 1H), 2.81-2.64 (m, 1H), 2.51-2.38 (m, 2H), 2.32 (s, 6H), 0.79-0.64 (m, 2H), 0.52 (s, 2H). |
| 78 | 2-(4-(2-((1- ((dimethylamino)methyl)cy- clopropyl)methoxy)-7- (8-methylnaphthalen-1- yl)-5,6,7,8- tetrahydropyrido[3,4- d]pyrimidin-4-yl)-1-(2- fluoroacryloyl)piperazin- 2-yl)acetonitrile | 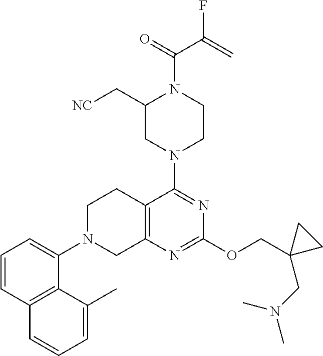 [SMILES: C=C(F)C(=O)N1CCN(c2nc(OCC3(CN(C)C)CC3)nc3c2CCN(c2cccc4cccc(C)c24)C3)CC1CC#N] | MS: 598 ¹H NMR (400 MHz, MeOD) δ 7.82-7.64 (m, 2H), 7.49-7.40 (m, 1H), 7.39-7.24 (m, 3H), 5.46- 5.24 (m, 2H), 4.45- 4.00 (m, 6H), 3.79-3.66 (m, 1H), 3.63-3.44 (m, 2H), 3.32-3.02 (m, 5H), 2.94 (s, 3H), 2.85-2.64 (m, 2H), 2.63-2.45 (m, 2H), 2.38 (s, 6H), 1.44- 1.27 (m, 1H), 0.83-0.66 (m, 2H), 0.62-0.46 (m, 2H). |
| 79 | 2-(4-(7-(8- chloronaphthalen-1-yl)-2- ((1- ((dimethylamino)methyl)cy- clopropyl)methoxy)- 5,6,7,8- tetrahydropyrido[3,4- d]pyrimidin-4-yl)-1-(2- fluoroacryloyl)piperazin- 2-yl)acetonitrile | 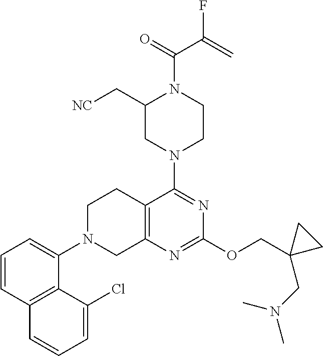 [SMILES: C=C(F)C(=O)N1CCN(c2nc(OCC3(CN(C)C)CC3)nc3c2CCN(c2cccc4cccc(Cl)c24)C3)CC1CC#N] | MS: 618 ¹H NMR (400 MHz, MeOD) δ 7.81 (d, J = 8.0 Hz, 1H), 7.67 (d, J = 8.0 Hz, 1H), 7.59-7.43 (m, 2H), 7.41-7.20 (m, 2H), 5.47-5.17 (m, 2H), 4.49- 3.95 (m, 6H), 3.79- 3.37 (m, 3H), 3.23-2.98 (m, 7H), 2.96-2.91 (m, 1H), 2.88 (s, 6H), 2.77- 2.59 (m, 1H), 2.10-1.96 (m, 1H), 0.94-0.66 (m, 4H). |
| 80 | 2-(4-(2-((1- ((dimethylamino)methyl)cy- clopropyl)methoxy)-7- (2,3-dimethylphenyl)- 5,6,7,8- tetrahydropyrido[3,4- d]pyrimidin-4-yl)-1-(2- fluoroacryloyl)piperazin- 2-yl)acetonitrile | 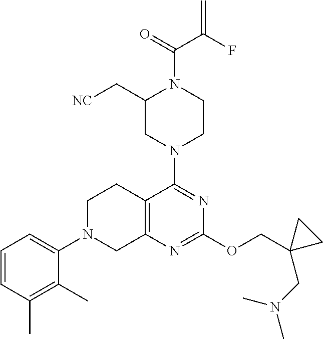 [SMILES: C=C(F)C(=O)N1CCN(c2nc(OCC3(CN(C)C)CC3)nc3c2CCN(c2cccc(C)c2C)C3)CC1CC#N] | MS: 562 ¹H NMR (400 MHz, MeOD) δ 7.11 (t, 1H), 7.04-6.92 (m, 2H), 5.45- 5.23 (m, 2H), 4.35 (d, 3H), 4.20 (d, 1H), 4.01 (d, 2H), 3.47-3.30 (m, 6H), 3.27-3.04 (m, 5H), 3.03- 2.77 (m, 8H), 2.32 (s, 6H), 0.97-0.87 (m, 4H). |
| 81 | 2-(4-(2-((1-((3,3- difluoropyrrolidin-1- yl)methyl)cyclopropyl)meth- oxy)-7-(8- methylnaphthalen-1-yl)- 5,6,7,8- tetrahydropyrido[3,4- d]pyrimidin-4-yl)-1-(2- fluoroacryloyl)piperazin- 2-yl)acetonitrile | 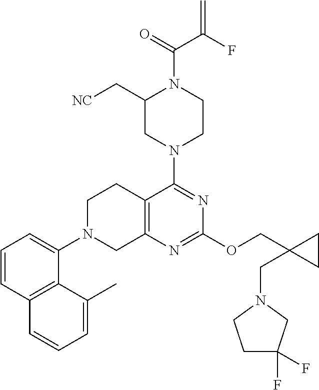 [SMILES: C=C(F)C(=O)N1CCN(c2nc(OCC3(CN4CCC(F)(F)C4)CC3)nc3c2CCN(c2cccc4cccc(C)c24)C3)CC1CC#N] | MS: 660 ¹H NMR (400 MHz, MeOD) δ 7.71 (d, 1H), 7.67 (d, 1H), 7.47-7.40 (m, 1H), 7.37-7.24 (m, 3H), 5.45-5.27 (m, 2H), 4.41-4.05 (m, 6H), 3.84- 3.42 (m, 4H), 3.33- 3.04 (m, 5H), 3.02-2.87 (m, 6H), 2.84-2.47 (m, 5H), 2.30-2.14 (m, 2H), 0.71-0.42 (m, 4H). |
| 82 | (E)-2-(1-(but-2-enoyl)-4- (2-((1- ((dimethylamino)methyl)cy- clopropyl)methoxy)-7- (2,3-dimethylphenyl)- 5,6,7,8- tetrahydropyrido[3,4- d]pyrimidin-4- yl)piperazin-2- yl)acetonitrile | 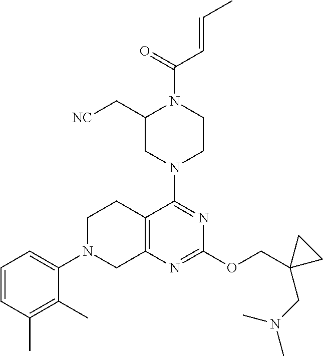 [SMILES: C/C=C/C(=O)N1CCN(c2nc(OCC3(CN(C)C)CC3)nc3c2CCN(c2cccc(C)c2C)C3)CC1CC#N] | MS: 558 ¹H NMR (400 MHz, MeOD) δ 7.09 (t, 1H), 7.02-6.88 (m, 3H), 5.25 (m, 2H), 4.31-4.14 (m, 4H), 3.95 (d, 2H), 3.33- 3.07 (m, 6H), 3.07-2.84 (m, 7H), 2.70 (m, 6H), 2.31 (m, 6H), 1.96 (d, 2H), 0.87-0.82 (m, 2H), 0.74-0.70 (m, 2H). |
| 83 | (S)-2-(4-(2-((1- ((dimethylamino)methyl)cy- clopropyl)methoxy)-7- (8-methylnaphthalen-1- yl)-5,6,7,8- tetrahydropyrido[3,4- d]pyrimidin-4-yl)-1-(2- fluoroacryloyl)piperazin- 2-yl)acetonitrile | 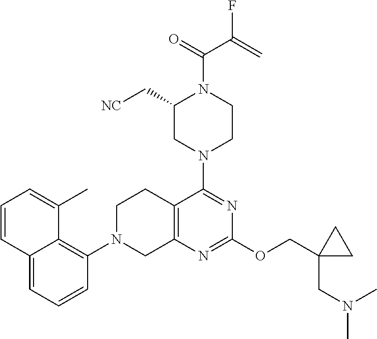 [SMILES: C=C(F)C(=O)N1CCN(c2nc(OCC3(CN(C)C)CC3)nc3c2CCN(c2cccc4cccc(C)c24)C3)C[C@@H]1CC#N] | MS: 598 ¹H NMR (400 MHz, MeOD) δ 7.72 (d, 1H), 7.68 (d, 1H), 7.49-7.40 (m, 1H), 7.38-7.30 (m, 2H), 7.27 (d, 1H), 5.43- 5.29 (m, 2H), 4.31-4.16 (m, 4H), 4.16-4.01 (m, 2H), 3.78-3.50 (m, 4H), 3.32-3.00 (m, 7H), 2.82- 2.67 (m, 2H), 2.55- 2.31 (m, 9H), 0.75-0.70 (m, 2H), 0.57-0.52 (m, 2H). |
| 84 | (S,E)-2-(4-(7-(8- chloronaphthalen-1-yl)-2- ((1- ((dimethylamino)methyl)cy- clopropyl)methoxy)- 5,6,7,8- tetrahydropyrido[3,4- d]pyrimidin-4-yl)-1-(4- fluorobut-2- enoyl)piperazin-2- yl)acetonitrile | 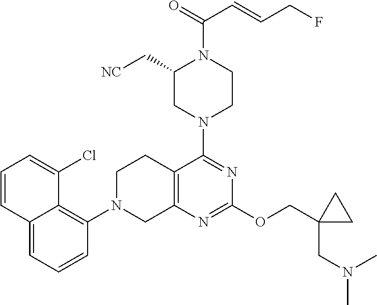 [SMILES: CN(C)CC1(COc2nc3c(c(N4CCN(C(=O)/C=C/CF)[C@@H](CC#N)C4)n2)CCN(c2cccc4cccc(Cl)c24)C3)CC1] | MS: 632 |

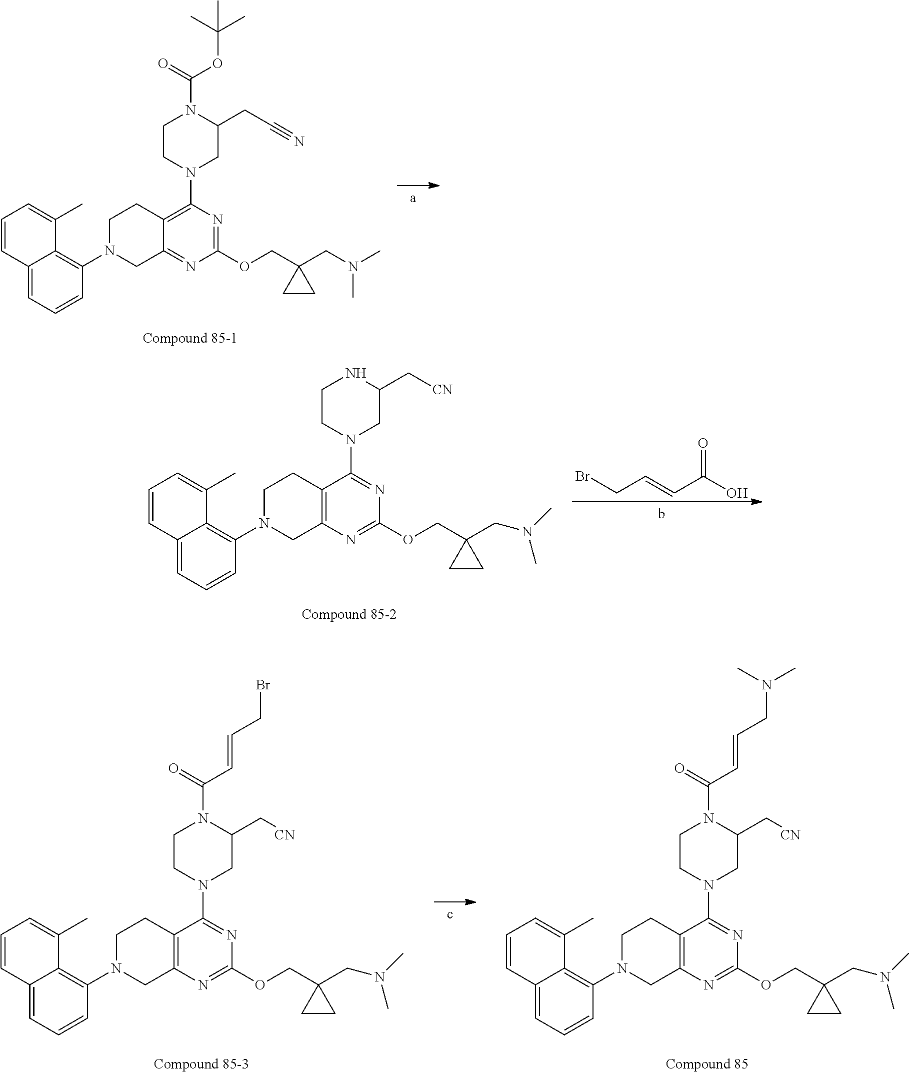

Step a: To a solution of Compound 85-1 (488 mg, 0.78 mmol) in DCM (10 mL) was added TFA (3 mL). The reaction mixture was stirred at 25° C. for 3 h. The reaction mixture was adjusted pH>7 by saturated sodium bicarbonate solution and extracted with DCM (50 mL) twice. The combined organic layers were dried over anhydrous Na₂SO₄, filtered and concentrated under reduced pressure to give Compound 85-2 (445 mg). MS: m/z 526 [M+1]⁺.

Step b: To a solution of Compound 85-2 (194 mg, 0.37 mmol) and (2E)-4-Bromo-2-butenoicacid (79 mg, 0.48 mmol) in DCM (10 mL) was added HATU (247 mg, 0.65 mmol). The mixture was stirred at RT for 2 h. Brine (30 mL) was added to the reaction mixture. The mixture was extracted with DCM (30 mL) twice. The combined organic layers were dried over anhydrous Na₂SO₄, filtered and concentrated under reduced pressure to give Compound 85-3 (286 mg). MS: m/z 672 and 674 [M+1]⁺.

Step c: A mixture of Compound 85-3 (286 mg, 0.43 mmol), dimethylamine (4 mol/L, 2 mL) and DIEA (2 mL) in DMF (5 mL) was stirred at RT for 1 h. Water (50 mL) was added to the reaction mixture. The mixture was extracted with EtOAc (30 mL) twice. The combined organic layers were washed with brine (50 mL) twice, dried over anhydrous Na₂SO₄, filtered and concentrated under reduced pressure. The residue was purified by pre-TLC and pre-HPLC to give Compound 85 (34 mg). MS: m/z 637 [M+1]⁺

The following examples were synthesized using the above procedure or modification procedure using the corresponding intermediates.

| Compound | Chemical name | Structure | MS: (M + H)⁺/¹HNMR |
|---|---|---|---|
| 86 | (E)-2-(1-(4- (dimethylamino)but-2- enoyl)-4-(7-(8- methylnaphthalen-1- yl)-2-((1-(pyrrolidin-1- ylmethyl)cyclopropyl) methoxy)-5,6,7,8- tetrahydropyrido[3,4- d]pyrimidin-4- yl)piperazin-2- yl)acetonitrile | 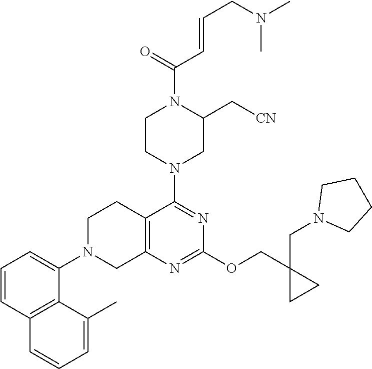 [SMILES: Cc1cccc2cccc(N3CCc4c(nc(OCC5(CN6CCCC6)CC5)nc4N4CCN(C(=O)/C=C/CN(C)C)C(CC#N)C4)C3)c12] | MS: 663 ¹H NMR (400 MeOD) δ 7.62 (d, J = 8.2 Hz, 1H), 7.58 (d, J = 7.8 Hz, 1H), 7.34 (dd, J = 13.5, 7.7 Hz, 1H), 7.23 (dd, J = 16.5, 8.7 Hz, 2H), 7.17 (d, J = 7.0 Hz, 1H), 6.83-6.70 (m, 1H), 6.61 (s, 1H), 4.33-4.08 (m, 4H), 4.02 (dd, J = 17.9, 10.3 Hz, 3H), 3.63 (t, J = 18.4 Hz, 2H), 3.43 (d, J = 15.5 Hz, 2H), 3.13 (s, 4H), 2.84 (s, 3H), 2.67 (d, J = 14.8 Hz, 7H), 2.23 (s, 6H), 1.78 (s, 3H), 1.22 (s, 4H), 0.63 (s, 2H), 0.50 (s, 2H). |

Compound 87-208 in the list below are prepared using commercially available intermediates following the methods outlined in the general reactions schemes and the teaching of the exemplified Examples.

| Com- | | | MS: |
|---|---|---|---|
| pound | Chemical name | Structure | (M + H)⁺ |
| 87 | (S)-2-(1-acryloyl-4-(7-(3-fluoro-2- (trifluoromethyl)phenyl)-2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 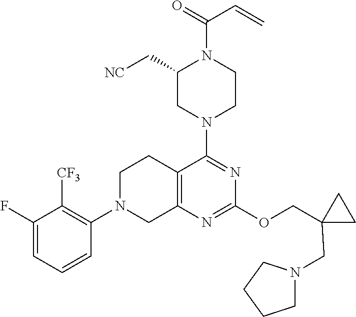 [SMILES: C=CC(=O)N1CCN(c2nc(OCC3(CN4CCCC4)CC3)nc3c2CCN(c2cccc(F)c2C(F)(F)F)C3)C[C@@H]1CC#N] | 628 |
| 88 | (S)-2-(1-acryloyl-4-(7-(2,3-dichlorophenyl)- 2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 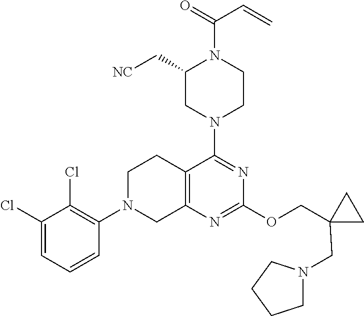 [SMILES: C=CC(=O)N1CCN(c2nc(OCC3(CN4CCCC4)CC3)nc3c2CCN(c2cccc(Cl)c2Cl)C3)C[C@@H]1CC#N] | 610 |
| 89 | (S)-2-(1-acryloyl-4-(7-(3-chloro-2- (trifluoromethyl)phenyl)-2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 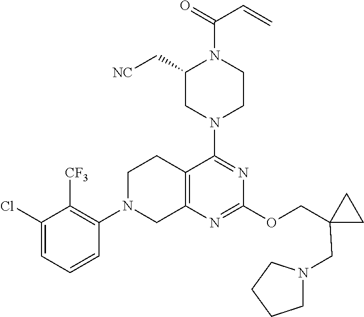 [SMILES: C=CC(=O)N1CCN(c2nc(OCC3(CN4CCCC4)CC3)nc3c2CCN(c2cccc(Cl)c2C(F)(F)F)C3)C[C@@H]1CC#N] | 644 |
| 90 | 2-(1-acryloyl-4-(7-(5-chloro-4- (trifluoromethyl)pyridin-3-yl)-2-((1- (pyrrolidin-1- ylmethyl)cyclopropyl)methoxy-5,6,7,8- tetrahydropyrido[3,4-d]pyrimidin-4- yl)piperazin-2-yl)acetonitrile | 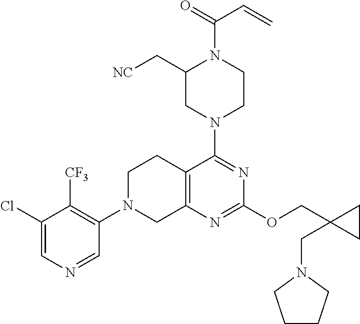 [SMILES: C=CC(=O)N1CCN(c2nc(OCC3(CN4CCCC4)CC3)nc3c2CCN(c2cncc(Cl)c2C(F)(F)F)C3)CC1CC#N] | 645 |
| 91 | (S)-2-(1-acryloyl-4-(7-(5-chloro-4- (trifluoromethyl)pyridin-3-yl)-2-((1- (pyrrolidin-1- ylmethyl)cyclopropyl)methoxy-5,6,7,8- tetrahydropyrido[3,4-d]pyrimidin-4- yl)piperazin-2-yl)acetonitrile | 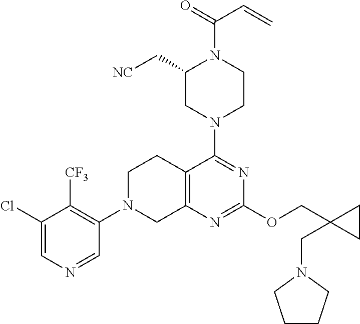 [SMILES: C=CC(=O)N1CCN(c2nc(OCC3(CN4CCCC4)CC3)nc3c2CCN(c2cncc(Cl)c2C(F)(F)F)C3)C[C@@H]1CC#N] | 645 |
| 92 | (S)-2-(1-(2-(fluoroacryloyl)-4-(2-((1- (pyrrolidin-1- ylmethyl)cyclopropyl)methoxy)-7-(2- (trifluoromethyl)pyridin-3-yl)-5,6,7,8- tetrahydropyrido[3,4-d]pyrimidin-4- yl)piperazin-2-yl)acetonitrile | 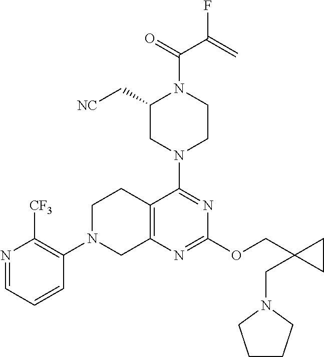 [SMILES: C=C(F)C(=O)N1CCN(c2nc(OCC3(CN4CCCC4)CC3)nc3c2CCN(c2cccnc2C(F)(F)F)C3)C[C@@H]1CC#N] | 629 |
| 93 | (S)-2-(1-(2-fluoroacryloyl)-4-(7-(8- methylnaphthalen-1-yl)-2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 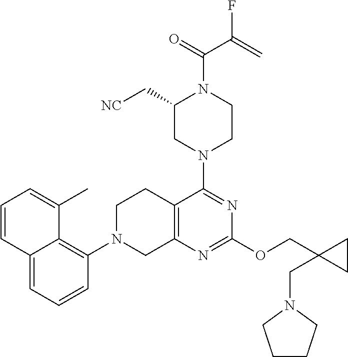 [SMILES: C=C(F)C(=O)N1CCN(c2nc(OCC3(CN4CCCC4)CC3)nc3c2CCN(c2cccc4cccc(C)c24)C3)C[C@@H]1CC#N] | 624 |
| 94 | 2-(4-(7-(8-chloronaphthalen-1-yl)-2-((1- (pyrrolidin-1- ylmethyl)cyclopropyl)methoxy)-5,6,7,8- tetrahydropyrido[3,4-d]pyrimidin-4-yl)-1-(2- fluoroacryloyl)piperazin-2-yl)acetonitrile | 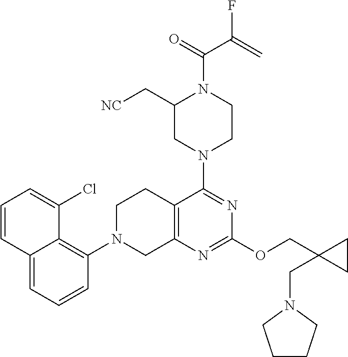 [SMILES: C=C(F)C(=O)N1CCN(c2nc(OCC3(CN4CCCC4)CC3)nc3c2CCN(c2cccc4cccc(Cl)c24)C3)CC1CC#N] | 645 |
| 95 | (S)-2-(4-(7-(8-chloronaphthalen-1-yl)-2-((1- (pyrrolidin-1- ylmethyl)cyclopropyl)methoxy)-5,6,7,8- tetrahydropyrido[3,4-d]pyrimidin-4-yl)-1-(2- fluoroacryloyl)piperazin-2-yl)acetonitrile | 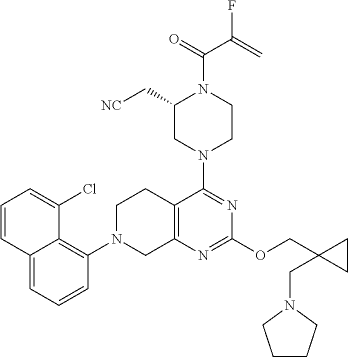 [SMILES: C=C(F)C(=O)N1CCN(c2nc(OCC3(CN4CCCC4)CC3)nc3c2CCN(c2cccc4cccc(Cl)c24)C3)C[C@@H]1CC#N] | 645 |
| 96 | 2-(1-acryloyl-4-(2-((1-((3,3- difluoropyrrolidin-1- yl)methyl)cyclopropyl)methoxy)-7-(3- methyl-2-(trifluoromethyl)phenyl)-5,6,7,8- tetrahydropyrido[3,4-d]pyrimidin-4- yl)piperazin-2-yl)acetonitrile | 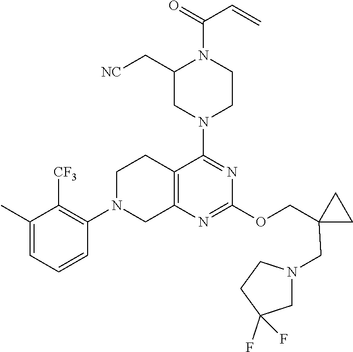 [SMILES: C=CC(=O)N1CCN(c2nc(OCC3(CN4CCC(F)(F)C4)CC3)nc3c2CCN(c2cccc(C)c2C(F)(F)F)C3)CC1CC#N] | 660 |
| 97 | 2-(1-acryloyl-4-(7-(3-chloro-2- (trifluoromethyl)phenyl)-2-((1-(((S)-3- fluoropyrrolidin-1- yl)methyl)cyclopropyl)methoxy)-5,6,7,8- tetrahydropyrido[3,4-d]pyrimidin-4- yl)piperazin-2-yl)acetonitrile | 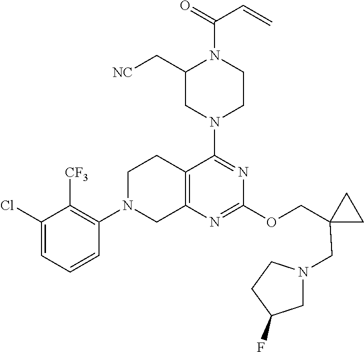 [SMILES: C=CC(=O)N1CCN(c2nc(OCC3(CN4CC[C@H](F)C4)CC3)nc3c2CCN(c2cccc(Cl)c2C(F)(F)F)C3)CC1CC#N] | 662 |
| 98 | (S)-2-(4-(7-(8-chloronaphthalen-1-yl)-2-((1- (pyrrolidin-1-yl)cyclopropyl)methoxy)- 5,6,7,8-tetrahydropyrido[3,4-d]pyrimidin-4- yl)-1-(2-fluoroacryloyl)piperazin-2- yl)acetonitrile | 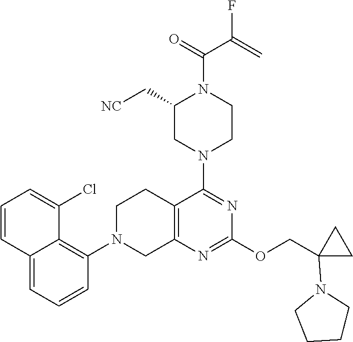 [SMILES: C=C(F)C(=O)N1CCN(c2nc(OCC3(N4CCCC4)CC3)nc3c2CCN(c2cccc4cccc(Cl)c24)C3)C[C@@H]1CC#N] | 630 |
| 99 | (S)-2-(4-(7-(8-chloro-7-fluoronaphthalen-1- yl)-2-((1-(pyrrolidin-1- yl)cyclopropyl)methoxy)-5,6,7,8- tetrahydropyrido[3,4-d]pyrimidin-4-yl)-1-(2- fluoroacryloyl)piperazin-2-yl)acetonitrile | 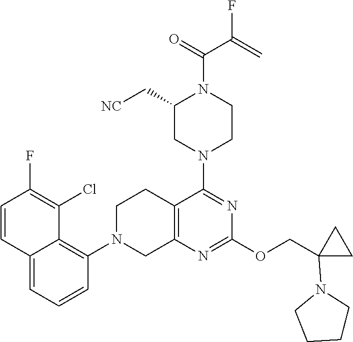 [SMILES: C=C(F)C(=O)N1CCN(c2nc(OCC3(N4CCCC4)CC3)nc3c2CCN(c2cccc4ccc(F)c(Cl)c24)C3)C[C@@H]1CC#N] | 648 |
| 100 | (S)-2-(1-acryloyl-4-(7-(3-methyl-2- (trifluoromethyl)phenyl)-2-((1-(pyrrolidin-1- yl)cyclopropyl)methoxy)-5,6,7,8- tetrahydropyrido[3,4-d]pyrimidin-4- yl)piperazin-2-yl)acetonitrile | 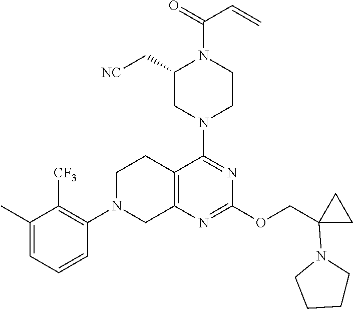 [SMILES: C=CC(=O)N1CCN(c2nc(OCC3(N4CCCC4)CC3)nc3c2CCN(c2cccc(C)c2C(F)(F)F)C3)C[C@@H]1CC#N] | 610 |
| 101 | (S)-2-(1-acryloyl-4-(7-(3-chloro-2- (trifluoromethyl)phenyl)-2-((1-(pyrrolidin-1- yl)cyclopropyl)methoxy)-5,6,7,8- tetrahydropyrido[3,4-d]pyrimidin-4- yl)piperazin-2-yl)acetonitrile | 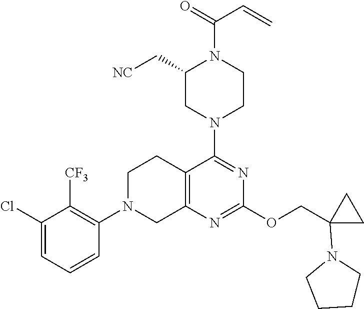 [SMILES: C=CC(=O)N1CCN(c2nc(OCC3(N4CCCC4)CC3)nc3c2CCN(c2cccc(Cl)c2C(F)(F)F)C3)C[C@@H]1CC#N] | 630 |
| 102 | 2-((S)-1-acryloyl-4-(7-(2,3-dichlorophenyl)- 2-((1-((S)-3-fluoropyrrolidin-1- yl)cyclopropyl)methoxy)-5,6,7,8- tetrahydropyrido[3,4-d]pyrimidin-4- yl)piperazin-2-yl)acetonitrile | 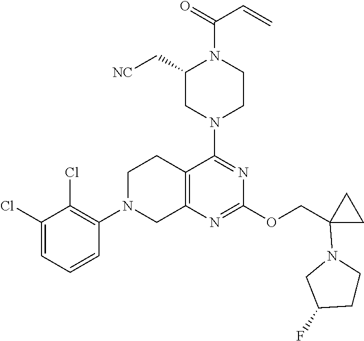 [SMILES: C=CC(=O)N1CCN(c2nc(OCC3(N4CC[C@H](F)C4)CC3)nc3c2CCN(c2cccc(Cl)c2Cl)C3)C[C@@H]1CC#N] | 614 |
| 103 | 2-(1-acryloyl-4-(2-((1-(azetidin-1- ylmethyl)cyclopropyl)methoxy)-7-(2,3- dichlorophenyl)-5,6,7,8-tetrahydropyrido[3,4- d]pyrimidin-4-yl)piperazin-2-yl)acetonitrile | 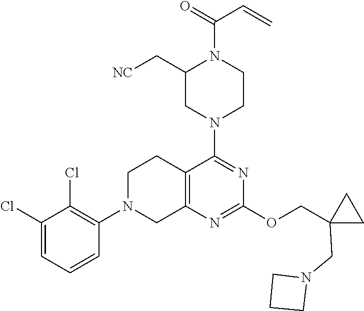 [SMILES: C=CC(=O)N1CCN(c2nc(OCC3(CN4CCC4)CC3)nc3c2CCN(c2cccc(Cl)c2Cl)C3)CC1CC#N] | 596 |
| 104 | (S)-2-(1-acryloyl-4-(2-((1-(azetidin-1- ylmethyl)cyclopropyl)methoxy)-7-(3-chloro- 2-(trifluoromethyl)phenyl)-5,6,7,8- tetrahydropyrido[3,4-d]pyrimidin-4- yl)piperazin-2-yl)acetonitrile | 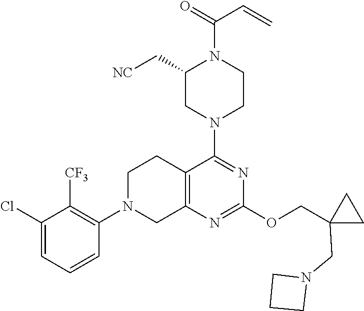 [SMILES: C=CC(=O)N1CCN(c2nc(OCC3(CN4CCC4)CC3)nc3c2CCN(c2cccc(Cl)c2C(F)(F)F)C3)C[C@@H]1CC#N] | 630 |
| 105 | (S)-2-(4-(2-((1-(azetidin-1- ylmethyl)cyclopropyl)methoxy)-7-(3-chloro- 2-(trifluoromethyl)phenyl)-5,6,7,8- tetrahydropyrido[3,4-d]pyrimidin-4-yl)-1-(2- fluoroacryloyl)piperazin-2-yl)acetonitrile | 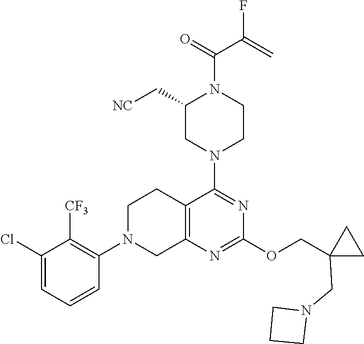 [SMILES: C=C(F)C(=O)N1CCN(c2nc(OCC3(CN4CCC4)CC3)nc3c2CCN(c2cccc(Cl)c2C(F)(F)F)C3)C[C@@H]1CC#N] | 648 |
| 106 | (S)-2-(4-(2-((1-(azetidin-1- ylmethyl)cyclopropyl)methoxy)-7-(8- chloronaphthalen-1-yl)-5,6,7,8- tetrahydropyrido[3,4-d]pyrimidin-4-yl)-1-(2- fluoroacryloyl)piperazin-2-yl)acetonitrile | 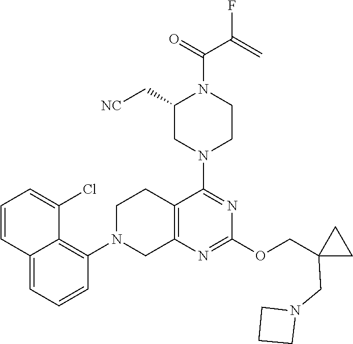 [SMILES: C=C(F)C(=O)N1CCN(c2nc(OCC3(CN4CCC4)CC3)nc3c2CCN(c2cccc4cccc(Cl)c24)C3)C[C@@H]1CC#N] | 630 |
| 107 | (S)-2-(4-(2-((1-(azetidin-1- ylmethyl)cyclopropyl)methoxy)-7-(8- methylnaphthalen-1-yl)-5,6,7,8- tetrahydropyrido[3,4-d]pyrimidin-4-yl)-1-(2- fluoroacryloyl)piperazin-2-yl)acetonitrile | 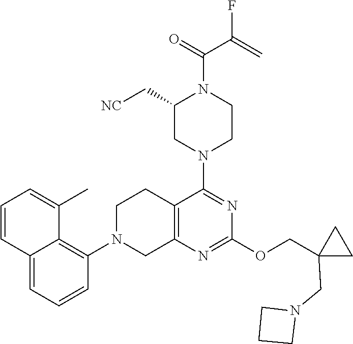 [SMILES: C=C(F)C(=O)N1CCN(c2nc(OCC3(CN4CCC4)CC3)nc3c2CCN(c2cccc4cccc(C)c24)C3)C[C@@H]1CC#N] | 610 |
| 108 | (S)-2-(1-acryloyl-4-(7-(8-methylnaphthalen- 1-yl)-2-((1- (morpholinomethyl)cyclopropyl)methoxy)- 5,6,7,8-tetrahydropyrido[3,4-d]pyrimidin-4- yl)piperazin-2-yl)acetonitrile | 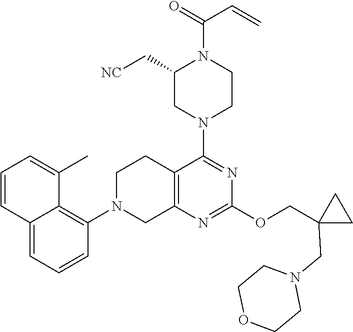 [SMILES: C=CC(=O)N1CCN(c2nc(OCC3(CN4CCOCC4)CC3)nc3c2CCN(c2cccc4cccc(C)c24)C3)C[C@@H]1CC#N] | 622 |
| 109 | (S)-2-(4-(7-(8-chloronaphthalen-1-yl)-2-((1- (morpholinomethyl)cyclopropyl)methoxy)- 5,6,7,8-tetrahydropyrido[3,4-d]pyrimidin-4- yl)-1-(2-fluoroacryloyl)piperazin-2- yl)acetonitrile | 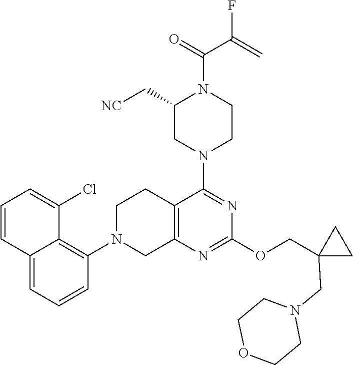 [SMILES: C=C(F)C(=O)N1CCN(c2nc(OCC3(CN4CCOCC4)CC3)nc3c2CCN(c2cccc4cccc(Cl)c24)C3)C[C@@H]1CC#N] | 660 |
| 110 | 2-((S)-1-acryloyl-4-(7-(8-chloronaphthalen-1- yl)-2-(((1R,2S)-2- (dimethylamino)cyclobutyl)methoxy)- 5,6,7,8-tetrahydropyrido[3,4-d]pyrimidin-4- yl)piperazin-2-yl)acetonitrile | 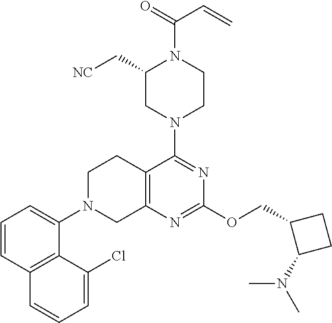 [SMILES: C=CC(=O)N1CCN(c2nc(OC[C@@H]3CC[C@@H]3N(C)C)nc3c2CCN(c2cccc4cccc(Cl)c24)C3)C[C@@H]1CC#N] | 600 |
| 111 | 2-((S)-4-(7-(8-chloronaphthalen-1-yl)-2- (((1S,2R)-2- (dimethylamino)cyclobutyl)methoxy)- 5,6,7,8-tetrahydropyrido[3,4-d]pyrimidin-4- yl)-1-(2-fluoroacryloyl)piperazin-2-yl) acetonitrile | 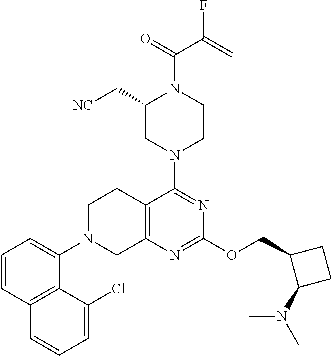 [SMILES: C=C(F)C(=O)N1CCN(c2nc(OC[C@H]3CC[C@H]3N(C)C)nc3c2CCN(c2cccc4cccc(Cl)c24)C3)C[C@@H]1CC#N] | 618 |
| 112 | 2-((S)-1-((E)-but-2-enoyl)-4-(7-(8- chloronaphthalen-1-yl)-2-(((1S,2S)-2- (dimethylamino)cyclobutyl)methoxy)- 5,6,7,8-tetrahydro[3,4-d]pyrimidin-4- yl)piperazin-2-yl)acetonitrile | 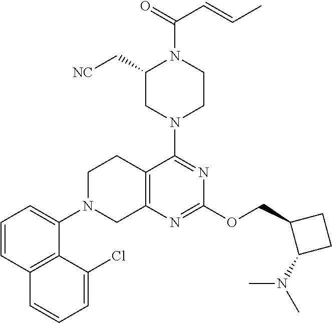 [SMILES: C/C=C/C(=O)N1CCN(c2nc(OC[C@H]3CC[C@@H]3N(C)C)nc3c2CCN(c2cccc4cccc(Cl)c24)C3)C[C@@H]1CC#N] | 614 |
| 113 | 2-((S)-4-(7-(8-chloronaphthalen-1-yl)-2- (((1R,2R)-2- (dimethylamino)cyclobutyl)methoxy)- 5,6,7,8-tetrahydropyrido[3,4-d]pyrimidin- 4-yl)-1-((E)-4-fluorobut-2-enoyl)piperazin- 2-yl)acetonitrile | 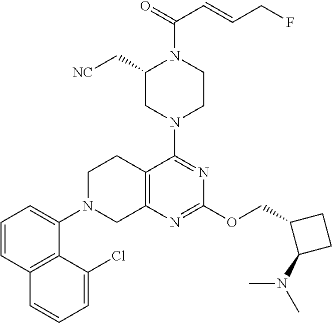 [SMILES: CN(C)[C@@H]1CC[C@H]1COc1nc2c(c(N3CCN(C(=O)/C=C/CF)[C@@H](CC#N)C3)n1)CCN(c1cccc3cccc(Cl)c13)C2] | 632 |
| 114 | 2-((S)-4-(7-(8-chloronaphthalen-1-yl)-2- (((1R,3S)-3-(dimethylamino)cyclopentyl) methoxy)-5,6,7,8-tetrahydropyrido[3,4-d] pyrimidin-4-yl)-1-(2-fluoroacryloyl) piperazin-2-yl)acetonitrile | 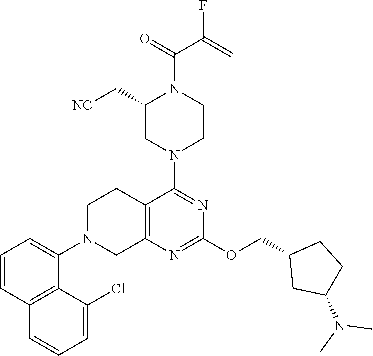 [SMILES: C=C(F)C(=O)N1CCN(c2nc(OC[C@@H]3CC[C@H](N(C)C)C3)nc3c2CCN(c2cccc4cccc(Cl)c24)C3)C[C@@H]1CC#N] | 632 |
| 115 | 2-((S)-1-((E)-but-2-enoyl)-4-(7-(3-chloro-2- (trifluoromethyl)phenyl)-2-(((1S,3R)-3- (dimethylamino)cyclopentyl)methoxy)- 5,6,7,8-tetrahydropyrido[3,4-d]pyrimidin-4- yl)piperazin-2-yl)acetonitrile | 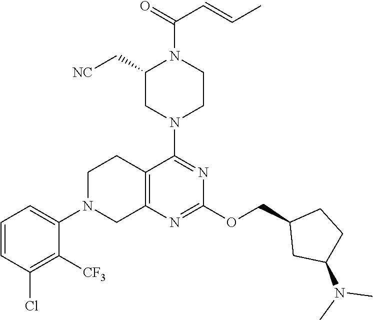 [SMILES: C/C=C/C(=O)N1CCN(c2nc(OC[C@H]3CC[C@@H](N(C)C)C3)nc3c2CCN(c2cccc(Cl)c2C(F)(F)F)C3)C[C@@H]1CC#N] | 646 |
| 116 | 2-((S)-1-acryloyl-4-(2-(((1R,3R)-3- (dimethylamino)cyclopentyl)methoxy)-7-(3- methyl-2-(trifluoromethyl)phenyl)-5,6,7,8- tetrahydropyrido[3,4-d]pyrimidin-4- yl)piperazin-2-yl)acetonitrile | 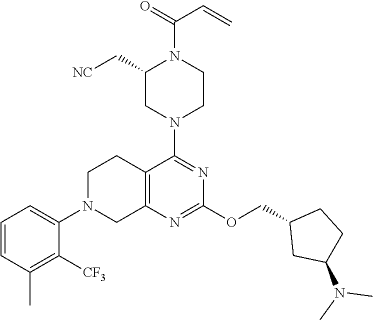 [SMILES: C=CC(=O)N1CCN(c2nc(OC[C@@H]3CC[C@@H](N(C)C)C3)nc3c2CCN(c2cccc(C)c2C(F)(F)F)C3)C[C@@H]1CC#N] | 612 |
| 117 | 2-((S)-4-(7-(2,3-dichlorophenyl)-2-(((1S,3S)- 3-(dimethylamino)cyclopentyl)methoxy)- 5,6,7,8-tetrahydropyrido[3,4-d]pyrimidin-4- yl)-1-((E)-4-fluorobut-2-enoyl)piperazin-2- yl)acetonitrile | 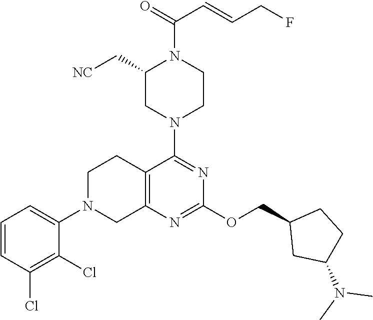 [SMILES: CN(C)[C@H]1CC[C@H](COc2nc3c(c(N4CCN(C(=O)/C=C/CF)[C@@H](CC#N)C4)n2)CCN(c2cccc(Cl)c2Cl)C3)C1] | 630 |
| 118 | 2-((S)-1-acryloyl-4-(2-(((1R,3S)-3- (dimethylamino)cyclopentyl)methoxy)-7-(3- methyl-2-(trifluoromethyl)phenyl)-5,6,7,8- tetrahydropyrido[3,4-d]pyrimidin-4- yl)piperazin-2-yl)acetonitrile | 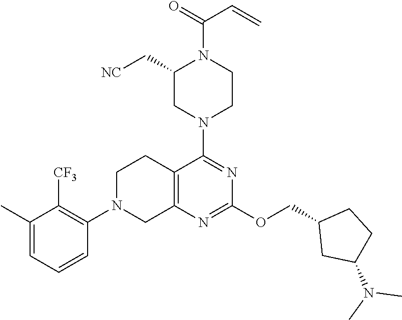 | 612 |
| 119 | 2-((S)-1-acryloyl-4-(2-(((1R,3R)-3- (dimethylamino)cyclopentyl)methoxy)-7-(4- (trifluoromethyl)pyridin-3-yl)-5,6,7,8- tetrahydropyrido[3,4-d]pyrimidin-4- yl)piperazin-2-yl)acetonitrile | 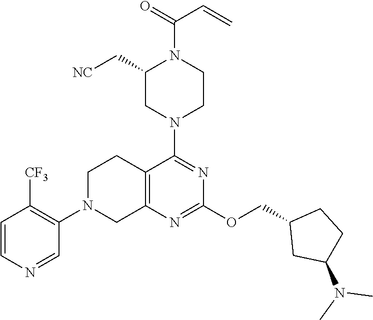 [SMILES: C=CC(=O)N1CCN(c2nc(OC[C@@H]3CC[C@@H](N(C)C)C3)nc3c2CCN(c2cnccc2C(F)(F)F)C3)C[C@@H]1CC#N] | 599 |
| 120 | 2-((S)-4-(7-(5-chloro-4- (trifluoromethyl)pyridin-3-yl)-2-(((1S,3S)-3- (dimethylamino)cyclopentyl)methoxy)- 5,6,7,8-tetrahydropyrido[3,4-d]pyrimidin-4- yl)-1-(2-fluoroacryloyl)piperazin-2- yl)acetonitrile | 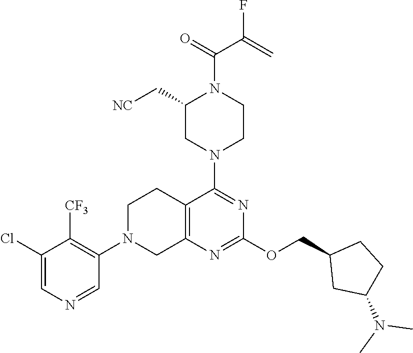 [SMILES: C=C(F)C(=O)N1CCN(c2nc(OC[C@H]3CC[C@H](N(C)C)C3)nc3c2CCN(c2cncc(Cl)c2C(F)(F)F)C3)C[C@@H]1CC#N] | 651 |
| 121 | 2-((S)-1-acryloyl-4-(7-(3-chloro-2- (trifluoromethyl)phenyl)-2-(((1S,3R)-3- (dimethylamino)cyclopentyl)methoxy)- 5,6,7,8-tetrahydropyrido[3,4-d]pyrimidin-4- yl)piperazin-2-yl)acetonitrile | 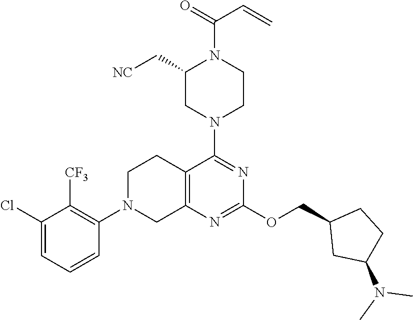 [SMILES: C=CC(=O)N1CCN(c2nc(OC[C@H]3CC[C@@H](N(C)C)C3)nc3c2CCN(c2cccc(Cl)c2C(F)(F)F)C3)C[C@@H]1CC#N] | 632 |
| 122 | 2-((S)-1-acryloyl-4-(7-(2,3-dichlorophenyl)- 2-(((1R,3R)-3- (dimethylamino)cyclopentyl)methoxy)- 5,6,7,8-tetrahydropyrido[3,4-d]pyrimidin-4- yl)piperazin-2-yl)acetonitrile | 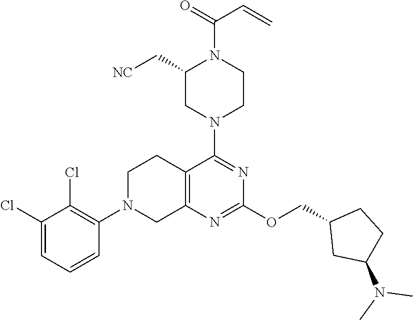 [SMILES: C=CC(=O)N1CCN(c2nc(OC[C@@H]3CC[C@@H](N(C)C)C3)nc3c2CCN(c2cccc(Cl)c2Cl)C3)C[C@@H]1CC#N] | 598 |
| 123 | 2-((S)-1-acryloyl-4-(2-(((1R,3R)-3- (dimethylamino)cyclopentyl)methoxy)-7-(8- methylnaphthalen-1-yl)-5,6,7,8- tetrahydropyrido[3,4-d]pyrimidin-4- yl)piperazin-2-yl)acetonitrile | 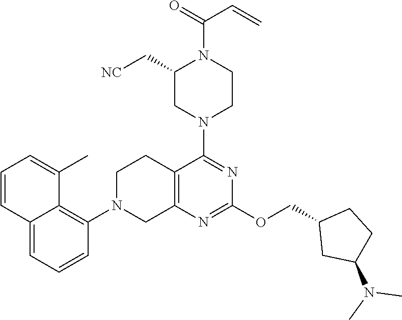 [SMILES: C=CC(=O)N1CCN(c2nc(OC[C@@H]3CC[C@@H](N(C)C)C3)nc3c2CCN(c2cccc4cccc(C)c24)C3)C[C@@H]1CC#N] | 594 |
| 124 | 2-((S)-4-(7-(8-chloronaphthalen-1-yl)-2- (((1S,3R)-3- (dimethylamino)cyclopentyl)methoxy)- 5,6,7,8-tetrahydropyrido[3,4-d]pyrimidin-4- yl)-1-(2-fluoroacryloyl)piperazin-2- yl)acetonitrile | 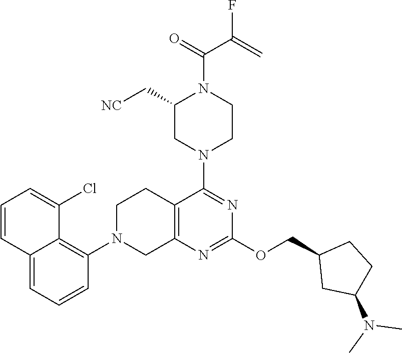 [SMILES: C=C(F)C(=O)N1CCN(c2nc(OC[C@H]3CC[C@@H](N(C)C)C3)nc3c2CCN(c2cccc4cccc(Cl)c24)C3)C[C@@H]1CC#N] | 632 |
| 125 | 2-(4-(7-(3-chloro-2-(trifluoromethyl)phenyl)- 2-((1-((3-methoxypyrrolidin-1- yl)methyl)cyclopropyl)methoxy)-5,6,7,8- tetrahydropyrido[3,4-d]pyrimidin-4-yl)-1-(2- fluoroacryloyl)piperazin-2-yl)acetonitrile | 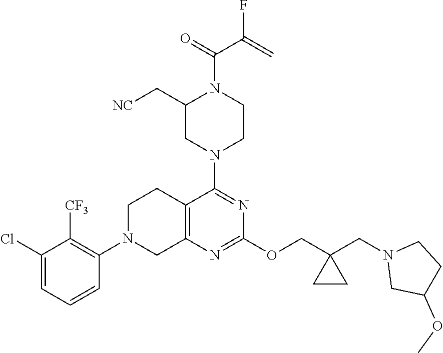 [SMILES: C=C(F)C(=O)N1CCN(c2nc(OCC3(CN4CCC(OC)C4)CC3)nc3c2CCN(c2cccc(Cl)c2C(F)(F)F)C3)CC1CC#N] | 692 |
| 126 | (E)-2-(1-(but-2-enoyl)-4-(7-(3-chloro-2- (trifluoromethyl)phenyl)-2-((1-((4- methylpiperazin-1- yl)methyl)cyclopropyl)methoxy)-5,6,7,8- tetrahydropyrido[3,4-d]pyrimidin-4- yl)piperazin-2-yl)acetonitrile | 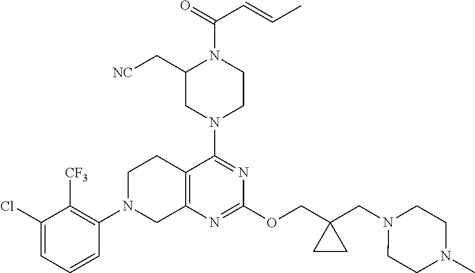 [SMILES: C/C=C/C(=O)N1CCN(c2nc(OCC3(CN4CCN(C)CC4)CC3)nc3c2CCN(c2cccc(Cl)c2C(F)(F)F)C3)CC1CC#N] | 687 |
| 127 | 2-(4-(2-((1-((6-azaspiro[2.5]octan-6- yl)methyl)cyclopropyl)methoxy)-7-(3-chloro- 2-methylphenyl)-5,6,7,8- tetrahydropyrido[3,4-d]pyrimidin-4-yl)-1- acryloylpiperazin-2-yl)acetonitrile | 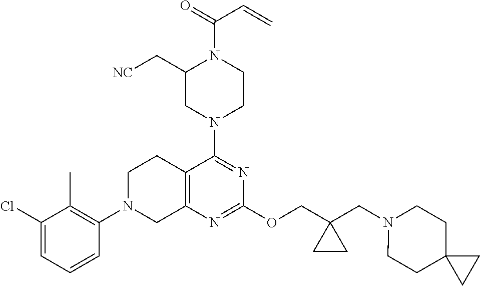 [SMILES: C=CC(=O)N1CCN(c2nc(OCC3(CN4CCC5(CC4)CC5)CC3)nc3c2CCN(c2cccc(Cl)c2C)C3)CC1CC#N] | 630 |
| 128 | 2-(1-acryloyl-4-(2-((1-((4-methoxypiperidin- 1-yl)methyl)cyclopropyl)methoxy)-7-(4- methyl-3,4-dihydro-2H-benzo[b][1,4]oxazin- 5-yl)-5,6,7,8-tetrahydropyrido[3,4- d]pyrimidin-4-yl)piperazin-2-yl)acetonitrile | 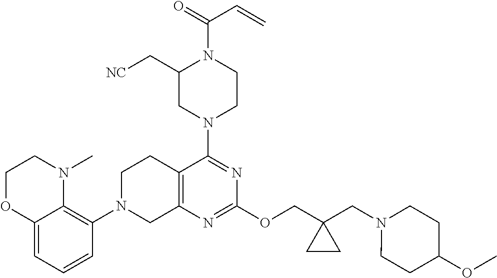 [SMILES: C=CC(=O)N1CCN(c2nc(OCC3(CN4CCC(OC)CC4)CC3)nc3c2CCN(c2cccc4c2N(C)CCO4)C3)CC1CC#N] | 657 |
| 129 | 2-(1-acryloyl-4-(2-((1-((4-methoxypiperidin- 1-yl)methyl)cyclopropyl)methoxy)-7-(1- methylindolin-7-yl)-5,6,7,8- tetrahydropyrido[3,4-d]pyrimidin-4- yl)piperazin-2-yl)acetonitrile | 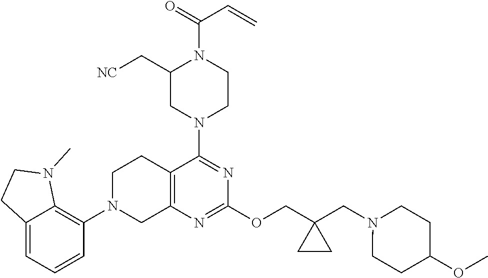 [SMILES: C=CC(=O)N1CCN(c2nc(OCC3(CN4CCC(OC)CC4)CC3)nc3c2CCN(c2cccc4c2N(C)CC4)C3)CC1CC#N] | 641 |
| 130 | 2-(4-(2-((1-(((1R,4R)-2-oxa-5- azabicyclo[2.2.1]heptan-5- yl)methyl)cyclopropyl)methoxy)-7-(8- chloronaphthalen-1-yl)-5,6,7,8- tetrahydropyrido[3,4-d]pyrimidin-4-yl)-1- acryloylpiperazin-2-yl)acetonitrile | 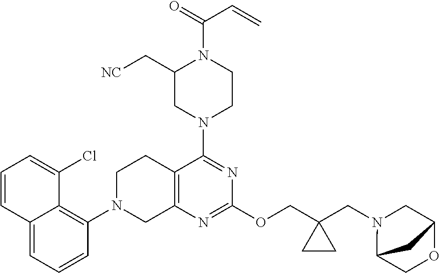 [SMILES: C=CC(=O)N1CCN(c2nc(OCC3(CN4C[C@H]5C[C@@H]4CO5)CC3)nc3c2CCN(c2cccc4cccc(Cl)c24)C3)CC1CC#N] | 654 |
| 131 | 2-(1-acryloyl-4-(7-(3-chloro-2- (trifluoromethyl)phenyl)-2-(1-(2-(pyrrolidin- 1-yl)ethyl)cyclopropoxy)-5,6,7,8- tetrahydropyrido[3,4-d]pyrimidin-4- yl)piperazin-2-yl)acetonitrile | 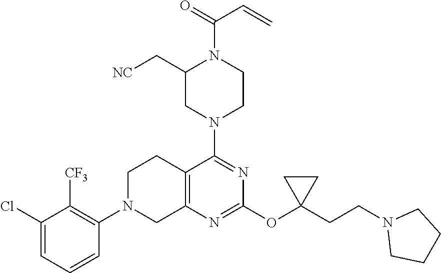 [SMILES: C=CC(=O)N1CCN(c2nc(OC3(CCN4CCCC4)CC3)nc3c2CCN(c2cccc(Cl)c2C(F)(F)F)C3)CC1CC#N] | 644 |
| 132 | 2-(1-acryloyl-4-(7-(3-methyl-2- (trifluoromethyl)phenyl)-2-(2-(1-(pyrrolidin- 1-yl)cyclopropyl)ethoxy)-5,6,7,8- tetrahydropyrido[3,4-d]pyrimidin-4- yl)piperazin-2-yl)acetonitrile | 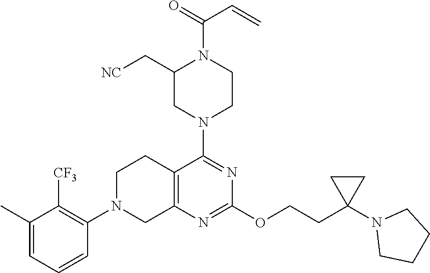 [SMILES: C=CC(=O)N1CCN(c2nc(OCCC3(N4CCCC4)CC3)nc3c2CCN(c2cccc(C)c2C(F)(F)F)C3)CC1CC#N] | 624 |
| 133 | 2-(1-acryloyl-4-(7-(3-chloro-2- (trifluoromethyl)phenyl)-2-((1-(1- methylpiperidin-3-yl)cyclopropyl)methoxy)- 5,6,7,8-tetrahydropyrido[3,4-d]pyrimidin-4- yl)piperazin-2-yl)acetonitrile | 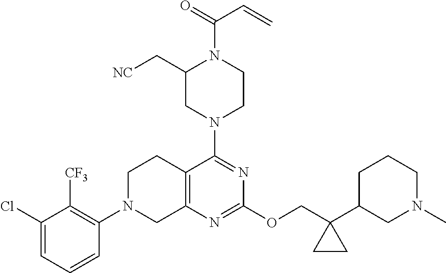 [SMILES: C=CC(=O)N1CCN(c2nc(OCC3(C4CCCN(C)C4)CC3)nc3c2CCN(c2cccc(Cl)c2C(F)(F)F)C3)CC1CC#N] | 658 |
| 134 | (E)-2-(1-(but-2-enoyl)-4-(7-(3-chloro-2- (trifluoromethyl)phenyl)-2-((1-((6-methyl- 2,6-diazaspiro[3.3]heptan-2- yl)methyl)cyclopropyl)methoxy)-5,6,7,8- tetrahydropyrido[3,4-d]pyrimidin-4- yl)piperazin-2-yl)acetonitrile | 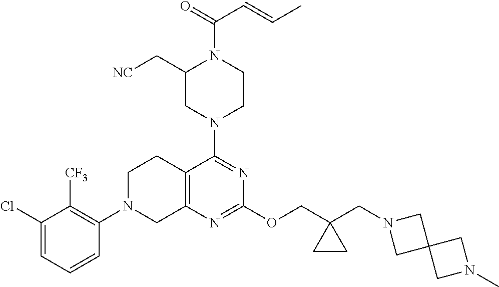 [SMILES: C/C=C/C(=O)N1CCN(c2nc(OCC3(CN4CC5(CN(C)C5)C4)CC3)nc3c2CCN(c2cccc(Cl)c2C(F)(F)F)C3)CC1CC#N] | 699 |
| 135 | 2-(1-acryloyl-4-(7-(3-chloro-2- (trifluoromethyl)phenyl)-2-((1-((diethyl- amino)methyl)cyclopropyl)methoxy)- 5,6,7,8-tetrahydropyrido[3,4-d]pyrimidin-4- yl)piperazin-2-yl)acetonitrile | 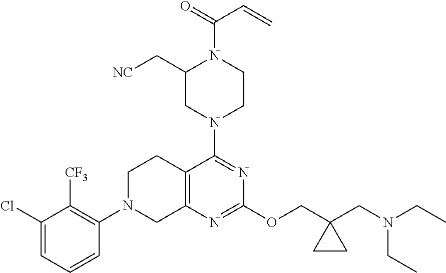 [SMILES: C=CC(=O)N1CCN(c2nc(OCC3(CN(CC)CC)CC3)nc3c2CCN(c2cccc(Cl)c2C(F)(F)F)C3)CC1CC#N] | 646 |
| 136 | 2-(1-acryloyl-4-(7-(8-chloronaphthalen-1-yl)- 2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 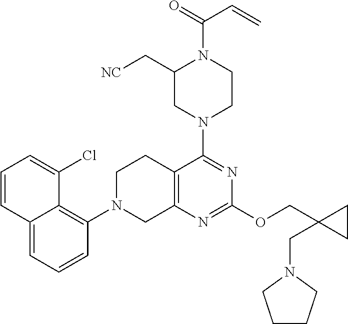 [SMILES: C=CC(=O)N1CCN(c2nc(OCC3(CN4CCCC4)CC3)nc3c2CCN(c2cccc4cccc(Cl)c24)C3)CC1CC#N] | 626 |
| 137 | (S)-2-(1-acryloyl-4-(7-(8-chloronaphthalen-1- yl)-2-((1-(pyrrolidin-1- ylmethyl)cyclopropyl)methoxy)-5,6,7,8- tetrahydropyrido[3,4-d]pyrimidin-4- yl)piperazin-2-yl)acetonitrile | 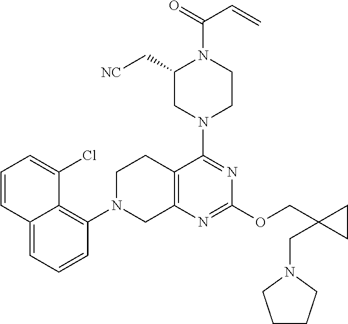 [SMILES: C=CC(=O)N1CCN(c2nc(OCC3(CN4CCCC4)CC3)nc3c2CCN(c2cccc4cccc(Cl)c24)C3)C[C@@H]1CC#N] | 626 |
| 138 | (S)-2-(1-acryloyl-4-(2-((1-(pyrrolidin-1- yl)cyclopropyl)methoxy)-7-(2- (trifluoromethyl)phenyl)-5,6,7,8- tetrahydropyrido[3,4-d]pyrimidin-4- yl)piperazin-2-yl)acetonitrile | 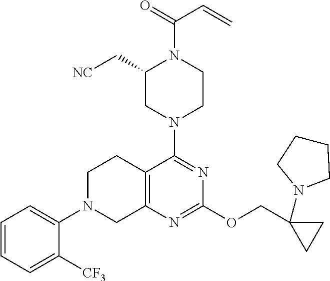 [SMILES: C=CC(=O)N1CCN(c2nc(OCC3(N4CCCC4)CC3)nc3c2CCN(c2ccccc2C(F)(F)F)C3)C[C@@H]1CC#N] | 596 |
| 139 | (S)-2-(1-acryloyl-4-(2-((1-(3,3- difluoropyrrolidin-1- yl)cyclopropyl)methoxy)-7-(3-methyl-2- (trifluoromethyl)phenyl)-5,6,7,8- tetrahydropyrido[3,4-d]pyrimidin-4- yl)piperazin-2-yl)acetonitrile | 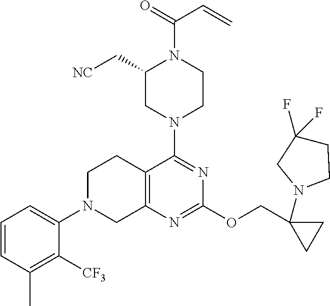 [SMILES: C=CC(=O)N1CCN(c2nc(OCC3(N4CCC(F)(F)C4)CC3)nc3c2CCN(c2cccc(C)c2C(F)(F)F)C3)C[C@@H]1CC#N] | 646 |
| 140 | 2-((2S)-1-acryloyl-4-(7-(3-fluoro-2- (trifluoromethyl)phenyl)-2-((1-(1- methylpyrrolidin-2-yl)cyclopropyl)methoxy)- 5,6,7,8-tetrahydro[3,4-d]pyrimidin-4- yl)piperazin-2-yl)acetonitrile | 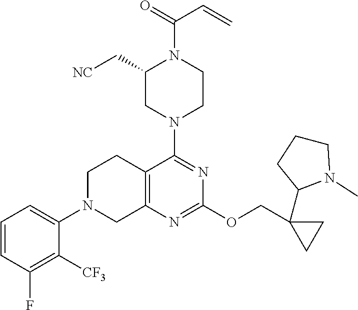 [SMILES: C=CC(=O)N1CCN(c2nc(OCC3(C4CCCN4C)CC3)nc3c2CCN(c2cccc(F)c2C(F)(F)F)C3)C[C@@H]1CC#N] | 628 |
| 141 | 2-((2S)-1-acryloyl-4-(7-(3-chloro-2- (trifluoromethyl)phenyl)-2-((1-(4-hydroxy-1- methylpyrrolidin-2-yl)cyclopropyl)methoxy)- 5,6,7,8-tetrahydropyrido[3,4-d]pyrimidin-4- yl)piperazin-2-yl)acetonitrile | 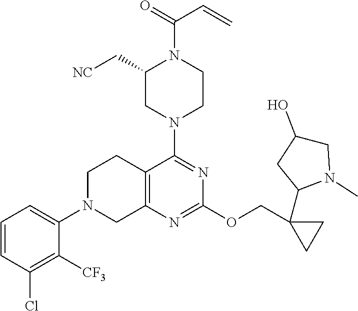 [SMILES: C=CC(=O)N1CCN(c2nc(OCC3(C4CC(O)CN4C)CC3)nc3c2CCN(c2cccc(Cl)c2C(F)(F)F)C3)C[C@@H]1CC#N] | 660 |
| 142 | 2-((2S)-1-acryloyl-4-(7-(2,3-dichlorophenyl)- 2-((2-methyl-1-(1-methylpyrrolidin-2- yl)cyclopropyl)methoxy)-5,6,7,8- tetrahydropyrido[3,4-d]pyrimidin-4- yl)piperazin-2-yl)acetonitrile | 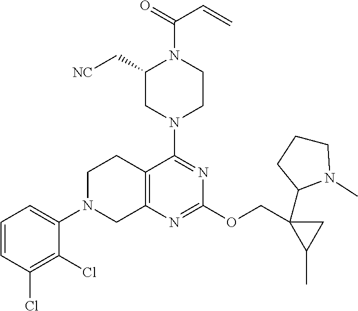 [SMILES: C=CC(=O)N1CCN(c2nc(OCC3(C4CCCN4C)CC3C)nc3c2CCN(c2cccc(Cl)c2Cl)C3)C[C@@H]1CC#N] | 624 |
| 143 | 2-(1-acryloyl-4-(7-(2,3-dichlorophenyl)-2-(1- (pyrrolidin-1-ylmethyl)cyclopropoxy)- 5,6,7,8-tetrahydropyrido[3,4-d]pyrimidin-4- yl)piperazin-2-yl)acetonitrile | 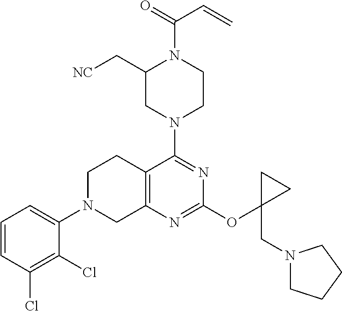 [SMILES: C=CC(=O)N1CCN(c2nc(OC3(CN4CCCC4)CC3)nc3c2CCN(c2cccc(Cl)c2Cl)C3)CC1CC#N] | 596 |
| 144 | 2-(1-acryloyl-4-(2-(1- ((dimethylamino)methyl)cyclopropoxy)-7- (naphthalen-1-yl)-5,6,7,8- tetrahydropyrido[3,4-d]pyrimidin-4- yl)piperazin-2-yl)acetonitrile | 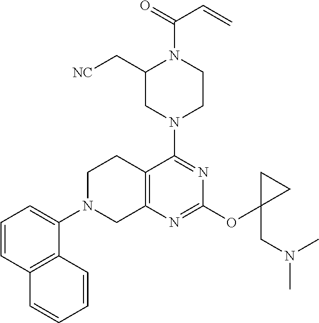 [SMILES: C=CC(=O)N1CCN(c2nc(OC3(CN(C)C)CC3)nc3c2CCN(c2cccc4ccccc24)C3)CC1CC#N] | 552 |
| 145 | 2-(1-acryloyl-4-(7-(8-methylnaphthalen-1- yl)-2-(1-(1-methylpyrrolidin-2- yl)cyclopropoxy)-5,6,7,8- tetrahydropyrido[3,4-d]pyrimidin-4- yl)piperazin-2-yl)acetonitrile | 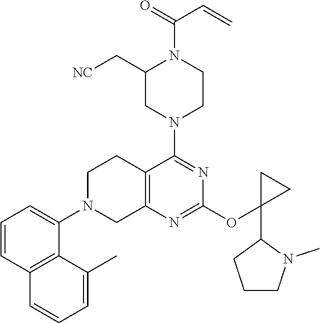 [SMILES: C=CC(=O)N1CCN(c2nc(OC3(C4CCCN4C)CC3)nc3c2CCN(c2cccc4cccc(C)c24)C3)CC1CC#N] | 592 |
| 146 | 2-((2S)-4-(7-(8-chloronaphthalen-1-yl)-2-(1- (4,4-difluoro-1-methylpyrrolidin-2- yl)cyclopropoxy)-5,6,7,8- tetrahydropyrido[3,4-d]pyrimidin-4-yl)-1-(2- fluoroacryloyl)piperazin-2-yl)acetonitrile | 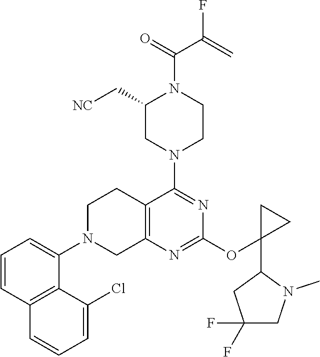 [SMILES: C=C(F)C(=O)N1CCN(c2nc(OC3(C4CC(F)(F)CN4C)CC3)nc3c2CCN(c2cccc4cccc(Cl)c24)C3)C[C@@H]1CC#N] | 666 |
| 147 | 2-((2S)-1-acryloyl-4-(7-(2-chlorophenyl)-2- (1-(4-hydroxy-1-methylpyrrolidin-2-yl)-2,2- dimethylcyclopropoxy)-5,6,7,8- tetrahydropyrido[3,4-d]pyrimidin-4- yl)piperazin-2-yl)acetonitrile | 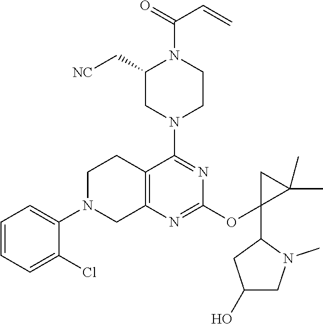 [SMILES: C=CC(=O)N1CCN(c2nc(OC3(C4CC(O)CN4C)CC3(C)C)nc3c2CCN(c2ccccc2Cl)C3)C[C@@H]1CC#N] | 606 |
| 148 | 2-((2S)-4-(7-(2-chloro-3-fluorophenyl)-2-(1- (4-methoxy-1-methylpyrrolidin-2- yl)cyclopropoxy)-5,6,7,8- tetrahydropyrido[3,4-d]pyrimidin-4-yl)-1- ((E)-4-(dimethylamino)but-2- enoyl)piperazin-2-yl)acetonitrile | 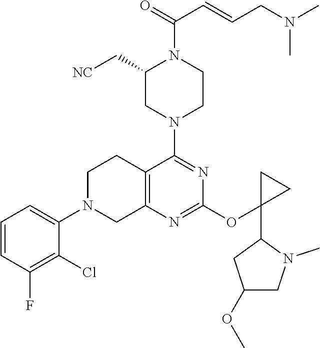 [SMILES: COC1CC(C2(Oc3nc4c(c(N5CCN(C(=O)/C=C/CN(C)C)[C@@H](CC#N)C5)n3)CCN(c3cccc(F)c3Cl)C4)CC2)N(C)C1] | 667 |
| 149 | 2-((2S)-4-(2-(2- (dimethylamino)cyclopropoxy)-7- (naphthalen-1-yl)-5,6,7,8- tetrahydropyrido[3,4-d]pyrimidin-4-yl)-1-(2- fluoroacryloyl)piperazin-2-yl)acetonitrile | 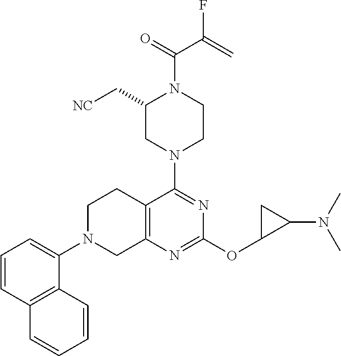 [SMILES: C=C(F)C(=O)N1CCN(c2nc(OC3CC3N(C)C)nc3c2CCN(c2cccc4ccccc24)C3)C[C@@H]1CC#N] | 556 |
| 150 | 2-((2S)-1-((E)-but-2-enoyl)-4-(2-(2- ((dimethylamino)methyl)cyclopropoxy)-7-(8- methylnaphthalen-1-yl)-5,6,7,8- tetrahydropyrido[3,4-d]pyrimidin-4- yl)piperazin-2-yl)acetonitrile | 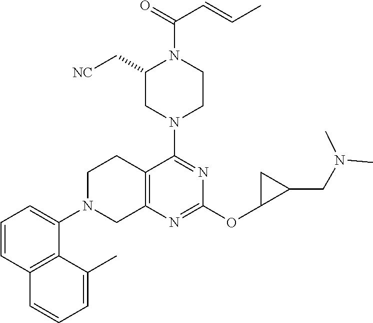 [SMILES: C/C=C/C(=O)N1CCN(c2nc(OC3CC3CN(C)C)nc3c2CCN(c2cccc4cccc(C)c24)C3)C[C@@H]1CC#N] | 580 |
| 151 | 2-((2S)-1-acryloyl-4-(2-((2- (dimethylamino)cyclopropyl)methoxy)-7- (naphthalen-1-yl)-5,6,7,8- tetrahydropyrido[3,4-d]pyrimidin-4- yl)piperazin-2-yl)acetonitrile | 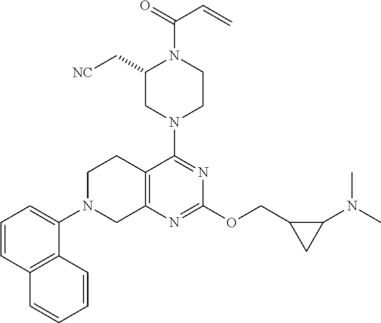 [SMILES: C=CC(=O)N1CCN(c2nc(OCC3CC3N(C)C)nc3c2CCN(c2cccc4ccccc24)C3)C[C@@H]1CC#N] | 552 |
| 152 | 2-((2S)-1-acryloyl-4-(7-(3-chloro-2- fluorophenyl)-2-((2- ((dimethylamino)methyl)cyclopropyl) methoxy)-5,6,7,8-tetrahydropyrido[3,4-d] pyrimidin-4-yl)piperazin-2-yl)acetonitrile | 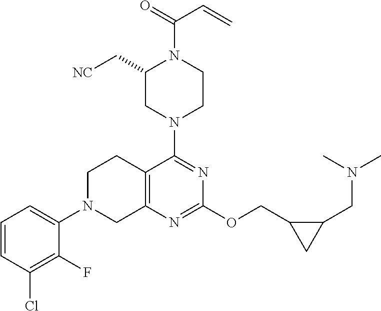 [SMILES: C=CC(=O)N1CCN(c2nc(OCC3CC3CN(C)C)nc3c2CCN(c2cccc(Cl)c2F)C3)C[C@@H]1CC#N] | 568 |
| 153 | 2-((2S)-1-acryloyl-4-(2-((1R)-1-(2- (pyrrolidin-1-ylmethyl)cyclopropyl)ethoxy)- 7-(o-tolyl)-5,6,7,8-tetrahydropyrido[3,4- d]pyrimidin-4-yl)piperazin-2-yl)acetonitrile | 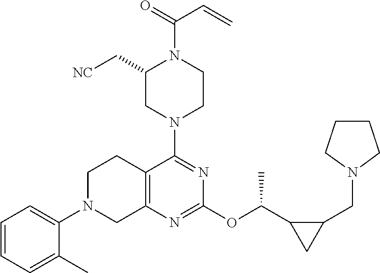 [SMILES: C=CC(=O)N1CCN(c2nc(O[C@H](C)C3CC3CN3CCCC3)nc3c2CCN(c2ccccc2C)C3)C[C@@H]1CC#N] | 570 |
| 154 | 2-((2S)-1-acryloyl-4-(7-(3-fluoro-2- methylphenyl)-2-((1R)-1-(2-((1- methylpyrrolidin-2- yl)methyl)cyclopropyl)ethoxy)-5,6,7,8- tetrahydropyrido[3,4-d]pyrimidin-4- yl)piperazin-2-yl)acetonitrile | 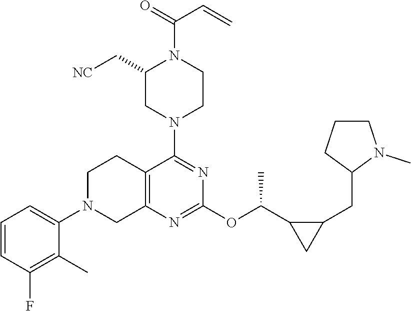 [SMILES: C=CC(=O)N1CCN(c2nc(O[C@H](C)C3CC3CC3CCCN3C)nc3c2CCN(c2cccc(F)c2C)C3)C[C@@H]1CC#N] | 602 |
| 155 | (S)-2-(1-acryloyl-4-(7-(2,3-dichlorophenyl)- 2-(1-(pyrrolidin-1-ylmethyl)cyclobutoxy)- 5,6,7,8-tetrahydropyrido[3,4-d]pyrimidin-4- yl)piperazin-2-yl)acetonitrile | 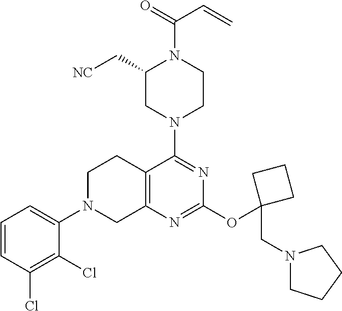 [SMILES: C=CC(=O)N1CCN(c2nc(OC3(CN4CCCC4)CCC3)nc3c2CCN(c2cccc(Cl)c2Cl)C3)C[C@@H]1CC#N] | 610 |
| 156 | 2-((2S)-1-acryloyl-4-(7-(3-chloro-2- (trifluoromethyl)phenyl)-2-(1-((1- methylpyrrolidin-2-yl)methyl)cyclobutoxy)- 5,6,7,8-tetrahydropyrido[3,4-d]pyrimidin-4- yl)piperazin-2-yl)acetonitrile | 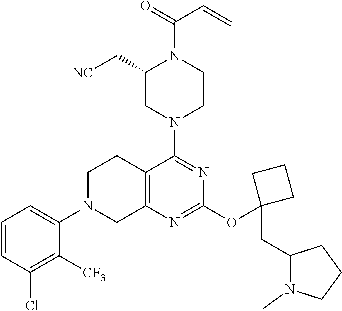 [SMILES: C=CC(=O)N1CCN(c2nc(OC3(CC4CCCN4C)CCC3)nc3c2CCN(c2cccc(Cl)c2C(F)(F)F)C3)C[C@@H]1CC#N] | 658 |
| 157 | 2-((2S)-1-acryloyl-4-(7-(3-chloro-2- (trifluoromethyl)phenyl)-2-(1-(1- methylpyrrolidin-2-yl)cyclobutoxy)-5,6,7,8- tetrahydropyrido[3,4-d]pyrimidin-4- yl)piperazin-2-yl)acetonitrile | 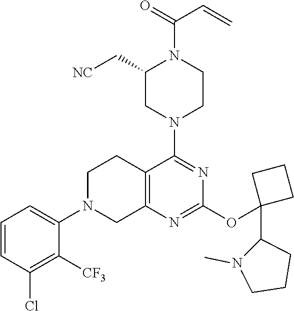 [SMILES: C=CC(=O)N1CCN(c2nc(OC3(C4CCCN4C)CCC3)nc3c2CCN(c2cccc(Cl)c2C(F)(F)F)C3)C[C@@H]1CC#N] | 644 |
| 158 | 2-((2S)-1-acryloyl-4-(2-(2- (dimethylamino)cyclobutoxy)-7-(2- (trifluoromethyl)phenyl)-5,6,7,8- tetrahydropyrido[3,4-d]pyrimidin-4- yl)piperazin-2-yl)acetonitrile | 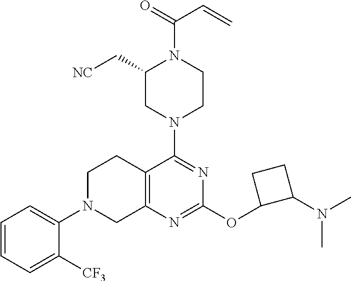 [SMILES: C=CC(=O)N1CCN(c2nc(OC3CCC3N(C)C)nc3c2CCN(c2ccccc2C(F)(F)F)C3)C[C@@H]1CC#N] | 570 |
| 159 | (S)-2-(1-acryloyl-4-(2-(3- (ethyl(methyl)amino)cyclobutoxy)-7- (naphthalen-1-yl)-5,6,7,8- tetrahydropyrido[3,4-d]pyrimidin-4- yl)piperazin-2-yl)acetonitrile | 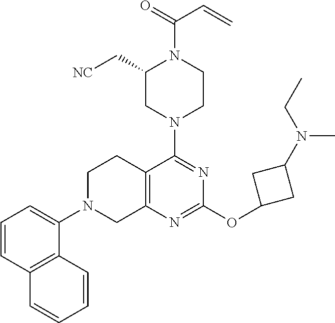 [SMILES: C=CC(=O)N1CCN(c2nc(OC3CC(N(C)CC)C3)nc3c2CCN(c2cccc4ccccc24)C3)C[C@@H]1CC#N] | 566 |
| 160 | (S)-2-(1-acryloyl-4-(2-(3-(ethyl(2- methoxyethyl)amino)cyclobutoxy)-7- (naphthalen-1-yl)-5,6,7,8- tetrahydropyrido[3,4-d]pyrimidin-4- yl)piperazin-2-yl)acetonitrile | 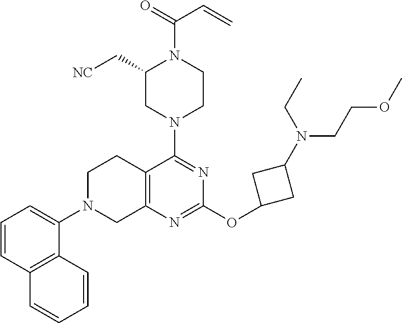 [SMILES: C=CC(=O)N1CCN(c2nc(OC3CC(N(CC)CCOC)C3)nc3c2CCN(c2cccc4ccccc24)C3)C[C@@H]1CC#N] | 610 |
| 161 | 2-((2S)-1-acryloyl-4-(2-(3-(dimethylamino)- 2-(2-methoxyethyl)cyclobutoxy)-7- (naphthalen-1-yl)-5,6,7,8- tetrahydropyrido[3,4-d]pyrimidin-4- yl)piperazin-2-yl)acetonitrile | 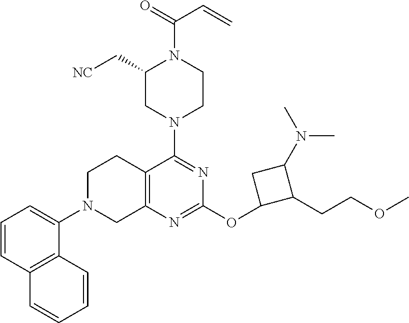 [SMILES: C=CC(=O)N1CCN(c2nc(OC3CC(N(C)C)C3CCOC)nc3c2CCN(c2cccc4ccccc24)C3)C[C@@H]1CC#N] | 610 |
| 162 | (S)-2-(1-acryloyl-4-(7-(naphthalen-1-yl)-2-(3- (pyrrolidin-1-yl)cyclobutoxy)-5,6,7,8- tetrahydropyrido[3,4-d]pyrimidin-4- yl)piperazin-2-yl)acetonitrile | 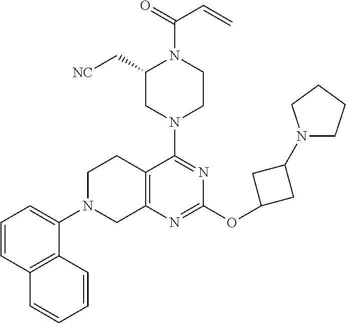 [SMILES: C=CC(=O)N1CCN(c2nc(OC3CC(N4CCCC4)C3)nc3c2CCN(c2cccc4ccccc24)C3)C[C@@H]1CC#N] | 578 |
| 163 | (S)-2-(1-acryloyl-4-(7-(2-chlorophenyl)-2- ((1-(pyrrolidin-1-yl)cyclobutyl)methoxy)- 5,6,7,8-tetrahydropyrido[3,4-d]pyrimidin-4- yl)piperazin-2-yl)acetonitrile | 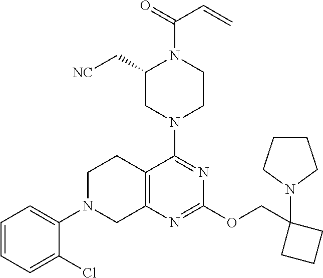 [SMILES: C=CC(=O)N1CCN(c2nc(OCC3(N4CCCC4)CCC3)nc3c2CCN(c2ccccc2Cl)C3)C[C@@H]1CC#N] | 576 |
| 164 | 2-((2S)-1-acryloyl-4-(2-((1-(1- methylpyrrolidin-3-yl)cyclobutyl)methoxy)- 7-(2-(trifluoromethyl)phenyl)-5,6,7,8- tetrahydropyrido[3,4-d]pyrimidin-4- yl)piperazin-2-yl)acetonitrile | 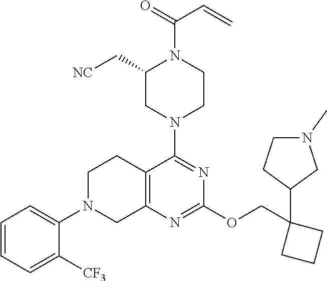 [SMILES: C=CC(=O)N1CCN(c2nc(OCC3(C4CCN(C)C4)CCC3)nc3c2CCN(c2ccccc2C(F)(F)F)C3)C[C@@H]1CC#N] | 624 |
| 165 | 2-((2S)-1-acryloyl-4-(2-((1-(1- methylpyrrolidin-2-yl)cyclobutyl)methoxy)- 7-(2-(trifluoromethyl)phenyl)-5,6,7,8- tetrahydropyrido[3,4-d]pyrimidin-4- yl)piperazin-2-yl)acetonitrile | 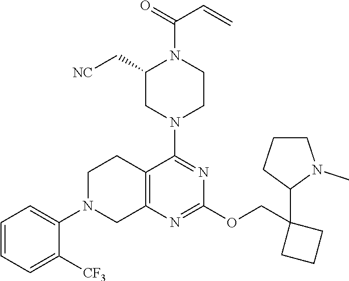 [SMILES: C=CC(=O)N1CCN(c2nc(OCC3(C4CCCN4C)CCC3)nc3c2CCN(c2ccccc2C(F)(F)F)C3)C[C@@H]1CC#N] | 624 |
| 166 | 2-((2S)-1-acryloyl-4-(2-((2- (dimethylamino)cyclobutyl)methoxy)-7- (naphthalen-1-yl)-5,6,7,8- tetrahydropyrido[3,4-d]pyrimidin-4- yl)piperazin-2-yl)acetonitrile | 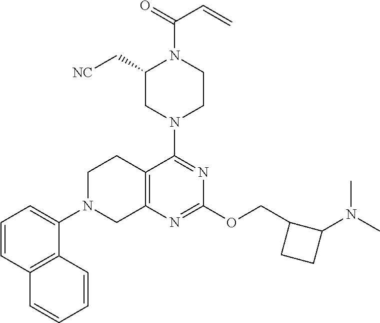 [SMILES: C=CC(=O)N1CCN(c2nc(OCC3CCC3N(C)C)nc3c2CCN(c2cccc4ccccc24)C3)C[C@@H]1CC#N] | 566 |
| 167 | (S)-2-(1-acryloyl-4-(7-(3-chloro-2- (trifluoromethyl)phenyl)-2-((3- (dimethylamino)cyclobutyl)methoxy)- 5,6,7,8-tetrahydropyrido[3,4-d]pyrimidin-4- yl)piperazin-2-yl)acetonitrile | 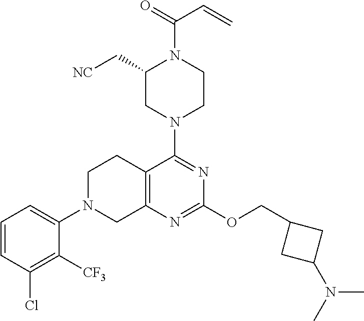 [SMILES: C=CC(=O)N1CCN(c2nc(OCC3CC(N(C)C)C3)nc3c2CCN(c2cccc(Cl)c2C(F)(F)F)C3)C[C@@H]1CC#N] | 618 |
| 168 | 2-((S)-1-acryloyl-4-(2-((R)-1-(3- (dimethylamino)cyclobutyl)ethoxy)-7-(3- fluoro-2-(trifluoromethyl)phenyl)-5,6,7,8- tetrahydropyrido[3,4-d]pyrimidin-4- yl)piperazin-2-yl)acetonitrile | 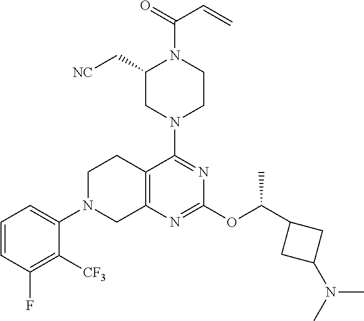 [SMILES: C=CC(=O)N1CCN(c2nc(O[C@H](C)C3CC(N(C)C)C3)nc3c2CCN(c2cccc(F)c2C(F)(F)F)C3)C[C@@H]1CC#N] | 616 |
| 169 | (S)-2-(1-acryloyl-4-(7-(2-chlorophenyl)-2- ((1-(dimethylamino)cyclopentyl)methoxy)- 5,6,7,8-tetrahydropyrido[3,4-d]pyrimidin-4- yl)piperazin-2-yl)acetonitrile | 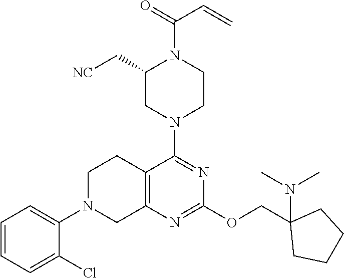 [SMILES: C=CC(=O)N1CCN(c2nc(OCC3(N(C)C)CCCC3)nc3c2CCN(c2ccccc2Cl)C3)C[C@@H]1CC#N] | 564 |
| 170 | 2-((2S)-1-acryloyl-4-(7-(2-chlorophenyl)-2- ((1-(dimethylamino)-3- (trifluoromethyl)cyclopentyl)methoxy)- 5,6,7,8-tetrahydropyrido[3,4-d]pyrimidin-4- yl)piperazin-2-yl)acetonitrile | 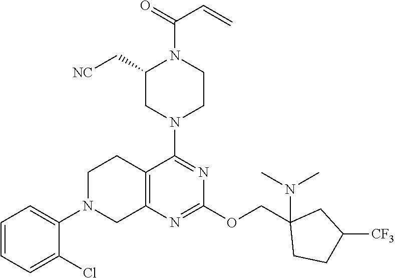 [SMILES: C=CC(=O)N1CCN(c2nc(OCC3(N(C)C)CCC(C(F)(F)F)C3)nc3c2CCN(c2ccccc2Cl)C3)C[C@@H]1CC#N] | 632 |
| 171 | 2-((2S)-1-acryloyl-4-(7-(2-chlorophenyl)-2- ((1-(dimethylamino)-3- (trifluoromethoxy)cyclopentyl)methoxy)- 5,6,7,8-tetrahydropyrido[3,4-d]pyrimidin-4- yl)piperazin-2-yl)acetonitrile | 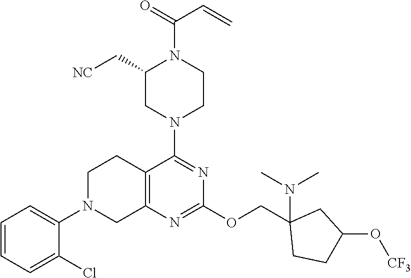 [SMILES: C=CC(=O)N1CCN(c2nc(OCC3(N(C)C)CCC(OC(F)(F)F)C3)nc3c2CCN(c2ccccc2Cl)C3)C[C@@H]1CC#N] | 648 |
| 172 | 3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)cyclopentane-1-carbonitrile | 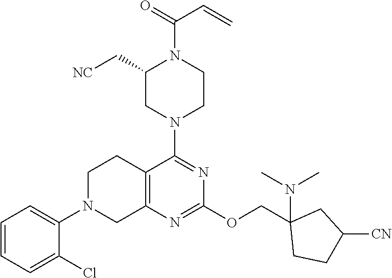 [SMILES: C=CC(=O)N1CCN(c2nc(OCC3(N(C)C)CCC(C#N)C3)nc3c2CCN(c2ccccc2Cl)C3)C[C@@H]1CC#N] | 589 |
| 173 | ethyl 3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)cyclopentane-1-carboxylate | 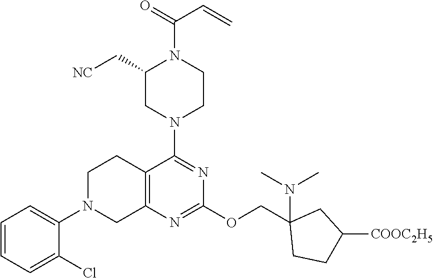 | 636 |
| 174 | 3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)-N,N-dimethylcyclopentane- 1-carboxamide | 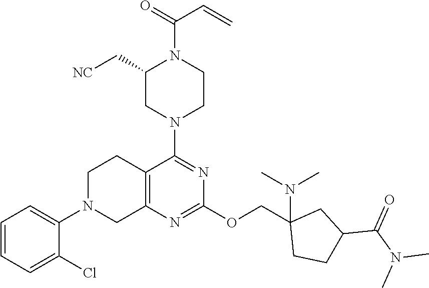 [SMILES: C=CC(=O)N1CCN(c2nc(OCC3(N(C)C)CCC(C(=O)N(C)C)C3)nc3c2CCN(c2ccccc2Cl)C3)C[C@@H]1CC#N] | 635 |
| 175 | 2-((2S)-1-acryloyl-4-(7-(2-chlorophenyl)-2- ((1-(dimethylamino)-3-(pyrrolidine-1- carbonyl)cyclopentyl)methoxy)-5,6,7,8- tetrahydropyrido[3,4-d]pyrimidin-4- yl)piperazin-2-yl)acetonitrile | 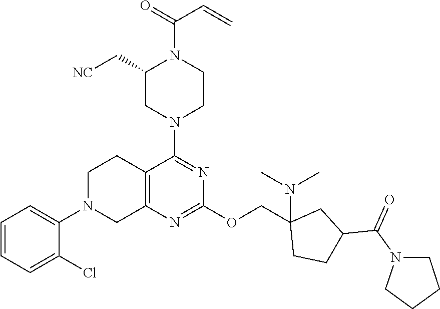 [SMILES: C=CC(=O)N1CCN(c2nc(OCC3(N(C)C)CCC(C(=O)N4CCCC4)C3)nc3c2CCN(c2ccccc2Cl)C3)C[C@@H]1CC#N] | 661 |
| 176 | 3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)-N-methylcyclopentane-1- carboxamide | 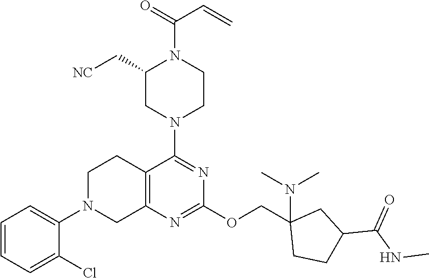 [SMILES: C=CC(=O)N1CCN(c2nc(OCC3(N(C)C)CCC(C(=O)NC)C3)nc3c2CCN(c2ccccc2Cl)C3)C[C@@H]1CC#N] | 621 |
| 177 | 2-((2S)-1-acryloyl-4-(7-(2-chlorophenyl)-2- ((1-(dimethylamino)-3-(thiazol-2- yl)cyclopentyl)methoxy)-5,6,7,8- tetrahydropyrido[3,4-d]pyrimidin-4- yl)piperazin-2-yl)acetonitrile | 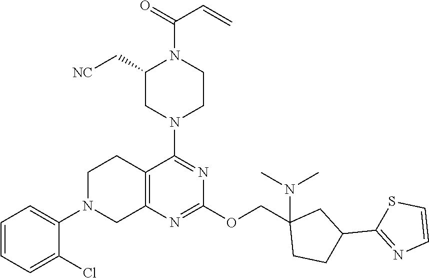 [SMILES: C=CC(=O)N1CCN(c2nc(OCC3(N(C)C)CCC(c4nccs4)C3)nc3c2CCN(c2ccccc2Cl)C3)C[C@@H]1CC#N] | 647 |
| 178 | (S)-2-(1-acryloyl-4-(7-(2,3-difluorophenyl)-2- ((1-(pyrrolidin-1-yl)cyclopentyl)methoxy)- 5,6,7,8-tetrahydropyrido[3,4-d]pyrimidin-4- yl)piperazin-2-yl)acetonitrile | 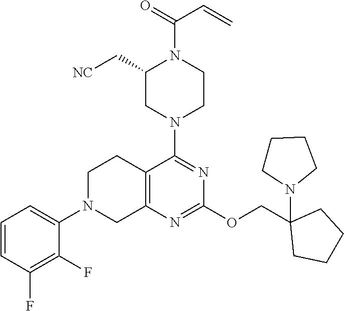 [SMILES: C=CC(=O)N1CCN(c2nc(OCC3(N4CCCC4)CCCC3)nc3c2CCN(c2cccc(F)c2F)C3)C[C@@H]1CC#N] | 592 |
| 179 | 2-((S)-1-acryloyl-4-(2-((1-(1- methylpyrrolidin-2-yl)cyclopentyl)methoxy)- 7-(naphthalen-1-yl)-5,6,7,8- tetrahydropyrido[3,4-d]pyrimidin-4- yl)piperazin-2-yl)acetonitrile | 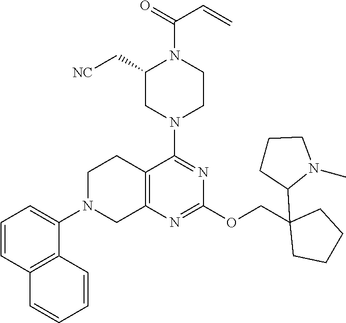 [SMILES: C=CC(=O)N1CCN(c2nc(OCC3(C4CCCN4C)CCCC3)nc3c2CCN(c2cccc4ccccc24)C3)C[C@@H]1CC#N] | 620 |
| 180 | 2-((2S)-1-acryloyl-4-(7-(2-chloro-3- fluorophenyl)-2-((2,2-difluoro-1-(1- methylpyrrolidin-2-yl)cyclopentyl)methoxy)- 5,6,7,8-tetrahydropyrido[3,4-d]pyrimidin-4- yl)piperazin-2-yl)acetonitrile | 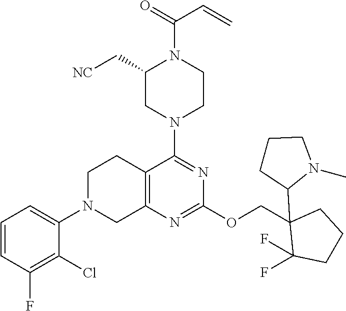 [SMILES: C=CC(=O)N1CCN(c2nc(OCC3(C4CCCN4C)CCCC3(F)F)nc3c2CCN(c2cccc(F)c2Cl)C3)C[C@@H]1CC#N] | 658 |
| 181 | 2-((2S)-1-acryloyl-4-(7-(2-chloro-3- fluorophenyl)-2-((1-(1-methylpyrrolidin-3- yl)cyclopentyl)methoxy)-5,6,7,8- tetrahydropyrido[3,4-d]pyrimidin-4- yl)piperazin-2-yl)acetonitrile | 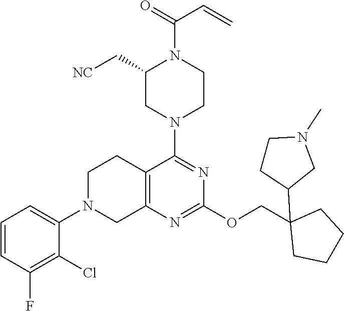 [SMILES: C=CC(=O)N1CCN(c2nc(OCC3(C4CCN(C)C4)CCCC3)nc3c2CCN(c2cccc(F)c2Cl)C3)C[C@@H]1CC#N] | 622 |
| 182 | 2-((2S)-1-acryloyl-4-(7-(2,3-dichlorophenyl)- 2-((1-(1-isopropylpyrrolidin-3- yl)cyclopentyl)methoxy)-5,6,7,8- tetrahydropyrido[3,4-d]pyrimidin-4- yl)piperazin-2-yl)acetonitrile | 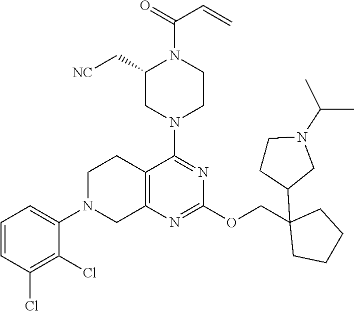 [SMILES: C=CC(=O)N1CCN(c2nc(OCC3(C4CCN(C(C)C)C4)CCCC3)nc3c2CCN(c2cccc(Cl)c2Cl)C3)C[C@@H]1CC#N] | 666 |
| 183 | (S)-2-(1-acryloyl-4-(7-(2-chlorophenyl)-2- ((1-((dimethylamino)methyl)cyclopentyl) oxy)-5,6,7,8-tetrahydropyrido[3,4-d] pyrimidin-4-yl)piperazin-2-yl)acetonitrile | 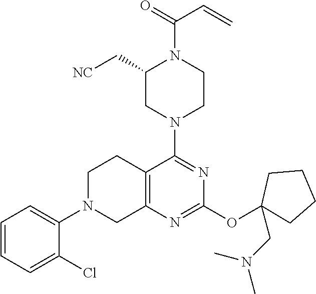 [SMILES: C=CC(=O)N1CCN(c2nc(OC3(CN(C)C)CCCC3)nc3c2CCN(c2ccccc2Cl)C3)C[C@@H]1CC#N] | 564 |
| 184 | (S)-2-(1-acryloyl-4-(2-((1-(azetidin-1- ylmethyl)cyclopentyl)oxy)-7-(2- (trifluoromethyl)phenyl)-5,6,7,8- tetrahydropyrido[3,4-d]pyrimidin-4- yl)piperazin-2-yl)acetonitrile | 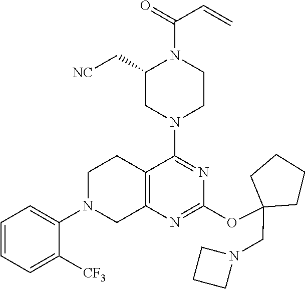 [SMILES: C=CC(=O)N1CCN(c2nc(OC3(CN4CCC4)CCCC3)nc3c2CCN(c2ccccc2C(F)(F)F)C3)C[C@@H]1CC#N] | 610 |
| 185 | (S)-2-(1-acryloyl-4-(7-(3-fluoro-2- (trifluoromethyl)phenyl)-2-((1-(pyrrolidin-1- ylmethyl)cyclopentyl)oxy)-5,6,7,8- tetrahydropyrido[3,4-d]pyrimidin-4- yl)piperazin-2-yl)acetonitrile | 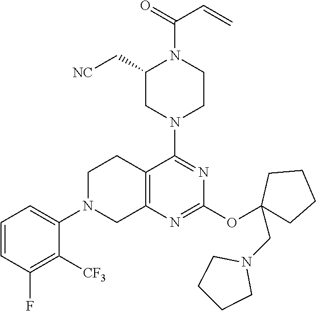 [SMILES: C=CC(=O)N1CCN(c2nc(OC3(CN4CCCC4)CCCC3)nc3c2CCN(c2cccc(F)c2C(F)(F)F)C3)C[C@@H]1CC#N] | 642 |
| 186 | 2-((S)-1-acryloyl-4-(7-(3-chloro-2- (trifluoromethyl)phenyl)-2-((1-((1- methylpyrrolidin-2- yl)methyl)cyclopentyl)oxy)-5,6,7,8- tetrahydropyrido[3,4-d]pyrimidin-4- yl)piperazin-2-yl)acetonitrile | 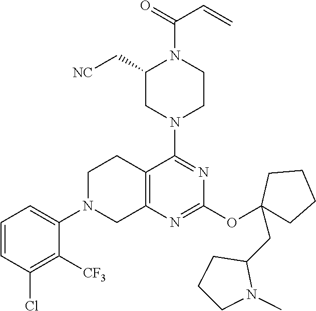 [SMILES: C=CC(=O)N1CCN(c2nc(OC3(CC4CCCN4C)CCCC3)nc3c2CCN(c2cccc(Cl)c2C(F)(F)F)C3)C[C@@H]1CC#N] | 672 |
| 187 | 2-((2S)-1-acryloyl-4-(2-((1-((4,4-difluoro-1- methylpyrrolidin-2- yl)methyl)cyclopentyl)oxy)-7-(naphthalen-1- yl)-5,6,7,8-tetrahydropyrido[3,4-d]pyrimidin- 4-yl)piperazin-2-yl)acetonitrile | 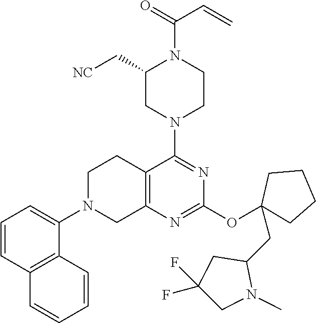 [SMILES: C=CC(=O)N1CCN(c2nc(OC3(CC4CC(F)(F)CN4C)CCCC3)nc3c2CCN(c2cccc4ccccc24)C3)C[C@@H]1CC#N] | 656 |
| 188 | 2-((2S)-1-acryloyl-4-(2-((1-(difluoro(1- methylpyrrolidin-2- yl)methyl)cyclopentyl)oxy)-7-(3-methyl-2- (trifluoromethyl)phenyl)-5,6,7,8- tetrahydropyrido[3,4-d]pyrimidin-4- yl)piperazin-2-yl)acetonitrile | 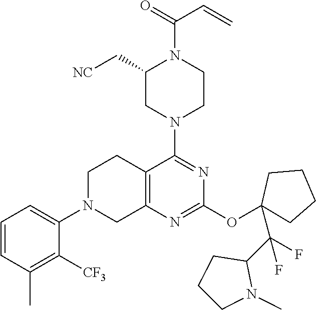 [SMILES: C=CC(=O)N1CCN(c2nc(OC3(C(F)(F)C4CCCN4C)CCCC3)nc3c2CCN(c2cccc(C)c2C(F)(F)F)C3)C[C@@H]1CC#N] | 688 |
| 189 | 2-((2S)-1-acryloyl-4-(2-((2- (dimethylamino)cyclopentyl)oxy)-7-(3- methyl-2-(trifluoromethyl)phenyl)-5,6,7,8- tetrahydropyrido[3,4-d]pyrimidin-4- yl)piperazin-2-yl)acetonitrile | 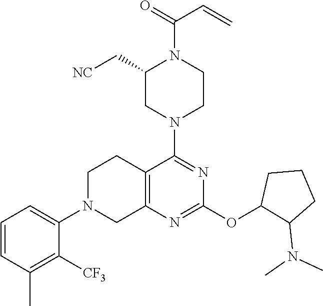 [SMILES: C=CC(=O)N1CCN(c2nc(OC3CCCC3N(C)C)nc3c2CCN(c2cccc(C)c2C(F)(F)F)C3)C[C@@H]1CC#N] | 598 |
| 190 | 2-((2S)-1-acryloyl-4-(2-((3- (dimethylamino)cyclopentyl)oxy)-7-(3- methyl-2-(trifluoromethyl)phenyl)-5,6,7,8- tetrahydropyrido[3,4-d]pyrimidin-4- yl)piperazin-2-yl)acetonitrile | 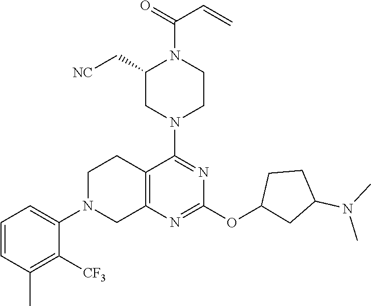 [SMILES: C=CC(=O)N1CCN(c2nc(OC3CCC(N(C)C)C3)nc3c2CCN(c2cccc(C)c2C(F)(F)F)C3)C[C@@H]1CC#N] | 598 |
| 191 | 2-((2S)-1-acryloyl-4-(2-((3- (dimethylamino)cyclopentyl)methoxy)-7-(3- methyl-2-(trifluoromethyl)phenyl)-5,6,7,8- tetrahydropyrido[3,4-d]pyrimidin-4- yl)piperazin-2-yl)acetonitrile | 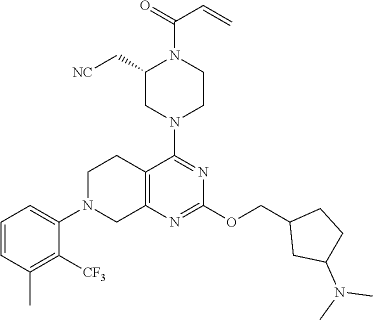 [SMILES: C=CC(=O)N1CCN(c2nc(OCC3CCC(N(C)C)C3)nc3c2CCN(c2cccc(C)c2C(F)(F)F)C3)C[C@@H]1CC#N] | 612 |
| 192 | 2-((2S)-1-acryloyl-4-(2-((2- (dimethylamino)cyclopentyl)methoxy)-7-(3- methyl-2-(trifluoromethyl)phenyl)-5,6,7,8- tetrahydropyrido[3,4-d]pyrimidin-4- yl)piperazin-2-yl)acetonitrile | 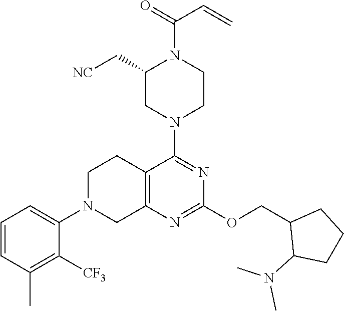 [SMILES: C=CC(=O)N1CCN(c2nc(OCC3CCCC3N(C)C)nc3c2CCN(c2cccc(C)c2C(F)(F)F)C3)C[C@@H]1CC#N] | 612 |
| 193 | (S)-2-(1-acryloyl-4-(7-(2-chlorophenyl)-2- ((1-((dimethylamino)methyl)cyclohexyl) oxy)-5,6,7,8-tetrahydropyrido[3,4-d] pyrimidin-4-yl)piperazin-2-yl)acetonitrile | 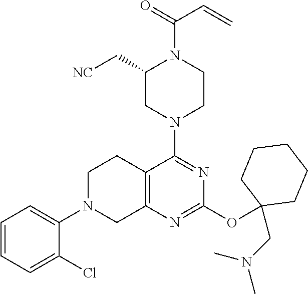 [SMILES: C=CC(=O)N1CCN(c2nc(OC3(CN(C)C)CCCCC3)nc3c2CCN(c2ccccc2Cl)C3)C[C@@H]1CC#N] | 578 |
| 194 | (S)-2-(1-acryloyl-4-(2-((1-(pyrrolidin-1- ylmethyl)cyclohexyl)oxy)-7-(2- (trifluoromethyl)phenyl)-5,6,7,8- tetrahydropyrido[3,4-d]pyrimidin-4- yl)piperazin-2-yl)acetonitrile | 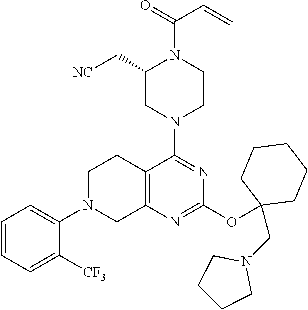 [SMILES: C=CC(=O)N1CCN(c2nc(OC3(CN4CCCC4)CCCCC3)nc3c2CCN(c2ccccc2C(F)(F)F)C3)C[C@@H]1CC#N] | 638 |
| 195 | 2-((2S)-1-acryloyl-4-(7-(2-chloro-3- fluorophenyl)-2-((2- (dimethylamino)cyclohexyl)oxy)-5,6,7,8- tetrahydropyrido[3,4-d]pyrimidin-4- yl)piperazin-2-yl)acetonitrile | 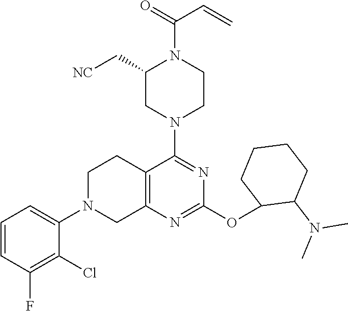 [SMILES: C=CC(=O)N1CCN(c2nc(OC3CCCCC3N(C)C)nc3c2CCN(c2cccc(F)c2Cl)C3)C[C@@H]1CC#N] | 582 |
| 196 | 2-((2S)-1-acryloyl-4-(7-(2,3-dichloro- phenyl)-2-((2-(1-methylazetidin-2- yl)cyclohexyl)oxy)-5,6,7,8- tetrahydropyrido[3,4-d]pyrimidin-4- yl)piperazin-2-yl)acetonitrile | 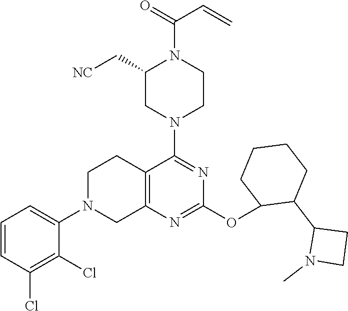 [SMILES: C=CC(=O)N1CCN(c2nc(OC3CCCCC3C3CCN3C)nc3c2CCN(c2cccc(Cl)c2Cl)C3)C[C@@H]1CC#N] | 624 |
| 197 | 2-((2S)-1-acryloyl-4-(7-(3-chloro-2- (trifluoromethyl)phenyl)-2-((2- (dimethylamino)cyclohexyl)methoxy)- 5,6,7,8-tetrahydropyrido[3,4-d]pyrimidin-4- yl)piperazin-2-yl)acetonitrile | 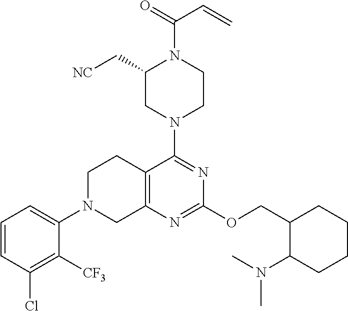 [SMILES: C=CC(=O)N1CCN(c2nc(OCC3CCCCC3N(C)C)nc3c2CCN(c2cccc(Cl)c2C(F)(F)F)C3)C[C@@H]1CC#N] | 646 |
| 198 | 2-((2S)-1-acryloyl-4-(2-((2- (dimethylamino)cyclohexyl)methoxy)-7- (naphthalen-1-yl)-5,6,7,8- tetrahydropyrido[3,4-d]pyrimidin-4- yl)piperazin-2-yl)acetonitrile | 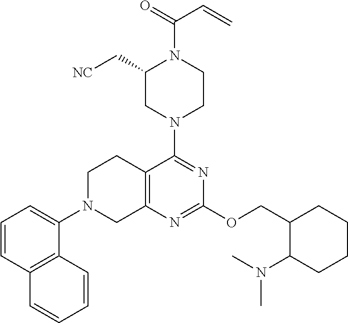 [SMILES: C=CC(=O)N1CCN(c2nc(OCC3CCCCC3N(C)C)nc3c2CCN(c2cccc4ccccc24)C3)C[C@@H]1CC#N] | 594 |
| 199 | (S)-2-(1-acryloyl-4-(7-(2-chlorophenyl)-2- ((1-(dimethylamino)cyclohexyl)methoxy)- 5,6,7,8-tetrahydropyrido[3,4-d]pyrimidin-4- yl)piperazin-2-yl)acetonitrile | 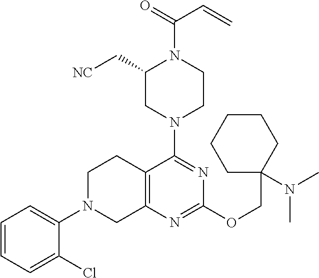 [SMILES: C=CC(=O)N1CCN(c2nc(OCC3(N(C)C)CCCCC3)nc3c2CCN(c2ccccc2Cl)C3)C[C@@H]1CC#N] | 578 |
| 200 | (S)-2-(1-acryloyl-4-(7-(2,3-difluorophenyl)- 2-((1-((dimethylamino) methyl)cyclohexyl)methoxy)-5,6,7,8- tetrahydropyrido[3,4-d]pyrimidin-4- yl)piperazin-2-yl)acetonitrile | 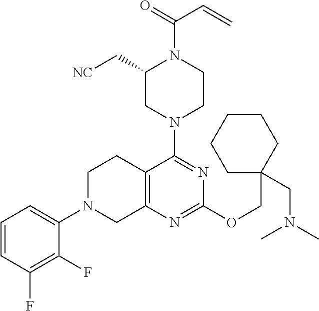 [SMILES: C=CC(=O)N1CCN(c2nc(OCC3(CN(C)C)CCCCC3)nc3c2CCN(c2cccc(F)c2F)C3)C[C@@H]1CC#N] | 594 |
| 201 | (S)-2-(1-acryloyl-4-(2-((4- (dimethylamino)cyclohexyl)methoxy)-7- (naphthalen-1-yl)-5,6,7,8- tetrahydropyrido[3,4-d]pyrimidin-4- yl)piperazin-2-yl)acetonitrile | 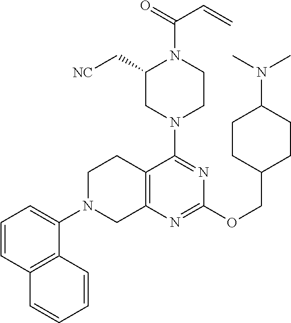 [SMILES: C=CC(=O)N1CCN(c2nc(OCC3CCC(N(C)C)CC3)nc3c2CCN(c2cccc4ccccc24)C3)C[C@@H]1CC#N] | 594 |
| 202 | 2-((2S)-1-acryloyl-4-(7-(3-chloro-2- (trifluoromethyl)phenyl)-2-((2-(pyrrolidin-1- yl)cyclohexyl)methoxy)-5,6,7,8- tetrahydropyrido[3,4-d]pyrimidin-4- yl)piperazin-2-yl)acetonitrile | 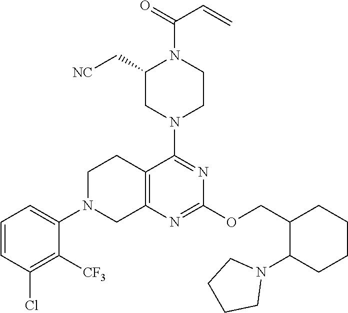 [SMILES: C=CC(=O)N1CCN(c2nc(OCC3CCCCC3N3CCCC3)nc3c2CCN(c2cccc(Cl)c2C(F)(F)F)C3)C[C@@H]1CC#N] | 672 |
| 203 | 2-((2S)-1-acryloyl-4-(7-(3-chloro-2- (trifluoromethyl)phenyl)-2-((3- (dimethylamino)cyclohexyl)methoxy)- 5,6,7,8-tetrahydropyrido[3,4-d]pyrimidin-4- yl)piperazin-2-yl)acetonitrile | 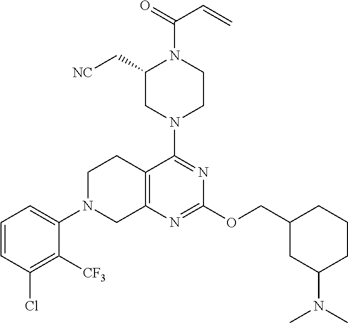 [SMILES: C=CC(=O)N1CCN(c2nc(OCC3CCCC(N(C)C)C3)nc3c2CCN(c2cccc(Cl)c2C(F)(F)F)C3)C[C@@H]1CC#N] | 646 |
| 204 | 2-((2S)-1-acryloyl-4-(7-(3-chloro-2- (trifluoromethyl)phenyl)-2-((3- (dimethylamino)cyclohexyl)oxy)-5,6,7,8- tetrahydropyrido[3,4-d]pyrimidin-4- yl)piperazin-2-yl)acetonitrile | 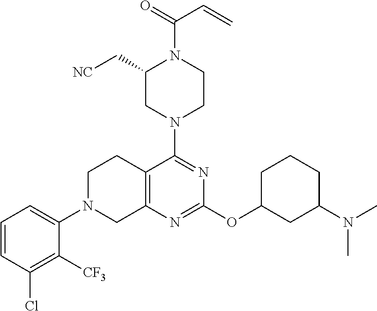 [SMILES: C=CC(=O)N1CCN(c2nc(OC3CCCC(N(C)C)C3)nc3c2CCN(c2cccc(Cl)c2C(F)(F)F)C3)C[C@@H]1CC#N] | 632 |
| 205 | N-(3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)cyclopentyl)acetamide | 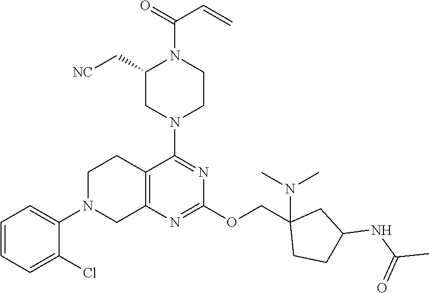 [SMILES: C=CC(=O)N1CCN(c2nc(OCC3(N(C)C)CCC(NC(C)=O)C3)nc3c2CCN(c2ccccc2Cl)C3)C[C@@H]1CC#N] | 621 |
| 206 | 3-(((4-((S)-4-acryloyl-3- (cyanomethyl)piperazin-1-yl)-7-(2- chlorophenyl)-5,6,7,8-tetrahydropyrido[3,4- d]pyrimidin-2-yl)oxy)methyl)-3- (dimethylamino)-N,N-dimethylcyclo- pentane-1-sulfonamide | 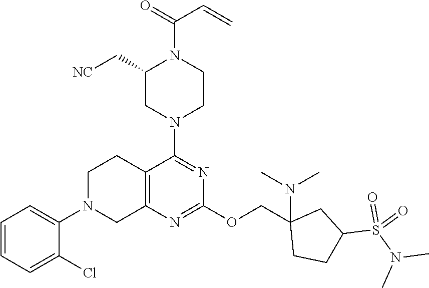 [SMILES: C=CC(=O)N1CCN(c2nc(OCC3(N(C)C)CCC(S(=O)(=O)N(C)C)C3)nc3c2CCN(c2ccccc2Cl)C3)C[C@@H]1CC#N] | 671 |
| 207 | 2-((2S)-1-acryloyl-4-(2-(((2- (dimethylamino)cyclobutyl)methyl)thio)-7- (naphthalen-1-yl)-5,6,7,8- tetrahydropyrido[3,4-d]pyrimidin-4- yl)piperazin-2-yl)acetonitrile | 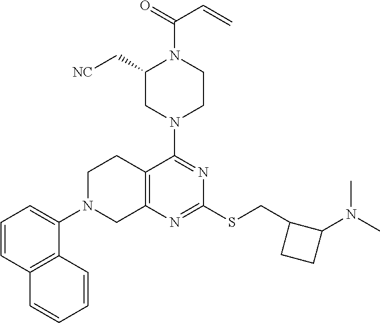 [SMILES: C=CC(=O)N1CCN(c2nc(SCC3CCC3N(C)C)nc3c2CCN(c2cccc4ccccc24)C3)C[C@@H]1CC#N] | 582 |
| 208 | 2-((2S)-1-acryloyl-4-(2-(((2- (dimethylamino)cyclobutyl)methyl)amino)- 7-(naphthalen-1-yl)-5,6,7,8- tetrahydropyrido[3,4-d]pyrimidin-4- yl)piperazin-2-yl)acetonitrile | 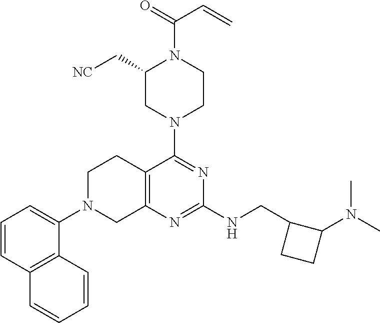 [SMILES: C=CC(=O)N1CCN(c2nc(NCC3CCC3N(C)C)nc3c2CCN(c2cccc4ccccc24)C3)C[C@@H]1CC#N] | 565 |

### Pharmacological Testing

1. SOS1 Catalyzed Nucleotide Exchange Assay

HIS-KRAS (G12C, aa 2-185, Sino biological) was diluted to 5 Min EDTA buffer (20 mM HEPES, pH 7.4, 50 mM NaCl, 10 mM EDA, 0.01% (v/v) Tween-20) and incubated for 30 min at 25° C. The EDTA pretreated HIS-KRAS(G12C) was diluted to 12 nM in assay buffer (25 mM HEPES, pH 7.4, 120 mM NaCl, 5 mM MgCl₂, 1 mM DTI, 0.01% (v/v) Tween 20, 0.1% (w/v) BSA) containing 120 nM GDP(Sigma) and Mab Anti 6HIS-Tb cryptate Gold(Cisbio) and incubated for 1 hour at 25° C. to prepare GDP-loaded HIS-KRAS(G12C). The GDP-loaded HIS-KRAS(G12C) was pre-incubation with diluted compounds in a 384-well plate (Greiner) for 1 hour, then purified SOS 1ExD (Flag tag, aa 564-1049) and BODIPY™FL GTP (Invitrogen) were added to the assay wells (Final concentration: 3 nM HIS-KRAS(G12C), 2 μM SOS1 ExD, 80 nM BODIPY™FL GTP, 21 ng/mL MAb Anti 6HS-Tb cryptate Gold) and incubated for 4 hours at 25° C. TR-FRET signals were then read on Tecan Spark multimode microplate reader. The parameters were F486: Excitation 340 (35) nm, Emission 486 (10) nm, Lag time 100 μs, Integration time 200 s; F515: Excitation 340 (35) nm, Emission 515 (10) nm, Lag time 100 s, Integration time 200 s. TR-FRET ratios for each individual wells were calculated by equation: TR-FRET ratio=(Signal F515/Signal F486)*10000. Then the data were analyzed using a 4-parameter logistic model to calculate IC₅₀ values. The results are as shown in Table 1.

2. Phospho-ERK1/2(THR202/TYR204) HTRF assay

NCI-H358 cells express KRAS G12C were cultured in RPMI 1640 medium (Gibco) containing 10% fetal bovine serum (Gibco). Cells in culture medium were plated in 96-well plates at a concentration of 40,000 cells/well and allowed to attach overnight. The next day, culture medium was removed and added compounds diluted in assay medium (RPMI 1640, 0.1% EFBS). After 2 hours incubation in a 37° C./5% CO₂ cell incubator, the assay medium was removed, then 50 μL of 1× supplemented lysis buffer (Cisbio) was added and the plates were incubated at 25° C. for 45 min with shaking. 10 μL of cell lysates from the 96-well plates were transferred to a 384-well plate (Greiner) containing 2.5 L/well HTRF® pre-mixed antibodies (Cisbio 64AERPEH).

Incubate 4 hours at 25° C. and then read HTRF signals on Tecan Spark multimode microplate reader. The data were analyzed using a 4-parameter logistic model to calculate IC₅₀ values. The results are as shown in Table 1.

3. Cell Growth inhibition assay

NCI-H358 cells express KRAS G12C were cultured in RPMI 1640 medium (Gibco) containing 10% fetal bovine serum (Gibco). Cells in culture medium were plated in 96-well plates at a concentration of 3000 cells/well (100 L/well) and allowed to attach overnight. The next day, compounds were diluted in culture medium and added to the plates. After 6 days incubation in a 37° C./5% CO₂ cell incubator, the medium was removed, then 100 μL of 1×CCK-8 (MCE) in culture medium was added in each well. The plates were incubated 1.5-2 hours in a 37° C./5% CO₂ cell incubator. OD450 signals were read on Tecan Spark multimode microplate reader and analyzed using a 4-parameter logistic model to calculate IC₅₀ values. The results are as shown in Table 1.

TABLE 1

| | SOS1 catalyzed nucleotide | p-ERK | NCI-H358 |
|---|---|---|---|
| | exchange assay | inhibition in | cell growth |
| | KRAS G12C | NCI-H358 | inhibition |
| Compound | IC₅₀, nM | IC₅₀, nM | IC₅₀, nM |
| 1 | ND | 3201 | ND |
| 2 | >500 | ND | ND |
| 3 | 12.08 | 452.9 | 408 |
| 4 | 18.91 | 97.7 | 93.27 |
| 5 | 19.59 | 330.2 | 173.6 |
| 6 | 178.5 | 591.9 | 579.5 |
| 7 | ND | ND | ND |
| 8 | 407.6 | 769.4 | 3469 |
| 9 | ND | 1152 | 888.3 |
| 10 | 1735 | 720 | 940.3 |
| 11 | ND | 9861 | ND |
| 12 | 176.2 | 518.1 | 644.6 |
| 13 | 683.8 | 1213 | ND |
| 14 | 54.15 | 234.4 | 216.6 |
| 15 | 333.4 | 876.5 | 497.1 |
| 16 | ND | 7056 | ND |
| 17 | 83.6 | 312.2 | 299.3 |
| 18 | 428.2 | 879 | 506.7 |
| 19 | 371.9 | 748.5 | ND |
| 20 | 19.4 | 127 | 101.7 |
| 21 | ND | >1000 | ND |
| 22 | 10.74 | 96.61 | 65.18 |
| 23 | 9.448 | 111.6 | 66.85 |
| 24 | 20.05 | 237.0 | 93.59 |
| 25 | 30.15 | 266.8 | 164.5 |
| 26 | 33.65 | 231.4 | 153.5 |
| 27 | 84.55 | 565.6 | 310.1 |
| 28 | 24.05 | ND | 138.0 |
| 29 | ND | >1000 | ND |
| 30 | ND | ND | ND |
| 31 | ND | ND | ND |
| 32 | ND | ND | 696.5 |
| 33 | ND | 302.3 | 652.4 |
| 34 | 15.87 | 54.55 | 78.56 |
| 35 | 17.92 | 85.35 | 105.8 |
| 36 | ND | ND | ND |
| 37 | 6.748 | 81.58 | 85.53 |
| 38 | 5.242 | 66.03 | 60.25 |
| 39 | 7.282 | 79.57 | 103.8 |
| 40 | 23.78 | ND | 360.2 |
| 41 | 13.47 | 149.3 | 178.8 |
| 42 | 5.242 | 63.56 | 118.5 |
| 43 | 5.045 | 71.83 | 134.8 |
| 44 | 33.24 | 220.4 | 741.9 |
| 45 | 13.32 | 216.4 | 590.1 |
| 46 | 2.573 | 14.72 | 26.9 |
| 47 | 1.158 | 23.69 | 26.44 |
| 48 | 3.309 | 65.98 | 94.93 |
| 49 | 3.124 | 38.35 | 64.2 |
| 50 | 8.658 | 83.83 | 202.4 |
| 51 | 3.282 | 77.12 | 99.89 |
| 52 | 484.8 | >500 | ND |
| 53 | 174.3 | >500 | ND |
| 54 | 82.82 | >500 | >1000 |
| 55 | >500 | ND | ND |
| 56 | 3.186 | 38.23 | 22.42 |
| 57 | 28.06 | 296.5 | 522.2 |
| 58 | 1.47 | 44.34 | 36.92 |
| 59 | 1.105 | 57.25 | 61.87 |
| 60 | ND | 7124 | ND |
| 61 | 1.438 | 16.21 | 25.05 |
| 62 | 2.339 | 43.03 | 66.94 |
| 63 | 30.86 | 533.2 | 860.7 |
| 64 | 1.689 | 123.7 | 74.52 |
| 65 | 1.462 | 87.81 | 32.84 |
| 66 | 1.457 | 13.57 | 25.11 |
| 67 | 1.84 | 22.4 | 20.4 |
| 68 | 1.603 | 69.25 | 51.49 |
| 69 | 2.734 | 157.0 | 77.56 |
| 70 | 3.412 | 172.3 | 195.1 |
| 71 | 7.573 | 505.1 | 369.3 |
| 72 | 11.84 | 214.8 | 378.5 |
| 73 | 16.96 | 962.7 | 883.1 |
| 74 | 66.77 | >500 | 1520 |
| 75 | 7.267 | 405.1 | 235.2 |
| 76 | 11.69 | 353.7 | 420.9 |
| 77 | 1.238 | 59.26 | 29.03 |
| 78 | 8.359 | 207.6 | 207.5 |
| 79 | 6.026 | 439 | 239.1 |
| 80 | 284.6 | >500 | ND |
| 81 | >500 | ND | ND |
| 82 | 211.2 | >500 | ND |
| 83 | 3.901 | 190.4 | 116.3 |
| 84 | 1.577 | 80.19 | 24.3 |
| 85 | 2.86 | 152.8 | 132.4 |
| 86 | >500 | 130.7 | 102.7 |

4. NCI-H1373 xenograft model

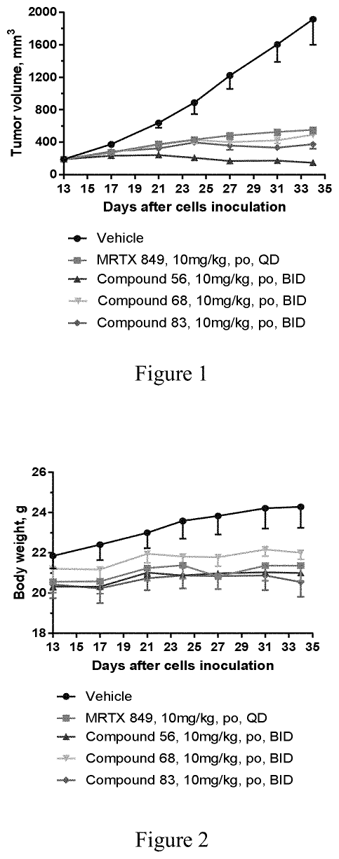

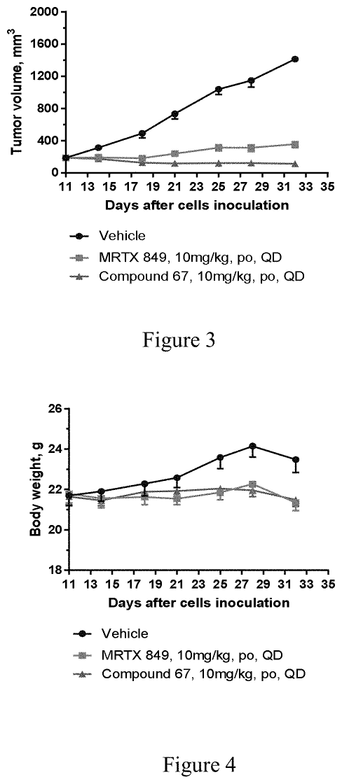

NCI-H1373 cells (5.0E+06 cells) were injected subcutaneously into the right flank of female BALB/c mice (6-8 weeks) in a mixture with Matrigel (BD Bioscience). Mice were monitored daily and caliper measurements began when tumors became visible. Tumor volume was calculated by measuring two perpendicular diameters using the following formula: (L*w²)/2 in which L and w refer to the length and width tumor diameter, respectively. When the average tumor volume reached 150-200 mm³, mice were grouped (n=6/group) and treated with compounds. Tumor volume and mice weight was measured twice a week during treatment (˜3 weeks). Tumor growth inhibition rates were calculated by TGI %=(1−(Vt−Vt₀)/(Vc−Vc₀))*100%, where Vc, Vt are the mean tumor volume of control and treated groups at the end of the study, and Vc₀ and Vt₀ are the mean tumor volume of control and treated groups at the start. The TGI % in each of compound 56, compound 68, compound 83, MRTX 849 and the vehicle groups are shown in Table 2. The tumor volume of mice varies with the number of days after cell inoculation is shown in FIG. 1. The weight of mice varies with the number of days after cell inoculation is shown in FIG. 2. The TGI % in each of compound 67, MRTX849 and the vehicle groups are shown in Table 3. The tumor volume of mice varies with the number of days after cell inoculation is shown in FIG. 3, and the weight of mice varies with the number of days after cell inoculation is shown in FIG. 4. The structure of MRTX 849 is as follows:

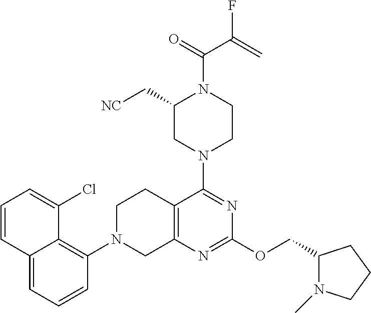

TABLE 2

| | Tumor volume | Tumor volume | |
|---|---|---|---|
| | at the start, | at the end | |
| Groups | mm³ | (Day 22), mm³ | TGI % |
| Vehicle | 191 | 1914 | — |
| MRTX 849, 10 mg/kg, QD | 191 | 553 | 79.0 |
| Compound 56, 10 mg/kg, BID | 191 | 148 | 102.5 |
| Compound 68, 10 mg/kg, BID | 191 | 490 | 82.6 |
| Compound 83 10 mg/kg, BID | 191 | 374 | 89.4 |

TABLE 3

| | Tumor volume | Tumor volume | |
|---|---|---|---|
| | at the start, | at the end | |
| Groups | mm³ | (Day 22), mm³ | TGI % |
| Vehicle | 188 | 1413 | — |
| MRTX 849, 10 mg/kg, QD | 188 | 355 | 86.3 |
| Compound 67, 10 mg/kg, QD | 188 | 114 | 106.1 |

From Table 2, Table 3 and FIGS. 1 to 4, it can be seen that the compounds of the present invention have good activity to inhibit tumor growth and have good safety.

It should be understood that if the present invention quotes any prior art publication, it should be understood that: such quotation does not mean that the publication is recognized as part of the common knowledge in the field in any country. Although for the sake of clear understanding, the present invention has been described in detail by way of examples, it is obvious to those skilled in the art that certain minor changes and modifications will be made. Therefore, the description and examples should not be construed as limiting the scope of the present invention.

What is claimed is:

1. A compound, a pharmaceutical acceptable salt thereof or stereoisomer thereof, wherein the compound is selected from:

| | |
|---|---|
| 1. | 2-(1-acryloyl-4-(2-(((1R,4r)-4-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 2. | 2-(1-acryloyl-4-(2-((1-((dimethylamino)methyl)cyclopropyl)methoxy)-7-(2-methyl-1-oxo-1,2- |
| | dihydroisoquinolin-8-yl)-5,6,7,8-tetrahydropyrido[3,4-d]pyrimidin-4-yl)piperazin-2-yl)acetonitrile; |
| 3. | 2-(1-acryloyl-4-(2-((1-((dimethylamino)methyl)cyclopropyl)methoxy)-7-(5-methyl-1H-indazol-4- |
| | yl)-5,6,7,8-tetrahydropyrido[3,4-d]pyrimidin-4-yl)piperazin-2-yl)acetonitrile; |
| 4. | 2-(1-acryloyl-4-(7-(naphthalen-1-yl)-2-((1-(pyrrolidin-1-ylmethyl)cyclopropyl)methoxy)-5,6,7,8- |
| | tetrahydropyrido[3,4-d]pyrimidin-4-yl)piperazin-2-yl)acetonitrile; |
| 5. | 2-(1-acryloyl-4-(2-((3-(dimethylamino)cyclohexyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 6. | 2-(1-acryloyl-4-(2-((3-(dimethylamino)cyclohexyl)methoxy)-7-(2-(trifluoromethyl)phenyl)-5,6,7,8- |
| | tetrahydropyrido[3,4-d]pyrimidin-4-yl)piperazin-2-yl)acetonitrile; |
| 7. | 2-(1-acryloyl-4-(2-(3-(dimethylamino)cyclobutoxy)-7-(naphthalen-1-yl)-5,6,7,8- |
| | tetrahydropyrido[3,4-d]pyrimidin-4-yl)piperazin-2-yl)acetonitrile; |
| 8. | 2-(1-acryloyl-4-(2-((3-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 9. | 2-(1-acryloyl-4-(2-((1-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 10. | 2-(1-acryloyl-4-(2-((3-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 11. | 2-(1-acryloyl-4-(2-((2-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 12. | 2-(1-acryloyl-4-(2-((1-(dimethylamino)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 13. | 2-(1-acryloyl-4-(2-((3-(dimethylamino)cyclopentyl)oxy)-7-(naphthalen-1-yl)-5,6,7,8- |
| | tetrahydropyrido[3,4-d]pyrimidin-4-yl)piperazin-2-yl)acetonitrile; |
| 14. | 2-(1-acryloyl-4-(2-((2-(dimethylamino)cyclopentyl)methoxy)-7-(naphthalen-1-yl)-5,6,7,8- |
| | tetrahydropyrido[3,4-d]pyrimidin-4-yl)piperazin-2-yl)acetonitrile; |
| 15. | 2-(1-acryloyl-4-(2-((3-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 16. | 2-(1-acryloyl-4-(2-((2-(4-methylpiperazin-1-yl)cyclopentyl)oxy)-7-(naphthalen-1-yl)-5,6,7,8- |
| | tetrahydropyrido[3,4-d]pyrimidin-4-yl)piperazin-2-yl)acetonitrile; |
| 17. | 2-(1-acryloyl-4-(2-((3-(dimethylamino)cyclopentyl)methoxy)-7-(naphthalen-1-yl)-5,6,7,8- |
| | tetrahydropyrido[3,4-d]pyrimidin-4-yl)piperazin-2-yl)acetonitrile; |
| 18. | 2-(1-acryloyl-4-(2-((2-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 19. | 2-(1-acryloyl-4-(2-((2-((dimethylamino)methyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 20. | 2-(1-acryloyl-4-(2-((1-(dimethylamino)cyclopropyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 21. | 2-(1-acryloyl-4-(2-(2-(2-(dimethylamino)cyclopentyl)ethoxy)-7-(naphthalen-1-yl)-5,6,7,8- |
| | tetrahydropyrido[3,4-d]pyrimidin-4-yl)piperazin-2-yl)acetonitrile; |
| 22. | 2-(1-acryloyl-4-(2-(((1R,3S)-3-(dimethylamino)cyclopentyl)methoxy)-7-(8-methylnaphthalen-1- |
| | yl)-5,6,7,8-tetrahydropyrido[3,4-d]pyrimidin-4-yl)piperazin-2-yl)acetonitrile; |
| 23. | 2-(1-acryloyl-4-(2-((2-(dimethylamino)cyclopentyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 24. | 2-(1-acryloyl-4-(2-((3-(dimethylamino)cyclopentyl)methoxy)-7-(8-methylnaphthalen-1-yl)-5,6,7,8- |
| | tetrahydropyrido[3,4-d]pyrimidin-4-yl)piperazin-2-yl)acetonitrile; |
| 25. | 2-(1-acryloyl-4-(7-(2,3-dichlorophenyl)-2-((2-(dimethylamino)cyclopentyl)methoxy)-5,6,7,8- |
| | tetrahydropyrido[3,4-d]pyrimidin-4-yl)piperazin-2-yl)acetonitrile; |
| 26. | 2-(1-acryloyl-4-(2-((2-(dimethylamino)cyclopentyl)methoxy)-7-(3-fluoro-2- |
| | (trifluoromethyl)phenyl)-5,6,7,8-tetrahydropyrido[3,4-d]pyrimidin-4-yl)piperazin-2-yl)acetonitrile; |
| 27. | 2-(1-acryloyl-4-(2-((2-(dimethylamino)cyclopentyl)methoxy)-7-(2-(trifluoromethyl)phenyl)- |
| | 5,6,7,8-tetrahydropyrido[3,4-d]pyrimidin-4-yl)piperazin-2-yl)acetonitrile; |
| 28. | 2-(1-acryloyl-4-(2-((2-(dimethylamino)cyclopentyl)methoxy)-7-(2,3-dimethylphenyl)-5,6,7,8- |
| | tetrahydropyrido[3,4-d]pyrimidin-4-yl)piperazin-2-yl)acetonitrile; |
| 29. | 2-(1-acryloyl-4-(7-(2-chloro-3-fluorophenyl)-2-((3-(dimethylamino)cyclopentyl)methoxy)-5,6,7,8- |
| | tetrahydropyrido[3,4-d]pyrimidin-4-yl)piperazin-2-yl)acetonitrile; |
| 30. | 2-(1-acryloyl-4-(2-((3-(dimethylamino)cyclopentyl)methoxy)-7-(4-(trifluoromethyl)106aphthal-3- |
| | yl)-5,6,7,8-tetrahydropyrido[3,4-d]pyrimidin-4-yl)piperazin-2-yl)acetonitrile; |
| 31. | 2-(1-acryloyl-4-(7-(2-chlorophenyl)-2-((3-(dimethylamino)cyclopentyl)methoxy)-5,6,7,8- |
| | tetrahydropyrido[3,4-d]pyrimidin-4-yl)piperazin-2-yl)acetonitrile; |
| 32. | 2-(1-acryloyl-4-(7-(naphthalen-1-yl)-2-((1-(pyrrolidin-1-yl)cyclopropyl)methoxy)-5,6,7,8- |
| | tetrahydropyrido[3,4-d]pyrimidin-4-yl)piperazin-2-yl)acetonitrile; |
| 33. | 2-(1-acryloyl-4-(7-(106aphthalene-1-yl)-2-((3-(pyrrolidin-1-yl)cyclopentyl)methoxy)-5,6,7,8- |
| | tetrahydropyrido[3,4-d]pyrimidin-4-yl)piperazin-2-yl)acetonitrile; |
| 34. | 2-(1-acryloyl-4-(7-(3-fluoro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 35. | 2-(1-acryloyl-4-(7-(2,3-dichlorophenyl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 36. | 2-(1-acryloyl-4-(2-((1-(pyrrolidin-1-ylmethyl)cyclopropyl)methoxy)-7-(2-(trifluoromethyl)pyridin- |
| | 3-yl)-5,6,7,8-tetrahydropyrido[3,4-d]pyrimidin-4-yl)piperazin-2-yl)acetonitrile; |
| 37. | 2-(1-acryloyl-4-(2-((1-((dimethylamino)methyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 38. | 2-(1-acryloyl-4-(7-(3-chloro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 39. | 2-(1-acryloyl-4-(2-((1-(azetidin-1-ylmethyl)cyclopropyl)methoxy)-7-(3-chloro-2- |
| | (trifluoromethyl)phenyl)-5,6,7,8-tetrahydropyrido[3,4-d]pyrimidin-4-yl)piperazin-2-yl)acetonitrile; |
| 40. | 2-(1-acryloyl-4-(7-(3-chloro-2-(trifluoromethyl)phenyl)-2-((1- |
| | (morpholinomethyl)cyclopropyl)methoxy)-5,6,7,8-tetrahydropyrido[3,4-d]pyrimidin-4- |
| | yl)piperazin-2-yl)acetonitrile; |
| 41. | 2-(1-acryloyl-4-(2-((1-(azetidin-1-ylmethyl)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 42. | 2-(1-acryloyl-4-(2-((1-(azetidin-1-ylmethyl)cyclopropyl)methoxy)-7-(8-chloronaphthalen-1-yl)- |
| | 5,6,7,8-tetrahydropyrido[3,4-d]pyrimidin-4-yl)piperazin-2-yl)acetonitrile; |
| 43. | 2-(1-acryloyl-4-(2-((1-(azetidin-1-ylmethyl)cyclopropyl)methoxy)-7-(8-methylnaphthalen-1-yl)- |
| | 5,6,7,8-tetrahydropyrido[3,4-d]pyrimidin-4-yl)piperazin-2-yl)acetonitrile; |
| 44. | 2-(1-acryloyl-4-(7-(naphthalen-1-yl)-2-((1-(pyrrolidin-1-ylmethyl)cyclobutyl)methoxy)-5,6,7,8- |
| | tetrahydropyrido[3,4-d]pyrimidin-4-yl)piperazin-2-yl)acetonitrile; |
| 45. | 2-(1-acryloyl-4-(7-(8-methylnaphthalen-1-yl)-2-((1-(pyrrolidin-1-ylmethyl)cyclobutyl)methoxy)- |
| | 5,6,7,8-tetrahydropyrido[3,4-d]pyrimidin-4-yl)piperazin-2-yl)acetonitrile; |
| 46. | 2-(1-acryloyl-4-(7-(8-chloronaphthalen-1-yl)-2-((1- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 47. | 2-(1-acryloyl-4-(2-((1-((dimethylamino)methyl)cyclopropyl)methoxy)-7-(8-methylnaphthalen-1- |
| | yl)-5,6,7,8-tetrahydropyrido[3,4-d]pyrimidin-4-yl)piperazin-2-yl)acetonitrile; |
| 48. | 2-(1-acryloyl-4-(2-((1-((dimethylamino)methyl)cyclopropyl)methoxy)-7-(2,3-dimethylphenyl)- |
| | 5,6,7,8-tetrahydropyrido[3,4-d]pyrimidin-4-yl)piperazin-2-yl)acetonitrile; |
| 49. | 2-(1-acryloyl-4-(2-((1-(((R)-3-fluoropyrrolidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 50. | 2-(1-acryloyl-4-(7-(benzo[b]thiophen-7-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 51. | 2-(1-acryloyl-4-(7-(benzo[b]thiophen-4-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 52. | 2-(1-acryloyl-4-(7-(benzo[d]thiazol-4-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 53. | 2-(1-acryloyl-4-(7-(2,3-dihydrobenzofuran-7-yl)-2-((1- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 54. | 2-(1-acryloyl-4-(7-(benzo[d]thiazol-7-yl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 55. | 2-(1-acryloyl-4-(7-(2-amino-6-fluorophenyl)-2-((1-((dimethylamino)methyl)cyclopropyl)methoxy)- |
| | 5,6,7,8-tetrahydropyrido[3,4-d]pyrimidin-4-yl)piperazin-2-yl)acetonitrile; |
| 56. | 2-(1-acryloyl-4-(7-(8-methylnaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 57. | 2-(1-acryloyl-4-(2-(1-(1-((dimethylamino)methyl)cyclopropyl)ethoxy)-7-(8-methylnaphthalen-1- |
| | yl)-5,6,7,8-tetrahydropyrido[3,4-d]pyrimidin-4-yl)piperazin-2-yl)acetonitrile; |
| 58. | 2-(1-acryloyl-4-(7-(8-methylnaphthalen-1-yl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 59. | 2-(1-acryloyl-4-(2-((1-((3-(dimethylamino)azetidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 60. | 2-(1-acryloyl-4-(2-(((1S,2S)-2-(dimethylamino)cyclohexyl)oxy)-7-(naphthalen-1-yl)-5,6,7,8- |
| | tetrahydropyrido[3,4-d]pyrimidin-4-yl)piperazin-2-yl)acetonitrile; |
| 61. | 2-(1-acryloyl-4-(7-(8-chloronaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 62. | 2-(1-acryloyl-4-(2-((1-((3-fluoroazetidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 63. | 2-(1-acryloyl-4-(7-(8-methylnaphthalen-1-yl)-2-((1-(4-methylpiperazine-1- |
| | carbonyl)cyclopropyl)methoxy)-5,6,7,8-tetrahydropyrido[3,4-d]pyrimidin-4-yl)piperazin-2- |
| | yl)acetonitrile; |
| 64. | 2-(1-acryloyl-4-(2-((1-(((2-(dimethylamino)ethyl)(methyl)amino)methyl)cyclopropyl)methoxy)-7- |
| | (8-methylnaphthalen-1-yl)-5,6,7,8-tetrahydropyrido[3,4-d]pyrimidin-4-yl)piperazin-2- |
| | yl)acetonitrile; |
| 65. | 2-(1-acryloyl-4-(2-((1-(((S)-3-(dimethylamino)pyrrolidin-1-yl)methyl)cyclopropyl)methoxy)-7-(8- |
| | methylnaphthalen-1-yl)-5,6,7,8-tetrahydropyrido[3,4-d]pyrimidin-4-yl)piperazin-2-yl)acetonitrile; |
| 66. | 2-(4-(2-((1-((4-acetylpiperazin-1-yl)methyl)cyclopropyl)methoxy)-7-(8-methylnaphthalen-1-yl)- |
| | 5,6,7,8-tetrahydropyrido[3,4-d]pyrimidin-4-yl)-1-acryloylpiperazin-2-yl)acetonitrile; |
| 67. | (S)-2-(1-acryloyl-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 68. | (S,E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-fluorobut-2-enoyl)piperazin-2-yl)acetonitrile; |
| 69. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-hydroxybut-2-enoyl)piperazin-2- |
| | yl)acetonitrile; |
| 70. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-methoxybut-2-enoyl)piperazin-2- |
| | yl)acetonitrile; |
| 71. | 2-(1-(2-fluoroacryloyl)-4-(7-(8-methylnaphthalen-1-yl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 72. | 2-(1-(2-fluoroacryloyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 73. | 2-(4-(2-((1-(azetidin-1-ylmethyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 74. | (E)-2-(1-(but-2-enoyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 75. | 2-(4-(2-((1-((3-(dimethylamino)azetidin-1-yl)methyl)cyclopropyl)methoxy)-7-(8- |
| | methylnaphthalen-1-yl)-5,6,7,8-tetrahydropyrido[3,4-d]pyrimidin-4-yl)-1-(2- |
| | fluoroacryloyl)piperazin-2-yl)acetonitrile; |
| 76. | 2-(4-(2-((1-((diethylamino)methyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 77. | (E)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(4-fluorobut-2-enoyl)piperazin-2-yl)acetonitrile; |
| 78. | 2-(4-(2-((1-((dimethylamino)methyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 79. | 2-(4-(7-(8-chloronaphthalen-1-yl)-2-((1-((dimethylamino)methyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 80. | 2-(4-(2-((1-((dimethylamino)methyl)cyclopropyl)methoxy)-7-(2,3-dimethylphenyl)-5,6,7,8- |
| | tetrahydropyrido[3,4-d]pyrimidin-4-yl)-1-(2-fluoroacryloyl)piperazin-2-yl)acetonitrile; |
| 81. | 2-(4-(2-((1-((3,3-difluoropyrrolidin-1-yl)methyl)cyclopropyl)methoxy)-7-(8-methylnaphthalen-1- |
| | yl)-5,6,7,8-tetrahydropyrido[3,4-d]pyrimidin-4-yl)-1-(2-fluoroacryloyl)piperazin-2-yl)acetonitrile; |
| 82. | (E)-2-(1-(but-2-enoyl)-4-(2-((1-((dimethylamino)methyl)cyclopropyl)methoxy)-7-(2,3- |
| | dimethylphenyl)-5,6,7,8-tetrahydropyrido[3,4-d]pyrimidin-4-yl)piperazin-2-yl)acetonitrile; |
| 83. | (S)-2-(4-(2-((1-((dimethylamino)methyl)cyclopropyl)methoxy)-7-(8-methylnaphthalen-1-yl)- |
| | 5,6,7,8-tetrahydropyrido[3,4-d]pyrimidin-4-yl)-1-(2-fluoroacryloyl)piperazin-2-yl)acetonitrile; |
| 84. | (S,E)-2-(4-(7-(8-chloronaphthalen-1-yl)-2-((1-((dimethylamino)methyl)cyclopropyl)methoxy)- |
| | 5,6,7,8-tetrahydropyrido[3,4-d]pyrimidin-4-yl)-1-(4-fluorobut-2-enoyl)piperazin-2-yl)acetonitrile; |
| 85. | (E)-2-(1-(4-(dimethylamino)but-2-enoyl)-4-(2-((1-((dimethylamino)methyl)cyclopropyl)methoxy)- |
| | 7-(8-methylnaphthalen-1-yl)-5,6,7,8-tetrahydropyrido[3,4-d]pyrimidin-4-yl)piperazin-2- |
| | yl)acetonitrile; |
| 86. | (E)-2-(1-(4-(dimethylamino)but-2-enoyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile. |
| 87. | (S)-2-(1-acryloyl-4-(7-(3-fluoro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 88. | (S)-2-(1-acryloyl-4-(7-(2,3-dichlorophenyl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 89. | (S)-2-(1-acryloyl-4-(7-(3-chloro-2-(trifluoromethyl)phenyl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 90. | 2-(1-acryloyl-4-(7-(5-chloro-4-(trifluoromethyl)pyridin-3-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 91. | (S)-2-(1-acryloyl-4-(7-(5-chloro-4-(trifluoromethyl)pyridin-3-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 92. | (S)-2-(1-(2-fluoroacryloyl)-4-(2-((1-(pyrrolidin-1-ylmethyl)cyclopropyl)methoxy)-7-(2- |
| | (trifluoromethyl)pyridin-3-yl)-5,6,7,8-tetrahydropyrido[3,4-d]pyrimidin-4-yl)piperazin-2- |
| | yl)acetonitrile; |
| 93. | (S)-2-(1-(2-fluoroacryloyl)-4-(7-(8-methylnaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 94. | 2-(4-(7-(8-chloronaphthalen-1-yl)-2-((1-(pyrrolidin-1-ylmethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 95. | (S)-2-(4-(7-(8-chloronaphthalen-1-yl)-2-((1-(pyrrolidin-1-ylmethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 96. | 2-(1-acryloyl-4-(2-((1-((3,3-difluoropyrrolidin-1-yl)methyl)cyclopropyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 97. | 2-(1-acryloyl-4-(7-(3-chloro-2-(trifluoromethyl)phenyl)-2-((1-(((S)-3-fluoropyrrolidin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 98. | (S)-2-(4-(7-(8-chloronaphthalen-1-yl)-2-((1-(pyrrolidin-1-yl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 99. | (S)-2-(4-(7-(8-chloro-7-fluoronaphthalen-1-yl)-2-((1-(pyrrolidin-1-yl)cyclopropyl)methoxy)- |
| | 5,6,7,8-tetrahydropyrido[3,4-d]pyrimidin-4-yl)-1-(2-fluoroacryloyl)piperazin-2-yl)acetonitrile; |
| 100. | (S)-2-(1-acryloyl-4-(7-(3-methyl-2-(trifluoromethyl)phenyl)-2-((1-(pyrrolidin-1- |
| | yl)cyclopropyl)methoxy)-5,6,7,8-tetrahydropyrido[3,4-d]pyrimidin-4-yl)piperazin-2-yl)acetonitrile; |
| 101. | (S)-2-(1-acryloyl-4-(7-(3-chloro-2-(trifluoromethyl)phenyl)-2-((1-(pyrrolidin-1- |
| | yl)cyclopropyl)methoxy)-5,6,7,8-tetrahydropyrido[3,4-d]pyrimidin-4-yl)piperazin-2-yl)acetonitrile; |
| 102. | 2-((S)-1-acryloyl-4-(7-(2,3-dichlorophenyl)-2-((1-((S)-3-fluoropyrrolidin-1- |
| | yl)cyclopropyl)methoxy)-5,6,7,8-tetrahydropyrido[3,4-d]pyrimidin-4-yl)piperazin-2-yl)acetonitrile; |
| 103. | 2-(1-acryloyl-4-(2-((1-(azetidin-1-ylmethyl)cyclopropyl)methoxy)-7-(2,3-dichlorophenyl)-5,6,7,8- |
| | tetrahydropyrido[3,4-d]pyrimidin-4-yl)piperazin-2-yl)acetonitrile; |
| 104. | (S)-2-(1-acryloyl-4-(2-((1-(azetidin-1-ylmethyl)cyclopropyl)methoxy)-7-(3-chloro-2- |
| | (trifluoromethyl)phenyl)-5,6,7,8-tetrahydropyrido[3,4-d]pyrimidin-4-yl)piperazin-2-yl)acetonitrile; |
| 105. | (S)-2-(4-(2-((1-(azetidin-1-ylmethyl)cyclopropyl)methoxy)-7-(3-chloro-2-(trifluoromethyl)phenyl)- |
| | 5,6,7,8-tetrahydropyrido[3,4-d]pyrimidin-4-yl)-1-(2-fluoroacryloyl)piperazin-2-yl)acetonitrile; |
| 106. | (S)-2-(4-(2-((1-(azetidin-1-ylmethyl)cyclopropyl)methoxy)-7-(8-chloronaphthalen-1-yl)-5,6,7,8- |
| | tetrahydropyrido[3,4-d]pyrimidin-4-yl)-1-(2-fluoroacryloyl)piperazin-2-yl)acetonitrile; |
| 107. | (S)-2-(4-(2-((1-(azetidin-1-ylmethyl)cyclopropyl)methoxy)-7-(8-methylnaphthalen-1-yl)-5,6,7,8- |
| | tetrahydropyrido[3,4-d]pyrimidin-4-yl)-1-(2-fluoroacryloyl)piperazin-2-yl)acetonitrile; |
| 108. | (S)-2-(1-acryloyl-4-(7-(8-methylnaphthalen-1-yl)-2-((1-(morpholinomethyl)cyclopropyl)methoxy)- |
| | 5,6,7,8-tetrahydropyrido[3,4-d]pyrimidin-4-yl)piperazin-2-yl)acetomtrile; |
| 109. | (S)-2-(4-(7-(8-chloronaphthalen-1-yl)-2-((1-(morpholinomethyl)cyclopropyl)methoxy)-5,6,7,8- |
| | tetrahydropyrido[3,4-d]pyrimidin-4-yl)-1-(2-fluoroacryloyl)piperazin-2-yl)acetonitrile; |
| 110. | 2-((S)-1-acryloyl-4-(7-(8-chloronaphthalen-1-yl)-2-(((1R,2S)-2- |
| | (dimethylamino)cyclobutyl)methoxy)-5,6,7,8-tetrahydropyrido[3,4-d]pyrimidin-4-yl)piperazin-2- |
| | yl)acetonitrile; |
| 111. | 2-((S)-4-(7-(8-chloronaphthalen-1-yl)-2-(((1S,2R)-2-(dimethylamino)cyclobutyl)methoxy)-5,6,7,8- |
| | tetrahydropyrido[3,4-d]pyrimidin-4-yl)-1-(2-fluoroacryloyl)piperazin-2-yl)acetonitrile; |
| 112. | 2-((S)-1-((E)-but-2-enoyl)-4-(7-(8-chloronaphthalen-1-yl)-2-(((1S,2S)-2- |
| | (dimethylamino)cyclobutyl)methoxy)-5,6,7,8-tetrahydropyrido[3,4-d]pyrimidin-4-yl)piperazin-2- |
| | yl)acetonitrile; |
| 113. | 2-((S)-4-(7-(8-chloronaphthalen-1-yl)-2-(((1R,2R)-2-(dimethylamino)cyclobutyl)methoxy)-5,6,7,8- |
| | tetrahydropyrido[3,4-d]pyrimidin-4-yl)-1-((E)-4-fluorobut-2-enoyl)piperazin-2-yl)acetonitrile; |
| 114. | 2-((S)-4-(7-(8-chloronaphthalen-1-yl)-2-(((1R,3S)-3-(dimethylamino)cyclopentyl)methoxy)- |
| | 5,6,7,8-tetrahydropyrido[3,4-d]pyrimidin-4-yl)-1-(2-fluoroacryloyl)piperazin-2-yl)acetonitrile; |
| 115. | 2-((S)-1-((E)-but-2-enoyl)-4-(7-(3-chloro-2-(trifluoromethyl)phenyl)-2-(((1S,3R)-3- |
| | (dimethylamino)cyclopentyl)methoxy)-5,6,7,8-tetrahydropyrido[3,4-d]pyrimidin-4-yl)piperazin-2- |
| | yl)acetonitrile; |
| 116. | 2-((S)-1-acryloyl-4-(2-(((1R,3R)-3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 117. | 2-((S)-4-(7-(2,3-dichlorophenyl)-2-(((1S,3S)-3-(dimethylamino)cyclopentyl)methoxy)-5,6,7,8- |
| | tetrahydropyrido[3,4-d]pyrimidin-4-yl)-1-((E)-4-fluorobut-2-enoyl)piperazin-2-yl)acetonitrile; |
| 118. | 2-((S)-1-acryloyl-4-(2-(((1R,3S)-3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 119. | 2-((S)-1-acryloyl-4-(2-(((1R,3R)-3-(dimethylamino)cyclopentyl)methoxy)-7-(4- |
| | (trifluoromethyl)pyridin-3-yl)-5,6,7,8-tetrahydropyrido[3,4-d]pyrimidin-4-yl)piperazin-2- |
| | yl)acetonitrile; |
| 120. | 2-((S)-4-(7-(5-chloro-4-(trifluoromethyl)pyridin-3-yl)-2-(((1S,3S)-3- |
| | (dimethylamino)cyclopentyl)methoxy)-5,6,7,8-tetrahydropyrido[3,4-d]pyrimidin-4-yl)-1-(2- |
| | fluoroacryloyl)piperazin-2-yl)acetonitrile; |
| 121. | 2-((S)-1-acryloyl-4-(7-(3-chloro-2-(trifluoromethyl)phenyl)-2-(((1S,3R)-3- |
| | (dimethylamino)cyclopentyl)methoxy)-5,6,7,8-tetrahydropyrido[3,4-d]pyrimidin-4-yl)piperazin-2- |
| | yl)acetonitrile; |
| 122. | 2-((S)-1-acryloyl-4-(7-(2,3-dichlorophenyl)-2-(((1R,3R)-3-(dimethylamino)cyclopentyl)methoxy)- |
| | 5,6,7,8-tetrahydropyrido[3,4-d]pyrimidin-4-yl)piperazin-2-yl)acetonitrile; |
| 123. | 2-((S)-1-acryloyl-4-(2-(((1R,3R)-3-(dimethylamino)cyclopentyl)methoxy)-7-(8-methylnaphthalen- |
| | 1-yl)-5,6,7,8-tetrahydropyrido[3,4-d]pyrimidin-4-yl)piperazin-2-yl)acetonitrile; |
| 124. | 2-((S)-4-(7-(8-chloronaphthalen-1-yl)-2-(((1S,3R)-3-(dimethylamino)cyclopentyl)methoxy)- |
| | 5,6,7,8-tetrahydropyrido[3,4-d]pyrimidin-4-yl)-1-(2-fluoroacryloyl)piperazin-2-yl)acetonitrile; |
| 125. | 2-(4-(7-(3-chloro-2-(trifluoromethyl)phenyl)-2-((1-((3-methoxypyrrolidin-1- |
| | yl)methyl)cyclopropyl)methoxy)-5,6,7,8-tetrahydropyrido[3,4-d]pyrimidin-4-yl)-1-(2- |
| | fluoroacryloyl)piperazin-2-yl)acetonitrile; |
| 126. | (E)-2-(1-(but-2-enoyl)-4-(7-(3-chloro-2-(trifluoromethyl)phenyl)-2-((1-((4-methylpiperazin-1- |
| | yl)methyl)cyclopropyl)methoxy)-5,6,7,8-tetrahydropyrido[3,4-d]pyrimidin-4-yl)piperazin-2- |
| | yl)acetonitrile; |
| 127. | 2-(4-(2-((1-((6-azaspiro[2.5]octan-6-yl)methyl)cyclopropyl)methoxy)-7-(3-chloro-2- |
| | methylphenyl)-5,6,7,8-tetrahydropyrido[3,4-d]pyrimidin-4-yl)-1-acryloylpiperazin-2- |
| | yl)acetonitrile; |
| 128. | 2-(1-acryloyl-4-(2-((1-((4-methoxypiperidin-1-yl)methyl)cyclopropyl)methoxy)-7-(4-methyl-3,4- |
| | dihydro-2H-benzo[b][1,4]oxazin-5-yl)-5,6,7,8-tetrahydropyrido[3,4-d]pyrimidin-4-yl)piperazin-2- |
| | yl)acetonitrile; |
| 129. | 2-(1-acryloyl-4-(2-((1-((4-methoxypiperidin-1-yl)methyl)cyclopropyl)methoxy)-7-(1- |
| | methylindolin-7-yl)-5,6,7,8-tetrahydropyrido[3,4-d]pyrimidin-4-yl)piperazin-2-yl)acetonitrile; |
| 130. | 2-(4-(2-((1-(((1R,4R)-2-oxa-5-azabicyclo[2.2.1]heptan-5-yl)methyl)cyclopropyl)methoxy)-7-(8- |
| | chloronaphthalen-1-yl)-5,6,7,8-tetrahydropyrido[3,4-d]pyrimidin-4-yl)-1-acryloylpiperazin-2- |
| | yl)acetonitrile; |
| 131. | 2-(1-acryloyl-4-(7-(3-chloro-2-(trifluoromethyl)phenyl)-2-(1-(2-(pyrrolidin-1- |
| | yl)ethyl)cyclopropoxy)-5,6,7,8-tetrahydropyrido[3,4-d]pyrimidin-4-yl)piperazin-2-yl)acetonitrile; |
| 132. | 2-(1-acryloyl-4-(7-(3-methyl-2-(trifluoromethyl)phenyl)-2-(2-(1-(pyrrolidin-1- |
| | yl)cyclopropyl)ethoxy)-5,6,7,8-tetrahydropyrido[3,4-d]pyrimidin-4-yl)piperazin-2-yl)acetonitrile; |
| 133. | 2-(1-acryloyl-4-(7-(3-chloro-2-(trifluoromethyl)phenyl)-2-((1-(1-methylpiperidin-3- |
| | yl)cyclopropyl)methoxy)-5,6,7,8-tetrahydropyrido[3,4-d]pyrimidin-4-yl)piperazin-2-yl)acetonitrile; |
| 134. | (E)-2-(1-(but-2-enoyl)-4-(7-(3-chloro-2-(trifluoromethyl)phenyl)-2-((1-((6-methyl-2,6- |
| | diazaspiro[3.3]heptan-2-yl)methyl)cyclopropyl)methoxy)-5,6,7,8-tetrahydropyrido[3,4- |
| | d]pyrimidin-4-yl)piperazin-2-yl)acetonitrile; |
| 135. | 2-(1-acryloyl-4-(7-(3-chloro-2-(trifluoromethyl)phenyl)-2-((1- |
| | ((diethylamino)methyl)cyclopropyl)methoxy)-5,6,7,8-tetrahydropyrido[3,4-d]pyrimidin-4- |
| | yl)piperazin-2-yl)acetonitrile; |
| 136. | 2-(1-acryloyl-4-(7-(8-chloronaphthalen-1-yl)-2-((1-(pyrrolidin-1-ylmethyl)cyclopropyl)methoxy)- |
| | 5,6,7,8-tetrahydropyrido[3,4-d]pyrimidin-4-yl)piperazin-2-yl)acetonitrile; |
| 137. | (S)-2-(1-acryloyl-4-(7-(8-chloronaphthalen-1-yl)-2-((1-(pyrrolidin-1- |
| | ylmethyl)cyclopropyl)methoxy)-5,6,7,8-tetrahydropyrido[3,4-d]pyrimidin-4-yl)piperazin-2- |
| | yl)acetonitrile; |
| 138. | (S)-2-(1-acryloyl-4-(2-((1-(pyrrolidin-1-yl)cyclopropyl)methoxy)-7-(2-(trifluoromethyl)phenyl)- |
| | 5,6,7,8-tetrahydropyrido[3,4-d]pyrimidin-4-yl)piperazin-2-yl)acetonitrile; |
| 139. | (S)-2-(1-acryloyl-4-(2-((1-(3,3-difluoropyrrolidin-1-yl)cyclopropyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 140. | 2-((2S)-1-acryloyl-4-(7-(3-fluoro-2-(trifluoromethyl)phenyl)-2-((1-(1-methylpyrrolidin-2- |
| | yl)cyclopropyl)methoxy)-5,6,7,8-tetrahydropyrido[3,4-d]pyrimidin-4-yl)piperazin-2-yl)acetonitrile; |
| 141. | 2-((2S)-1-acryloyl-4-(7-(3-chloro-2-(trifluoromethyl)phenyl)-2-((1-(4-hydroxy-1-methylpyrrolidin- |
| | 2-yl)cyclopropyl)methoxy)-5,6,7,8-tetrahydropyrido[3,4-d]pyrimidin-4-yl)piperazin-2- |
| | yl)acetonitrile; |
| 142. | 2-((2S)-1-acryloyl-4-(7-(2,3-dichlorophenyl)-2-((2-methyl-1-(1-methylpyrrolidin-2- |
| | yl)cyclopropyl)methoxy)-5,6,7,8-tetrahydropyrido[3,4-d]pyrimidin-4-yl)piperazin-2-yl)acetonitrile; |
| 143. | 2-(1-acryloyl-4-(7-(2,3-dichlorophenyl)-2-(1-(pyrrolidin-1-ylmethyl)cyclopropoxy)-5,6,7,8- |
| | tetrahydropyrido[3,4-d]pyrimidin-4-yl)piperazin-2-yl)acetonitrile; |
| 144. | 2-(1-acryloyl-4-(2-(1-((dimethylamino)methyl)cyclopropoxy)-7-(naphthalen-1-yl)-5,6,7,8- |
| | tetrahydropyrido[3,4-d]pyrimidin-4-yl)piperazin-2-yl)acetonitrile; |
| 145. | 2-(1-acryloyl-4-(7-(8-methylnaphthalen-1-yl)-2-(1-(1-methylpyrrolidin-2-yl)cyclopropoxy)-5,6,7,8- |
| | tetrahydropyrido[3,4-d]pyrimidin-4-yl)piperazin-2-yl)acetonitrile; |
| 146. | 2-((2S)-4-(7-(8-chloronaphthalen-1-yl)-2-(1-(4,4-difluoro-1-methylpyrrolidin-2-yl)cyclopropoxy)- |
| | 5,6,7,8-tetrahydropyrido[3,4-d]pyrimidin-4-yl)-1-(2-fluoroacryloyl)piperazin-2-yl)acetonitrile; |
| 147. | 2-((2S)-1-acryloyl-4-(7-(2-chlorophenyl)-2-(1-(4-hydroxy-1-methylpyrrolidin-2-yl)-2,2- |
| | dimethylcyclopropoxy)-5,6,7,8-tetrahydropyrido[3,4-d]pyrimidin-4-yl)piperazin-2-yl)acetonitrile; |
| 148. | 2-((2S)-4-(7-(2-chloro-3-fluorophenyl)-2-(1-(4-methoxy-1-methylpyrrolidin-2-yl)cyclopropoxy)- |
| | 5,6,7,8-tetrahydropyrido[3,4-d]pyrimidin-4-yl)-1-((E)-4-(dimethylamino)but-2-enoyl)piperazin-2- |
| | yl)acetonitrile; |
| 149. | 2-((2S)-4-(2-(2-(dimethylamino)cyclopropoxy)-7-(naphthalen-1-yl)-5,6,7,8-tetrahydropyrido[3,4- |
| | d]pyrimidin-4-yl)-1-(2-fluoroacryloyl)piperazin-2-yl)acetonitrile; |
| 150. | 2-((2S)-1-((E)-but-2-enoyl)-4-(2-(2-((dimethylamino)methyl)cyclopropoxy)-7-(8- |
| | methylnaphthalen-1-yl)-5,6,7,8-tetrahydropyrido[3,4-d]pyrimidin-4-yl)piperazin-2-yl)acetonitrile; |
| 151. | 2-((2S)-1-acryloyl-4-(2-((2-(dimethylamino)cyclopropyl)methoxy)-7-(naphthalen-1-yl)-5,6,7,8- |
| | tetrahydropyrido[3,4-d]pyrimidin-4-yl)piperazin-2-yl)acetonitrile; |
| 152. | 2-((2S)-1-acryloyl-4-(7-(3-chloro-2-fluorophenyl)-2-((2- |
| | ((dimethylamino)methyl)cyclopropyl)methoxy)-5,6,7,8-tetrahydropyrido[3,4-d]pyrimidin-4- |
| | yl)piperazin-2-yl)acetonitrile; |
| 153. | 2-((2S)-1-acryloyl-4-(2-((1R)-1-(2-(pyrrolidin-1-ylmethyl)cyclopropyl)ethoxy)-7-(o-tolyl)-5,6,7,8- |
| | tetrahydropyrido[3,4-d]pyrimidin-4-yl)piperazin-2-yl)acetonitrile; |
| 154. | 2-((2S)-1-acryloyl-4-(7-(3-fluoro-2-methylphenyl)-2-((1R)-1-(2-((1-methylpyrrolidin-2- |
| | yl)methyl)cyclopropyl)ethoxy)-5,6,7,8-tetrahydropyrido[3,4-d]pyrimidin-4-yl)piperazin-2- |
| | yl)acetonitrile; |
| 155. | (S)-2-(1-acryloyl-4-(7-(2,3-dichlorophenyl)-2-(1-(pyrrolidin-1-ylmethyl)cyclobutoxy)-5,6,7,8- |
| | tetrahydropyrido[3,4-d]pyrimidin-4-yl)piperazin-2-yl)acetonitrile; |
| 156. | 2-((2S)-1-acryloyl-4-(7-(3-chloro-2-(trifluoromethyl)phenyl)-2-(1-((1-methylpyrrolidin-2- |
| | yl)methyl)cyclobutoxy)-5,6,7,8-tetrahydropyrido[3,4-d]pyrimidin-4-yl)piperazin-2-yl)acetonitrile; |
| 157. | 2-((2S)-1-acryloyl-4-(7-(3-chloro-2-(trifluoromethyl)phenyl)-2-(1-(1-methylpyrrolidin-2- |
| | yl)cyclobutoxy)-5,6,7,8-tetrahydropyrido[3,4-d]pyrimidin-4-yl)piperazin-2-yl)acetonitrile; |
| 158. | 2-((2S)-1-acryloyl-4-(2-(2-(dimethylamino)cyclobutoxy)-7-(2-(trifluoromethyl)phenyl)-5,6,7,8- |
| | tetrahydropyrido[3,4-d]pyrimidin-4-yl)piperazin-2-yl)acetonitrile; |
| 159. | (S)-2-(1-acryloyl-4-(2-(3-(ethyl(methyl)amino)cyclobutoxy)-7-(naphthalen-1-yl)-5,6,7,8- |
| | tetrahydropyrido[3,4-d]pyrimidin-4-yl)piperazin-2-yl)acetonitrile; |
| 160. | (S)-2-(1-acryloyl-4-(2-(3-(ethyl(2-methoxyethyl)amino)cyclobutoxy)-7-(naphthalen-1-yl)-5,6,7,8- |
| | tetrahydropyrido[3,4-d]pyrimidin-4-yl)piperazin-2-yl)acetonitrile; |
| 161. | 2-((2S)-1-acryloyl-4-(2-(3-(dimethylamino)-2-(2-methoxyethyl)cyclobutoxy)-7-(naphthalen-1-yl)- |
| | 5,6,7,8-tetrahydropyrido[3,4-d]pyrimidin-4-yl)piperazin-2-yl)acetonitrile; |
| 162. | (S)-2-(1-acryloyl-4-(7-(naphthalen-1-yl)-2-(3-(pyrrolidin-1-yl)cyclobutoxy)-5,6,7,8- |
| | tetrahydropyrido[3,4-d]pyrimidin-4-yl)piperazin-2-yl)acetonitrile; |
| 163. | (S)-2-(1-acryloyl-4-(7-(2-chlorophenyl)-2-((1-(pyrrolidin-1-yl)cyclobutyl)methoxy)-5,6,7,8- |
| | tetrahydropyrido[3,4-d]pyrimidin-4-yl)piperazin-2-yl)acetonitrile; |
| 164. | 2-((2S)-1-acryloyl-4-(2-((1-(1-methylpyrrolidin-3-yl)cyclobutyl)methoxy)-7-(2- |
| | (trifluoromethyl)phenyl)-5,6,7,8-tetrahydropyrido[3,4-d]pyrimidin-4-yl)piperazin-2-yl)acetonitrile; |
| 165. | 2-((2S)-1-acryloyl-4-(2-((1-(1-methylpyrrolidin-2-yl)cyclobutyl)methoxy)-7-(2- |
| | (trifluoromethyl)phenyl)-5,6,7,8-tetrahydropyrido[3,4-d]pyrimidin-4-yl)piperazin-2-yl)acetonitrile; |
| 166. | 2-((2S)-1-acryloyl-4-(2-((2-(dimethylamino)cyclobutyl)methoxy)-7-(naphthalen-1-yl)-5,6,7,8- |
| | tetrahydropyrido[3,4-d]pyrimidin-4-yl)piperazin-2-yl)acetonitrile; |
| 167. | (S)-2-(1-acryloyl-4-(7-(3-chloro-2-(trifluoromethyl)phenyl)-2-((3- |
| | (dimethylamino)cyclobutyl)methoxy)-5,6,7,8-tetrahydropyrido[3,4-d]pyrimidin-4-yl)piperazin-2- |
| | yl)acetonitrile; |
| 168. | 2-((S)-1-acryloyl-4-(2-((R)-1-(3-(dimethylamino)cyclobutyl)ethoxy)-7-(3-fluoro-2- |
| | (trifluoromethyl)phenyl)-5,6,7,8-tetrahydropyrido[3,4-d]pyrimidin-4-yl)piperazin-2-yl)acetonitrile; |
| 169. | (S)-2-(1-acryloyl-4-(7-(2-chlorophenyl)-2-((1-(dimethylamino)cyclopentyl)methoxy)-5,6,7,8- |
| | tetrahydropyrido[3,4-d]pyrimidin-4-yl)piperazin-2-yl)acetonitrile; |
| 170. | 2-((2S)-1-acryloyl-4-(7-(2-chlorophenyl)-2-((1-(dimethylamino)-3- |
| | (trifluoromethyl)cyclopentyl)methoxy)-5,6,7,8-tetrahydropyrido[3,4-d]pyrimidin-4-yl)piperazin-2- |
| | yl)acetonitrile; |
| 171. | 2-((2S)-1-acryloyl-4-(7-(2-chlorophenyl)-2-((1-(dimethylamino)-3- |
| | (trifluoromethoxy)cyclopentyl)methoxy)-5,6,7,8-tetrahydropyrido[3,4-d]pyrimidin-4-yl)piperazin- |
| | 2-yl)acetonitrile; |
| 172. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentane-1-carbonitrile; |
| 173. | ethyl 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentane-1-carboxylate; |
| 174. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N,N-dimethylcyclopentane- |
| | 1-carboxamide; |
| 175. | 2-((2S)-1-acryloyl-4-(7-(2-chlorophenyl)-2-((1-(dimethylamino)-3-(pyrrolidine-1- |
| | carbonyl)cyclopentyl)methoxy)-5,6,7,8-tetrahydropyrido[3,4-d]pyrimidin-4-yl)piperazin-2- |
| | yl)acetonitrile; |
| 176. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N-methylcyclopentane-1- |
| | carboxamide; |
| 177. | 2-((2S)-1-acryloyl-4-(7-(2-chlorophenyl)-2-((1-(dimethylamino)-3-(thiazol-2- |
| | yl)cyclopentyl)methoxy)-5,6,7,8-tetrahydropyrido[3,4-d]pyrimidin-4-yl)piperazin-2-yl)acetonitrile; |
| 178. | (S)-2-(1-acryloyl-4-(7-(2,3-difluorophenyl)-2-((1-(pyrrolidin-1-yl)cyclopentyl)methoxy)-5,6,7,8- |
| | tetrahydropyrido[3,4-d]pyrimidin-4-yl)piperazin-2-yl)acetonitrile; |
| 179. | 2-((2S)-1-acryloyl-4-(2-((1-(1-methylpyrrolidin-2-yl)cyclopentyl)methoxy)-7-(naphthalen-1-yl)- |
| | 5,6,7,8-tetrahydropyrido[3,4-d]pyrimidin-4-yl)piperazin-2-yl)acetonitrile; |
| 180. | 2-((2S)-1-acryloyl-4-(7-(2-chloro-3-fluorophenyl)-2-((2,2-difluoro-1-(1-methylpyrrolidin-2- |
| | yl)cyclopentyl)methoxy)-5,6,7,8-tetrahydropyrido[3,4-d]pyrimidin-4-yl)piperazin-2-yl)acetonitrile; |
| 181. | 2-((2S)-1-acryloyl-4-(7-(2-chloro-3-fluorophenyl)-2-((1-(1-methylpyrrolidin-3- |
| | yl)cyclopentyl)methoxy)-5,6,7,8-tetrahydropyrido[3,4-d]pyrimidin-4-yl)piperazin-2-yl)acetonitrile; |
| 182. | 2-((2S)-1-acryloyl-4-(7-(2,3-dichlorophenyl)-2-((1-(1-isopropylpyrrolidin-3- |
| | yl)cyclopentyl)methoxy)-5,6,7,8-tetrahydropyrido[3,4-d]pyrimidin-4-yl)piperazin-2-yl)acetonitrile; |
| 183. | (S)-2-(1-acryloyl-4-(7-(2-chlorophenyl)-2-((1-((dimethylamino)methyl)cyclopentyl)oxy)-5,6,7,8- |
| | tetrahydropyrido[3,4-d]pyrimidin-4-yl)piperazin-2-yl)acetonitrile; |
| 184. | (S)-2-(1-acryloyl-4-(2-((1-(azetidin-1-ylmethyl)cyclopentyl)oxy)-7-(2-(trifluoromethyl)phenyl)- |
| | 5,6,7,8-tetrahydropyrido[3,4-d]pyrimidin-4-yl)piperazin-2-yl)acetonitrile; |
| 185. | (S)-2-(1-acryloyl-4-(7-(3-fluoro-2-(trifluoromethyl)phenyl)-2-((1-(pyrrolidin-1- |
| | ylmethyl)cyclopentyl)oxy)-5,6,7,8-tetrahydropyrido[3,4-d]pyrimidin-4-yl)piperazin-2- |
| | yl)acetonitrile; |
| 186. | 2-((2S)-1-acryloyl-4-(7-(3-chloro-2-(trifluoromethyl)phenyl)-2-((1-((1-methylpyrrolidin-2- |
| | yl)methyl)cyclopentyl)oxy)-5,6,7,8-tetrahydropyrido[3,4-d]pyrimidin-4-yl)piperazin-2- |
| | yl)acetonitrile; |
| 187. | 2-((2S)-1-acryloyl-4-(2-((1-((4,4-difluoro-1-methylpyrrolidin-2-yl)methyl)cyclopentyl)oxy)-7- |
| | (naphthalen-1-yl)-5,6,7,8-tetrahydropyrido[3,4-d]pyrimidin-4-yl)piperazin-2-yl)acetonitrile; |
| 188. | 2-((2S)-1-acryloyl-4-(2-((1-(difluoro(1-methylpyrrolidin-2-yl)methyl)cyclopentyl)oxy)-7-(3- |
| | methyl-2-(trifluoromethyl)phenyl)-5,6,7,8-tetrahydropyrido[3,4-d]pyrimidin-4-yl)piperazin-2- |
| | yl)acetonitrile; |
| 189. | 2-((2S)-1-acryloyl-4-(2-((2-(dimethylamino)cyclopentyl)oxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 190. | 2-((2S)-1-acryloyl-4-(2-((3-(dimethylamino)cyclopentyl)oxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 191. | 2-((2S)-1-acryloyl-4-(2-((3-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 192. | 2-((2S)-1-acryloyl-4-(2-((2-(dimethylamino)cyclopentyl)methoxy)-7-(3-methyl-2- |
| | (trifluoromethyl)phenyl)-5,6,7,8-tetrahydropyrido[3,4-d]pyrimidin-4-yl)piperazin-2-yl)acetonitrile; |
| 193. | (S)-2-(1-acryloyl-4-(7-(2-chlorophenyl)-2-((1-((dimethylamino)methyl)cyclohexyl)oxy)-5,6,7,8- |
| | tetrahydropyrido[3,4-d]pyrimidin-4-yl)piperazin-2-yl)acetonitrile; |
| 194. | (S)-2-(1-acryloyl-4-(2-((1-(pyrrolidin-1-ylmethyl)cyclohexyl)oxy)-7-(2-(trifluoromethyl)phenyl)- |
| | 5,6,7,8-tetrahydropyrido[3,4-d]pyrimidin-4-yl)piperazin-2-yl)acetonitrile; |
| 195. | 2-((2S)-1-acryloyl-4-(7-(2-chloro-3-fluorophenyl)-2-((2-(dimethylamino)cyclohexyl)oxy)-5,6,7,8- |
| | tetrahydropyrido[3,4-d]pyrimidin-4-yl)piperazin-2-yl)acetonitrile; |
| 196. | 2-((2S)-1-acryloyl-4-(7-(2,3-dichlorophenyl)-2-((2-(1-methylazetidin-2-yl)cyclohexyl)oxy)-5,6,7,8- |
| | tetrahydropyrido[3,4-d]pyrimidin-4-yl)piperazin-2-yl)acetonitrile; |
| 197. | 2-((2S)-1-acryloyl-4-(7-(3-chloro-2-(trifluoromethyl)phenyl)-2-((2- |
| | (dimethylamino)cyclohexyl)methoxy)-5,6,7,8-tetrahydropyrido[3,4-d]pyrimidin-4-yl)piperazin-2- |
| | yl)acetonitrile; |
| 198. | 2-((2S)-1-acryloyl-4-(2-((2-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 199. | (S)-2-(1-acryloyl-4-(7-(2-chlorophenyl)-2-((1-(dimethylamino)cyclohexyl)methoxy)-5,6,7,8- |
| | tetrahydropyrido[3,4-d]pyrimidin-4-yl)piperazin-2-yl)acetonitrile; |
| 200. | (S)-2-(1-acryloyl-4-(7-(2,3-difluorophenyl)-2-((1-((dimethylamino)methyl)cyclohexyl)methoxy)- |
| | 5,6,7,8-tetrahydropyrido[3,4-d]pyrimidin-4-yl)piperazin-2-yl)acetonitrile; |
| 201. | (S)-2-(1-acryloyl-4-(2-((4-(dimethylamino)cyclohexyl)methoxy)-7-(naphthalen-1-yl)-5,6,7,8- |
| | tetrahydropyrido[3,4-d]pyrimidin-4-yl)piperazin-2-yl)acetonitrile; |
| 202. | 2-((2S)-1-acryloyl-4-(7-(3-chloro-2-(trifluoromethyl)phenyl)-2-((2-(pyrrolidin-1- |
| | yl)cyclohexyl)methoxy)-5,6,7,8-tetrahydropyrido[3,4-d]pyrimidin-4-yl)piperazin-2-yl)acetonitrile; |
| 203. | 2-((2S)-1-acryloyl-4-(7-(3-chloro-2-(trifluoromethyl)phenyl)-2-((3- |
| | (dimethylamino)cyclohexyl)methoxy)-5,6,7,8-tetrahydropyrido[3,4-d]pyrimidin-4-yl)piperazin-2- |
| | yl)acetonitrile; |
| 204. | 2-((2S)-1-acryloyl-4-(7-(3-chloro-2-(trifluoromethyl)phenyl)-2-((3- |
| | (dimethylamino)cyclohexyl)oxy)-5,6,7,8-tetrahydropyrido[3,4-d]pyrimidin-4-yl)piperazin-2- |
| | yl)acetonitrile; |
| 205. | N-(3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)cyclopentyl)acetamide; |
| 206. | 3-(((4-((S)-4-acryloyl-3-(cyanomethyl)piperazin-1-yl)-7-(2-chlorophenyl)-5,6,7,8- |
| | tetrahydropyrido[3,4-d]pyrimidin-2-yl)oxy)methyl)-3-(dimethylamino)-N,N-dimethylcyclopentane- |
| | 1-sulfonamide; |
| 207. | 2-((2S)-1-acryloyl-4-(2-(((2-(dimethylamino)cyclobutyl)methyl)thio)-7-(naphthalen-1-yl)-5,6,7,8- |
| | tetrahydropyrido[3,4-d]pyrimidin-4-yl)piperazin-2-yl)acetonitrile; or |
| 208. | 2-((2S)-1-acryloyl-4-(2-(((2-(dimethylamino)cyclobutyl)methyl)amino)-7-(naphthalen-1-yl)- |
| | 5,6,7,8-tetrahydropyrido[3,4-d]pyrimidin-4-yl)piperazin-2-yl)acetonitrile. |

2. The compound, the pharmaceutical acceptable salt thereof or stereoisomer thereof according to claim 1, wherein, the compound is 2-(1-acryloyl-4-(7-(8-methylnaphthalen-1-yl)-2-((1-(pyrrolidin-1-ylmethyl) cyclopropyl)methoxy)-5,6,7,8-tetrahydropyrido[3,4-d]pyrimidin-4-yl)piperazin-2-yl)acetonitrile.

3. The compound, the pharmaceutical acceptable salt thereof or stereoisomer thereof according to claim 1, wherein, the compound is (S,E)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile.

4. The compound, the pharmaceutical acceptable salt thereof or stereoisomer thereof according to claim 1, wherein, the compound is (S)-2-(1-acryloyl-4-(7-(8-methylnaphthalen-1-yl)-2-((1-(pyrrolidin-1-ylmethyl) cyclopropyl)methoxy)-5,6,7,8-tetrahydropyrido[3,4-d]pyrimidin-4-yl)piperazin-2-yl)acetonitrile.

5. The compound, the pharmaceutical acceptable salt thereof or stereoisomer thereof according to claim 1, wherein, the compound is (S)-2-(4-(2-((1-((dimethylamino)methyl)cyclopropyl)methoxy)-7-(8-methyl naphthalen-1-yl)-5,6,7,8-tetrahydropyrido[3,4-d]pyrimidin-4-yl)-1-(2-fluoroacryloyl)piperazin-2-yl)acetonitrile.

6. A pharmaceutical composition comprising at least one compound, the pharmaceutically acceptable salt thereof or stereoisomer thereof according to claim 1, and at least one pharmaceutically acceptable excipient.

7. The pharmaceutical composition according to claim 6, wherein the compound is 2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

8. The pharmaceutical composition according to claim 6, wherein the compound is (S,E)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile.

9. The pharmaceutical composition according to claim 6, wherein, the compound is (S)-2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

10. The pharmaceutical composition according to claim 6, wherein, the compound is (S)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(2-fluoroacryloyl)piperazin-2-yl)acetonitrile.

11. A method of treating a subject having a cancer related to KRAS G12C mutant protein, said method comprising administering to the subject a therapeutically effective amount of at least one compound, a pharmaceutically acceptable salt thereof or stereoisomer thereof according to claim 1.

12. The method according to claim 11, wherein, the cancer is selected from blood cancer, pancreatic cancer, colon cancer, rectal cancer, colorectal cancer or lung cancer.

13. The method according to claim 12, wherein, the blood cancer is selected from acute myeloid leukemia or acute lymphocytic leukemia; the lung cancer is selected from non-small cell lung cancer or small cell lung cancer.

14. The method according to claim 11, wherein, the compound is 2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

15. The method according to claim 11, wherein, the compound is (S,E)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(4-fluorobut-2-enoyl)piperazin-2-yl)acetonitrile.

16. The method according to claim 11, wherein, the compound is (S)-2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

17. The method according to claim 11, wherein, the compound is (S)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(2-fluoroacryloyl)piperazin-2-yl)acetonitrile.

18. The method according to claim 13, wherein, the compound is 2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

19. The method according to claim 13, the compound is (S)-2-(1-acryloyl-4-(7-(8-methylnaphthalen-1-yl)-2-((1-(pyrrolidin-1-ylmethyl)cyclopropyl)methoxy)-5,6,7,8-tetrahydropyrido[3,4-d]pyrimidin-4-yl)piperazin-2-yl)acetonitrile.

20. The method according to claim 13, wherein, the compound is (S)-2-(4-(2-((1-((dimethylamino)methyl)cyclopropyl)methoxy)-7-(8-methylnaphthalen-1-yl)-5,6,7,8-tetrahydropyrido[3,4-d]pyrimidin-4-yl)-1-(2-fluoroacryloyl)piperazin-2-yl)acetonitrile.
